SEQUENCE LISTING (128 sequences)

SEQ ID NO: 1 - DNA, 1740 bp, Arabidopsis thaliana
atggctcaagtggttgctaccaggtcaattcaaggctcgatgttatctcccaacggtggatctgtgtctacaagatccgagaagctattgaaaccagcgagttttgcagtgaaggttcttggcaacgaagcaaagagaagtggaagagtctctgtaagaagcagaagagtggttgatactactgtgagatccgctcgtgttgagactgaagtcattcctgtttctcctgaagatgtgcctaacagagaggagcagcttgagaggttgttggaaatgcagcagtttggtgatacatcggtagggatgtggtcgaagccgacagtgaggaggaagacaaagattgtttgcaccgttggtccgtcgaccaacacacgagaaatgatatggaaattggctgaagctgggatgaatgttgctaggatgaatatgtctcatggagatcatgcttcacataagaaggttattgatttggttaaagaatacaatgcacaaactaaagacaacactattgctatcatgcttgacaccaagggtccggaagttaggagtggagatttacctcagccaattatgttagatcctggtcaagagtttacctttacaattgagagaggagtcagcacaccaagttgtgtcagtgttaactatgatgatttcgttaatgacgtggaagcgggtgacatgcttcttgttgatggtggtatgatgtcgtttatggtgaagtcaaagaccaaagactctgtcaaatgtgaagttgttgatggtggagaacttaagtcaaggagacacctgaatgtccgaggaaagagtgcaactttaccttcaatcactgagaaggactgggaggatattaaatttggagtggagaacaaagttgacttttatgcagtttcctttgtcaaagatgctcaagttgtacacgagttgaagaaataccttcaaaatagtggtgctgatatacacgtgatagtgaaaattgagagtgcagactccatacctaacttgcactccattatcacagcatcagatggggcaatggttgcaagaggtgatcttggtgcagagcttccaattgaagaagtccccattcttcaggaggagatcattaacctgtgccgtagtatgggaaaagctgttattgttgcgactaacatgcttgagagtatgatagttcatccaactccaacccgggcagaggtctcagacattgctatcgctgttagagaaggtgctgatgcggtaatgctttcaggagaaactgctcacggaaagttcccattgaaagctgctggagtgatgcacactgttgcattgcgaacagaagcaaccattactagcggtgaaatgccacctaatcttggtcaagccttcaagaaccatatgagtgagatgtttgcataccatgcaaccatgatgtcaaacacacttggaacttcaactgttgtcttcaccagaaccggtttcatggccatattgttaagtcactatcgtccttccggcacaatctatgccttcacaaatgagaaaaaaatacaacaaagattagctttgtatcaaggtgtatgccccatatatatggagttcacagatgatgcagaagaaacttttgctaatgctttggctacattactgaaacaaggaatggtgaagaagggagaggaaatagcaatcgtacagagcggtacacagccaatctggcgatctcaatcgacacataacatccaagtccgcaaggtttaa

SEQ ID NO: 2 - protein, 579 aa, Arabidopsis thaliana
MAQVVATRSIQGSMLSPNGGSVSTRSEKLLKPASFAVKVLGNEAKRSGRVSVRSRRVVDTTVRSARVETEVIPVSPEDVPNREEQLERLLEMQQFGDTSVGMWSKPTVRRKTKIVCTVGPSTNTREMIWKLAEAGMNVARMNMSHGDHASHKKVIDLVKEYNAQTKDNTIAIMLDTKGPEVRSGDLPQPIMLDPGQEFTFTIERGVSTPSCVSVNYDDFVNDVEAGDMLLVDGGMMSFMVKSKTKDSVKCEVVDGGELKSRRHLNVRGKSATLPSITEKDWEDIKFGVENKVDFYAVSFVKDAQVVHELKKYLQNSGADIHVIVKIESADSIPNLHSIITASDGAMVARGDLGAELPIEEVPILQEEIINLCRSMGKAVIVATNMLESMIVHPTPTRAEVSDIAIAVREGADAVMLSGETAHGKFPLKAAGVMHTVALRTEATITSGEMPPNLGQAFKNHMSEMFAYHATMMSNTLGTSTVVFTRTGFMAILLSHYRPSGTIYAFTNEKKIQQRLALYQGVCPIYMEFTDDAEETFANALATLLKQGMVKKGEEIAIVQSGTQPIWRSQSTHNIQVRKV

SEQ ID NO: 3 - DNA, 1551 bp, Arabidopsis thaliana
tccgctcgtgttgagactgaagtcattcctgtttctcctgaagatgtgcctaacagagaggagcagcttgagaggttgttggaaatgcagcagtttggtgatacatcggtagggatgtggtcgaagccgacagtgaggaggaagacaaagattgtttgcaccgttggtccgtcgaccaacacacgagaaatgatatggaaattggctgaagctgggatgaatgttgctaggatgaatatgtctcatggagatcatgcttcacataagaaggttattgatttggttaaagaatacaatgcacaaactaaagacaacactattgctatcatgcttgacaccaagggtccggaagttaggagtggagatttacctcagccaattatgttagatcctggtcaagagtttacctttacaattgagagaggagtcagcacaccaagttgtgtcagtgttaactatgatgatttcgttaatgacgtggaagcgggtgacatgcttcttgttgatggtggtatgatgtcgtttatggtgaagtcaaagaccaaagactctgtcaaatgtgaagttgttgatggtggagaacttaagtcaaggagacacctgaatgtccgaggaaagagtgcaactttaccttcaatcactgagaaggactgggaggatattaaatttggagtggagaacaaagttgacttttatgcagtttcctttgtcaaagatgctcaagttgtacacgagttgaagaaataccttcaaaatagtggtgctgatatacacgtgatagtgaaaattgagagtgcagactccatacctaacttgcactccattatcacagcatcagatggggcaatggttgcaagaggtgatcttggtgcagagcttccaattgaagaagtccccattcttcaggaggagatcattaacctgtgccgtagtatgggaaaagctgttattgttgcgactaacatgcttgagagtatgatagttcatccaactccaacccgggcagaggtctcagacattgctatcgctgttagagaaggtgctgatgcggtaatgctttcaggagaaactgctcacggaaagttcccattgaaagctgctggagtgatgcacactgttgcattgcgaacagaagcaaccattactagcggtgaaatgccacctaatcttggtcaagccttcaagaaccatatgagtgagatgtttgcataccatgcaaccatgatgtcaaacacacttggaacttcaactgttgtcttcaccagaaccggtttcatggccatattgttaagtcactatcgtccttccggcacaatctatgccttcacaaatgagaaaaaaatacaacaaagattagctttgtatcaaggtgtatgccccatatatatggagttcacagatgatgcagaagaaacttttgctaatgctttggctacattactgaaacaaggaatggtgaagaagggagaggaaatagcaatcgtacagagcggtacacagccaatctggcgatctcaatcgacacataacatccaagtccgcaaggtttaa

SEQ ID NO: 4 - protein, 516 aa, Arabidopsis thaliana
SARVETEVIPVSPEDVPNREEQLERLLEMQQFGDTSVGMWSKPTVRRKTKIVCTVGPSTNTREMIWKLAEAGMNVARMNMSHGDHASHKKVIDLVKEYNAQTKDNTIAIMLDTKGPEVRSGDLPQPIMLDPGQEFTFTIERGVSTPSCVSVNYDDFVNDVEAGDMLLVDGGMMSFMVKSKTKDSVKCEVVDGGELKSRRHLNVRGKSATLPSITEKDWEDIKFGVENKVDFYAVSFVKDAQVVHELKKYLQNSGADIHVIVKIESADSIPNLHSIITASDGAMVARGDLGAELPIEEVPILQEEIINLCRSMGKAVIVATNMLESMIVHPTPTRAEVSDIAIAVREGADAVMLSGETAHGKFPLKAAGVMHTVALRTEATITSGEMPPNLGQAFKNHMSEMFAYHATMMSNTLGTSTVVFTRTGFMAILLSHYRPSGTIYAFTNEKKIQQRLALYQGVCPIYMEFTDDAEETFANALATLLKQGMVKKGEEIAIVQSGTQPIWRSQSTHNIQVRKV

SEQ ID NO: 5 - DNA, 1962 bp, Arabidopsis thaliana
atgcagaagggcgaattcgacccaagtttgtacaaaaaagcaggctccaccatggcttctacactctctaccctctcggtgagcgcatcgttgttgccaaagcaacaaccgatggtcgcctcatcgctaccaaccaacatgggccaagccttgtttggactgaaagccggttctcgtggcagagtgactgcaatggccacataccatatgcggcgcgccaccatggctcaagtggttgctaccaggtcaattcaaggctcgatgttatctcccaacggtggatctgtgtctacaagatccgagaagctattgaaaccagcgagttttgcagtgaaggttcttggcaacgaagcaaagagaagtggaagagtctctgtaagaagcagaagagtggttgatactactgtgagatccgctcgtgttgagactgaagtcattcctgtttctcctgaagatgtgcctaacagagaggagcagcttgagaggttgttggaaatgcagcagtttggtgatacatcggtagggatgtggtcgaagccgacagtgaggaggaagacaaagattgtttgcaccgttggtccgtcgaccaacacacgagaaatgatatggaaattggctgaagctgggatgaatgttgctaggatgaatatgtctcatggagatcatgcttcacataagaaggttattgatttggttaaagaatacaatgcacaaactaaagacaacactattgctatcatgcttgacaccaagggtccggaagttaggagtggagatttacctcagccaattatgttagatcctggtcaagagtttacctttacaattgagagaggagtcagcacaccaagttgtgtcagtgttaactatgatgatttcgttaatgacgtggaagcgggtgacatgcttcttgttgatggtggtatgatgtcgtttatggtgaagtcaaagaccaaagactctgtcaaatgtgaagttgttgatggtggagaacttaagtcaaggagacacctgaatgtccgaggaaagagtgcaactttaccttcaatcactgagaaggactgggaggatattaaatttggagtggagaacaaagttgacttttatgcagtttcctttgtcaaagatgctcaagttgtacacgagttgaagaaataccttcaaaatagtggtgctgatatacacgtgatagtgaaaattgagagtgcagactccatacctaacttgcactccattatcacagcatcagatggggcaatggttgcaagaggtgatcttggtgcagagcttccaattgaagaagtccccattcttcaggaggagatcattaacctgtgccgtagtatgggaaaagctgttattgttgcgactaacatgcttgagagtatgatagttcatccaactccaacccgggcagaggtctcagacattgctatcgctgttagagaaggtgctgatgcggtaatgctttcaggagaaactgctcacggaaagttcccattgaaagctgctggagtgatgcacactgttgcattgcgaacagaagcaaccattactagcggtgaaatgccacctaatcttggtcaagccttcaagaaccatatgagtgagatgtttgcataccatgcaaccatgatgtcaaacacacttggaacttcaactgttgtcttcaccagaaccggtttcatggccatattgttaagtcactatcgtccttccggcacaatctatgccttcacaaatgagaaaaaaatacaacaaagattagctttgtatcaaggtgtatgccccatatatatggagttcacagatgatgcagaagaaacttttgctaatgctttggctacattactgaaacaaggaatggtgaagaagggagaggaaatagcaatcgtacagagcggtacacagccaatctggcgatctcaatcgacacataacatccaagtccgcaaggtttaa

SEQ ID NO: 6 - protein, 653 aa, Arabidopsis thaliana
MQKGEFDPSLYKKAGSTMASTLSTLSVSASLLPKQQPMVASSLPTNMGQALFGLKAGSRGRVTAMATYHMRRATMAQVVATRSIQGSMLSPNGGSVSTRSEKLLKPASFAVKVLGNEAKRSGRVSVRSRRVVDTTVRSARVETEVIPVSPEDVPNREEQLERLLEMQQFGDTSVGMWSKPTVRRKTKIVCTVGPSTNTREMIWKLAEAGMNVARMNMSHGDHASHKKVIDLVKEYNAQTKDNTIAIMLDTKGPEVRSGDLPQPIMLDPGQEFTFTIERGVSTPSCVSVNYDDFVNDVEAGDMLLVDGGMMSFMVKSKTKDSVKCEVVDGGELKSRRHLNVRGKSATLPSITEKDWEDIKFGVENKVDFYAVSFVKDAQVVHELKKYLQNSGADIHVIVKIESADSIPNLHSIITASDGAMVARGDLGAELPIEEVPILQEEIINLCRSMGKAVIVATNMLESMIVHPTPTRAEVSDIAIAVREGADAVMLSGETAHGKFPLKAAGVMHTVALRTEATITSGEMPPNLGQAFKNHMSEMFAYHATMMSNTLGTSTVVFTRTGFMAILLSHYRPSGTIYAFTNEKKIQQRLALYQGVCPIYMEFTDDAEETFANALATLLKQGMVKKGEEIAIVQSGTQPIWRSQSTHNIQVRKV

SEQ ID NO: 7 - DNA, 1740 bp, Artificial sequence
  codon optimized sequence of AtPK920 for expression in Zea mays
atggcccaggtcgtggccacccgttcgatccaaggttctatgttatctccgaacggtgggagtgtctccacgcgttcggaaaaattgctcaagcctgcgtcttttgccgtcaaggttctcggcaatgaagctaagcgctcgggaagagtttccgttcgctctagacgtgtcgtagacactaccgtcaggtccgcaagggtggaaactgaggtgattcccgtgtctccagaggacgtcccaaaccgagaggaacagttggagcgtcttctcgagatgcaacagtttggtgatacgagcgtgggtatgtggtctaagcccactgtccgccgaaagacgaagatcgtctgcactgtgggaccaagcacaaacacgcgtgagatgatctggaagttagcggaggctggtatgaacgtcgctaggatgaatatgtcacacggtgatcatgcgtctcacaagaaagtgatcgacctagttaaggagtacaacgcccaaaccaaggacaatacaatagcgataatgctggacactaaggggcctgaggttcgtagtggggatctccctcaaccgatcatgctggaccctggacaagagttcactttcacgatagaaaggggagtatctacgccgagttgcgtcagcgtgaactacgatgacttcgtcaacgacgttgaagccggcgatatgcttctggttgatgggggcatgatgagcttcatggtgaagagcaaaacgaaggactcggtgaagtgtgaagtggttgatggcggtgagctgaaatcaaggaggcatctgaacgtaagagggaaatcggcaactttgccgtcgataaccgaaaaggactgggaggatatcaagttcggtgtggagaataaggttgacttctatgctgtctcgttcgtgaaggacgcccaagtggtccacgagttgaagaaatacctacagaacagcggtgccgatatccatgtgatcgtaaagatcgagagcgcagattcaatccctaatcttcatagtattatcaccgcgtcagacggagcaatggtggcacgtggcgacctcggtgctgagttgccaattgaggaagtcccgatccttcaagaggaaattatcaacctctgtaggtcaatggggaaagcggtgatcgttgccacaaacatgcttgagtctatgatcgtccaccctaccccaacccgagctgaggtctcggatatcgctatagcggttcgagagggtgctgacgcggtgatgctatctggtgagacagctcacggcaagttccccttgaaagccgctggtgttatgcataccgtcgcgttgcgcacagaagctacgatcacaagcggcgagatgcctcccaacctcggtcaagccttcaagaaccacatgagcgagatgttcgcttaccacgcgacaatgatgagcaacacgctgggtacaagtacagtggtctttacacgcacgggctttatggcgatactgctctcccactatcgtccttctggcactatctacgctttcaccaacgagaagaaaatccaacagcgcctcgctctctaccaaggagtctgcccaatttacatggagtttactgacgatgcggaggagacgttcgcaaatgcgttggcaaccctcctgaagcaaggaatggtcaagaaaggagaagagattgcaatcgtacaaagtggaacccagccgatttggagatcccaatcaactcacaatatccaagtgaggaaagtgtga

SEQ ID NO: 8 - protein, 579 aa, Arabidopsis thaliana; identical to SEQ ID NO: 2

SEQ ID NO: 9 - DNA, 1716 bp, Arabidopsis thaliana
atggctgcttatggtcaaatctcctcgggaatgactgtagatcctcaggttctctcttcctccagaaacattggagtttccctatcacctctccggagaacactaatcggcgccggagttaggtctactagtatctctctccgtcaatgttctctctccgttagatcgattaaaatctccgaagatagccgcaaacctaaagcttatgcagagaacggtgcttttgatgtgggagttttggattcttcatcatatagattggctgattcaagaacaagtagtaatgattcaaggaggaagactaagattgtgtgtacgattggaccgtcttcgagttctagggaaatgatttggaaactcgcggaagctggaatgaatgtggctcgtttgaatatgtctcatggtgatcatgcttctcatcagataactattgatttagttaaggagtataattctttgtttgttgacaaagctattgctattatgttggatacaaagggtcctgaggttcgaagcggggatgtaccgcagccgatatttcttgaagagggtcaagagtttaactttactatcaagagaggtgtttcgcttaaagacactgttagtgtaaattatgatgattttgtgaacgatgttgaagttggggatatacttttggtggatggtggaatgatgtcgttagctgttaaatcaaagacgagtgatttggtgaagtgtgtggttattgatggtggagagcttcaatctagacgtcacttgaatgttcgaggaaagagtgcgactcttccatccattacagacaaagattgggaagacataaaatttggagtggacaaccaagtcgatttctacgccgtctcctttgttaaggatgctaaagttgtccatgagttgaagaactatctcaaaacctgcagtgcagacatatcggtgattgtgaaaattgaaagtgcagactctataaagaatcttccttctatcatatctgcttgtgatggggcaatggttgctcgtggagatcttggagctgaacttcccattgaagaggtcccgttgttacaggaagaaataatcagaaggtgtagaagcattcataaaccagtgattgttgccacaaacatgctagagagtatgattaatcatccaacgcctacaagagctgaagtctctgacattgcaattgcagtacgtgaaggcgcagatgctatcatgctttctggtgaaaccgcacatggaaagtttccgctgaaagctgttaacgtaatgcatactgtggcgttgagaaccgaggcaagtctacctgtcagaacctcggcatcccgtaccactgcttacaagggtcacatgggccaaatgtttgcttttcatgcttctataatggcaaatacactgagctcaccgctaattgtatttacgagaaccggatccatggcagtgcttctaagccactaccgcccatctgcaacaattttcgccttcacaaaccagagaagaataatgcaaaggcttgctctttatcaaggtgtcatgcctatatatatggagttctcggatgatgcagaagatacatatgcccggtctctcaaactcttacaggacgagaatatgctcaaggaaggacaacatgtaactcttgtccaaagtggctcgcaacccatttggcgtgaagaatcaacacatctcatacaagtccgtaagataaagataggtggatga

SEQ ID NO: 10 - protein, 571 aa, Arabidopsis thaliana
MAAYGQISSGMTVDPQVLSSSRNIGVSLSPLRRTLIGAGVRSTSISLRQCSLSVRSIKISEDSRKPKAYAENGAFDVGVLDSSSYRLADSRTSSNDSRRKTKIVCTIGPSSSSREMIWKLAEAGMNVARLNMSHGDHASHQITIDLVKEYNSLFVDKAIAIMLDTKGPEVRSGDVPQPIFLEEGQEFNFTIKRGVSLKDTVSVNYDDFVNDVEVGDILLVDGGMMSLAVKSKTSDLVKCVVIDGGELQSRRHLNVRGKSATLPSITDKDWEDIKFGVDNQVDFYAVSFVKDAKVVHELKNYLKTCSADISVIVKIESADSIKNLPSIISACDGAMVARGDLGAELPIEEVPLLQEEIIRRCRSIHKPVIVATNMLESMINHPTPTRAEVSDIAIAVREGADAIMLSGETAHGKFPLKAVNVMHTVALRTEASLPVRTSASRTTAYKGHMGQMFAFHASIMANTLSSPLIVFTRTGSMAVLLSHYRPSATIFAFTNQRRIMQRLALYQGVMPIYMEFSDDAEDTYARSLKLLQDENMLKEGQHVTLVQSGSQPIWREESTHLIQVRKIKIGG

SEQ ID NO: 11 - DNA, 1551 bp, Arabidopsis thaliana
tcgattaaaatctccgaagatagccgcaaacctaaagcttatgcagagaacggtgcttttgatgtgggagttttggattcttcatcatatagattggctgattcaagaacaagtagtaatgattcaaggaggaagactaagattgtgtgtacgattggaccgtcttcgagttctagggaaatgatttggaaactcgcggaagctggaatgaatgtggctcgtttgaatatgtctcatggtgatcatgcttctcatcagataactattgatttagttaaggagtataattctttgtttgttgacaaagctattgctattatgttggatacaaagggtcctgaggttcgaagcggggatgtaccgcagccgatatttcttgaagagggtcaagagtttaactttactatcaagagaggtgtttcgcttaaagacactgttagtgtaaattatgatgattttgtgaacgatgttgaagttggggatatacttttggtggatggtggaatgatgtcgttagctgttaaatcaaagacgagtgatttggtgaagtgtgtggttattgatggtggagagcttcaatctagacgtcacttgaatgttcgaggaaagagtgcgactcttccatccattacagacaaagattgggaagacataaaatttggagtggacaaccaagtcgatttctacgccgtctcctttgttaaggatgctaaagttgtccatgagttgaagaactatctcaaaacctgcagtgcagacatatcggtgattgtgaaaattgaaagtgcagactctataaagaatcttccttctatcatatctgcttgtgatggggcaatggttgctcgtggagatcttggagctgaacttcccattgaagaggtcccgttgttacaggaagaaataatcagaaggtgtagaagcattcataaaccagtgattgttgccacaaacatgctagagagtatgattaatcatccaacgcctacaagagctgaagtctctgacattgcaattgcagtacgtgaaggcgcagatgctatcatgctttctggtgaaaccgcacatggaaagtttccgctgaaagctgttaacgtaatgcatactgtggcgttgagaaccgaggcaagtctacctgtcagaacctcggcatcccgtaccactgcttacaagggtcacatgggccaaatgtttgcttttcatgcttctataatggcaaatacactgagctcaccgctaattgtatttacgagaaccggatccatggcagtgcttctaagccactaccgcccatctgcaacaattttcgccttcacaaaccagagaagaataatgcaaaggcttgctctttatcaaggtgtcatgcctatatatatggagttctcggatgatgcagaagatacatatgcccggtctctcaaactcttacaggacgagaatatgctcaaggaaggacaacatgtaactcttgtccaaagtggctcgcaacccatttggcgtgaagaatcaacacatctcatacaagtccgtaagataaagataggtggatga

SEQ ID NO: 12 - protein, 516 aa, Arabidopsis thaliana
SIKISEDSRKPKAYAENGAFDVGVLDSSSYRLADSRTSSNDSRRKTKIVCTIGPSSSSREMIWKLAEAGMNVARLNMSHGDHASHQITIDLVKEYNSLFVDKAIAIMLDTKGPEVRSGDVPQPIFLEEGQEFNFTIKRGVSLKDTVSVNYDDFVNDVEVGDILLVDGGMMSLAVKSKTSDLVKCVVIDGGELQSRRHLNVRGKSATLPSITDKDWEDIKFGVDNQVDFYAVSFVKDAKVVHELKNYLKTCSADISVIVKIESADSIKNLPSIISACDGAMVARGDLGAELPIEEVPLLQEEIIRRCRSIHKPVIVATNMLESMINHPTPTRAEVSDIAIAVREGADAIMLSGETAHGKFPLKAVNVMHTVALRTEASLPVRTSASRTTAYKGHMGQMFAFHASIMANTLSSPLIVFTRTGSMAVLLSHYRPSATIFAFTNQRRIMQRLALYQGVMPIYMEFSDDAEDTYARSLKLLQDENMLKEGQHVTLVQSGSQPIWREESTHLIQVRKIKIGG

SEQ ID NO: 13 - DNA, 1716 bp, Artificial sequence
  Codon optimized AtPK440 sequence for expression in Zea mays
atggccgcgtatgggcaaatctcctcgggtatgacagtcgatccacaagtactcagctcgtctcgcaatattggtgtttcgctctcacccctaagaaggacgttgatcggtgctggagtgcggtcaacttcgatttcccttcgccagtgtagccttagcgttaggtctatcaagatatcggaagatagccgcaagcccaaggcttacgccgagaacggtgcgtttgacgtgggggtattggactcgagttcctacagactggctgattctagaacaagctcaaatgattctcgacggaagactaagatcgtttgcactatcggcccaagtagctcgagccgtgagatgatatggaagctagcggaggctggaatgaacgtcgccagactcaatatgagccatggtgaccatgcttcccatcaaattaccatagacctggtcaaagagtacaatagtctcttcgttgataaggctatcgctattatgctggatactaaaggacctgaggtcagatccggtgacgtgcctcaaccgatattcttggaggagggccaagagtttaacttcacgatcaagcgtggtgtgtcactcaaagacacggtgagcgtcaactacgacgatttcgtcaatgacgtcgaagtgggtgacatacttctggtcgacgggggtatgatgtcacttgctgttaagtccaaaacgtccgacctcgtcaagtgcgtcgtgattgatggaggtgaacttcaatcccgtcgccacttgaatgtccgtggaaagtccgctacgttgccaagcataactgacaaggactgggaggatatcaagttcggagtggacaaccaagtggacttctacgccgtgagcttcgtgaaagatgcaaaggtggttcacgagctgaagaactacctcaagacctgctctgctgacatctccgttatcgtgaagatcgagagtgccgattctatcaagaacctgccaagcataatcagtgcgtgtgacggtgcgatggtagctagaggtgatctgggtgctgagttgccaatagaggaagtaccgctgcttcaagaggagatcatacgtcgctgccgtagtattcacaagcctgtgatagtcgctacgaacatgctcgagtctatgatcaatcaccccacaccaacgagggccgaagtatccgacattgccatcgctgtgagggagggagcagacgcgattatgctctctggcgaaactgcccacgggaagttccccttgaaagctgtcaacgttatgcacacggtggccttacgaaccgaagcttcactcccggtcagaacttctgcgagccgtaccacagcttataagggccacatgggtcagatgtttgcattccacgcgtctattatggcgaatacgctatcttcaccgctgatcgttttcacccgtactggcagtatggctgtgctactctcgcactaccggccgtctgccacaatctttgctttcacgaatcagagacgaataatgcaaaggctcgccctgtaccaaggtgtgatgcctatctacatggagttctccgatgacgctgaagacacctacgctcgctcgctgaagctactccaagatgagaacatgctcaaggaaggtcagcatgtgaccctcgtccagagcggaagtcagccgatctggcgtgaggaatcaacccacctcatccaagtccgaaagatcaagatcggaggttga

SEQ ID NO: 14 - protein, 571 aa, Arabidopsis thaliana; identical to SEQ ID NO: 10

SEQ ID NO: 15 - DNA, 1698 bp, Linum usipatissimum
atggcggctcaagtggtagctaccaaatccatccacacttctcttctctcccccgttgcttcttctgggtcgcttcaaaaccgtgtcaaaaaccttaaagcttcaaactttaccaccaatgtcggaggacttgctcgtagaaccaacccaactatttccgccgtaaagactgatgtagtcatccccgtctcacccgaagatgtcccaaagagggcggagaggtttacggagctgcagaaatttggggacacgtcagtgggggtgtggtctaagccaacggtgaagcgcaagacgaagatagtgtgcacaattggtccttctaccaacactagggagatgatatggaagctggctgaggcaggaatgaatgttgctaggttgaacatgtctcatggggatcatgcttcccaccagaaagtgattgacttggtcaaggagtataatgctcagtccaaggacaatgtcattgccattatgattgataccaagggtcctgaggttaggagtggtgacttgccacaaccgataatgttggcgcctggtcaggagttcactttcactattcgtagaggtgttgggactggtgattgtgttagtgtgaactatgatgatttcgtcaatgatgttgaagatggtgacatgcttctcgttgatggggggatgatgtctctggtcgtgaagtcaaagactgaagattcagtgaagtgtgaggttgttgatgggggagaactgaagtctaggcgtcatcttaacgttcgaggaaagagtgcaacattgccttccatcactgataaggattgggatgacataaagtttggagtgaacaacaaggttgatttctatgctgtttcattcgtcaaagatgcacaagtcgttcatgagttgaagaactatctcaagagctgcggtgcagatattcacgtgattgtaaaaatcgagagtgctgattctattcctaatttgcactcgataatcacagcgtcagatggggcaatggttgcacggggcgatcttggtgcagagctcccaattgaagaagttccacttcttcaggaagagataatccggttatgccggggcatgggaaaagctgttattgtggctacaaatatgttggaaagtatgattgttcatcctactccaaccagagcggaggtgtcagatatcgccattgctgttcgggaaggtgcagatgcggtgatgctttctggagaaactgctcacggaaagttcccattgaaggctgtaaaggttatgcatactgtctcgttacgcactgaagcaactttgtctagtgggccaatgccagttaatcttggacaagctttcaagaatcacatgagtgagatgtttgcataccatgcaaccatgatgtcgaacactcttgggacgtcaatcgttgtcttcaccagaactggcttcatggctgttttgcttagtcattatcgaccttcggggacaatatttgctttcactaatgacaaaacgatacaacagaggttggcagtctatcaaggagtatgtcccatttacctcgagttctcggacgatgctgaagaaacctttgctgatgccttaacgctacttaagaatcatggaatggttaagcaaggtgaagaggttgcactcgttcaaagtgggaaaaaacccatctggcgattccagtcgacccacaatattcaggtgcgtgaagtatag

SEQ ID NO: 16 - protein, 565 aa, Linum usipatissimum
MAAQVVATKSIHTSLLSPVASSGSLQNRVKNLKASNFTTNVGGLARRTNPTISAVKTDVVIPVSPEDVPKRAERFTELQKFGDTSVGVWSKPTVKRKTKIVCTIGPSTNTREMIWKLAEAGMNVARLNMSHGDHASHQKVIDLVKEYNAQSKDNVIAIMIDTKGPEVRSGDLPQPIMLAPGQEFTFTIRRGVGTGDCVSVNYDDFVNDVEDGDMLLVDGGMMSLVVKSKTEDSVKCEVVDGGELKSRRHLNVRGKSATLPSITDKDWDDIKFGVNNKVDFYAVSFVKDAQVVHELKNYLKSCGADIHVIVKIESADSIPNLHSIITASDGAMVARGDLGAELPIEEVPLLQEEIIRLCRGMGKAVIVATNMLESMIVHPTPTRAEVSDIAIAVREGADAVMLSGETAHGKFPLKAVKVMHTVSLRTEATLSSGPMPVNLGQAFKNHMSEMFAYHATMMSNTLGTSIVVFTRTGFMAVLLSHYRPSGTIFAFTNDKTIQQRLAVYQGVCPIYLEFSDDAEETFADALTLLKNHGMVKQGEEVALVQSGKKPIWRFQSTHNIQVREV

SEQ ID NO: 17 - DNA, 1698 bp, Artificial sequence
  Codon optimized LuPK001 sequence for expression in Zea mays
atggccgcccaggtggtggccaccaagagcatccacaccagcctgctgagcccggtggccagcagcggcagcctgcagaacagggtgaagaacctgaaggccagcaacttcaccaccaacgtgggcggcctggccaggaggaccaacccgaccatcagcgccgtgaagaccgacgtggtgatcccggtgagcccggaggacgtgccgaagagggccgagaggttcaccgagctgcagaagttcggcgacaccagcgtgggcgtgtggagcaagccgaccgtgaagaggaagaccaagatcgtgtgcaccatcggcccgagcaccaacaccagggagatgatctggaagctggccgaggccggcatgaacgtggccaggctgaacatgagccacggcgaccacgccagccaccagaaggtgatcgacctggtgaaggagtacaacgcccagagcaaggacaacgtgatcgccatcatgatcgacaccaagggcccggaggtgaggagcggcgacctgccgcagccgatcatgctggccccgggccaggagttcaccttcaccatcaggaggggcgtgggcaccggcgactgcgtgagcgtgaactacgacgacttcgtgaacgacgtggaggacggcgacatgctgctggtggacggcggcatgatgagcctggtggtgaagagcaagaccgaggacagcgtgaagtgcgaggtggtggacggcggcgagctgaagagcaggaggcacctgaacgtgaggggcaagagcgccaccctgccgagtatcaccgacaaggactgggacgatatcaagttcggcgtgaacaacaaggtggacttctacgccgtgagcttcgtgaaggacgcccaggtggtgcacgagctgaagaactacctgaagagctgcggcgccgacatccacgtgatcgtgaagatcgagagcgccgacagtatcccgaacctgcacagtataatcaccgccagcgacggcgcgatggtggccaggggcgacctgggcgccgagctgccgatcgaggaggtgccgctgctgcaggaggagattatcaggctgtgcagggggatgggcaaggccgtgatcgtggccaccaatatgctggagagtatgatcgtgcacccgaccccgaccagggccgaggtgagcgatatcgcgatcgccgtgagggagggcgccgacgccgtgatgctgagcggcgagaccgcccacggcaagttcccgctgaaggccgtgaaggtgatgcacaccgtgagcctgaggaccgaggccaccctgagcagcggcccgatgccggtgaacctgggccaggccttcaagaaccatatgagcgagatgttcgcctaccacgccaccatgatgagcaacaccctgggcaccagcatcgtggtgttcaccaggaccggcttcatggccgtgctgctgagccactacaggccgagcggcaccatcttcgccttcaccaacgacaagaccatccagcagaggctggccgtgtaccagggcgtgtgcccgatctacctggagttcagcgacgacgccgaggagaccttcgccgacgccctgaccctgctgaagaaccacggcatggtgaagcagggcgaggaggtggccctggtgcagagcggcaagaagccgatctggaggttccagagcacccacaatatccaggtgagggaggtgtga

SEQ ID NO: 18 - protein, 565 aa, Linum usipatissimum; identical to SEQ ID NO: 16

SEQ ID NO: 19 - DNA, 1740 bp, Arabidopsis thaliana
atggctcaagtggttgctaccaggtcaattcaaggctcgatgttatctcccaacggtggatctgtgtctacaagatccgagaagctattgaaaccagcgagttttgcagtgaaggttcttggcaacgaagcaaagagaagtggaagagtctctgtaagaagcagaagagtggttgatactactgtgagatccgctcgtgttgagactgaagtcattcctgtttctcctgaagatgtgcctaacagagaggagcagcttgagaggttgttggaaatgcagcagtttggtgatacatcggtagggatgtggtcgaagccgacagtgaggaggaagacaaagattgtttgcaccgttggtccgtcgaccaacacacgagaaatgatatggaaattggctgaagctgggatgaatgttgctaggatgaatatgtctcatggagatcatgcttcacataagaaggttattgatttggttaaagaatacaatgcacaaactaaagacaacactattgctatcatgcttgacaccaagggtccggaagttaggagtggagatttacctcagccaattatgttagatcctggtcaagagtttacctttacaattgagagaggagtcagcacaccaagttgtgtcagtgttaactatgatgatttcgttaatgacgtggaagcgggtgacatgcttcttgttgatggtggtatgatgtcgtttatggtgaagtcaaagaccaaagactctgtcaaatgtgaagttgttgatggtggagaacttaagtcaaggagacacctgaatgtccgaggaaagagtgcaactttaccttcaatcactgagaaggactgggaggatattaaatttggagtggagaacaaagttgacttttatgcagtttcctttgtcaaagatgctcaagttgtacacgagttgaagaaataccttcaaaatagtggtgctgatatacacgtgatagtgaaaattgagagtgcagactccatacctaacttgcactccattatcacagcaccagatggggcaatggttgcaagaggtgatcttggtgcagagcttccaattgaagaagtccccattcttcaggaggagatcattaacctgtgccgtagtatgggaaaagctgttattgttgcgactaacatgcttgagagtatgatagttcatccaactccaacccgggcagaggtctcagacattgctatcgctgttagagaaggtgctgatgcggtaatgctttcaggagaaactgctcacggaaagttcccattgaaagctgctggagtgatgcacactgttgcattgcgaacagaagcaaccattactagcggtgaaatgccacctaatcttggtcaagccttcaagaaccatatgagtgagatgtttgcataccatgcaaccatgatgtcaaacacacttggaacttcaactgttgtcttcaccagaaccggtttcatggccatattgttaagtcactatcgtccttccggcacaatctatgccttcacaaatgagaaaaaaatacaacaaagattagctttgtatcaaggtgtatgccccatatatatggagttcacagatgatgcagaagaaacttttgctaatgctttggctacattactgaaacaaggaatggtgaagaagggagaggaaatagcaatcgtacagagcggtacacagccaatctggcgatctcaatcgacacataacatccaagtccgcaaggtttaa

SEQ ID NO: 20 - protein, 579 aa, Arabidopsis thaliana
MAQVVATRSIQGSMLSPNGGSVSTRSEKLLKPASFAVKVLGNEAKRSGRVSVRSRRVVDTTVRSARVETEVIPVSPEDVPNREEQLERLLEMQQFGDTSVGMWSKPTVRRKTKIVCTVGPSTNTREMIWKLAEAGMNVARMNMSHGDHASHKKVIDLVKEYNAQTKDNTIAIMLDTKGPEVRSGDLPQPIMLDPGQEFTFTIERGVSTPSCVSVNYDDFVNDVEAGDMLLVDGGMMSFMVKSKTKDSVKCEVVDGGELKSRRHLNVRGKSATLPSITEKDWEDIKFGVENKVDFYAVSFVKDAQVVHELKKYLQNSGADIHVIVKIESADSIPNLHSIITAPDGAMVARGDLGAELPIEEVPILQEEIINLCRSMGKAVIVATNMLESMIVHPTPTRAEVSDIAIAVREGADAVMLSGETAHGKFPLKAAGVMHTVALRTEATITSGEMPPNLGQAFKNHMSEMFAYHATMMSNTLGTSTVVFTRTGFMAILLSHYRPSGTIYAFTNEKKIQQRLALYQGVCPIYMEFTDDAEETFANALATLLKQGMVKKGEEIAIVQSGTQPIWRSQSTHNIQVRKV

SEQ ID NO: 21 - DNA, 1740 bp, Arabidopsis thaliana
atggctcaagtggttgctaccaggtcaattcaaggctcgatgttatctcccaacggtggatctgtgtctacaagatccgagaagctattgaaaccagcgagttttgcagtgaaggttcttggcaacgaagcaaagagaagtggaagagtctctgtaagaagcagaagagtggttgatactactgtgagatccgctcgtgttgagactgaagtcattcctgtttctcctgaagatgtgcctaacagagaggagcagcttgagaggttgttggaaatgcagcagtttggtgatacatcggtagggatgtggtcgaagccgacagtgaggaggaagacaaagattgtttgcaccgttggtccgtcgaccaacacacgagaaatgatatggaaattggctgaagctgggatgaatgttgctaggatgaatatgtctcatggagatcatgcttcacataagaaggttattgatttggttaaagaatacaatgcacaaactaaagacaacactattgctatcatgcttgacaccaagggtccggaagttaggagtggagatttacctcagccaattatgttagatcctggtcaagagtttacctttacaattgagagaggagtcagcacaccaagttgtgtcagtgttaactatgatgatttcgttaatgacgtggaagcgggtgacatgcttcttgttgatggtggtatgatgtcgtttatggtgaagtcaaagaccaaagactctgtcaaatgtgaagttgttgatggtggagaacttaagtcaaggagacacctgaatgtccgaggaaagagtgcaactttaccttcaatcactgagaaggactgggaggatattaaatttggagtggagaacaaagttgacttttatgcagtttcctttgtcaaagatgctcaagttgtacacgagttgaagaaataccttcaaaatagtggtgctgatatacacgtgatagtgaaaattgagagtgcagactccatacctaacttgcactccattatcacagcatcagatggggcaatggttgcaagaggtgatcttggtgcagagcttccaattgaagaagtccccattcttcaggaggagatcattaacctgtgccgtagtatgggaaaagctgttattgttgcggctaacatgcttgagagtatgatagttcatccaactccaacccgggcagaggtctcagacattgctatcgctgttagagaaggtgctgatgcggtaatgctttcaggagaaactgctcacggaaagttcccattgaaagctgctggagtgatgcacactgttgcattgcgaacagaagcaaccattactagcggtgaaatgccacctaatcttggtcaagccttcaagaaccatatgagtgagatgtttgcataccatgcaaccatgatgtcaaacacacttggaacttcaactgttgtcttcaccagaaccggtttcatggccatattgttaagtcactatcgtccttccggcacaatctatgccttcacaaatgagaaaaaaatacaacaaagattagctttgtatcaaggtgtatgccccatatatatggagttcacagatgatgcagaagaaacttttgctaatgctttggctacattactgaaacaaggaatggtgaagaagggagaggaaatagcaatcgtacagagcggtacacagccaatctggcgatctcaatcgacacataacatccaagtccgcaaggtttaa

SEQ ID NO: 22 - protein, 579 aa, Arabidopsis thaliana
MAQVVATRSIQGSMLSPNGGSVSTRSEKLLKPASFAVKVLGNEAKRSGRVSVRSRRVVDTTVRSARVETEVIPVSPEDVPNREEQLERLLEMQQFGDTSVGMWSKPTVRRKTKIVCTVGPSTNTREMIWKLAEAGMNVARMNMSHGDHASHKKVIDLVKEYNAQTKDNTIAIMLDTKGPEVRSGDLPQPIMLDPGQEFTFTIERGVSTPSCVSVNYDDFVNDVEAGDMLLVDGGMMSFMVKSKTKDSVKCEVVDGGELKSRRHLNVRGKSATLPSITEKDWEDIKFGVENKVDFYAVSFVKDAQVVHELKKYLQNSGADIHVIVKIESADSIPNLHSIITASDGAMVARGDLGAELPIEEVPILQEEIINLCRSMGKAVIVAANMLESMIVHPTPTRAEVSDIAIAVREGADAVMLSGETAHGKFPLKAAGVMHTVALRTEATITSGEMPPNLGQAFKNHMSEMFAYHATMMSNTLGTSTVVFTRTGFMAILLSHYRPSGTIYAFTNEKKIQQRLALYQGVCPIYMEFTDDAEETFANALATLLKQGMVKKGEEIAIVQSGTQPIWRSQSTHNIQVRKV

SEQ ID NO: 23 - DNA, 1740 bp, Arabidopsis thaliana
atggctcaagttgttgctaccaggtcaattcaaggctcgatgttgtgtcccaacggtggatctgtgtctacaagatccgagaagctattgaagccggcgagttttgcagtgaaggttcttggaaacgaagctaagagaagtggaagagtctctgttagaagtagaagagtggttgatactacagtgagatctgctcgtgttgagactgaagtgattcctgtttctcctgaagatgtgcctaacagagaggagcagctcgagaggttgttggaaatgcagcagtttggtgatacatcggtagggatgtggtcgaaaccgacagtgaggaggaagacaaagatcgtttgcaccgttggtccgtcgaccaacacacgagaaatgatatggaaattggctgaagctgggatgaatgttgctaggatgaatatgtctcatggagatcatgcttcacataagaaggttattgatttggttaaagagtacaatgcacaaacgaaagacaacactattgctatcatgcttgataccaagggtccggaagttaggagtggagatttaccacaaccaattatgttagatccaggtcaagagtttacttttactattgagagaggagtcagtacaccaagttgtgtcagtgttaactatgatgatttcgtcaatgacgtggaagccggtgacatgcttcttgttgatggtggtatgatgtcatttatggtgaagtctaagaccaaagactctgtcaaatgtgaagttgttgatggtggagaacttaagtcaaggagacatttgaatgtccgaggaaagagtgcaacattaccttcaatcactgagaaggattgggaagatattaaatttggagtggagaataaagttgacttttatgcagtttcctttgtcaaagatgctcaagtggtacacgaattgaagaaataccttcaaaattctggtgctgatatacatgtgatagtgaaaattgaaagtgcagactccatacctaacttgcattccattatcacagcatcagatggggcaatggttgctagaggtgatcttggtgcagagcttccaattgaagaagtccccattcttcaggaggagatcattaacctatgccggagcatgggaaaagctgtaattgttgcgactaacatgcttgagagtatgatagttcatccaactccaacccgggcagaggtctcagacattgctatcgctgttagagaaggtgctgatgcagttatgctttcaggagaaactgctcacggaaagttcccattgaaagctgctggggtgatgcacacggttgcattgcgaacagaagccaccattactagtggtgaaatgccacctaatcttggtcaagccttcaagaaccatatgagcgagatgtttgcatatcatgcaaccatgatgtcaaacacacttggaacttcaactgttgtcttcaccagaactggtttcatggccattctgctaagtcactatcgtccttccggcacaatctatgccttcacaaatgagaaaaaaatacaacaaagattagcattgtatcaaggtgtatgccccatatatatggagttttcagatgatgcagaagagacttttgctaatgccttggctacattactgaaacaaggaatggtgaagaagggagaggaaatagcaatcgtacagagcggtacacagccaatctggcgatctcaatcgacgcataacatccaagtccgcaaggtttaa

SEQ ID NO: 24 - protein, 579 aa, Arabidopsis thaliana
MAQVVATRSIQGSMLCPNGGSVSTRSEKLLKPASFAVKVLGNEAKRSGRVSVRSRRVVDTTVRSARVETEVIPVSPEDVPNREEQLERLLEMQQFGDTSVGMWSKPTVRRKTKIVCTVGPSTNTREMIWKLAEAGMNVARMNMSHGDHASHKKVIDLVKEYNAQTKDNTIAIMLDTKGPEVRSGDLPQPIMLDPGQEFTFTIERGVSTPSCVSVNYDDFVNDVEAGDMLLVDGGMMSFMVKSKTKDSVKCEVVDGGELKSRRHLNVRGKSATLPSITEKDWEDIKFGVENKVDFYAVSFVKDAQVVHELKKYLQNSGADIHVIVKIESADSIPNLHSIITASDGAMVARGDLGAELPIEEVPILQEEIINLCRSMGKAVIVATNMLESMIVHPTPTRAEVSDIAIAVREGADAVMLSGETAHGKFPLKAAGVMHTVALRTEATITSGEMPPNLGQAFKNHMSEMFAYHATMMSNTLGTSTVVFTRTGFMAILLSHYRPSGTIYAFTNEKKIQQRLALYQGVCPIYMEFSDDAEETFANALATLLKQGMVKKGEEIAIVQSGTQPIWRSQSTHNIQVRKV

SEQ ID NO: 25 - DNA, 1743 bp, Artificial sequence
  protein kinase homolog
atggctcaggtcgttgctacaaggtcgattcaaggctctatgttgagtcccaacggtggatctgtttccacgagatccgacaagcttctgaagccggcgagtttcgcagtgaaggttcttggcaacgaatccaagaagagcggaagagtgtccgtaagaggaggaagaaaggttgataccactgtgagatccgctcgtgtggagaccgaagtcattccagtgtctcccgaggatgttccaaacagagaggagcaactggagcggtttttggaaatgcagaagtttagtgacacatcggtagggatgtggtcgaaaccgacagtgaggaggaagactaagattgtgtgcaccgttggtccttctaccaacacacgagagatgatatggaaactggctgaagctgggatgaatgttgctaggatgaacatgtctcatggggatcatgcttctcataagaaggttattgatttggtcaaagagtacaatgcgcaatctaaagataacaccattgctatcatgcttgataccaagggtccagaagttaggagtggagatttaccccagccgattatgttagaccctggtcaagagtttacttttacaattgagagaggagtcagcaccccaagttgtgtcagtgttaactatgatgattttgtcaacgatgtggaggcgggagacatgctccttgttgatggtggtatgatgtcgtttatggtgaagtctaagactaaagagactgtcatatgtgaggttgttgatggtggagaactaaagtcaaggagacacttgaatgtccgagggaaaagtgcaacattaccttcaatcactgagaaggattgggaggatattaagttcggagtggagaacaaagttgacttctatgcggtttcttttgtcaaagatgcacaagtggtgcatgaactgaagaattacctccaaggttgtggtgctgatattcacgtgatagtaaaaattgaaagcgcagactccatacctaacttgcattccattatcaccgcatcagatggggcaatggttgcaagaggtgatcttggtgctgagcttcctattgaggaagtacccattcttcaggagaggatcattaacctatgccgtagcatgggaaaagctgttattgttgcaactaacatgcttgagagtatgatagttcatccgactccaacccgagcggaggtttctgatattgctatagctgttagagaaggtgctgatgcagtcatgctttcaggagaaactgctcacggaaagttcccactgaaagctgctggagtgatgcatacagtcgcacttcgaacagaagcaaccattactactagtactgaaatgccacctaatcttggtcaagccttcaagaaccatatgagtgagatgtttgcataccatgcaaccatgatgtcaaacacgcttggaacttcaactgttgtcttcaccagaactggtttcatggccatacttttaagtcactatcgcccttctggcaccatctacgccttcacaaaaaaaaaaaaaatacaacaaagattagctttgtatcaaggtgtatgccccatatatatggagttcacagatgatgcagaagaaacttttgctaatgctttggctacattactgaaacaaggaatggtgaagaagggagaggaaatagcaatcgtacagagcggtacacagccaatctggcgatctcaatcgacacataacatccaagtccgcaaggtttaa

SEQ ID NO: 26 - protein, 580 aa, Artificial sequence
  protein kinase homolog
MAQVVATRSIQGSMLSPNGGSVSTRSDKLLKPASFAVKVLGNESKKSGRVSVRGGRKVDTTVRSARVETEVIPVSPEDVPNREEQLERFLEMQKFSDTSVGMWSKPTVRRKTKIVCTVGPSTNTREMIWKLAEAGMNVARMNMSHGDHASHKKVIDLVKEYNAQSKDNTIAIMLDTKGPEVRSGDLPQPIMLDPGQEFTFTIERGVSTPSCVSVNYDDFVNDVEAGDMLLVDGGMMSFMVKSKTKETVICEVVDGGELKSRRHLNVRGKSATLPSITEKDWEDIKFGVENKVDFYAVSFVKDAQVVHELKNYLQGCGADIHVIVKIESADSIPNLHSIITASDGAMVARGDLGAELPIEEVPILQERIINLCRSMGKAVIVATNMLESMIVHPTPTRAEVSDIAIAVREGADAVMLSGETAHGKFPLKAAGVMHTVALRTEATITTSTEMPPNLGQAFKNHMSEMFAYHATMMSNTLGTSTVVFTRTGFMAILLSHYRPSGTIYAFTKKKKIQQRLALYQGVCPIYMEFTDDAEETFANALATLLKQGMVKKGEEIAIVQSGTQPIWRSQSTHNIQVRKV

SEQ ID NO: 27 - DNA, 1743 bp, Brassica napus
atggctcaggtcgttgctacaaggtcgattcaaggctcgatgttgagtcccaacggtggatctgtttccacgagatccgacaagcttctgaagccggcgagtttcgcagtgaaggttcttggcaacgaatccaagaagagcggaagagtgtccgtaagaggaggaagaaaggttgataccactgtgagatccgctcgtgtggagaccgaagtcattccagtgtctcccgaggatgttccaaacagagaggagcaactggagcggtttttggaaatgcagaagtttagtgacacatcggtagggatgtggtcgaaaccgacagtgaggaggaagactaagattgtgtgcaccgttggtccttctaccaacacacgagagatgatatggaaactagctgaagctgggatgaatgttgcaaggatgaacatgtctcatggggatcatgcttctcataagaaggttattgatttggtcaaagagtacaatgcgcagtctaaggacaacaccattgctatcatgctggatactaagggtccagaagttaggagtggagatttaccccagccgattatgttagaccctggtcaagagtttacttttacaattgagagaggagtcagcacaccaagctgtgtcagtgttaactatgatgattttgtcaacgatgtggaggccggagacatgctccttgttgatggtggtatgatgtcgtttatggtgaagtctaagactaaagagactgtcatatgtgaggttgttgatggtggagagctaaagtcaaggagacacttgaatgtccgaaggaaaagtgcaacattaccttcaatcactgagaaggattgggaggatattaagttcggagtggagaacaaagttgacttctatgctgtttcttttgtcaaagatgcacaagtggtgcatgaactgaagaattacctccaaggttgtggtgctgatattcacgtgatagtaaaaattgaaagcgcagactccatacctaacttgcattccattatcaccgcatcagatggggcaatggttgcaagaggtgatcttggtgcagagcttcctattgaagaagtacccattcttcaggaaaagatcattaacctatgccgtagcatgggaaaagctgttattgttgcaactaacatgcttgagagtatgatagttcatccgactccaacccgagcggaggtttctgatattgctatagctgttagagaaggtgctgatgcagtcatgctttcaggagaaactgctcacggaaagttcccattgaaagctgctggagtgatgcatacagttgcactgcgaacagaagcaaccattactactagtactgaaatgccacctaatcttggtcaagccttcaagaaccatatgagtgagatgtttgcataccatgcaaccatgatgtcaaacacacttggaacttcaactgttgtcttcacaagaactggtttcatggccatactactaagtcactatcgcccttccggcacaatctacgccttcacgaacgagaaaaaaatacagcaaagattagccttgtatcaaggtgtatgccccatatatatggagttctcagatgatgcagaagacactttcactaaagctttggctacactactgaaacaaggaatggtgaagaagggagaggaaatagcgattgtacagagcgggtcacaaccaatctggcggtctcaatcgactcataacatccaagtccgcaaggtgtaa

SEQ ID NO: 28 - protein, 580 aa, Brassica napus
MAQVVATRSIQGSMLSPNGGSVSTRSDKLLKPASFAVKVLGNESKKSGRVSVRGGRKVDTTVRSARVETEVIPVSPEDVPNREEQLERFLEMQKFSDTSVGMWSKPTVRRKTKIVCTVGPSTNTREMIWKLAEAGMNVARMNMSHGDHASHKKVIDLVKEYNAQSKDNTIAIMLDTKGPEVRSGDLPQPIMLDPGQEFTFTIERGVSTPSCVSVNYDDFVNDVEAGDMLLVDGGMMSFMVKSKTKETVICEVVDGGELKSRRHLNVRRKSATLPSITEKDWEDIKFGVENKVDFYAVSFVKDAQVVHELKNYLQGCGADIHVIVKIESADSIPNLHSIITASDGAMVARGDLGAELPIEEVPILQEKIINLCRSMGKAVIVATNMLESMIVHPTPTRAEVSDIAIAVREGADAVMLSGETAHGKFPLKAAGVMHTVALRTEATITTSTEMPPNLGQAFKNHMSEMFAYHATMMSNTLGTSTVVFTRTGFMAILLSHYRPSGTIYAFTNEKKIQQRLALYQGVCPIYMEFSDDAEDTFTKALATLLKQGMVKKGEEIAIVQSGSQPIWRSQSTHNIQVRKV

SEQ ID NO: 29 - DNA, 1743 bp, Brassica napus
atggctcaggtcgttgctacaaggtcgattcaaggctcgatgttgagtcccaacggtggatctgtttccacgagatccgacaagcttctgaagccggcgagtttcgcagtgaaggttcttggcaacgaatccaagaagagcggaagagtgtccgtaagaggaggaagaaaggttgataccactgtgagatccgctcgtgtggagaccgaagtcattccagtgtctcccgaggatgttccaaacagagaggagcaactggagcggtttttggaaatgcagaagtttagtgacacatcggtagggatgtggtcgaaaccgacagtgaggaggaagactaagattgtgtgcaccgttggtccttctaccaacacacgagagatgatatggaaactagctgaagctgggatgaatgttgcaaggatgaacatgtctcatggggatcatgcttctcataagaaggttattgatttggtcaaagagtacaatgcgcagtctaaggacaacaccattgctatcatgctggatactaagggtccagaagttaggagtggagatttaccccagccgattatgttagaccctggtcaagagtttacttttacaattgagagaggagtcagcacaccaagctgtgtcagtgttaactatgatgattttgtcaacgatgtggaggccggagacatgctccttgttgatggtggtatgatgtcgtttatggtgaagtctaagactaaagagactgtcatatgtgaggttgttgatggtggagagcttaagtcaaggagacacttgaatgtccgagggaaaagtgcaacattaccgtcaatcactgagaaggattgggaggatattaagttcggagtggagaacaaagttgacttttatgcagtttcttttgtcaaagatgcacaagtggtacacgaactcaagaattacctcaaaggttgtggtgctgatattcacgtgatagtaaaaattgaaagcgcagactccatacctaacttgaattccattatcaccgcatcagatggggcaatggttgcaagaggtgatcttggtgcagagcttcctattgaagaagtacccattcttcaggagaggatcattaacctatgccgtagcatgggaaaagctgttattgttgcaactaacatgcttgagagtatgatagttcatccgactccaacccgagcggaggtttctgatattgctatagctgttagagaaggtgctgatgcagtcatgctttcaggagaaactgctcacggaaagttcccactgaaagctgctggagtgatgcatacagtcgcactgcgaacagaagcaaccattactactagtactgaaatgccacctaatcttggtcaagccttcaagaaccatatgagtgagatgtttgcataccatgcaaccatgatgtcaaacacgcttggaacttcaactgttgtcttcaccagaactggtttcatggccatacttttaagtcactatcgcccttctggcaccatctacgccttcacgaatgagaaaaaaatacagcaaagattagccttgtatcaaggggtgtgccccatatatatggagttctcagatgatgcagaggacactttcactaaagctttggctacactactgaaacaaggaatggtgaagaagggagaggaaatagcgattgtacagagcgggtcacaaccaatctggcggtctcaatcgactcataacatccaagtccgcaaggtgtaa

SEQ ID NO: 30 - protein, 580 aa, Brassica napus
MAQVVATRSIQGSMLSPNGGSVSTRSDKLLKPASFAVKVLGNESKKSGRVSVRGGRKVDTTVRSARVETEVIPVSPEDVPNREEQLERFLEMQKFSDTSVGMWSKPTVRRKTKIVCTVGPSTNTREMIWKLAEAGMNVARMNMSHGDHASHKKVIDLVKEYNAQSKDNTIAIMLDTKGPEVRSGDLPQPIMLDPGQEFTFTIERGVSTPSCVSVNYDDFVNDVEAGDMLLVDGGMMSFMVKSKTKETVICEVVDGGELKSRRHLNVRGKSATLPSITEKDWEDIKFGVENKVDFYAVSFVKDAQVVHELKNYLKGCGADIHVIVKIESADSIPNLNSIITASDGAMVARGDLGAELPIEEVPILQERIINLCRSMGKAVIVATNMLESMIVHPTPTRAEVSDIAIAVREGADAVMLSGETAHGKFPLKAAGVMHTVALRTEATITTSTEMPPNLGQAFKNHMSEMFAYHATMMSNTLGTSTVVFTRTGFMAILLSHYRPSGTIYAFTNEKKIQQRLALYQGVCPIYMEFSDDAEDTFTKALATLLKQGMVKKGEEIAIVQSGSQPIWRSQSTHNIQVRKV

SEQ ID NO: 31 - DNA, 1743 bp, Ricinus communis
atggctcaagttgtggctaccagatcgattcacacttcattgctgtgtcctacctctggatcctcactgcaagaccgggcagataagctaaagccttcaagttttgcgtcaaagttactgtcacgcgaagacaacaagagaaataagaacagcagccttacaatttctcgcagaaacactcaaatccaagctgctaacagatccacacgtgtcgaaactgaagttattcctgtatcgcctgaggatgtaccgcagagagaggagcaggtattgcatttacagcaattaggcgacacatcagtgagtatgtggtccaagcctgtagttagacggaagacaaagattgtatgcaccataggtccatcaacaaacacgagggagatgatttggaagctggctgaagcaggaatgaacgtagctcgactgaatatgtctcatggagaccatgcatctcatcagaaagttatagatttagttaaagaatataatgcacagtccaaagataatgtcattgctatcatgcttgacactaagggtcctgaagttaggagtggcgacttgcctcaaccgatcatgttatcacctggacaggaatttacttttacaattcgtaggggggttggcactgctgattgcgttagtgttaactatgatgattttgttaatgatgtagaagttggtgacatgcttcttgttgatggtggtatgatgtcacttctggtgaagtccaagacagaagattcagtgaggtgtgaagtaattgatggaggagaactaaagtctaggcgtcatttgaatgttagaggaaaaagcgcgacactgccttccatcactgaaaaggattgggatgacataaaatttggagtggacaataaggtagacttctatgcagtttcctttgttaaagatgcacaggttgttcatgaattaaagaattatttgcaaggctgtggtgcagacatacatgtgattgtgaaaattgaaagtgcagattctatcccaaatctgcattcaataatcacagcatctgatggggcaatggttgcaagaggagaccttggtgcagagcttcctatcgaggaggttccactcttgcaggaagaaataatcagactgtgtcggagtatggggaaagctgttattgtggcaacaaatatgctggaaagcatgatagttcatccaactccaacccgagccgaggtatctgacattgctattgctgttcgggagggttctgatgcagtcatgctttctggagaaactgctcatgggaagttcccattgaaagctgtgaaagtaatgcatactgttgcattacgaactgaagcaaccatagttggtggtacaatgccaccaaatcttggacaagccttcaagaaccacatgagcgagatgtttgcatatcacgcaaccatgatgtcaaacacacttggaacctcaattgttgtctttaccaggacaggcttcatggctatattattgagccattatcgaccttctggcactatttttgcattcacaaatgagaagagaatacaacagagactggctctataccaaggagtatgtcccatatacatgcaattttcagatgatgctgaagaaacctttgcaaatgccttatcagtgctcaagaaccaagggatggtaaaggaaggagaggaagttgctcttgttcagagtggcagacagccaatctggcgattccaatctactcacaacattcaggtcaggaaagtgtag

SEQ ID NO: 32 - protein, 580 aa, Ricinus communis
MAQVVATRSIHTSLLCPTSGSSLQDRADKLKPSSFASKLLSREDNKRNKNSSLTISRRNTQIQAANRSTRVETEVIPVSPEDVPQREEQVLHLQQLGDTSVSMWSKPVVRRKTKIVCTIGPSTNTREMIWKLAEAGMNVARLNMSHGDHASHQKVIDLVKEYNAQSKDNVIAIMLDTKGPEVRSGDLPQPIMLSPGQEFTFTIRRGVGTADCVSVNYDDFVNDVEVGDMLLVDGGMMSLLVKSKTEDSVRCEVIDGGELKSRRHLNVRGKSATLPSITEKDWDDIKFGVDNKVDFYAVSFVKDAQVVHELKNYLQGCGADIHVIVKIESADSIPNLHSIITASDGAMVARGDLGAELPIEEVPLLQEEIIRLCRSMGKAVIVATNMLESMIVHPTPTRAEVSDIAIAVREGSDAVMLSGETAHGKFPLKAVKVMHTVALRTEATIVGGTMPPNLGQAFKNHMSEMFAYHATMMSNTLGTSIVVFTRTGFMAILLSHYRPSGTIFAFTNEKRIQQRLALYQGVCPIYMQFSDDAEETFANALSVLKNQGMVKEGEEVALVQSGRQPIWRFQSTHNIQVRKV

SEQ ID NO: 33 - DNA, 1734 bp, Vitis vinifera
atggctcaggtggtggcgactcgcgcgattcagagctcgattctgtgccctagctctggatccgtgcatgagcgatttgagaagctgaagccctccggtttcgctgcgaaagtgttggctcgcgaagagcggaggagccggagattggcttggaggggcggtccaattgcggcggcgaagagatctgtgggagctgagacggaggttgttccggtcacaccagaggatgcgaagaacggagaggagcaatatcatcttttacggggaattcaacagcttggtgagacatctgttggtatgtggtctaagccaacggttagacgcaagacaaagattgtttgcacaattggtccatccaccaacacacgggaaatgatatggaagctggctgaggctggaatgaatgtcgcccgcctgaatatgtctcatggagatcatgcatctcatcagaaggttattgatttggtaaaagagtataatgcacaatccaaagataatgtcattgcaatcatgcttgataccaagggccctgaggtcaggagtggggacttgccccagccaatcatgttaaaaagtgggcaggaattcaccttcacaattcaaagaggcgttgggacagcagattgtgttagtgttaactatgatgattttgtcaatgatgtagaaatgggcgacatgcttctcgtggatggtggtatgatgtcattaatggtcaagtccaagactggagattcagtgaaatgtgaagttgttgatggtggagaactcaagtctagaaggcatctaaatgttcgaggaaaaagtgcaacactgccatcaattactgaaaaggactgggatgatatcaagtttggtgttgacaataaagtggacttttatgcagtctcctttgttaaagatgcaaaagtagtccatgaattgaagaactatctgaaaagctgtaatgcagatatccatgtcattgttaaaattgaaagtgccgactccattcccaatctacattcaattattaccgcatctgatggggcaatggttgctagaggggatcttggtgccgagcttcctattgaggaggtcccattgctacaggaagagataatcaggatatgtcgtagcatgggaaaagctgttattgtagcaaccaatatgctggaaagcatgattgttcacccaacaccaacccgagcagaggtatcagacattgccattgctgtccgagagggtgctgatgcagttatgctttctggagaaactgctcatggaaagtttcccttgaaagctgtgaaagtcatgcatacagtttccttaagaactgaagcaaccttggtcggtagcccaatgccaccaaatcttggtcaggctttcaagaaccatatgagcgaaatgtttgctttccatgcaaccatgatgtccaatactctcagtacttcaatagttgttttcactagaactggtttcatggctatccttctgagccattatcgaccttctggcaccatatttgcctttacaaatgaggagagagtacaacagagactagccgtgtaccaaggggtatgtcccatatacatgcagttctcagacgatgccgaagaaacctttgcaaatgccttgactttgttgcagaagcaagggatggtgaaggaaggagaagaggtggctcttgttcagagtggcaggcagccgatctggcggttccaatccacacacaatatccaggtcaggaaagtttaa

SEQ ID NO: 34 - protein, 577 aa, Vitis vinifera
MAQVVATRAIQSSILCPSSGSVHERFEKLKPSGFAAKVLAREERRSRRLAWRGGPIAAAKRSVGAETEVVPVTPEDAKNGEEQYHLLRGIQQLGETSVGMWSKPTVRRKTKIVCTIGPSTNTREMIWKLAEAGMNVARLNMSHGDHASHQKVIDLVKEYNAQSKDNVIAIMLDTKGPEVRSGDLPQPIMLKSGQEFTFTIQRGVGTADCVSVNYDDFVNDVEMGDMLLVDGGMMSLMVKSKTGDSVKCEVVDGGELKSRRHLNVRGKSATLPSITEKDWDDIKFGVDNKVDFYAVSFVKDAKVVHELKNYLKSCNADIHVIVKIESADSIPNLHSIITASDGAMVARGDLGAELPIEEVPLLQEEIIRICRSMGKAVIVATNMLESMIVHPTPTRAEVSDIAIAVREGADAVMLSGETAHGKFPLKAVKVMHTVSLRTEATLVGSPMPPNLGQAFKNHMSEMFAFHATMMSNTLSTSIVVFTRTGFMAILLSHYRPSGTIFAFTNEERVQQRLAVYQGVCPIYMQFSDDAEETFANALTLLQKQGMVKEGEEVALVQSGRQPIWRFQSTHNIQVRKV

SEQ ID NO: 35 - DNA, 1731 bp, Vitis vinifera
atggctcaggtggtggcgactcgcgcgattcagagctcgattctgtgccctagctctggatccgtgcatgagcgatttgagaagctgaagccctccggtttcgctgcgaaagtgttggctcgcgaagagcggaggagccggagattggcttggaggggcggtccaattgcggcggcgaagagatctgtgggagctgagacggaggttgttccggtcacaccagaggatgcgaagggagaggagcaatatcatcttttacggggaattcaacagcttggtgagacatctgttggtatgtggtctaagccaacggttagacgcaagacaaagattgtttgcacaattggtccatccaccaacacacgggaaatgatatggaagctggctgaggctggaatgaatgtcgcccgcctgaatatgtctcatggagatcatgcatctcatcagaaggttattgatttggtaaaagagtataatgcacaatccaaagataatgtcattgcaatcatgcttgataccaagggccctgaggtcaggagtggggacttgccccagccaatcatgttaaaaagtgggcaggaattcaccttcacaattcaaagaggcgttgggacagcagattgtgttagtgttaactatgatgattttgtcaatgatgtagaaatgggcgacatgcttctcgtggatggtggtatgatgtcattaatggtcaagtccaagactggagattcagtgaaatgtgaagttgttgatggtggagaactcaagtctagaaggcatctaaatgttcgaggaaaaagtgcaacactgccatcaattactgaaaaggactgggatgatatcaagtttggtgttgacaataaagtggacttttatgcagtctcctttgttaaagatgcaaaagtagtccatgaattgaagaactatctgaaaagctgtaatgcagatatccatgtcattgttaaaattgaaagtgccgactccattcccaatctacattcaattattaccgcatctgatggggcaatggttgctagaggggatcttggtgccgagcttcctattgaggaggtcccattgctacaggaagagataatcaggatatgtcgtagcatgggaaaagctgttattgtagcaaccaatatgctggaaagcatgattgttcacccaacaccaacccgagcagaggtatcagacattgccattgctgtccgagagggtgctgatgcagttatgctttctggagaaactgctcatggaaagtttcccttgaaagctgtgaaagtcatgcatacagtttccttaagaactgaagcaaccttggtcggtagcccaatgccaccaaatcttggtcaggctttcaagaaccatatgagcgaaatgtttgctttccatgcaaccatgatgtccaatactctcagtacttcaatagttgttttcactagaactggtttcatggctatccttctgagccattatcgaccttctggcaccatatttgcctttacaaatgaggagagagtacaacagagactagccgtgtaccaaggggtatgtcccatatacatgcagttctcagacgatgccgaagaaacctttgcaaatgccttgactttgttgcagaagcaagggatggtgaaggaaggagaagaggtggctcttgttcagagtggcaggcagccgatctggcggttccaatccacacacaatatccaggtcaggaaagtttaa

SEQ ID NO: 36 - protein, 576 aa, Vitis vinifera
MAQVVATRAIQSSILCPSSGSVHERFEKLKPSGFAAKVLAREERRSRRLAWRGGPIAAAKRSVGAETEVVPVTPEDAKGEEQYHLLRGIQQLGETSVGMWSKPTVRRKTKIVCTIGPSTNTREMIWKLAEAGMNVARLNMSHGDHASHQKVIDLVKEYNAQSKDNVIAIMLDTKGPEVRSGDLPQPIMLKSGQEFTFTIQRGVGTADCVSVNYDDFVNDVEMGDMLLVDGGMMSLMVKSKTGDSVKCEVVDGGELKSRRHLNVRGKSATLPSITEKDWDDIKFGVDNKVDFYAVSFVKDAKVVHELKNYLKSCNADIHVIVKIESADSIPNLHSIITASDGAMVARGDLGAELPIEEVPLLQEEIIRICRSMGKAVIVATNMLESMIVHPTPTRAEVSDIAIAVREGADAVMLSGETAHGKFPLKAVKVMHTVSLRTEATLVGSPMPPNLGQAFKNHMSEMFAFHATMMSNTLSTSIVVFTRTGFMAILLSHYRPSGTIFAFTNEERVQQRLAVYQGVCPIYMQFSDDAEETFANALTLLQKQGMVKEGEEVALVQSGRQPIWRFQSTHNIQVRKV

SEQ ID NO: 37 - DNA, 1749 bp, Populus trichocarpa
atggcgcaagtagtggctaccaccagatcgattcacagttctatcctatcatctccttcctctggatccttacaagaccgagttgataagctactcaaaccttccactctctcttcgaaatttctttcaagtcatgacaagaagaaaattatcagcagcgtttcttctcataggaacactcatattgtggcggcggctgttaagagatccgccgaacctgaagtcattcctgtctcacccgaggatgtaccaaagagggaggagcaatttgtgcaattagagcaactaggtgacacgacagcagctgttggaatgtggacgaagcctactgttaggagaaagacgaagattgtatgtactattggaccgtcaactaacacgaaggagatgatatggaagttggctgaagcgggaatgaatgttgctagaatgaatatgtcacatggagaccatgcttctcatcagaaagtcattgatttggttaaagagtataatgcacaagcgaaagataatgtcattgcaatcatgcttgatactaagggtcctgaagttaggagtggtgacttgcctcagcctatcatgctatcacctggacaggaattcacttttacgattcgtaggggtgttggaacatctgattgtgttagtgtgaactatgatgattttgttagtgatgtagaagcaggcgacatgcttcttgttgatggcggtatgatgtcacttctggtgaagtccaaaacagaggattcagtgaaatgtgaagttgttgatggaggagagcttaagtccaggcgtcatctaaatgttcgaggaaaaagcgcgacattgccttccatcactgacaaggattgggatgatataaaattcggagtggagaacaaagttgacttctatgctgtttcctttgttaaagatgcacaagtggttcatgaattgaagaattatctacaaagctgtggtgcagatatacatgtgattgtaaaaattgaaagtgcagattctataccaaatttgcattcgataatcacagcatctgatggggctatggttgcaagaggagatcttggtgcagagctccctattgaggaggttcccctgctgcaggaagagataatcaggctgtgccggagcatgggaaaagctgttattgtagcaacgaatatgctggaaagcatgattgttcatccaactcctaccagagcagaggtatcagacattgccattgctgttcgtgagggtgctgatgcagtcatgctttctggagaaactgcacatggaaaattcccactgaaagctgttaaagttatgcacacagtctccttgcgaactgaagcaaccatagctggtggtgaaatgccttctaatcttggtcaagcattcaagaaccatacgagtgagatgtttgcataccatgcaaccatgatgtcaaacactcttggaacctcaattgttgtcttcacaagaactggcttcatgtctatattactgagccattatcgaccttcaggcactgtttttgcattcacaaatgagaagaggatacaacagagactggctttgtatcaaggagtgtgtcccatatacatgcagttttcagatgatgctgaagaaacctttgcaaatgccttgtcagtgctcaagaatcaagggatggtaaaggaaggagaagaggtcgcacttgttcaaagtggaagacaaccaatctggcggttccaatctacccataatattcaggtccgcaaagtataa

SEQ ID NO: 38 - protein, 582 aa, Populus trichocarpa
MAQVVATTRSIHSSILSSPSSGSLQDRVDKLLKPSTLSSKFLSSHDKKKIISSVSSHRNTHIVAAAVKRSAEPEVIPVSPEDVPKREEQFVQLEQLGDTTAAVGMWTKPTVRRKTKIVCTIGPSTNTKEMIWKLAEAGMNVARMNMSHGDHASHQKVIDLVKEYNAQAKDNVIAIMLDTKGPEVRSGDLPQPIMLSPGQEFTFTIRRGVGTSDCVSVNYDDFVSDVEAGDMLLVDGGMMSLLVKSKTEDSVKCEVVDGGELKSRRHLNVRGKSATLPSITDKDWDDIKFGVENKVDFYAVSFVKDAQVVHELKNYLQSCGADIHVIVKIESADSIPNLHSIITASDGAMVARGDLGAELPIEEVPLLQEEIIRLCRSMGKAVIVATNMLESMIVHPTPTRAEVSDIAIAVREGADAVMLSGETAHGKFPLKAVKVMHTVSLRTEATIAGGEMPSNLGQAFKNHTSEMFAYHATMMSNTLGTSIVVFTRTGFMSILLSHYRPSGTVFAFTNEKRIQQRLALYQGVCPIYMQFSDDAEETFANALSVLKNQGMVKEGEEVALVQSGRQPIWRFQSTHNIQVRKV

SEQ ID NO: 39 - DNA, 1743 bp, Vitis vinifera
  Vitis vinifera Hom 88
atggctcaggtggtggcgactcgcgcgattcagagctcgattctgtgccctagctctggatccgtgcatgagcgatttgagaagctgaagccctccggtttcgctgcgaaagtgttggctcgcgaagagcggaggagccggagagtggcttggaggggcggtccgattgcggcggcgaagagatctgtgggagctgagayggaggttgttccggtcacaccagaggatgcgaagggagaggagcaatatcatcgtttacggggaattcaacagcttggtgagacatctgttggtatgtggtctaagccaacagttagacgcaagacaaagattgtttgcactattggtccatccaccaacacacgggaaatgatatggaagctggctgaagctggaatgaatgtagcccgcctgaatatgtctcatggagatcatgcatctcatcagaaggttattgatttggtaaaagagtataatgcacaatccaaagataatgtcattgcaatcatgcttgataccaaggcaagttttctaggccctgaggtcaggagtggggacttgccccagccaatcatgttaaaaagtgggcaggaattcaccttcacaattcaaagaggcgttgggacagcagattgtgttagtgttaactatgatgattttgtcaatgatgtagaaatgggcgacatgcttcttgtggatggtggtatgatgtcattaatggtcaagtccaagactggagattcagtgaaatgtgaagttgttgatggaggagagctcaagtctagaaggcatctaaatgttcgaggaaaaagtgcaacactgccatcaattactgaaaaggactgggatgatatcaagtttggtgttgacaataaagtggacttttatgcagtctcctttgttaaagatgcgaaagtagtccatgaattgaagaactatctgaaaagctgtaatgcagatatccatgtcattgttaaaattgaaagtgccgactccattcccaatctacattcaattattaccgcatctgatggggcaatggttgctagaggggatcttggtgccgagcttcctattgaggaggtcccattgctacaggaagagataatcaggatatgtcgtagcatgggaaaagctgttattgtagcaaccaatatgctggaaagcatgattgttcacccaacaccaacccgagcagaggtatcagacattgccattgctgtccgagagggtgctgatgcagttatgctttctggagaaactgctcatggaaagtttcccttgaaagctgtgaaagtcatgcatacagtttccttaagaactgaagcaaccttggtyggtagcccaatgccaccaaatcttggtcaggctttcaagaaccatatgagcgaaatgtttgctttccatgcaaccatgatgtccaatactctcggtacttcaatagttgttttcactagaactggtttcatggctatccttctgagccattatcgaccttctggcaccatatttgcctttacaaatgaggagagagtacaacagagactagccgtgtaccaaggggtatgtcccatatacatgcagttctcagacgatgctgaagaaacctttgcaaatgccttgtctttgttgcagaagcaagggatggtgaaggaaggagaagaggtggctcttgttcagagtggcaggcagccgatctggcggttccaatccacacacaatatccaggtcaggaaagtttaa

SEQ ID NO: 40 - protein, 580 aa, Vitis vinifera
  misc_feature (67)..(67): Xaa can be any naturally occurring amino acid
MAQVVATRAIQSSILCPSSGSVHERFEKLKPSGFAAKVLAREERRSRRVAWRGGPIAAAKRSVGAEXEVVPVTPEDAKGEEQYHRLRGIQQLGETSVGMWSKPTVRRKTKIVCTIGPSTNTREMIWKLAEAGMNVARLNMSHGDHASHQKVIDLVKEYNAQSKDNVIAIMLDTKASFLGPEVRSGDLPQPIMLKSGQEFTFTIQRGVGTADCVSVNYDDFVNDVEMGDMLLVDGGMMSLMVKSKTGDSVKCEVVDGGELKSRRHLNVRGKSATLPSITEKDWDDIKFGVDNKVDFYAVSFVKDAKVVHELKNYLKSCNADIHVIVKIESADSIPNLHSIITASDGAMVARGDLGAELPIEEVPLLQEEIIRICRSMGKAVIVATNMLESMIVHPTPTRAEVSDIAIAVREGADAVMLSGETAHGKFPLKAVKVMHTVSLRTEATLVGSPMPPNLGQAFKNHMSEMFAFHATMMSNTLGTSIVVFTRTGFMAILLSHYRPSGTIFAFTNEERVQQRLAVYQGVCPIYMQFSDDAEETFANALSLLQKQGMVKEGEEVALVQSGRQPIWRFQSTHNIQVRKV

SEQ ID NO: 41 - DNA, 1734 bp, Glycine max
atggctcaggtagctgcctcacgatccattcaaagcacccctttgtgccccacttctggatctgcacgtgacagagcccaaaacctgttaaagcctccatcttttgcttccaaggtgtttccttttgtagggaacaacaacaaacgctccaaactcagcctcagaggccttcagatcagtgcaaggaaatcagccccttctgaagtcatccccgtgtcacccgaagatgatccaaagattgaggagcatttgcaacatttacgtggggtgcagccatttggtgagaactctgttggtatgtggtctaagcccacgtttaggcgcaaaacgaagatagtgtgtactattggaccttctaccaatacgagggaaatgatttggaagctggctgaggctggcatgaatgttgctcggttgaatatgtctcatggagaccatgcttctcatcagaaagttattgacttggttaaggaatataatgcttctcatggagacaatgtgattgcaatcatgcttgacacaaagggtcctgaagttaggagtggggatttgccacaaccaattacattaatgcctggtcaggaattcacttttactattcagagaggtgttggaactgctgattgtgttagtgtgaactacgatgattttgtcaatgatgtggaagtgggggacatgcttcttgtcgatggtggtatgatgtcaatggttgttaagtctaagacaaaggattctgtgaaatgtgaagttgttgatggaggagagctcaagtcaaggcgacatttgaacgttagaggaaaaagtgcaactctgccttcaatcactgagaaggattgggatgatatcaagtttggagtggataacaaagttgacttttatgctgtttcctttgttaaggatgctgaagttgttcatgagctgaagaattatttgaagagctgtggtgctgatatacatgtcatcgtaaaaatcgaaagtgcagactctataccaaatttgcattcaattatcactgcgtctgatggggctatggttgccagaggagatcttggcgctgagctccctattgaagaggttccacttttgcaggaagagataatcaatttgtgtcgtagcatgggaaaggctgttattgtggcaacaaatatgctagaaagcatgattgttcacccaactccaaccagagcagaggtatcggatatagcaattgctgttcgagaaggttctgatggaataatgttgtctggagaaacagctcatggaaagttcccactaaaagctgtgaaagtaatgcacacagtagcattacggactgaagctactataccaggtggtcaaatgccacctaatattggtccagtgttgaagaaccacatgagtgagatgtttgcataccatgcaaccatgatgtctaacactcttggaacctcaactgttgtcttcactagaacgggtttcatggctgtccttctgagccattatcgtccttcgggcaccatatttgcttttacagatgagaagagggtacaacagaggctggctttgtatcaaggagtctgtccaatatacatggaattctgtgacgattctgaggcaactttcagaagagccttgaatttgctgcagaagcaaggaatggtgaaggaaggagaagaagtagcacttgtacaaagtggtaggcaacccatatggaggttccaatccacgcacaatatccaggttaggaaagtgtaa

SEQ ID NO: 42 - protein, 577 aa, Glycine max
MAQVAASRSIQSTPLCPTSGSARDRAQNLLKPPSFASKVFPFVGNNNKRSKLSLRGLQISARKSAPSEVIPVSPEDDPKIEEHLQHLRGVQPFGENSVGMWSKPTFRRKTKIVCTIGPSTNTREMIWKLAEAGMNVARLNMSHGDHASHQKVIDLVKEYNASHGDNVIAIMLDTKGPEVRSGDLPQPITLMPGQEFTFTIQRGVGTADCVSVNYDDFVNDVEVGDMLLVDGGMMSMVVKSKTKDSVKCEVVDGGELKSRRHLNVRGKSATLPSITEKDWDDIKFGVDNKVDFYAVSFVKDAEVVHELKNYLKSCGADIHVIVKIESADSIPNLHSIITASDGAMVARGDLGAELPIEEVPLLQEEIINLCRSMGKAVIVATNMLESMIVHPTPTRAEVSDIAIAVREGSDGIMLSGETAHGKFPLKAVKVMHTVALRTEATIPGGQMPPNIGPVLKNHMSEMFAYHATMMSNTLGTSTVVFTRTGFMAVLLSHYRPSGTIFAFTDEKRVQQRLALYQGVCPIYMEFCDDSEATFRRALNLLQKQGMVKEGEEVALVQSGRQPIWRFQSTHNIQVRKV

SEQ ID NO: 43 - DNA, 1728 bp, Glycine max
atgtctcaggtagtggccactcgatccattcactcctccctcacgcgccccacctcaggatctgcacaccacagggcccaaacgttgttgaagcctccaacttttgcttccaaattgttcggagcacaaaggaacaacccctccaaagtttgctcccgaagttgcctcgtcaatgcgaggaaatctgcacccgctaaagttgttcccgtgtcacccgaggatgattcaaagattgaggaagagttgcagcacttgcgtggtatgcagcaacttggcgacacttctgttggaatgtggtcaaaacccacgtttaggaggaagacaaaggttgtttgcaccattggtccttctaccaacaccagggaaatgatttggaagctggctgaggctgggatgaatgttgcccgattgaatatgtctcacggagaccatgcttctcatcagaaaattattgatttggttaaagaatataatgctcaatccaaggacaacgtaattgcaattatgcttgataccaagggtcctgaggttaggagtggggatttgccacaaccaatcaatttaacaactgggcaggaattcacttttaccatccggaggggtgttggaactgcagattgtgttagtgtgaactatgacgatttcgtcaatgatgtggatgtgggagacatgcttcttgttgatggtggtatgatgtctttggtggttaagtctaagacagaggattctgtgaaatgtgaagttgttgatggaggagagctcaagtcaaggagacatttgaatgttagaggaaaaagtgcaacactgccttccataactgagaaggattgggatgacatcaaatttggagtggataacaaagttgacttctatgctgtttcttttgttaaggatgcacaagtagttcatgaactgaagaattatttgaaaagctgtgatgctgatatacacgtcattgtaaaaattgaaagtgcagactctataccaaacttgcattcaattattacagcgtctgatggggccatggttgcaagaggagatcttggtgcagaactccctattgaagaggttccacttttgcaggaagaaataatcaccatatgtcgtagcatgggaaaggccgttattgtggcaacaaatatgctggaaagcatgattgttcacccgacaccaaccagagccgaggtatccgatattgcaattgctgttcgagaaggttctgatgcaataatgctttctggggaaactgctcatggaaagttcccactaaaagccgtgaaagtaatgcacaccgtagcattacggacagaagccactatacctggtggtcaaatgccaccaaatattggtcaagtattcaagaaccacatgagtgagatgtttgcttaccatgcaaccatgatgtctaatacccttggaacctcaactgttgtcttcactagatcaggcttcatggctatccttttgagccactatcgaccttcaggcaccatatttgcttttacagatcaaaagaggatacaacagaggttggctttgtatcaaggagtctgtcctatttacatggaattctctgaagatgctgaagagactttcacaagggccttggatttgctgcagaagcaaggaatggtgaaatcaggagaagaagtagcactagtacaaagtggcacgcaacccatatggaggttccaatccactcacaatatccaggtccgaacagtgtaa

SEQ ID NO: 44 - protein, 575 aa, Glycine max
MSQVVATRSIHSSLTRPTSGSAHHRAQTLLKPPTFASKLFGAQRNNPSKVCSRSCLVNARKSAPAKVVPVSPEDDSKIEEELQHLRGMQQLGDTSVGMWSKPTFRRKTKVVCTIGPSTNTREMIWKLAEAGMNVARLNMSHGDHASHQKIIDLVKEYNAQSKDNVIAIMLDTKGPEVRSGDLPQPINLTTGQEFTFTIRRGVGTADCVSVNYDDFVNDVDVGDMLLVDGGMMSLVVKSKTEDSVKCEVVDGGELKSRRHLNVRGKSATLPSITEKDWDDIKFGVDNKVDFYAVSFVKDAQVVHELKNYLKSCDADIHVIVKIESADSIPNLHSIITASDGAMVARGDLGAELPIEEVPLLQEEIITICRSMGKAVIVATNMLESMIVHPTPTRAEVSDIAIAVREGSDAIMLSGETAHGKFPLKAVKVMHTVALRTEATIPGGQMPPNIGQVFKNHMSEMFAYHATMMSNTLGTSTVVFTRSGFMAILLSHYRPSGTIFAFTDQKRIQQRLALYQGVCPIYMEFSEDAEETFTRALDLLQKQGMVKSGEEVALVQSGTQPIWRFQSTHNIQVRTV

SEQ ID NO: 45 - DNA, 1728 bp, Glycine max
atgtctcaggtagtggccactcgatccattcactcctccctcacgcgccccacctcaggatctgcacaccacagggcccaaacgttgttgaagcctccaacttttgcttccaaattgttcggagcacaaaggaacaacccctccaaagtttgctcccgaagttgcctcgtcaatgcgaggaaatctgcacccgctaaagttgttcccgtgtcacccgaggatgattcaaagattgaggaagagttgcagcacttgcgtggtatgcagcaacttggcgacacttctgttggaatgtggtcaaaacccacgtttaggaggaagacaaaggttgtttgcaccattggtccttctaccaacaccagggaaatgatttggaagctggctgagactgggatgaatgttgcccgattgaatatgtctcacggagaccatgcttctcatcagaaaattattgatttggttaaagaatataatgctcaatccaaggacaacgtaattgcaattatgcttgataccaagggtcctgaggttaggagtggggatttgccacaaccaatcaatttaacaactgggcaggaattcacttttaccatccggaggggtgttggaactgcagattgtgttagtgtgaactatgacgatttcgtcaatgatgtggatgtgggagacatgcttcttgttgatggtggtatgatgtctttggtggttaagtctaagacagaggattctgtgaaatgtgaagttgttgatggaggagagctcaagtcaaggagacatttgaatgttagaggaaaaagtgcaacactgccttccataactgagaaggattgggatgacatcaaatttggagtggataacaaagttgacttctatgctgtttcttttgttaaggatgcacaagtagttcatgaactgaagaattatttgaaaagctgtgatgctgatatacacgtcattgtaaaaattgaaagtgcagactctataccaaacttgcattcaattattacagcgtctgatggggccatggttgcaagaggagatcttggtgcagaactccctattgaagaggttccacttttgcaggaagaaataatcaccatatgtcgtagcatgggaaaggccgttattgtggcaacaaatatgctggaaagcatgattgttcacccgacaccaaccagagccgaggtatccgatattgcaattgctgttcgagaaggttctgatgcaataatgctttctggggaaactgctcatggaaagttcccactaaaagccgtgaaagtaatgcacaccgtagcattacggacagaagccactatacctggtggtcaaatgccaccaaatattggtcaagtattcaagaaccacatgagtgagatgtttgcttaccatgcaaccatgatgtctaatacccttggaacctcaactgttgtcttcactagatcaggcttcatggctatccttttgagccactatcgaccttcaggcaccatatttgcttttacagatcaaaagaggatacaacagaggttggctttgtatcaaggagtctgtcctatttacatggaattctctgaagatgctgaagagactttcacaagggccttggatttgctgcagaagcaaggaatggtgaaatcaggagaagaagtagcactagtacaaagtggcacgcaacccatatggaggttccaatccactcacaatatccaggtccgaacagtgtaa

SEQ ID NO: 46 - protein, 575 aa, Glycine max
MSQVVATRSIHSSLTRPTSGSAHHRAQTLLKPPTFASKLFGAQRNNPSKVCSRSCLVNARKSAPAKVVPVSPEDDSKIEEELQHLRGMQQLGDTSVGMWSKPTFRRKTKVVCTIGPSTNTREMIWKLAETGMNVARLNMSHGDHASHQKIIDLVKEYNAQSKDNVIAIMLDTKGPEVRSGDLPQPINLTTGQEFTFTIRRGVGTADCVSVNYDDFVNDVDVGDMLLVDGGMMSLVVKSKTEDSVKCEVVDGGELKSRRHLNVRGKSATLPSITEKDWDDIKFGVDNKVDFYAVSFVKDAQVVHELKNYLKSCDADIHVIVKIESADSIPNLHSIITASDGAMVARGDLGAELPIEEVPLLQEEIITICRSMGKAVIVATNMLESMIVHPTPTRAEVSDIAIAVREGSDAIMLSGETAHGKFPLKAVKVMHTVALRTEATIPGGQMPPNIGQVFKNHMSEMFAYHATMMSNTLGTSTVVFTRSGFMAILLSHYRPSGTIFAFTDQKRIQQRLALYQGVCPIYMEFSEDAEETFTRALDLLQKQGMVKSGEEVALVQSGTQPIWRFQSTHNIQVRTV

SEQ ID NO: 47 - DNA, 1734 bp, Glycine max
atggctcaggtcgtggcttcacgatccattcaaagcaccccattgtgccccacttctggatctgcacgtgacagaacccaaaacctgttaaagcctccatcttttgcttccaaggtgttcccattgggagagaacaataacaaacgctccaaactcaggctcagaagccttcagatcagtgcaaggaaatctgccccttctgaagtcatccccatgtcacccgaagatgatccaaaaattgaggagcatttgcaacatttacgtggggtgcagccatttggtgagaactctgttggaatgtggtctaagccaacgtttaggcgcaagacgaagatagtgtgcaccattggaccttctaccaatacgagggaaatgatttggaagctggctgaggctggcatgaatgttgctcggttgaatatgtctcatggggaccatgcttctcatcagaaagttattgacttggttaaggagtataatgcttctcatgaagacaatgtgattgcaatcatgcttgacacaaagggtcctgaagttaggagtggggatttgccacaaccaattacattaatgcctgggcaggaattcacttttactattcagagaggtgttggaactgctgattgtgttagtgtgaactacgatgattttgtgaatgatgtggaaatgggggacatgcttcttgtcgatggtggtatgatgtctatggttgttaagtctaagacagaggattctgtgaaatgtgaagttgttgatggaggagagctcaagtcaaggcgacatttgaacgttagaggaaaaagtgcaacactaccttcaatcactgagaaggattgggatgatatcaagtttggagtggataacaaagttgacttctatgctgtttcctttgttaaggatgctgaagttgttcatgaactgaagaattatttgaagagctgcggtgctgatatacatgtcatcgtaaaaattgaaagtgcagactctataccaaatttgcattcaattatcactgcatctgatggggcaatggttgccagaggagatcttggcgctgagctccctattgaagaggttccacttttgcaggaagagataatcaacttgtgtcgtagcatgggaaaggctgttattgtggcaacaaatatgctagaaagcatgattgttcacccaactccaaccagagcagaggtatcagatatagcaattgcagttcgtgaaggttctgatggaataatgttgtctggagaaacagctcatggaaagttcccactaaaagctgtgcaagtaatgcacacagtagcattgcggacagaagccactataccaggtggtaaaatgccacctaacattggtcaagtgttgaagaaccacatgagtgagatgtttgcataccatgcaaccatgatgtctaacacccttggaacctcaactgttgtcttcactagaacgggtttcatggctgtccttctgagtcactatcgtccttctgggaccatatttgcttttacagatgagaagagggtacaacagaggctggctttgtatcaaggagtctgtcccatatacatggaattctgtgatgattctgaggcaacttttagaagagccttggatttgctgcagaagcaagcaatggtgaaggaaggagaagaagtagcacttgtacaaagtggtaggcaacccatatggaggttccaatccacgcacaatatccaggttaggaaagtgtaa

SEQ ID NO: 48 - protein, 577 aa, Glycine max
MAQVVASRSIQSTPLCPTSGSARDRTQNLLKPPSFASKVFPLGENNNKRSKLRLRSLQISARKSAPSEVIPMSPEDDPKIEEHLQHLRGVQPFGENSVGMWSKPTFRRKTKIVCTIGPSTNTREMIWKLAEAGMNVARLNMSHGDHASHQKVIDLVKEYNASHEDNVIAIMLDTKGPEVRSGDLPQPITLMPGQEFTFTIQRGVGTADCVSVNYDDFVNDVEMGDMLLVDGGMMSMVVKSKTEDSVKCEVVDGGELKSRRHLNVRGKSATLPSITEKDWDDIKFGVDNKVDFYAVSFVKDAEVVHELKNYLKSCGADIHVIVKIESADSIPNLHSIITASDGAMVARGDLGAELPIEEVPLLQEEIINLCRSMGKAVIVATNMLESMIVHPTPTRAEVSDIAIAVREGSDGIMLSGETAHGKFPLKAVQVMHTVALRTEATIPGGKMPPNIGQVLKNHMSEMFAYHATMMSNTLGTSTVVFTRTGFMAVLLSHYRPSGTIFAFTDEKRVQQRLALYQGVCPIYMEFCDDSEATFRRALDLLQKQAMVKEGEEVALVQSGRQPIWRFQSTHNIQVRKV

SEQ ID NO: 49 - DNA, 1728 bp, Glycine max
atgtctcatgtagtggtcactcgatccattcacacctccctcacgcgccccacctcaggatctgcacaccacagagcccaaacgttgttgaagcctccaacttttgcttccaaagtgttcccacaacaaaggaacaacccctccaaagtttgctcccgaagttgcctcgtcaatgcgaggaaatctgcacccactgaagttgttcccgtctcacccgaggatgattcaaagattgaggaagagttgcagcactcgcgtggtatgcggcaacttggtgacacttctgttggaatgtggtcaaaacccacttttaggaggaagacaaaggttgtttgcaccattggtccttctaccaacaccagggaaatgatttggaagctggctgaggctgggatgaatgttgcccgattgaatatgtctcatggagaccatgcttctcatcagaaaattattgatttggttaaagaatataatgctcaatccaaggacaatgtaattgcaattatgcttgataccaagggtcctgaggttaggagtggggatttgccacaaccaatcaatttaacaactgggcaggaattcacatttaccatccggaggggtgttggaactgcagattgtgttagtgtgaactatgacgatttcgtcaatgatgtggatgtgggagacatgcttcttgttgatggtggtatgatgtctttggtggttaagtctaagacagaggattctgtgaaatgtgaagttgttgatggaggagagctcaagtcaaggagacatttgaacgttagaggaaaaagtgcaacactgccttcaatcactgagaaggattgggatgacatcaaatttggagtggataacaaagttgacttctatgctgtttcttttgttaaggatgcacaagtagttcatgaactgaagaattatttgaaaagctgtgatgctgatatacacgtcattgtaaaaattgaaagtgcagactctataccaaacttgcattcaattattacagcgtctgatggggccatggttgcaagaggagatcttggtgcagaactccctattgaagaggttccacttttgcaggaagaaataatcaccatatgtcgtagcatgggaaaggccgttattgtggcaacaaatatgctggaaagcatgattgttcacccgacaccaaccagagccgaggtatccgatattgcaattgctgttcgagaaggttctgatgcaataatgctttctggggaaactgctcatggaaagttcccactaaaagccgtgaaagtaatgcacaccgtagcattacggacagaagccactatacctggtggtcaaatgccaccaaatattggtcaagtattcaagaaccacatgagtgagatgtttgcttaccatgcaaccatgatgtctaatacccttggaacctcaactgttgtcttcactagatcaggcttcatggctatccttttgagccactatcgaccttcaggcaccatatttgcttttacagatcaaaagaggatacaacagaggttggctttgtatcaaggagtctgtcctatttacatggaattctctgaagatgctgaagagactttcacaagggccttggatttgctgcagaagcaaggaatggtgaaatcaggagaagaagtagcactagtacaaagtggcacgcaacccatatggaggttccaatccactcacaatatccaggtccgaacagtgtaa

SEQ ID NO: 50 - protein, 575 aa, Glycine max
MSHVVVTRSIHTSLTRPTSGSAHHRAQTLLKPPTFASKVFPQQRNNPSKVCSRSCLVNARKSAPTEVVPVSPEDDSKIEEELQHSRGMRQLGDTSVGMWSKPTFRRKTKVVCTIGPSTNTREMIWKLAEAGMNVARLNMSHGDHASHQKIIDLVKEYNAQSKDNVIAIMLDTKGPEVRSGDLPQPINLTTGQEFTFTIRRGVGTADCVSVNYDDFVNDVDVGDMLLVDGGMMSLVVKSKTEDSVKCEVVDGGELKSRRHLNVRGKSATLPSITEKDWDDIKFGVDNKVDFYAVSFVKDAQVVHELKNYLKSCDADIHVIVKIESADSIPNLHSIITASDGAMVARGDLGAELPIEEVPLLQEEIITICRSMGKAVIVATNMLESMIVHPTPTRAEVSDIAIAVREGSDAIMLSGETAHGKFPLKAVKVMHTVALRTEATIPGGQMPPNIGQVFKNHMSEMFAYHATMMSNTLGTSTVVFTRSGFMAILLSHYRPSGTIFAFTDQKRIQQRLALYQGVCPIYMEFSEDAEETFTRALDLLQKQGMVKSGEEVALVQSGTQPIWRFQSTHNIQVRTV

SEQ ID NO: 51 - DNA, 1728 bp, Glycine max
atgtctcatgtagtggtcactcgatccattcacacctccctcacgcgccccacctcaggatctgcacaccacagagcccaaacgttgttgaagcctccaacttttgcttccaaagtgttcccacaacaaaggaacaacccctccaaagtttgctcccgaagttgcctcgtcaatgcgaggaaatctgcacccgctaaagttgttcccgtctcacccgaggatgattcaaagattgaggaagagttgcagcactcgcgtggtatgcagcaacttggcgacacttctgttggaatgtggtcaaaacccacgtttaggaggaagacaaaggttgtttgcaccattggtccttctaccaacaccagggaaatgatttggaagctggctgaggctgggatgaatgttgcccgattgaatatgtctcacggagaccatgcttctcatcagaaaattattgatttggttaaagaatataatgctcaatccaaggacaacgtaattgcaattatgcttgataccaagggtcctgaggttaggagtggggatttgccacaaccaatcaatttaacaactgggcaggaattcacatttaccatccggaggggtgttggaactgcagattgtgttagtgtgaactatgacgatttcgtcaatgatgtggatgtgggagacatgcttcttgttgatggtggtatgatgtctttggtggttaagtctaagacagaggattctgtgaaatgtgaagttgttgatggaggagagctcaagtcaaggagacatttgaatgttagaggaaaaagtgcaacactgccttccataactgagaaggattgggatgacatcaaatttggagtggataacaaagttgacttctatgctgtttcttttgttaaggatgcacaagtagttcatgaactgaagaattatttgaaaagctgtgatgctgatatacacgtcattgtaaaaattgaaagtgcagactctataccaaacttgcattcaattattacagcgtctgatggggccatggttgcaagaggagatcttggtgcagaactccctattgaagaggttccacttttgcaggaagaaataatcaccatatgtcgtagcatgggaaaggccgttattgtggcaacaaatatgctggaaagcatgattgttcacccgacaccaaccagagccgaggtatccgatattgcaattgctgttcgagaaggttctgatgcaataatgctttctggggaaactgctcatggaaagttcccactaaaagccgtgaaagtaatgcacaccgtagcattacggacagaagccactatacctggtggtcaaatgccaccaaatattggtcaagtattcaagaaccacatgagtgagatgtttgcttaccatgcaaccatgatgtctaatacccttggaacctcaactgttgtcttcactagatcaggcttcatggctatccttttgagccactatcgaccttcaggcaccatatttgcttttacagatcaaaagaggatacaacagaggttggctttgtatcaaggagtctgtcctatttacatggaattctctgaagatgctgaagagactttcacaagggccttggatttgctgcagaagcaaggaatggtgaaatcaggagaagaagtagcactagtacaaagtggcacgcaacccatatggaggttccaatccactcacaatatccaggtccgaacagtgtaa

SEQ ID NO: 52 - protein, 575 aa, Glycine max
MSHVVVTRSIHTSLTRPTSGSAHHRAQTLLKPPTFASKVFPQQRNNPSKVCSRSCLVNARKSAPAKVVPVSPEDDSKIEEELQHSRGMQQLGDTSVGMWSKPTFRRKTKVVCTIGPSTNTREMIWKLAEAGMNVARLNMSHGDHASHQKIIDLVKEYNAQSKDNVIAIMLDTKGPEVRSGDLPQPINLTTGQEFTFTIRRGVGTADCVSVNYDDFVNDVDVGDMLLVDGGMMSLVVKSKTEDSVKCEVVDGGELKSRRHLNVRGKSATLPSITEKDWDDIKFGVDNKVDFYAVSFVKDAQVVHELKNYLKSCDADIHVIVKIESADSIPNLHSIITASDGAMVARGDLGAELPIEEVPLLQEEIITICRSMGKAVIVATNMLESMIVHPTPTRAEVSDIAIAVREGSDAIMLSGETAHGKFPLKAVKVMHTVALRTEATIPGGQMPPNIGQVFKNHMSEMFAYHATMMSNTLGTSTVVFTRSGFMAILLSHYRPSGTIFAFTDQKRIQQRLALYQGVCPIYMEFSEDAEETFTRALDLLQKQGMVKSGEEVALVQSGTQPIWRFQSTHNIQVRTV

SEQ ID NO: 53 - DNA, 1737 bp, Glycine max
atgtctcatgtagtggtcactcgatccattcacacctccctcacgcgccccacctcaggatctgcacaccacagagcccaaacgttgttgaagcctccaacttttgcttccaaagtgttcccacaacaaaggaacaacccctccaaagtttgctcccgaagttgcctcgtcaatgcgaggaaatctgcacccactgaagttgttcccgtctcacccgaggatgattcaaagattgaggaagagttgcagcactcgcgtggtatgcggcaacttggtgacacttctgttggaatgtggtcaaaacccacttttaggaggaagacaaaggttgtttgcaccattggtccttctaccaacaccagggaaatgatttggaagctggctgaggctgggatgaatgttgcccgattgaatatgtctcatggagaccatgcttctcatcagaaaattattgatttggttaaagaatataatgctcaatccaaggacaatgtaattgcaattatgcttgataccaagggtcctgaggttaggagtggggatttgccacaaccaatcaatttaacaactgggcaggaattcacatttaccatccggaggggtgttggaactgcagattgtgttagtgtgaactatgacgatttcgtcaatgatgtggatgtgggggacatgcttcttgttgatggtggtatgatgtctttggtggttaagtctaagacagaggattctgtgaaatgtgaagttgttgatggaggagagctcaagtcaaggagacatttgaatgttagaggaaaaagtgcaacacttccttccatcactgagaaggattgggatgacatcaaatttggagtggataacaaagttgacttctatgctgtttcttttgttaaagatgcacaagtagttcatgaactgaagaattatttgaaaagctgtgatgctgatatacatgtcattgtaaaaattgaaagtgcagactctataccaaacttgcattcaattattacagcgtctgatggggccatggttgcaagaggagatcttggtgcagagctccctattgaagaggttccacttttgcaggaagagataataagcatatgccgtagcatgggaaaggctgttattgtggcaacaaatatgctggaaagcatgattgttcacccaacaccaaccagagccgaggtatccgatattgcaattgctgttcgagaaggttctgatgcaataatgctttctggggaaactgctcacggaaagttcccgctaaaagccgtgaaagtaatgcataccgtagcattacggacagaagccaccatacccggtggtcagatgccaccaaatattggtcaagtattcaagaaccacatgagtgagatgtttgcttaccatgcaaccatgatgtctaataccctcggaacctcaactgttgtcttcactagatcaggcttcatggctatccttttgagccactatcgaccttcaggcaccatatttgcttttacagatcaaaagaggatacaacagaggttggctttgtatcaaggagtctgtcctatttacatggaattctctgaagatgctgaagagactttcacaagggccttggatttgctgcagaagcaaggaatggtgaaatcaggagaagaagtagcactagtacaaagtggcaggcaacccatatggaggttccaatccactcacaatatccaggtccgaacagtgaaaacaaaataa

SEQ ID NO: 54 - protein, 578 aa, Glycine max
MSHVVVTRSIHTSLTRPTSGSAHHRAQTLLKPPTFASKVFPQQRNNPSKVCSRSCLVNARKSAPTEVVPVSPEDDSKIEEELQHSRGMRQLGDTSVGMWSKPTFRRKTKVVCTIGPSTNTREMIWKLAEAGMNVARLNMSHGDHASHQKIIDLVKEYNAQSKDNVIAIMLDTKGPEVRSGDLPQPINLTTGQEFTFTIRRGVGTADCVSVNYDDFVNDVDVGDMLLVDGGMMSLVVKSKTEDSVKCEVVDGGELKSRRHLNVRGKSATLPSITEKDWDDIKFGVDNKVDFYAVSFVKDAQVVHELKNYLKSCDADIHVIVKIESADSIPNLHSIITASDGAMVARGDLGAELPIEEVPLLQEEIISICRSMGKAVIVATNMLESMIVHPTPTRAEVSDIAIAVREGSDAIMLSGETAHGKFPLKAVKVMHTVALRTEATIPGGQMPPNIGQVFKNHMSEMFAYHATMMSNTLGTSTVVFTRSGFMAILLSHYRPSGTIFAFTDQKRIQQRLALYQGVCPIYMEFSEDAEETFTRALDLLQKQGMVKSGEEVALVQSGRQPIWRFQSTHNIQVRTVKTK

SEQ ID NO: 55 - DNA, 1704 bp, Arabidopsis thaliana
atggctgcttatggtcaaatctcctcgggaatgactgtagatcctcaggttctctcttcctccagaaacattggagtttccctatcacctctccggagaacactaatcggcgccggagttaggtctactagtatctctctccgtcaatgttctctctccgttagatcgattaaaatctccgaagatagccgcaaacctaaagcttatgcagagaacggtgcttttgatgtgggagttttggattcttcatcatatagattggctgattcaagaacaagtagtaatgattcaaggaggaagactaagattgtgtgtacgattggaccgtcttcgagttctagggaaatgatttggaaactcgcggaagctggaatgaatgtggctcgtttgaatatgtctcatggtgatcatgcttctcatcagataactattgatttagttaaggagtataattctttgtttgttgacaaagctattgctattatgttggatacaaagggtcctgaggttcgaagcggggatgtaccgcagccgatatttcttgaagagggtcaagagtttaactttactatcaagagaggtgtttcgcttaaagacactgttagtgtaaattatgatgattttgtgaacgatgttgaagttggggatatacttttggtggatggtggaatgatgtcgttagctgttaaatcaaagacgagtgatttggtgaagtgtgtggttattgatggtggagagcttcaatctagacgtcacttgaatgttcgaggaaagagtgcgactcttccatccattacagacaaagattgggaagacataaaatttggagtggacaaccaagtcgatttctacgccgtctcctttgttaaggatgctaaagttgtccatgagttgaagaactatctcaaaaacatatcggtgattgtgaaaattgaaagtgcagactctataaagaatcttccttctatcatatctgcttgtgatggggcaatggttgctcgtggagatcttggagctgaacttcccattgaagaggtcccgttgttacaggaagaaataatcagaaggtgtagaagcattcataaaccagtgattgttgccacaaacatgctagagagtatgattaatcatccaacgcctacaagagctgaagtctctgacattgcaattgcagtacgtgaaggcgcagatgctatcatgctttctggtgaaaccgcacatggaaagtttccgctgaaagctgttaacgtaatgcatactgtggcgttgagaaccgaggcaagtctacctgtcagaacctcggcatcccgtaccactgcttacaagggtcacatgggccaaatgtttgcttttcatgcttctataatggcaaatacactgagctcaccgctaattgtatttacgagaaccggatccatggcagtgcttctaagccactaccgcccatctgcaacaattttcgccttcacaaaccagagaagaataatgcaaaggcttgctctttatcaaggtgtcatgcctatatatatggagttctcggatgatgcagaagatacatatgcccggtctctcaaactcttacaggacgagaatatgctcaaggaaggacaacatgtaactcttgtccaaagtggctcgcaacccatttggcgtgaagaatcaacacatctcatacaagtccgtaagataaagataggtggatga

SEQ ID NO: 56 - protein, 567 aa, Arabidopsis thaliana
MAAYGQISSGMTVDPQVLSSSRNIGVSLSPLRRTLIGAGVRSTSISLRQCSLSVRSIKISEDSRKPKAYAENGAFDVGVLDSSSYRLADSRTSSNDSRRKTKIVCTIGPSSSSREMIWKLAEAGMNVARLNMSHGDHASHQITIDLVKEYNSLFVDKAIAIMLDTKGPEVRSGDVPQPIFLEEGQEFNFTIKRGVSLKDTVSVNYDDFVNDVEVGDILLVDGGMMSLAVKSKTSDLVKCVVIDGGELQSRRHLNVRGKSATLPSITDKDWEDIKFGVDNQVDFYAVSFVKDAKVVHELKNYLKNISVIVKIESADSIKNLPSIISACDGAMVARGDLGAELPIEEVPLLQEEIIRRCRSIHKPVIVATNMLESMINHPTPTRAEVSDIAIAVREGADAIMLSGETAHGKFPLKAVNVMHTVALRTEASLPVRTSASRTTAYKGHMGQMFAFHASIMANTLSSPLIVFTRTGSMAVLLSHYRPSATIFAFTNQRRIMQRLALYQGVMPIYMEFSDDAEDTYARSLKLLQDENMLKEGQHVTLVQSGSQPIWREESTHLIQVRKIKIGG

SEQ ID NO: 57 - DNA, 1713 bp, Arabidopsis lyrata
atggcggctttaggtcaaatctcctcgggaatgactgtagatcctcaggttctctcctcctccagaaacattggagtttcactatcaccactccggcgaacactaatcggcgccggaggtaggtctactatctccctccgtcaatgttctctctccgttagatcgattaaaatcaccgaagataaccgcaaagttaaagcttatgctgataacggcgctttcgatatggattcttcagttgattcatcgtatagattggcggattcaagaacaagtaataatgattcgaggaggaagacgaagattgtgtgtacgattggaccgtcatcgagttctcgggagatgatttggaagctcgcggaagcgggaatgaatgtggctcgtttgaatatgtctcatggtgatcatgcttctcatcagaagactattgatttagttaaggaatataattctttgtttgttgacaaagctattgctattatgttggatacaaagggtcctgaggttcgaagcggggatgtacctcagccgatatttcttgaagagggtcaagagtttaattttactatcaagagaggtgtttcgcttaaagacactgttagtgtaaattatgatgattttgttaatgatgttgaagttggggacatacttttggtggatggcggaatgatgtcattagctgttaaatcaaagacgagtgatttggtgaagtgtgtggttattgatggtggagagcttcaatctagacgtcacttgaatgttcgtggaaagagtgcgactcttccttccataacagacaaagattgggaggacataaaatttggagtggacaaccaggtcgatttctatgccgtctcctttgttaaggatgctaaagttgtccatgagttgaagaactatctcaaaacctgcagtgcggatatatcggtaatcgtgaagattgaaagcgctgactctataaagaatcttccttctatcatatctgcttgtgatggggcaatggttgctcgtggagatcttggagctgaacttcccattgaagaggtcccattgttacaggaagagataatcagaaggtgtagaagcatccataaaccagtgattgtcgccacaaacatgctagagagtatgattaatcatccaacgcctacaagagctgaagtctctgacattgctattgcagtacgtgaaggtgcagatgctatcatgctttctggtgaaaccgcacacggaaagtttccgctcaaagctgttaacgtaatgcatactgtggcgttgagaactgaggcaagtctacctgtcagaacctctgcaacccgtaccactgcttacaagggtcacatgggccaaatgtttgcttttcatgcttctataatggcaaatacactgagctcaccgctaattgtattcacgagaaccggatccatggcagtgcttctaagccattaccgcccatccgcaacaattttcgcctttacaaaccagagaaggataatgcaaaggttggctctttatcaaggtgtcatgcctatatatatggagttctcggatgatgcagaagatacatatgcccggtctctgaaactcttacaggacgagcatatgctgaaggaaggacaacatgtaactcttgtccaaagcggctcgcaacccatttggcgtgaagaatcaacacatctcatacaagtccgtaagacaaatataggtggatga

SEQ ID NO: 58 - protein, 570 aa, Arabidopsis lyrata
MAALGQISSGMTVDPQVLSSSRNIGVSLSPLRRTLIGAGGRSTISLRQCSLSVRSIKITEDNRKVKAYADNGAFDMDSSVDSSYRLADSRTSNNDSRRKTKIVCTIGPSSSSREMIWKLAEAGMNVARLNMSHGDHASHQKTIDLVKEYNSLFVDKAIAIMLDTKGPEVRSGDVPQPIFLEEGQEFNFTIKRGVSLKDTVSVNYDDFVNDVEVGDILLVDGGMMSLAVKSKTSDLVKCVVIDGGELQSRRHLNVRGKSATLPSITDKDWEDIKFGVDNQVDFYAVSFVKDAKVVHELKNYLKTCSADISVIVKIESADSIKNLPSIISACDGAMVARGDLGAELPIEEVPLLQEEIIRRCRSIHKPVIVATNMLESMINHPTPTRAEVSDIAIAVREGADAIMLSGETAHGKFPLKAVNVMHTVALRTEASLPVRTSATRTTAYKGHMGQMFAFHASIMANTLSSPLIVFTRTGSMAVLLSHYRPSATIFAFTNQRRIMQRLALYQGVMPIYMEFSDDAEDTYARSLKLLQDEHMLKEGQHVTLVQSGSQPIWREESTHLIQVRKTNIGG

SEQ ID NO: 59 - DNA, 1716 bp, Brassica napus
atggcggcgacaggtcaaatctcgacgagaatgacggtggatcgcactctgtcctcctccaggaacgctggactttccctttcaccatcaccccagagaacgctaatcggcgtcgccggtaggtctggtatcgctcaccgtcaactgtctctctccgtcagagcgattaacaccaatgaagatagccggaaagtcaaggtttatgcagagaacggcgctttcgatttgggagtgatggatccttcagtggagccatataaatttgcggagccaagaacaagtcataatgattcgaggaggaaaaccaagattgtgtgtaccattggaccttcctctagctctcgtgaaatgatttggaaactcgcggaagcaggaatgaatgtggctcgtttgaatatgtctcatggagatcatgcttctcatcagattactattgatcttgttaaagaatacaactctctctttgttgacaaagccattgctatcatgttggatacaaagggtcctgaggttcgaagtggggatgtacctcagccgatcttccttgaagagggtcaagagtttaattttaccatcaagagaggtgtctcaatgaaagacaccgtcagtgtcaactatgatgattttgttaacgatgtcgaagttggagacatacttttggtcgatggtggaatgatgtcactagctgttaagtccaagacgagcgatttggtgaaatgtgtagttattgacggtggagagcttcaatctagacgtcacttgaatgttcgaggaaagagtgctactcttccttccataacagacaaagattgggaagacatcaaatttggagtggacaaccaagttgatttctatgctgtctcttttgttaaggatgctaaagttgtccatgagttgaaaaactacctcaaaagctgcagtgcggatatctctgtgattgtgaaaattgaaagcgcagattctataaagaatcttccttccattatatctgcttgtgatggggcaatggttgctcgtggagatcttggagctgaacttcccattgaggaggttcccttgttacaggaggagataatcacaaggtgtaggaacattcataaaccagtgattgtcgccacaaacatgctagagagtatgattaatcatccaacacctacaagagctgaagtatctgacattgcaattgcagtgcgcgaaggagcagatgcaatcatgctttctggtgaaaccgcacacggaaagtttcctctgaaagctgttaacgtgatgcataccgtggctttgagaactgaggctagtctacctgttagaacctcagcaatccggaccactgcttacaagggtcgcatgggccaaatgtttgctttccatgcttctataatggcgaatacactgaacacaccgatcattgtgttcacaagaactggatccatggcagtgcttctgagtcactaccgcccgtcctcaacaattttcgccttcacaaaccagaggagaataatgcaaaggctggctctttaccaaggtgtcatgcctatatacatggagttttctgatgatgcagaagatacatatgcccgttccttgaaactcctacaggacgagaatatgcttaaggaaggacaacatgtaactcttgtccaaagtggtgcgcaacccatttggcgtgaagaatcaacacatctcatacaagtccgtaagattaagataggttga

SEQ ID NO: 60 - protein, 571 aa, Brassica napus
MAATGQISTRMTVDRTLSSSRNAGLSLSPSPQRTLIGVAGRSGIAHRQLSLSVRAINTNEDSRKVKVYAENGAFDLGVMDPSVEPYKFAEPRTSHNDSRRKTKIVCTIGPSSSSREMIWKLAEAGMNVARLNMSHGDHASHQITIDLVKEYNSLFVDKAIAIMLDTKGPEVRSGDVPQPIFLEEGQEFNFTIKRGVSMKDTVSVNYDDFVNDVEVGDILLVDGGMMSLAVKSKTSDLVKCVVIDGGELQSRRHLNVRGKSATLPSITDKDWEDIKFGVDNQVDFYAVSFVKDAKVVHELKNYLKSCSADISVIVKIESADSIKNLPSIISACDGAMVARGDLGAELPIEEVPLLQEEIITRCRNIHKPVIVATNMLESMINHPTPTRAEVSDIAIAVREGADAIMLSGETAHGKFPLKAVNVMHTVALRTEASLPVRTSAIRTTAYKGRMGQMFAFHASIMANTLNTPIIVFTRTGSMAVLLSHYRPSSTIFAFTNQRRIMQRLALYQGVMPIYMEFSDDAEDTYARSLKLLQDENMLKEGQHVTLVQSGAQPIWREESTHLIQVRKIKIG

SEQ ID NO: 61 - DNA, 1647 bp, Zea mays
atggccaccgctgcccgctccctccacctccctggtccgacgcctcctaaatcctctaccccctcctcccaccgcctccctgtcccttgccacctccgcccccaacgccgccgcctggccgcctcgtcctccgacctcacctccttccccgaaccgccccccgccaccgaggcggagctgcgggagaacgggttccggagcacgcgccgcaccaagctcgtctgcaccgtgggccccgccacctgcggcgccgccgagctcgaggcgctcgccgtcggcggcatgaacgtcgcgcgggtcaacatgtgccacggcgaccgcgagtggcaccgcgaagtcatccgcgctgtccggaggctcaacgacgagaaagggttcgcggtcgccgtgatgatggacaccgagggcagcgagatccacatgggcgaccttggcggagcgccctccgcgaaggccgaggatggagaagtttggacatttagcgttagatcttctgacacgtcgctcccagatcgaatcattcatgtgaactatgatggctttgctgaagatgtcaaagctggtgatgaactatttgcggacggtggaatggccaggtttgaggtgattgaaaagttagggccagatgtgaagtgccgctgcactgatcctggtctgttgctgccacgggctaatcttactatatggcgcgatggcagtgtagtgcgagagaggaacgctatgcttcctacaatttcgtcgaaggattggattgacatagattttggaattgctgaaggtgttgatttcatcgccgtgtcatttgtcaagtctgctgaagtaattaatcatttgaagagttacattgctgcgaggagccgtggaagtgatataggggtcattgcaaagattgagagcatcgatgctttgaagaacctggaggagattatccgtgcatcagatggagtaatggtagccagaggggacttgggggcacaaatccccctggaacaggtcccttccatacagcaaagaatagttagaatgtgcagacagctcaacaagccagtcattgttgcttctcagcttctggaatcaatgatcgagtatcctacacctactagggctgaggttgctgatgtttctgaggcagtccgccagcgtgcagatgctctcatgctttctggtgagtcggccatggggaggtacccagagaaggctctcagcgtccttaggagtgttagcctgaggatcgaaaggtggtggagagaggagaagcgccaagaggcactggagcttcaaggtgtctcgtcttccttttctgacaagatatcagaggaaatatgcaattcagcagctaaaatggccaacaacttaggagtcgacgccgttttcgtttacacgaaggatggctacatgggctccctgctctcacgatgccgccccgactgcccgatcttcgccttcacgtcttcgacgtctgtcaggagacggctgaacctccagtggggcctcatcccgttccggctgagcgagtccgacgacatggagagcaacctcaaccgcaccttctccctgctgaaggccaggggcatggtgcagtctggagacctggtgatcgcgctctccgacatgctgcagtccatccaagtggtgaatgtgccctag

SEQ ID NO: 62 - protein, 548 aa, Zea mays
MATAARSLHLPGPTPPKSSTPSSHRLPVPCHLRPQRRRLAASSSDLTSFPEPPPATEAELRENGFRSTRRTKLVCTVGPATCGAAELEALAVGGMNVARVNMCHGDREWHREVIRAVRRLNDEKGFAVAVMMDTEGSEIHMGDLGGAPSAKAEDGEVWTFSVRSSDTSLPDRIIHVNYDGFAEDVKAGDELFADGGMARFEVIEKLGPDVKCRCTDPGLLLPRANLTIWRDGSVVRERNAMLPTISSKDWIDIDFGIAEGVDFIAVSFVKSAEVINHLKSYIAARSRGSDIGVIAKIESIDALKNLEEIIRASDGVMVARGDLGAQIPLEQVPSIQQRIVRMCRQLNKPVIVASQLLESMIEYPTPTRAEVADVSEAVRQRADALMLSGESAMGRYPEKALSVLRSVSLRIERWWREEKRQEALELQGVSSSFSDKISEEICNSAAKMANNLGVDAVFVYTKDGYMGSLLSRCRPDCPIFAFTSSTSVRRRLNLQWGLIPFRLSESDDMESNLNRTFSLLKARGMVQSGDLVIALSDMLQSIQVVNVP

SEQ ID NO: 63 - DNA, 1722 bp, Helianthus annuus
atggcacaatctctcaatttcttcgtatcaacctctccatccccagacttttattcccactccaaactcgcccttcaaaccccccatcatcatcatcgactcctcttccccttcaccaacaaacccccccttcgtttatgcgcatctgccgacaacggtgtatccgccgtgacaaccgattacgtcacccaaaacaacggggtatcagtatctgcgggttcgatcgaggtggatgcggtgacggaggcagagctgaaggagaacgggttcaggagcacgcggaggacgaagctggtgtgcacgatcggaccggcgacatgtgggttcgagcagctggaggcgttggctgttggggggatgaatgtggctcggattaatatgtgtcatgggacacgtgagtggcacaagagcgtgattgataaagtgaggaggttgaatgaggagaaagggtatgctgttgctatcatgatggatactgaaggtagtgagattcatatgggggatcttggtggtgcttcttctgctaaagctgaggatggacaagtatggactttcagtgttcgggcttatgatactcttcgtcctgagcgctgcatcactgtgaactatgatggttttgcagaagatgttaaagttggggatgaactccttgttgatggtggaatggtgaggtttgaggtgatagaaaagattggtccagatgtcaagtgcttgtgtactgaccccggacttttgctacctcgggctaacttgactttctggcgagatggtagcttagttcgtgaacgtaatgccatgcttccaacaatttcttcaaaggattggttggatattgattttgggattgcagaaggtgttgatttcattgcagtatcatttgtcaagtcagcagaagtgattaatcatcttaaaagttacatcaaagcacggtctcgtgatggggatatagcggtaatttcaaagatcgagagcatcgactcattgaaaaacttggaggagataattctagcttctgacggagccatggtagccagaggggatctgggtgcacaaatcccactggaacaggttccgttggctcaacagaagatcgttgaagtgtgcagacagttgaacaaaccagtaatagttgcttctcaactactagaatctatgatcgagtacccgacacctaccagagctgaagtggctgatgtttctgaagcagtgagacaacgagctgatgctcttatgctgtctggtgagtcggccatgggtcagttccctgacaaggctttgaccgtcttgagaagtgttagtttaagaattgagaaatggtggagggaagagaaacgtcatgaagttatggatcttcctgatatcgcttcttctttctctgatagtatttctgaggaaatctgcaattctgctgccaaaatggctaataacttggaggtagatgcactttttgtctacacgaaagacggacacatggcatctctcttgtcgcgtagccgcccggattgccccattttcgctttcacaaccgatacattggttagaagacgtctaaatctacaatggggtctaattccgttccgccttgacttctcagacgacatggaaaccaatctcaacaagactttctctctcctcaaggctcgaggtatgatcaaatctggcgatcttgttattgctgtatctgatatgttgcagtcgattcaagttatgaatgtgccataa

SEQ ID NO: 64 - protein, 573 aa, Helianthus annuus
MAQSLNFFVSTSPSPDFYSHSKLALQTPHHHHRLLFPFTNKPPLRLCASADNGVSAVTTDYVTQNNGVSVSAGSIEVDAVTEAELKENGFRSTRRTKLVCTIGPATCGFEQLEALAVGGMNVARINMCHGTREWHKSVIDKVRRLNEEKGYAVAIMMDTEGSEIHMGDLGGASSAKAEDGQVWTFSVRAYDTLRPERCITVNYDGFAEDVKVGDELLVDGGMVRFEVIEKIGPDVKCLCTDPGLLLPRANLTFWRDGSLVRERNAMLPTISSKDWLDIDFGIAEGVDFIAVSFVKSAEVINHLKSYIKARSRDGDIAVISKIESIDSLKNLEEIILASDGAMVARGDLGAQIPLEQVPLAQQKIVEVCRQLNKPVIVASQLLESMIEYPTPTRAEVADVSEAVRQRADALMLSGESAMGQFPDKALTVLRSVSLRIEKWWREEKRHEVMDLPDIASSFSDSISEEICNSAAKMANNLEVDALFVYTKDGHMASLLSRSRPDCPIFAFTTDTLVRRRLNLQWGLIPFRLDFSDDMETNLNKTFSLLKARGMIKSGDLVIAVSDMLQSIQVMNVP

SEQ ID NO: 65 - DNA, 1722 bp, Artificial sequence
  Codon optimized HaPK001 for expression in Zea mays
atggcccagagcctgaacttcttcgtgagcaccagcccgagcccggacttctacagccacagcaagctggccctgcagaccccgcaccaccaccacaggctgctgttcccgttcaccaacaagccgccgctgaggctgtgcgccagcgccgacaacggcgtgagcgccgtgaccaccgactacgtgacccagaacaacggcgtgagcgtgagcgccggcagcatcgaggtggacgccgtgaccgaggccgagctgaaggagaacggcttcaggagcaccaggaggaccaagctggtgtgcacgatcggcccggccacctgcggcttcgagcagctggaggccctggccgtgggcgggatgaacgtggccaggatcaatatgtgccacggcaccagggagtggcacaagagcgtgatcgacaaggtgaggaggctgaacgaggagaagggctacgccgtggccatcatgatggacaccgagggcagcgagatccacatgggcgacctgggcggcgccagcagcgccaaggccgaggacggccaggtgtggaccttcagcgtgagggcctacgacaccctgaggccggagaggtgcatcaccgtgaactacgacggcttcgccgaggacgtgaaggtgggcgacgagctgctggtggacggcggcatggtgaggttcgaggtgatcgagaagatcggcccggacgtgaagtgcctgtgcaccgacccgggcctgctgctgccgagggccaacctgaccttctggagggacggcagcctggtgagggagaggaacgcgatgctgccgacgatcagcagcaaggactggctggatatcgacttcgggatcgccgagggcgtggactttatcgccgtgagcttcgtgaagagcgccgaggtgatcaaccacctgaagagctatatcaaggccaggagcagggacggcgacatcgccgtgatcagcaagatcgagagcatcgacagcctgaagaacctggaggagatcatcctggccagcgacggcgcgatggtggccaggggcgacctgggcgcccagatcccgctggagcaggtgccgctggcccagcagaagatcgtggaggtgtgcaggcagctgaacaagccggtgatcgtggccagccagctgctggagagtatgatcgagtacccgaccccgaccagggccgaggtggccgacgtgagcgaggccgtgaggcagagggccgacgccctgatgctgagcggcgagagcgcgatgggccagttcccggacaaggccctgaccgtgctgaggagcgtgagcctgaggatcgagaagtggtggagggaggagaagaggcacgaggtgatggacctgccggatatcgccagcagcttcagcgacagtatcagcgaggagatctgcaacagcgccgccaagatggccaacaacctggaggtggacgccctgttcgtgtacaccaaggacggccatatggccagcctgctgagcaggagcaggccggactgcccgatcttcgccttcaccaccgacaccctggtgaggaggaggctgaacctgcagtggggcctgatcccgttcaggctggacttcagcgacgatatggagaccaacctgaacaagaccttcagcctgctgaaggccaggggtatgatcaagagcggcgacctggtgatcgccgtgagcgatatgctgcagagtatccaggtgatgaacgtgccgtga

SEQ ID NO: 66 - protein, 573 aa, Helianthus annuus; identical to SEQ ID NO: 64

SEQ ID NO: 67 - DNA, 1743 bp, Helianthus annuus
atggcggcgcaagtggttgcttcgaagctgacacacagttcctttgcatgccctaattcttccaaaaaccagattcagaagatcaaacaacctagtggttttggttctaaagctgtgattggtaacaatgaacaagatcaaaccagactgacttacaaacatcgttgtgttgttgttagcgtcacttcaaggagacaacaagagctccaagttgttgttccggttacacctgaagacgtacctaagattgcagagccagattatgatacaccggaagttttgcagcagggggacagatcggtaagcatgtggtcaaggcccttagtgaagcgcaaaacaaaaatcgtttgcacaatcggtccatccacaaacaccaaggaaatgatatggaaactggctgaggcggggatgaacgtagcaaggttaaacatgtcccacggagaccacgcgtcccatcagaaagttatcgaccttgttaaggaatacaacgctcaatctaaagataacgttattgcaattatgctcgacacaaaggggcccgaagtcaggagtggagacttgcctcagccggttaatttagtaagtggccaagaattcacgttcacgatcaaacggggcgttggcacatcggagtgtgtaagcgttaactatgatgattttgtgaatgacgtggaagctggtgacatgcttctggttgatggtggtatgatgtcattgttggtgaaatccaagaccgaagattcagtaacatgtgaagttgtcgatggcggagagctcaaatcgagacgccacttaaatgttagaggaaaaagtgcaactttgccatccatcactgaaaaagattgggatgatatcaaatttggagtggacaatgaagttgatttctacgcggtctcctttgttaaagatgcggaagtcattcatgagttaaagaattacctcaaaagctgcggtgcaaatatccaagtaattccaaaaattgaaagtgcagactcgataccgaacttacattcaattatcacagcatcagacggtgcaatggttgcaagaggagatcttggtgcagagctgcctattgaagaggttccactgttgcaggaagagatcataagaacatgcaggagcatggggaaagcggtgatagttgcaacgaacatgcttgaaagcatgatagtccatccaacaccaacgagagcggaggtatctgacatcgctattgctgtcagagagggtgctgatgcggttatgctttctggagaaacagctcatggaaagtttcctttaaaagctgttaaagttatgcacacagtgtcgttgcgaactgaagcaagcattacgggtggtgtcacaccctctaatcttggtcaagcctttaagaaccatatgagtgaaatgttcgcgtttcatgccacatcaatgtcaaacactcttggaacctcactcgtggtttttactagaaccggtttcatggctattctattgagtcattatcgacccactggcaccatctttgccttcacaaaccagaaacgagtgcaacagaagttagctttgtatcaaggagtttgccccatctatatggagttctctaatgatgctgatgagaccttcgaaaacgctctcagcaccttgaagaaacaagggatggtaaaagaaggtgatgaggtagcgcttgttcaaagcgggaggcagccgatctggcggttccaatccacacataatattcaggtcaggaaggtgtaa

SEQ ID NO: 68 - protein, 580 aa, Helianthus annuus
MAAQVVASKLTHSSFACPNSSKNQIQKIKQPSGFGSKAVIGNNEQDQTRLTYKHRCVVVSVTSRRQQELQVVVPVTPEDVPKIAEPDYDTPEVLQQGDRSVSMWSRPLVKRKTKIVCTIGPSTNTKEMIWKLAEAGMNVARLNMSHGDHASHQKVIDLVKEYNAQSKDNVIAIMLDTKGPEVRSGDLPQPVNLVSGQEFTFTIKRGVGTSECVSVNYDDFVNDVEAGDMLLVDGGMMSLLVKSKTEDSVTCEVVDGGELKSRRHLNVRGKSATLPSITEKDWDDIKFGVDNEVDFYAVSFVKDAEVIHELKNYLKSCGANIQVIPKIESADSIPNLHSIITASDGAMVARGDLGAELPIEEVPLLQEEIIRTCRSMGKAVIVATNMLESMIVHPTPTRAEVSDIAIAVREGADAVMLSGETAHGKFPLKAVKVMHTVSLRTEASITGGVTPSNLGQAFKNHMSEMFAFHATSMSNTLGTSLVVFTRTGFMAILLSHYRPTGTIFAFTNQKRVQQKLALYQGVCPIYMEFSNDADETFENALSTLKKQGMVKEGDEVALVQSGRQPIWRFQSTHNIQVRKV

SEQ ID NO: 69 - DNA, 1743 bp, Artificial sequence
  codon optimized sequence of HaPK002 for expression in Zea mays
atggccgcccaggtggtggccagcaagctgacccacagcagcttcgcctgcccgaacagcagcaagaaccagatccagaagatcaagcagccgagcggcttcggcagcaaggccgtgatcggcaacaacgagcaggaccagaccaggctgacctacaagcacaggtgcgtggtggtgagcgtgaccagcaggaggcagcaggagctgcaggtggtggtgccggtgaccccggaggacgtgccgaagatcgccgagccggactacgacaccccggaggtgctgcagcagggcgacaggagcgtgagcatgtggagcaggccgctggtgaagaggaagaccaagatcgtgtgcacgatcggcccgagcaccaacaccaaggagatgatctggaagctggccgaggccgggatgaacgtggccaggctgaatatgagccacggcgaccacgccagccaccagaaggtgatcgacctggtgaaggagtacaacgcccagagcaaggacaacgtgatcgcgataatgctggacaccaagggcccggaggtgaggagcggcgacctgccgcagccggtgaacctggtgagcggccaggagttcaccttcacgatcaagaggggcgtgggcaccagcgagtgcgtgagcgtgaactacgacgacttcgtgaacgacgtggaggccggcgacatgctgctggtggacggcggcatgatgagcctgctggtgaagagcaagaccgaggacagcgtgacctgcgaggtggtggacggcggcgagctgaagagcaggaggcacctgaacgtgaggggcaagagcgccaccctgccgagtatcaccgagaaggactgggacgatatcaagttcggcgtggacaacgaggtggacttctacgccgtgagcttcgtgaaggacgccgaggtgatccacgagctgaagaactacctgaagagctgcggcgccaacatccaggtgatcccgaagatcgagagcgccgacagcatcccgaacctgcacagcattatcaccgccagcgacggcgcgatggtggccaggggcgacctgggcgccgagctgccgatcgaggaggtgccgctgctgcaggaggagataatcaggacctgcaggagtatgggcaaggccgtgatcgtggccaccaatatgctggagagtatgatcgtgcacccgaccccgaccagggccgaggtgagcgatatcgcgatcgccgtgagggagggcgccgacgccgtgatgctgagcggcgagaccgcccacggcaagttcccgctgaaggccgtgaaggtgatgcacaccgtgagcctgaggaccgaggccagtatcaccggcggcgtgaccccgagcaacctgggccaggccttcaagaaccatatgagcgagatgttcgccttccacgccaccagtatgagcaacaccctgggcaccagcctggtggtgttcaccaggaccggctttatggcgatcctgctgagccactacaggccgaccggcaccatcttcgccttcaccaaccagaagagggtgcagcagaagctggccctgtaccagggcgtgtgcccgatctacatggagttcagcaacgacgccgacgagaccttcgagaacgccctgagcaccctgaagaagcagggcatggtgaaggagggcgacgaggtggccctggtgcagagcggcaggcagccgatctggaggttccagagcacccacaatatccaggtgaggaaggtgtga

SEQ ID NO: 70 - protein, 580 aa, Helianthus annuus; identical to SEQ ID NO: 68

SEQ ID NO: 71 - DNA, 1518 bp, Helianthus annuus
atgaagactgatcagagtgcgcaacagatcggcgtgaagaggccgaaaacgaagatcgtttgcactcttggaccggcttccagatctgttctcatggtggagaagttgctgaaagcgggaatgaacgttgccaggttcaacttctcgcatgggtcttacgagtatcatcaggagacacttgataatctgaggaccgctatggatagtactggtattctttgtgcggttatgcttgatacaaagggtccagagattagaaccggttttctaaaagatggcaagcccatccagctaaaacaaggccaagaaatcaccatctcaaccgattacgacataaaaggcgacgaaaccatgatctgcatgagctacaaaaagttagcctacgacgtaaagccagggagcgtcatactatgcgcagatggcactatttcatttactgttcttgcttgtgacaccgaaaatggtttggtccgttgtcgctgtgagaacactgcggttcttggtgagcgaaagaatgttaatcttcctggagtgatcgtcgacttaccaactttgactgagaaagacaaagaggatatactcacatggggagttccgaacaagattgatatgattgcgctgtcgtttgttcgcaaaggctcggatttggtcgaggttcgaaagctgttgggtaagcatgctaatagcattcttcttatgtcaaaggttgagaatcaagaaggggtagcaaacttcgacgaaatcctagccaactccgacgcgttcatggtggcgcgtggtgatctcggaatggagattccgatcgagaagatattcttagcccaaaaggtcatgatctacaaatgcaacatacaaggaaagccggtggtaaccgccactcaaatgctcgagtcaatgatcaaatctccaagaccaactcgagccgaagctacagatgtggccaacgcagttctagatggtacagactgtgtcatgcttagcggtgaaacagccgctggagcctacccagagctagcggtcctaaccatggctaggatttgtgttgaagccgagaacacaatcgattacccggatgttttcaaaagaattgcggcaaacgcacccatacccatgagcccactcgagagcttagcctctgctgcggtccgaaccgccaactcatctagagcaagcctcattctggttctgaccagaggcggaagcacagccaagctggtggctaagtacagaccgggcatgcctgtattatctgtagtggttcccgaaatccaaaccgattctttcgattggtcatgcagtgacgaaacgccagctaggcatagtcttattttcagaggtttggtcccggttctatctgcagggtccactagagcttcctatgctgagtcaaccgaagaggttttggactttgcattgcaacatgcgaaactcaagggactctgcaaggaaggcgatgctgttgtggctttacatcgtgtgggaatcgcttctgttattaagataattaacgtcaagtaa

SEQ ID NO: 72 - protein, 505 aa, Helianthus annuus
MKTDQSAQQIGVKRPKTKIVCTLGPASRSVLMVEKLLKAGMNVARFNFSHGSYEYHQETLDNLRTAMDSTGILCAVMLDTKGPEIRTGFLKDGKPIQLKQGQEITISTDYDIKGDETMICMSYKKLAYDVKPGSVILCADGTISFTVLACDTENGLVRCRCENTAVLGERKNVNLPGVIVDLPTLTEKDKEDILTWGVPNKIDMIALSFVRKGSDLVEVRKLLGKHANSILLMSKVENQEGVANFDEILANSDAFMVARGDLGMEIPIEKIFLAQKVMIYKCNIQGKPVVTATQMLESMIKSPRPTRAEATDVANAVLDGTDCVMLSGETAAGAYPELAVLTMARICVEAENTIDYPDVFKRIAANAPIPMSPLESLASAAVRTANSSRASLILVLTRGGSTAKLVAKYRPGMPVLSVVVPEIQTDSFDWSCSDETPARHSLIFRGLVPVLSAGSTRASYAESTEEVLDFALQHAKLKGLCKEGDAVVALHRVGIASVIKIINVK

SEQ ID NO: 73 - DNA, 1518 bp, Artificial sequence
  codon optimized sequence of HaPK003 for expression in Zea mays
atgaagaccgaccagagcgcccagcagatcggcgtgaagaggccgaagaccaagatcgtgtgcaccctgggcccggccagcaggagcgtgctgatggtggagaagctgctgaaggccggcatgaacgtggccaggttcaacttcagccacggcagctacgagtaccaccaggagaccctggacaacctgaggaccgccatggacagcaccggcatcctgtgcgccgtgatgctggacaccaagggcccggagatcaggaccggcttcctgaaggacggcaagccgatccagctgaagcagggccaggagatcacgatcagcaccgactacgacatcaagggcgacgagaccatgatctgcatgagctacaagaagctggcctacgacgtgaagccgggcagcgtgatcctgtgcgccgacggcaccatcagcttcaccgtgctggcctgcgacaccgagaacggcctggtgaggtgcaggtgcgagaacaccgccgtgctgggcgagaggaagaacgtgaacctgccgggcgtgatcgtggacctgccgaccctgaccgagaaggacaaggaggatatcctgacctggggcgtgccgaacaagatcgatatgatcgccctgagcttcgtgaggaagggcagcgacctggtggaggtgaggaagctgctgggcaagcacgccaacagtatcctgctgatgagcaaggtggagaaccaggagggcgtggccaacttcgacgagatcctggccaacagcgacgcctttatggtggccaggggcgacctgggaatggagatcccgatcgagaagatcttcctggcccagaaggtgatgatctacaagtgcaacatccagggcaagccggtggtgaccgccacccagatgctggagagcatgatcaagagcccgaggccgaccagggccgaggccaccgacgtggccaacgccgtgctggacggcaccgactgcgtgatgctgagcggcgagaccgccgccggcgcctacccggagctggccgtgctgaccatggccaggatctgcgtggaggccgagaacaccatcgactacccggacgtgttcaagaggatcgccgccaacgccccgatcccgatgagcccgctggagagcctggccagcgccgccgtgaggaccgccaacagcagcagggccagcctgatcctggtgctgaccaggggcggcagcaccgccaagctggtggccaagtacaggccgggaatgccggtgctgagcgtggtggtgccggagatccagaccgacagcttcgactggagctgcagcgacgagaccccggccaggcacagcctgatcttcaggggcctggtgccggtgctgagcgccggcagcaccagggccagctacgccgagagcaccgaggaggtgctggacttcgccctgcagcacgccaagctgaagggcctgtgcaaggagggcgacgccgtggtggccctgcacagggtggggatcgccagcgtgatcaagataatcaacgtgaagtga

SEQ ID NO: 74 - protein, 505 aa, Helianthus annuus; identical to SEQ ID NO: 72

SEQ ID NO: 75 - DNA, 1443 bp, Pectobacterium wasabiae
atgagcaggcgtctaaggcgtaccaagatcgtcacaaccttaggacctgcgaccgatcgggacaataacctggagaaaatcatcaatgccggcgccaatgtggtaagaatgaactttagccatggcactgctgaagaccaccagctccgtgccaacaaagttcgagagatagcagcgaagctcggaaggcacgtcgcgattctaggcgatcttcaaggacccaaaattcgagtgagcacgttcaaggagggcaagatcttcctgaatatcggtgacaagttccttctggacgccaacctcgctaagggagagggagataaggaaaaggtcggtattgactacaagggactcccgagcgacgtggtccctggtgatatcttgcttctcgacgacgggagggttcagttgaaagttttgcaagttgagggactcaaggtttataccgaggtgaccgtgggtggaccactttcgaacaataaggggatcaacaagcttggtggaggcctctctgctgaagcgctgacggagaaggacaaagccgatattatcacggccgcgaagattggggtggattacctggctgtgagcttcccaagaacgggagaggatctcaactacgcgagacggctggctagagacgcgggttgcaacgctaagatagtctccaaggtagagagagctgaggctgtgtgctcagacgctgcaatggacgatataatcctggcctcggatgtcgtgatggtggctcgtggtgatctaggcgtcgaaatcggcgatcccgagctggtagggatacagaagaaactgattcgtagagcgcgacagctaaaccgtgcggtgataactgccactcagatgatggagtccatgattacaaacccgatgcccacacgtgctgaggtgatggacgttgcaaatgccgtccttgatggtactgatgctgtgatgctatctgccgaaactgctgcgggacagtatccagcagagaccgttgcggctatggccaaagtctgtttgggagccgagaagatcccgagtatcaacgtctctaagcaccggttggacgttcagttcgacaacaccgaagaatctatcgcaatgtcctcaatgtacgctgccaaccatcttaagggagttaccgctctgattgctatgacggaatcgggaagaaccgctttgatgatgtcccgaatctccagtgggctccctatcttcgcaatgagtcggcacgagcacacactgaacttgacggctctctaccgtggcgtcacaccggtccacttcgatagctataccgatggagtcgcggccgctaacgatgccgtcatacgcttacgggataagggcttcctgatgtcgggagacttggtgatcgtgacacaaggcgatattatgggaacggtgggcactacgaatactatccgtatcctgagggtggagtag

SEQ ID NO: 76 - protein, 480 aa, Pectobacterium wasabiae
MSRRLRRTKIVTTLGPATDRDNNLEKIINAGANVVRMNFSHGTAEDHQLRANKVREIAAKLGRHVAILGDLQGPKIRVSTFKEGKIFLNIGDKFLLDANLAKGEGDKEKVGIDYKGLPSDVVPGDILLLDDGRVQLKVLQVEGLKVYTEVTVGGPLSNNKGINKLGGGLSAEALTEKDKADIITAAKIGVDYLAVSFPRTGEDLNYARRLARDAGCNAKIVSKVERAEAVCSDAAMDDIILASDVVMVARGDLGVEIGDPELVGIQKKLIRRARQLNRAVITATQMMESMITNPMPTRAEVMDVANAVLDGTDAVMLSAETAAGQYPAETVAAMAKVCLGAEKIPSINVSKHRLDVQFDNTEESIAMSSMYAANHLKGVTALIAMTESGRTALMMSRISSGLPIFAMSRHEHTLNLTALYRGVTPVHFDSYTDGVAAANDAVIRLRDKGFLMSGDLVIVTQGDIMGTVGTTNTIRILRVE

SEQ ID NO: 77 - DNA, 1428 bp, Zymomonas mobilis
atgactgaaggacttttcccacgcggccgcaaggtacgtgttgtttctacgctgggtcctgccagctcgacggcagaacagattcgtgatcgtttcttggctggtgcggatgtattccgtatcaacatgagccatggtacccatgacgaaaagaaggtcatcgttgacaacattcgtgcgcttgaaaaagaattcaaccgcccgaccacgatccttttcgatcttcagggtccgaagctccgcgttggtgacttcaaagaaggcaaggttcagttgaaagaaggccagaccttcactttcgatcaggacccaactctcggtgacgaaacccgcgtcaacctgccgcatcctgaaatcttcaaggctttagacaaaggtcatcgcctgcttttggatgatggtaagatcgttgttcgttgcgtcgaatcttccccgaccaaaatcgttacccgcgtcgaagttccgggtccgctttccgaccataaaggtttcaacgttcctgacgttgtgattccgctggccgctctgaccccgaaagatcgcaaagatctcgactttgctcttaaggaaaaagctgactgggttgctcttagctttgtgcagcgcgtcgaagacgtcatcgaagccaaagaactcatcaaaggtcgcgcaccgcttctcgttaaactcgaaaagccagctgccattgaaaacctcgaatccatcttggctgcaaccgatgctgtcatggttgctcgtggtgacctcggtgttgaatgcttgccggaatccgttcccccgacgcagaagcgtatcgttgaacgctcccgccagctgggtaagccggttgttgttgcaaccgctatgctcgaatccatgatcaaagctccggctccgacccgcgctgaagtaagtgacgttgcaaacgccatttacgaaggtgctgatggtatcatgctttctgcagaatccgccgccggtgattggccgcatgaagctgtcaacatgatgcatcgtattgcttcttatgttgaaaatgctccgggttacatcgaacgcgttcgcttcaccccgactccggcagaaccgacgacggttgacgctctggctgaaaacgccagcaaaaccgctgaaacggttggcgcaaaagcgatcatcgtcttcaccgaaaccggtaagaccgcacagcgcgtttcccgcgcccgtccggttgctccgatcctgtctctgaccccggatgccgaagttgctcgtcgtcttggtttggtctggggcgcacagccggttcaggtttccacggttaagaccctcgacgaagccaagaagctggctgctgaaaccgctaaaaaatacggtttcgccaaagccggtgacaagctggttgttgttgcaggtgaacctttcggtaaagctggaaccaccaacattgttgatgttatcgaagcctaa

SEQ ID NO: 78 - protein, 475 aa, Zymomonas mobilis
MTEGLFPRGRKVRVVSTLGPASSTAEQIRDRFLAGADVFRINMSHGTHDEKKVIVDNIRALEKEFNRPTTILFDLQGPKLRVGDFKEGKVQLKEGQTFTFDQDPTLGDETRVNLPHPEIFKALDKGHRLLLDDGKIVVRCVESSPTKIVTRVEVPGPLSDHKGFNVPDVVIPLAALTPKDRKDLDFALKEKADWVALSFVQRVEDVIEAKELIKGRAPLLVKLEKPAAIENLESILAATDAVMVARGDLGVECLPESVPPTQKRIVERSRQLGKPVVVATAMLESMIKAPAPTRAEVSDVANAIYEGADGIMLSAESAAGDWPHEAVNMMHRIASYVENAPGYIERVRFTPTPAEPTTVDALAENASKTAETVGAKAIIVFTETGKTAQRVSRARPVAPILSLTPDAEVARRLGLVWGAQPVQVSTVKTLDEAKKLAAETAKKYGFAKAGDKLVVVAGEPFGKAGTTNIVDVIEA

SEQ ID NO: 79 - DNA, 1446 bp, Photobacterium profundum
atgtccgagcagcttcggcgcacgaagatcgttactaccctgggacctgccaccgatcgcgacaacaatcttgagaagattatcgcggctggcgcgaacgtggtaagaatgaacttctcgcacggctcacctgaggaccatattcagcgtaccaagatggtcagagagatcgctgcgaagctcggcaagcacattgccatcctgggagatctgcaaggtcccaagatcagggtgagcacattcaaggacggcaagatccagttggcgatcggcgacaagttcacactggattccgacctccccaagggagagggacaccaggatgcggtcggactcgattacaaggagcttccgaacgacgtggtaacgggtgacctgttgcttctcgacgatgggagggttcagctcaaggttactgctgtttctggcaacaaggttcacacggaggtgaccgtgggtggcccactgtctaacaataagggcatcaataagaagggtggaggcctaagcgcggaagcgctcaccgagaaggacaaggccgatataatcaccgccgcgaagatgggtgtggattacctggctgtctcgttcccacgtaatggggaggatatgaaatacgctcgatcgctggctcttgacgcgggactcgaggctaagctggtcgccaaagtcgagcgtgctgaggctgttgctaccacggaggcaatggacgatataattatggcctccgacgtggttatggttgcacgtggggacttaggagtcgaaatcggcgatcccgaactggtcggcgtccagaagaagctgatccgtagagcaagatcgcttaaccgcacggtgatcacggccacccagatgatggagtcgatgattacctcccctatgccgacacgagcggaggtgatggacgttgcaaacgccgtactggacggtactgacgctgtaatgctgtctgccgaaaccgctgccggagacttcccagtagagaccgtccaagctatgagctcagtttgcgtcggggccgagaaggagccttcgatcaacgtgtcaaaccaccgcttggctagaacgttcgctagccccgaagagactatcgcaatgagtacgatgtacgcggccaaccatatggaaggcgtcaaggcgatggtggcaatgacagagtcgggtagaacgcctctaatgatgagtcggatatccagcgggctaccggtcttcgcgatgtctcggaacgagaacacacttaaccgctctagtctctaccgtggcgtcactccggtctacttcgatcgggatagtgatgccggcctcgaagccgcgaagcacgccttggctgttctgaaggagcacggttatctcaccaccggcgacctcgtgattataacgcaaggcgatgtcatggacacgataggtagcaccaataacatgcgaatcctgactgtggagtag

SEQ ID NO: 80 - protein, 481 aa, Photobacterium profundum
MSEQLRRTKIVTTLGPATDRDNNLEKIIAAGANVVRMNFSHGSPEDHIQRTKMVREIAAKLGKHIAILGDLQGPKIRVSTFKDGKIQLAIGDKFTLDSDLPKGEGHQDAVGLDYKELPNDVVTGDLLLLDDGRVQLKVTAVSGNKVHTEVTVGGPLSNNKGINKKGGGLSAEALTEKDKADIITAAKMGVDYLAVSFPRNGEDMKYARSLALDAGLEAKLVAKVERAEAVATTEAMDDIIMASDVVMVARGDLGVEIGDPELVGVQKKLIRRARSLNRTVITATQMMESMITSPMPTRAEVMDVANAVLDGTDAVMLSAETAAGDFPVETVQAMSSVCVGAEKEPSINVSNHRLARTFASPEETIAMSTMYAANHMEGVKAMVAMTESGRTPLMMSRISSGLPVFAMSRNENTLNRSSLYRGVTPVYFDRDSDAGLEAAKHALAVLKEHGYLTTGDLVIITQGDVMDTIGSTNNMRILTVE

SEQ ID NO: 81 - DNA, 1425 bp, Arabidopsis thaliana
atgtccgtggctcgattcgatttctcttggtgcgatgctgattatcaccaggagacgctggagaatctgaagatagctgtgaagagcactaagaagctttgtgctgttatgctagacactgtaggacctgagttgcaagttattaacaagactgagaaagctatttctcttaaagctgatggccttgtaactttgactccgagtcaagatcaagaagcctcctctgaagtccttcccattaattttgatgggttagcgaaggcggttaagaaaggagacactatctttgttggacaatacctcttcactggtagtgaaacaacttcagtttggcttgaggttgaagaagttaaaggagatgatgtcatttgtatttcaaggaatgctgctactctgggtggtccgttattcacattgcacgtctctcaagttcacattgatatgccaaccctaactgagaaggataaggaggttataagtacatggggagttcagaataagatcgactttctctcattatcttattgtcgacatgcagaagatgttcgccaggcccgtgagttgcttaacagttgtggtgacctctctcaaacacaaatatttgcgaagattgagaatgaagagggactaacccactttgacgaaattctacaagaagcagatggcattattctttctcgtgggaatttgggtatcgatctacctccggaaaaggtgtttttgttccaaaaggctgctctttacaagtgtaacatggctggaaagcctgccgttcttactcgtgttgtagacagtatgacagacaatctgcggccaactcgtgcagaggcaactgatgttgctaatgctgttttagatggaagtgatgcaattcttcttggtgctgagactcttcgtggattgtaccctgttgaaaccatatcaactgttggtagaatctgttgtgaggcagagaaagttttcaaccaagatttgttctttaagaagactgtcaagtatgttggagaaccaatgactcacttggaatctattgcttcttctgctgtacgggcagcaatcaaggttaaggcatccgtaattatatgcttcacctcgtctggcagagcagcaaggttgattgccaaataccgtccaactatgcccgttctctctgttgtcattccccgacttacgacaaatcagctgaagtggagctttagcggagcctttgaggcaaggcagtcacttattgtcagaggtcttttccccatgcttgctgatcctcgtcaccctgcggaatcaacaagtgcaacaaatgagtcggttcttaaagtggctctagaccatgggaagcaagccggagtgatcaagtcacatgacagagttgtggtctgtcagaaagtgggagatgcgtccgtggtcaaaatcatcgagctagaggattag

SEQ ID NO: 82 - protein, 474 aa, Arabidopsis thaliana
MSVARFDFSWCDADYHQETLENLKIAVKSTKKLCAVMLDTVGPELQVINKTEKAISLKADGLVTLTPSQDQEASSEVLPINFDGLAKAVKKGDTIFVGQYLFTGSETTSVWLEVEEVKGDDVICISRNAATLGGPLFTLHVSQVHIDMPTLTEKDKEVISTWGVQNKIDFLSLSYCRHAEDVRQARELLNSCGDLSQTQIFAKIENEEGLTHFDEILQEADGIILSRGNLGIDLPPEKVFLFQKAALYKCNMAGKPAVLTRVVDSMTDNLRPTRAEATDVANAVLDGSDAILLGAETLRGLYPVETISTVGRICCEAEKVFNQDLFFKKTVKYVGEPMTHLESIASSAVRAAIKVKASVIICFTSSGRAARLIAKYRPTMPVLSVVIPRLTTNQLKWSFSGAFEARQSLIVRGLFPMLADPRHPAESTSATNESVLKVALDHGKQAGVIKSHDRVVVCQKVGDASVVKIIELED

SEQ ID NO: 83 - DNA, 1791 bp, Arabidopsis thaliana
atgtctcagtctattcaattctccactccttcacacactcctcaccttctccatctccctcactcacaattcaaccgtcctctctcctctatctccttccgtcgcttccctctaacaaccatcaaatacacttccatcagagcctcctcgtcatcatctccttcaccggatctcgattcatcgtcctcatcatcatcctcgcaagtacttctctcacctaacggtactggtgctgtgaagtctgatgagagatccgttgtcgctacggcggttacgactgatacgtctgggattgaggttgatactgtgacggaagctgagcttaaggagaatggatttagaagtacgaggaggacgaagctgatctgtacgatcggaccggcgacttgtggatttgagcagcttgaggcgcttgctgtgggaggtatgaatgtggcaaggcttaatatgtgtcacggtacgcgtgattggcaccgcggtgtgattcgtagtgttcggaggcttaatgaggagaaaggctttgcggttgctattatgatggatactgaaggtagtgagattcatatgggagatcttggtggtgaagcttcagctaaagcagaggatggtgaggtttggactttcactgttagagcttttgattcttctcgtcctgaacgtaccattagtgttagctacgatggtttcgctgaagatgtaagagttggggatgaacttttggttgatggtgggatggtgagatttgaagtgattgagaagattggtcctgatgttaagtgtctatgtaccgatcctggattgttgcttcctcgagctaacttgacgttttggagagatggaagtcttgtacgagagcgtaatgccatgcttccaacaatttcttccaaggactggttggatattgattttggaattgctgaaggtgtggatttcattgctgtatcgtttgtcaagtcggctgaagtcattaatcaccttaaaagttatcttgctgctcgttcccgtggaggggaaattggagtgattgcaaagatcgagagtatcgattcactgaccaatttggaagaaattattctagcatcagatggggccatggttgcaagaggagatctgggagctcagatacctcttgagcaagttccagcagctcaacagagaatcgtccaagtatgcagagctcttaacaaacccgtcattgtcgcttcacagctattggagtccatgattgagtacccaactccaaccagagcagaagttgccgacgtgtctgaagcagtaagacaaagatcagatgcattgatgctctctggagaatcagctatgggacaattcccagacaaggcgctcacggttctaaggactgtcagtttaagaatcgagagatggtggagggaagagaaacgccatgagtctgtaccgcttcaagccataggctcttcattttcagacaaaatctcagaagagatctgtaactcagctgctaaaatggctaacaatcttggagtggacgcggttttcgtttacacaacgagcggacacatggcatcactggtctcccgatgtcgcccggactgcccgatctttgctttcacaaccacaacctcagtgagaagacgcttaaacctacaatggggacttatcccattccgtctcagcttctcagacgacatggaaagcaacttgaacaaaacattctcgttactgaaatcaagaggtatgatcaaatctggtgacctcgtgatcgcagtctcggacatgctgcaatcaatccaggtaatgaacgtcccgtaa

SEQ ID NO: 84 - protein, 596 aa, Arabidopsis thaliana
MSQSIQFSTPSHTPHLLHLPHSQFNRPLSSISFRRFPLTTIKYTSIRASSSSSPSPDLDSSSSSSSSQVLLSPNGTGAVKSDERSVVATAVTTDTSGIEVDTVTEAELKENGFRSTRRTKLICTIGPATCGFEQLEALAVGGMNVARLNMCHGTRDWHRGVIRSVRRLNEEKGFAVAIMMDTEGSEIHMGDLGGEASAKAEDGEVWTFTVRAFDSSRPERTISVSYDGFAEDVRVGDELLVDGGMVRFEVIEKIGPDVKCLCTDPGLLLPRANLTFWRDGSLVRERNAMLPTISSKDWLDIDFGIAEGVDFIAVSFVKSAEVINHLKSYLAARSRGGEIGVIAKIESIDSLTNLEEIILASDGAMVARGDLGAQIPLEQVPAAQQRIVQVCRALNKPVIVASQLLESMIEYPTPTRAEVADVSEAVRQRSDALMLSGESAMGQFPDKALTVLRTVSLRIERWWREEKRHESVPLQAIGSSFSDKISEEICNSAAKMANNLGVDAVFVYTTSGHMASLVSRCRPDCPIFAFTTTTSVRRRLNLQWGLIPFRLSFSDDMESNLNKTFSLLKSRGMIKSGDLVIAVSDMLQSIQVMNVP

SEQ ID NO: 85 - DNA, 1413 bp, Escherichia coli
atgaaaaagaccaaaattgtttgcaccatcggaccgaaaaccgaatctgaagagatgttagctaaaatgctggacgctggcatgaacgttatgcgtctgaacttctctcatggtgactatgcagaacacggtcagcgcattcagaatctgcgcaacgtgatgagcaaaactggtaaaaccgccgctatcctgcttgataccaaaggtccggaaatccgcaccatgaaactggaaggcggtaacgacgtttctctgaaagctggtcagacctttactttcaccactgataaatctgttatcggcaacagcgaaatggttgcggtaacgtatgaaggtttcactactgacctgtctgttggcaacaccgtactggttgacgatggtctgatcggtatggaagttaccgccattgaaggtaacaaagttatctgtaaagtgctgaacaacggtgacctgggcgaaaacaaaggtgtgaacctgcctggcgtttccattgctctgccagcactggctgaaaaagacaaacaggacctgatctttggttgcgaacaaggcgtagactttgttgctgcttcctttattcgtaagcgttctgacgttatcgaaatccgtgagcacctgaaagcgcacggcggcgaaaacatccacatcatctccaaaatcgaaaaccaggaaggcctcaacaacttcgacgaaatcctcgaagcctctgacggcatcatggttgcgcgtggcgacctgggtgtagaaatcccggtagaagaagttatcttcgcccagaagatgatgatcgaaaaatgtatccgtgcacgtaaagtcgttatcactgcgacccagatgctggattccatgatcaaaaacccacgcccgactcgcgcagaagccggtgacgttgcaaacgccatcctcgacggtactgacgcagtgatgctgtctggtgaatccgcaaaaggtaaatacccgctggaagcggtttctatcatggcgaccatctgcgaacgtaccgaccgcgtgatgaacagccgtctcgagttcaacaatgacaaccgtaaactgcgcattaccgaagcggtatgccgtggtgccgttgaaactgctgaaaaactggatgctccgctgatcgtggttgctactcagggcggtaaatctgctcgcgcagtacgtaaatacttcccggatgccaccatcctggcactgaccaccaacgaaaaaacggctcatcagttggtactgagcaaaggcgttgtgccgcagcttgttaaagagatcacttctactgatgatttctaccgtctgggtaaagaactggctctgcagagcggtctggcacacaaaggtgacgttgtagttatggtttctggtgcactggtaccgagcggcactactaacaccgcatctgttcacgtcctgtaa

SEQ ID NO: 86 - protein, 470 aa, Escherichia coli
MKKTKIVCTIGPKTESEEMLAKMLDAGMNVMRLNFSHGDYAEHGQRIQNLRNVMSKTGKTAAILLDTKGPEIRTMKLEGGNDVSLKAGQTFTFTTDKSVIGNSEMVAVTYEGFTTDLSVGNTVLVDDGLIGMEVTAIEGNKVICKVLNNGDLGENKGVNLPGVSIALPALAEKDKQDLIFGCEQGVDFVAASFIRKRSDVIEIREHLKAHGGENIHIISKIENQEGLNNFDEILEASDGIMVARGDLGVEIPVEEVIFAQKMMIEKCIRARKVVITATQMLDSMIKNPRPTRAEAGDVANAILDGTDAVMLSGESAKGKYPLEAVSIMATICERTDRVMNSRLEFNNDNRKLRITEAVCRGAVETAEKLDAPLIVVATQGGKSARAVRKYFPDATILALTTNEKTAHQLVLSKGVVPQLVKEITSTDDFYRLGKELALQSGLAHKGDVVVMVSGALVPSGTTNTASVHVL

SEQ ID NO: 87 - DNA, 1443 bp, Escherichia coli
atgtccagaaggcttcgcagaacaaaaatcgttaccacgttaggcccagcaacagatcgcgataataatcttgaaaaagttatcgcggcgggtgccaacgttgtacgtatgaacttttctcacggctcgcctgaagatcacaaaatgcgcgcggataaagttcgtgagattgccgcaaaactggggcgtcatgtggctattctgggtgacctccaggggcccaaaatccgtgtatccacctttaaagaaggcaaagttttcctcaatattggggataaattcctgctcgacgccaacctgggtaaaggtgaaggcgacaaagaaaaagtcggtatcgactacaaaggcctgcctgctgacgtcgtgcctggtgacatcctgctgctggacgatggtcgcgtccagttaaaagtactggaagttcagggcatgaaagtgttcaccgaagtcaccgtcggtggtcccctctccaacaataaaggtatcaacaaacttggcggcggtttgtcggctgaagcgctgaccgaaaaagacaaagcagacattaagactgcggcgttgattggcgtagattacctggctgtctccttcccacgctgtggcgaagatctgaactatgcccgtcgcctggcacgcgatgcaggatgtgatgcgaaaattgttgccaaggttgaacgtgcggaagccgtttgcagccaggatgcaatggatgacatcatcctcgcctctgacgtggtaatggttgcacgtggcgacctcggtgtggaaattggcgacccggaactggtcggcattcagaaagcgttgatccgtcgtgcgcgtcagctaaaccgagcggtaatcacggcgacccagatgatggagtcaatgattactaacccgatgccgacgcgtgcagaagtcatggacgtagcaaacgccgttctggatggtactgacgctgtgatgctgtctgcagaaactgccgctgggcagtatccgtcagaaaccgttgcagccatggcgcgcgtttgcctgggtgcggaaaaaatcccgagcatcaacgtttctaaacaccgtctggacgttcagttcgacaatgtggaagaagctattgccatgtcagcaatgtacgcagctaaccacctgaaaggcgttacggcgatcatcaccatgaccgaatcgggtcgtaccgcgctgatgacctcccgtatcagctctggtctgccaattttcgccatgtcgcgccatgaacgtacgctgaacctgactgctctctatcgtggcgttacgccggtgcactttgatagcgctaatgacggcgtagcagctgccagcgaagcggttaatctgctgcgcgataaaggttacttgatgtctggtgacctggtgattgtcacccagggcgacgtgatgagtaccgtgggttctactaataccacgcgtattttaacggtagagtaa

SEQ ID NO: 88 - protein, 480 aa, Escherichia coli
MSRRLRRTKIVTTLGPATDRDNNLEKVIAAGANVVRMNFSHGSPEDHKMRADKVREIAAKLGRHVAILGDLQGPKIRVSTFKEGKVFLNIGDKFLLDANLGKGEGDKEKVGIDYKGLPADVVPGDILLLDDGRVQLKVLEVQGMKVFTEVTVGGPLSNNKGINKLGGGLSAEALTEKDKADIKTAALIGVDYLAVSFPRCGEDLNYARRLARDAGCDAKIVAKVERAEAVCSQDAMDDIILASDVVMVARGDLGVEIGDPELVGIQKALIRRARQLNRAVITATQMMESMITNPMPTRAEVMDVANAVLDGTDAVMLSAETAAGQYPSETVAAMARVCLGAEKIPSINVSKHRLDVQFDNVEEAIAMSAMYAANHLKGVTAIITMTESGRTALMTSRISSGLPIFAMSRHERTLNLTALYRGVTPVHFDSANDGVAAASEAVNLLRDKGYLMSGDLVIVTQGDVMSTVGSTNTTRILTVE

SEQ ID NO: 89 - DNA, 1443 bp, Artificial sequence
  codon optimized B1854 sequence for expression in Zea mays
atgagcaggcgtcttcggcgcaccaagatcgttactacccttggacctgcgactgatcgggacaacaatctggagaaagttattgcggccggtgcgaatgtggtacgtatgaacttcagccacggaagtcctgaggaccacaagatgcgagccgacaaggttagagagattgctgcgaagcttggaaggcacgtagcgatactcggtgatctgcaaggtcccaagatccgggtgagcacgttcaaggaggggaaggtgttcctgaatatcggtgacaagtttcttctggacgctaacctcggcaagggagagggagacaaggagaaggtgggaatcgattacaagggacttccggctgacgtagtgcctggtgatatcttgctcctggacgacgggagagtgcaactcaaggtgttggaagttcaaggcatgaaggttttcaccgaggttaccgttggtggaccactgtccaacaacaaggggatcaacaaactcggaggtggcctgtctgcggaagcgcttaccgagaaggacaaagctgatattaagactgctgcgttgattggggtggactacctggcagtgagtttccctcgttgcggagaggatctgaactacgcacgacgcttggctcgtgacgcgggttgcgacgcaaagatcgtcgcgaaagtcgaacgagctgaggcagtctgctctcaagatgcaatggatgacatcatactcgccagcgacgtggtaatggtcgcacgtggtgatctaggggtggaaatcggagatcccgagctggttgggatccaaaaggcactgattcgtcgcgctaggcagctcaatcgagccgtgattaccgccacgcaaatgatggagtcaatgattacaaacccgatgccaacaagggccgaggtgatggatgttgcaaatgcggttctggatgggacggacgctgtgatgctttctgccgaaacggctgcgggacagtatccgtccgagactgttgccgctatggcaagggtttgcttaggggctgagaaaatcccctcgatcaacgtctcgaagcaccgtctcgacgtgcaattcgacaacgtcgaagaggcaatcgcaatgtcagcgatgtatgctgccaatcatctaaagggggtgaccgccataatcacgatgactgagtccggaagaaccgcactcatgacctcgaggatttcaagcggactcccgatctttgctatgtctcgccatgagcggactttgaaccttaccgcactctaccgtggggttacgccggtccactttgatagcgctaatgatggcgtcgctgcggcaagtgaagcagtcaacctactgagggataagggctacttgatgtcgggagacttggtgatcgtgacacaaggagacgtcatgtcaacggtcggctcaaccaatacaacgcgtatcttaacggtggagtga

SEQ ID NO: 90 - protein, 480 aa, Escherichia coli; identical to SEQ ID NO: 88

SEQ ID NO: 91 - DNA, 1443 bp, Escherichia coli
atgtccagaaggcttcgcagaacaaaaatcgttaccacgttaggcccagcaacagatcgcgataataatcttgaaaaagttatcgcggcgggtgccaacgttgtacgtatgaacttttctcacggctcgcctgaagatcacaaaatgcgcgcggataaagttcgtgagattgccgcaaaactggggcgtcatgtggctattctgggtgacctccaggggcccaaaatccgtgtatccacctttaaagaaggcaaagttttcctcaatattggggataaattcctgctcgacgccaacctgggtaaaggtgaaggcgacaaagaaaaagtcggtatcgactacaaaggcctgcctgcagacgtcgtgcctggtgacatcctgctgctggacgatggtcgcgtccagttaaaagtacgggaagttcagggcatgaaagtgttcaccgaagtcaccgtcggtggtcccctctccaacaataaaggtatcaacaaacttggcggcggtttgtcggctgaagcgctgaccgaaaaagacaaagcagacattaagactgcggcgttgattggcgtagattacctggctgtctccttcccacgctgtggcgaagatctgaactatgcccgtcgcctggcacgcgatgcaggatgtgatgcgaaaattgttgccaaggttgaacgtgcggaagccgtttgcagccaggatgcaatggatgacatcatcctcgcctctgacgtggtaatggttgcacgtggcgacctcggtgtggaaattggcgacccggaactggtcggcattcagaaagcgttgatccgtcgtgcgcgtcagctaaaccgagcggtaatcacggcgacccagatgatggagtcaatgattactaacccgatgccgacgcgtgcagaagtcatggacgtagcaaacgccgttctggatggtactgacgctgtgatgctgtctgcagaaactgccgctgggcagtatccgtcagaaactgttgcagccatggcgcgcgtttgcctgggtgcggaaaaaatcccgagcatcaacgtttctaaacaccgtctggacgttcagttcgacaatgtggaagaagctattgccatgtcagcaatgtacgcagctaaccacctgaaaggcgttacggcgatcattaccatgaccgaatcgggtcgtaccgcgctgatgacctcccgtatcagctctggtctaccaattttcgccatgtcgcgccatgaacgtacgctgaacctgactgctctctatcgtggcgttacgccggtgcactttgatagcgctaatgacggcgtagcagctgccagcgaagcggttaatctgctgcgcgataaaggttacttgatgtctggtgacctggtgattgtcacccagggcgacgtgatgagtaccgtgggttctactaataccacgcgtattttaacggtagagtaa

SEQ ID NO: 92 - protein, 480 aa, Escherichia coli
MSRRLRRTKIVTTLGPATDRDNNLEKVIAAGANVVRMNFSHGSPEDHKMRADKVREIAAKLGRHVAILGDLQGPKIRVSTFKEGKVFLNIGDKFLLDANLGKGEGDKEKVGIDYKGLPADVVPGDILLLDDGRVQLKVREVQGMKVFTEVTVGGPLSNNKGINKLGGGLSAEALTEKDKADIKTAALIGVDYLAVSFPRCGEDLNYARRLARDAGCDAKIVAKVERAEAVCSQDAMDDIILASDVVMVARGDLGVEIGDPELVGIQKALIRRARQLNRAVITATQMMESMITNPMPTRAEVMDVANAVLDGTDAVMLSAETAAGQYPSETVAAMARVCLGAEKIPSINVSKHRLDVQFDNVEEAIAMSAMYAANHLKGVTAIITMTESGRTALMTSRISSGLPIFAMSRHERTLNLTALYRGVTPVHFDSANDGVAAASEAVNLLRDKGYLMSGDLVIVTQGDVMSTVGSTNTTRILTVE

SEQ ID NO: 93 - DNA, 1443 bp, Escherichia coli
atgtccagaaggcttcgcagaactaaaatcgttaccacgttaggcccggcaacagatcgcgataataaccttgaaaaagttatcgcggcgggtgccaacgttgtacgtatgaatttttctcacggctcgcctgaagatcacaaaatgcgcgcggataaagttcgtgagattgcctcaaaactggggcgtcatgtggctattctgggtgacctccaggggcccaaaatccgtgtatccacctttaaagaaggcaaagttttcctcaatattggggataaattcctgctcgacgccaacctgggtaaaggtgaaggcgacaaagaaaaagtcggtatcgactacaaaggcctgcctgctgacgttgtgcctggtgacatcctgctgctggacgatggtcgcgtccagttaaaagtactggaagtccagggcatgaaagtgttcaccgaagtgaccgtcggtggtcccctctccaacaataaaggtatcaacaaacttggcggcggtttgtcagctgaagcgctgaccgaaaaagacaaagcagacattaagactgcggcgttgattggcgtagattacctggctgtctccttcccacgctgcggcgaagatctgaactatgcccgtcgcctggcacgcgatgcaggatgtgatgcgaaaattgttgccaaggttgaacgtgcggaagccgtttgcagccaggaggcaatggatgacatcatcctcgcctctgacgtggtaatggttgctcgcggcgacctcggtgtggaaattggcgatccggaactggtcggcattcagaaagcgttgatccgtcgtgcgcgtcagctaaaccgcgcggtaatcacggcgacccagatgatggaatcaatgattactaacccgatgccgacgcgtgcagaagtcatggacgtagcaaacgccgttctggatggtactgacgctgtgatgctgtcggcagaaactgctgctggtcaatacccgtcagaaactgttgcagccatggcgcgcgtttgcctgggggcggaaaaaatcccgagcatcaacgtttctaaacaccgtctggacgttcagttcgacaatgtggaagaagctattgccatgtcagcaatgtacgcggctaaccacttgaaaggcgttacggcgatcatcaccatgaccgaatcgggtcgtaccgcgctgatgacctcccgtatcagctctggtctgccaattttcgccatgtcacgccatgaacgtacgctgaacctgactgctctctatcgcggcgttacgccggtgcactttgatagcgctaatgacggtgtagcagctgccagcgaagcggttaatctgctgcgtgataaaggttacttgatgtctggtgacctggtgattgtcacccagggcgacgtgatgagtaccgtgggttctactaataccacgcgtattttaacggtagagtaa

SEQ ID NO: 94 - protein, 480 aa, Escherichia coli
MSRRLRRTKIVTTLGPATDRDNNLEKVIAAGANVVRMNFSHGSPEDHKMRADKVREIASKLGRHVAILGDLQGPKIRVSTFKEGKVFLNIGDKFLLDANLGKGEGDKEKVGIDYKGLPADVVPGDILLLDDGRVQLKVLEVQGMKVFTEVTVGGPLSNNKGINKLGGGLSAEALTEKDKADIKTAALIGVDYLAVSFPRCGEDLNYARRLARDAGCDAKIVAKVERAEAVCSQEAMDDIILASDVVMVARGDLGVEIGDPELVGIQKALIRRARQLNRAVITATQMMESMITNPMPTRAEVMDVANAVLDGTDAVMLSAETAAGQYPSETVAAMARVCLGAEKIPSINVSKHRLDVQFDNVEEAIAMSAMYAANHLKGVTAIITMTESGRTALMTSRISSGLPIFAMSRHERTLNLTALYRGVTPVHFDSANDGVAAASEAVNLLRDKGYLMSGDLVIVTQGDVMSTVGSTNTTRILTVE

SEQ ID NO: 95 - DNA, 1443 bp, Photorhabdus luminescens
  Photorhabdus luminescens subsp. laumondii TTO1
atgtccagacggctcagaagaactaagattgttactacacttggtccggctaccgaccgcgataataatcttgaaaagattattgctgcgggtgcaaacgtagtccgccttaacttttcccacggtaccgccgaagatcatctccagcgagccagcaaagtccgcgagattgcagcccgcctggggtgccatgtcgctattcttggcgatcttcaaggacctaaaatccgcgtatcaacgtttaaagagggcaaaattttcctgaatatcggagataaatttctccttgatgccaatcttggtaaaggcgaaggcgataaggagaaagtcggtatcgattacaaaggactacccacggatgttgtaccgggcgatattttgttgctggatgacggacgtgttcaactcaaagtattagacgttcagggaatgaaagtcttcacggaagtcactgtaggcggcccgctatctaacaataaaggcattaacaaactgggtggtggattatccgcagaagccctaactgaaaaagataaagaagatattattacggcggccagaatcggcgttgattaccttgccgtctctttccctcgcaccggtgaagatcttaactatgcccgccgtctggcacgagacgcaggctgtgaaacgcagatcgtggcaaaagttgaacgcgcagaagccgttagcagtgataaaaccatcgatgaaatcattctggcttctgatgtggtcatggttgcccgtggtgaccttggcgtagaaatcggcgatccagagttagttggcgttcagaaaaaattaattcgccgtgcccgccagctaaatcgtgtagttattaccgcgacccagatgatggaatctatgatcaccaatccaatgcccacccgtgcagaagtgatggatgtggcaaatgccgttctcgacggtactgacgccgtaatgctctccgctgaaaccgcagcgggccaataccctgccgaaaccgttgccgcaatggcacgagtctgtctcggagcagaaaaaatgcctggcctcaacgtatcaaaacatcggctagacacaatttttgacagtattgaagaagccattgcgatgtctaccatgtacgcggcaaaccacttaaatggagttaaagccattattgccatgaccgaatcaggccgcaccactcgcatcatgtctcgcattagttctggtctcccgatattctctatgtcccgtcatgagaaaaccttaaaccaaacggcactttaccgtggcgttacaccggtttattgcagtagtcatactgacggcattacggcggccaatgaagcagttaatcgcctgcgtgataaaggttatctagtttccggtgatcttgttctggttactcagggtgatcagatggggacgattggcagtactaacacctgccgtattcttgaagtggaataa

SEQ ID NO: 96 - protein, 480 aa, Photorhabdus luminescens
  Photorhabdus luminescens subsp. laumondii TTO1
MSRRLRRTKIVTTLGPATDRDNNLEKIIAAGANVVRLNFSHGTAEDHLQRASKVREIAARLGCHVAILGDLQGPKIRVSTFKEGKIFLNIGDKFLLDANLGKGEGDKEKVGIDYKGLPTDVVPGDILLLDDGRVQLKVLDVQGMKVFTEVTVGGPLSNNKGINKLGGGLSAEALTEKDKEDIITAARIGVDYLAVSFPRTGEDLNYARRLARDAGCETQIVAKVERAEAVSSDKTIDEIILASDVVMVARGDLGVEIGDPELVGVQKKLIRRARQLNRVVITATQMMESMITNPMPTRAEVMDVANAVLDGTDAVMLSAETAAGQYPAETVAAMARVCLGAEKMPGLNVSKHRLDTIFDSIEEAIAMSTMYAANHLNGVKAIIAMTESGRTTRIMSRISSGLPIFSMSRHEKTLNQTALYRGVTPVYCSSHTDGITAANEAVNRLRDKGYLVSGDLVLVTQGDQMGTIGSTNTCRILEVE

SEQ ID NO: 97 - DNA, 1443 bp, Photorhabdus asymbiotica
  Photorhabdus asymbiotica subsp. asymbiotica ATCC 43949
atgtccagacggctcagaagaactaagattgttactacccttggtccggctaccgaccgcgataataaccttgaaaagattattacagcaggtgcaaacgtagtccgcctcaacttttcccacggtacagcagaagatcacatccagcgtgccaataaagtgcgcgaaatagctgcccgccttggccgccatgttgctattcttggcgatcttcaaggacctaaaattcgcgtatcaacttttaaagagggcaaaattttcctgaatgtcggcgataaatttctacttgatgccaaccttggtaaaggcgaaggcaataaagaaaaagtcggcatcgattacaaaggattacccgctgatgtcgtaccgggtgatattctgctgctagatgatggacgtgttcaactgaaagtattagaagtgcaggccatgaaagtattcacggaagttactgttggtggcccgctgtctaacaataaaggcatcaacaagctcggtggtggtttatccgctgaagcgctgaccgaaaaggataaagaagacattattaccgccgccagaattggtgttgattacctcgcaatctcttttcctcgtactggtgaagatctcaattatgcccgtcgtttggcacgggatgctggttgtgaaacgcaaatcgtggcaaaagttgagcgtgcagaagccgtcagcagtgatgaaatcattgatgaaattattctggcttccgatgttgtgatggttgcccgtggtgatctcggcgtggaaatcggtgatcctgaactggttggcgttcagaaaaaactcattcgccgtgcacgccagttgaatcgtgtggtcattaccgctacccaaatgatggaatccatgatcaccaatccaatgcctacccgcgcagaagttatggacgtagcgaacgcagtacttgacggtaccgacgcagtgatgctttctgctgaaacagcctcaggtcaataccctgccgaaacggttgccgctatggctcaggtctgtctgggtgccgaaaaaatgccaggactcaatgtatccaaacatcggttagacaccattttcgaaagtaccgaagaagccattgctatgtccaccatgtacgccgcaaaccatctaaaaggtgtgaaagcgattatcgcactgaccgaatcaggtcgcaccacacggatgatgtcgcgcatcagttccggcctgcacattttctctatgtcccgtcatgaaaagacgctgaatcaaacagcgctttaccgcggcgttacaccggtttattgcagtagtcatactgacggtataacagccgctaatgaagccatcaaccgtctgcgtgataaaggttatctgatttccggcgaccttgttctggttactcaaggcgaccagatgggcacaattggcagcactaacacctgccgtattcttgaagtagaataa

SEQ ID NO: 98 - protein, 480 aa, Photorhabdus asymbiotica
  Photorhabdus asymbiotica subsp. asymbiotica ATCC 43949
MSRRLRRTKIVTTLGPATDRDNNLEKIITAGANVVRLNFSHGTAEDHIQRANKVREIAARLGRHVAILGDLQGPKIRVSTFKEGKIFLNVGDKFLLDANLGKGEGNKEKVGIDYKGLPADVVPGDILLLDDGRVQLKVLEVQAMKVFTEVTVGGPLSNNKGINKLGGGLSAEALTEKDKEDIITAARIGVDYLAISFPRTGEDLNYARRLARDAGCETQIVAKVERAEAVSSDEIIDEIILASDVVMVARGDLGVEIGDPELVGVQKKLIRRARQLNRVVITATQMMESMITNPMPTRAEVMDVANAVLDGTDAVMLSAETASGQYPAETVAAMAQVCLGAEKMPGLNVSKHRLDTIFESTEEAIAMSTMYAANHLKGVKAIIALTESGRTTRMMSRISSGLHIFSMSRHEKTLNQTALYRGVTPVYCSSHTDGITAANEAINRLRDKGYLISGDLVLVTQGDQMGTIGSTNTCRILEVE

SEQ ID NO: 99 - DNA, 1437 bp, Actinobacillus succinogenes
  Actinobacillus succinogenes 130Z
atgtctagaagattaagaagaacgaaaatcgtatgtacgatgggacctgcgaccgacaaaggcaacaaccttgaaaaaatcattgccgcaggtgcaaacgtagtacgtatgaacttctcccacggaacgccggaagatcatatcgcccgtgccgataaagtacgagaaattgcgaaaaaattaggcaaacacgtcgcaatcttgggtgatttacaaggccctaaaatccgggtttccactttcaaagaaggcaaaattttcttaagtatcggtgataaatttattttagatgcggatttaccgaaaggtgaaggtaatcaggaagcggtcggattggattacaaaactttaccgcaggatgtcgtgccgggcgatattttattgttagacgacggacgcgttcagttaaaagtattatctacggacggcgcgaaagtgtttaccgaagtaacggtaggcggtcctttgtcaaacaacaaaggtatcaataaattaggcggcggtttatcggcggacgcattaaccgagaaagataaagcggatatcgttaccgcggcacgtatcggcgtagattatctggcggtgtctttcccgcgttcaagcgcagacttaaattatgcccgcgaattggcggaagccgccggcttagatgcaaaaatcgtagcgaaagtagaacgtgcggaaaccgtggcgactgacgaagctatggacgatattatcttagcgtcggacgttatcatggttgcccgcggtgacttgggggtggaaatcggtgacccggaattagtgggcgttcagaaaaaattaatccgtcgttcacgtcagttaaaccgagtggttatcactgcgactcagatgatggaatcgatgatcagcaacccgatgcctacccgtgcggaagtaatggacgtcgcgaatgccgtgttggacggtaccgatgccgtaatgctttcagcggaaaccgcagccggtcaatatccgtccgaaacggtggcggctatggcgaaagtggcgttgggggcagaaaaaatgccgagtatcaacgtatctaaacaccgtatggacggtcaattcgccactatcgaagaatccgtagcgatgtcttccatgtatgcggcaaaccacatgaaaggtgtggcggcgattattaccttgaccaacagcggccgtacggcgcgtttaatgtcgcgtatcagttccggtttaccgatttttgccttatcgcgtaacgaatccactttaaacttatgcgcgttataccgcggtgtaacgccgattctgttcgaacaggaatcccgcaccgtacacagcgcgaaagcggcggtcaatctgttgaaagaaaaaggattcttggtgtcgggcgacttggtgttattaactcagggggacgcactaggcacatcaactaacgtatgccgcacattaatcgttgaataa

SEQ ID NO: 100 - protein, 478 aa, Actinobacillus succinogenes
  Actinobacillus succinogenes 130Z
MSRRLRRTKIVCTMGPATDKGNNLEKIIAAGANVVRMNFSHGTPEDHIARADKVREIAKKLGKHVAILGDLQGPKIRVSTFKEGKIFLSIGDKFILDADLPKGEGNQEAVGLDYKTLPQDVVPGDILLLDDGRVQLKVLSTDGAKVFTEVTVGGPLSNNKGINKLGGGLSADALTEKDKADIVTAARIGVDYLAVSFPRSSADLNYARELAEAAGLDAKIVAKVERAETVATDEAMDDIILASDVIMVARGDLGVEIGDPELVGVQKKLIRRSRQLNRVVITATQMMESMISNPMPTRAEVMDVANAVLDGTDAVMLSAETAAGQYPSETVAAMAKVALGAEKMPSINVSKHRMDGQFATIEESVAMSSMYAANHMKGVAAIITLTNSGRTARLMSRISSGLPIFALSRNESTLNLCALYRGVTPILFEQESRTVHSAKAAVNLLKEKGFLVSGDLVLLTQGDALGTSTNVCRTLIVE

SEQ ID NO: 101 - protein, 13 aa, Artificial Sequence
  Pyruvate kinase active site
XXXXXKXEXXXXX

SEQ ID NO: 102 - protein, 92 aa, Artificial sequence
  Pfam domain PF00224 consensus sequence
GXXGXXXXXXXXXXXQXXXXXXXXXXXXXXXXXXXXXXSMXXXXXPTRAEXXDXXXAXXXXXDXXXLXXEXXXGXXPXXXXXXXXXXXXXXX

SEQ ID NO: 103 - protein, 22 aa, Artificial Sequence
  Pfam domain PF02887 consensus sequence
TXXGXXXXXXXXXRXXXXXXXX

SEQ ID NO: 104 - DNA, 1198 bp, Zea mays
tcccgtgtccgtcaatgtgatactactagcatagtactagtaccatgcatacacacagcaggtcggccgcctggatggatcgatgatgatactacatcatcctgtcatccatccaggcgatctagaaggggcgtggctagctagcaaactgtgaccggtttttctacgccgataataatactttgtcatggtacagacgtacagtactggttatatatatctgtagatttcaactgaaaagctaggatagctagattaattcctgagaaacacagataaaattcgagcttggctatagatgacaaaacggaagacgcatgcattggacgacgtatgcaatgcgagcgcgtctcgtgtcgtcccgtccaagtctggcgatctcacgccacgtgctcaacagctcaaggactgttcgtcaccagcgttaaattcattgaagggatgacgcatttcggcatttgtcattgcttgtagctatatatatatatccaacagatttctctcaagcttttgtatgcgtgaatgtaaagtctagcttatacgacagcacgtgcagatatattaacgtcattattaggtggagagcaagatgcatgatctggtagaaattgtcgaaaacacaagagagagtgaagtgcacacttctggtataggagtgtatacgccgctggttggtgggcaatgcgcgccgcaatattggccaatgaaacctagcaacgcccactcgccacgccccatgaatggcccccgcacgacagcgagccagccagtgcccgcgcgcggcccagccggagtcggcggaacgcgccacgggggacaaggcgcccgagggccgaggcagcgcggcatggcaagcaagccgaagcgggcaagcgacctgcatgcagcccctgcacctcgccctcgtcagtcgtcccagcctcccactggaatccacccaacccgcccttcctctacaaagcacgcgccccgcgactcgcctccgcctacgtgtcggcagcgtccccgccggtcgcccacgtaccccgccccgttctcccacgtgcccctccctctgcgcgcgtccgattggctgacccgcccttcttaagccgcgccagcctcctgtccgggccccaacgccgtgctccgtcgtcgtctccgcccccagagtgatcgagcccactgacctggcccccgagcctcagctcgtgagtcc

SEQ ID NO: 105 - DNA, 1526 bp, Zea mays
cagggagagctatgaggcgtatgtcctcaaagccactttgcattgtgtgaaaccaatatcgatctttgttacttcatcatgcgtgaacatttgtggaaactactagcttacaagcattagtgacagctcagaaaaaagttatctctgaaaggtttcatgtgtaccgtgggaaatgagaaatgttgccaactcaaacaccttcaatatgttgtttgcaggcaaactcttctggaagaaaggtgtctaaaactatgaacgggttacagaaaggtataaaccacggctgtgcattttggaagtatcatctatagatgtctgttgaggggaaagccgtacgccaacgttatttactcagaaacagcttcaacacacagttgtctgctttatgatggcatctccacccaggcacccaccatcacctatctctcgtgcctgtttattttcttgccctttctgatcataaaaaatcattaagagtttgcaaacatgcataggcatatcaataattcaatatgctcatttattaatttgctagcagatcatcttcctactctttactttatttattgtttgaaaaatatgtcctgcacctagggagctcgtatacagtaccaatgcatcttcattaaatgtgaatttcagaaaggaagtaggaacctatgagagtatttttcaaaattaattagcggcttctattatgtttatagcaaaggccaagggcaaaattggaacactaatgatggttggttgcatgagtctgtcgattacttgcaagaaatgtgaacctttgtttctgtgcgtgggcataaaacaaacagcttctagcctcttttacggtacttgcacttgcaagaaatgtgaactccttttcatttctgtatgtggacataatgccaaagcatccaggctttttcatggttgttgatgtctttacacagttcatctccaccagtatgccctcctcatactctatataaacacatcaacagcatcgcaattagccacaagatcacttcgggaggcaagtgcgattttgatcttgcagccacctttttttgttctgttgtaagtatactttcccttaccatctttatctgttagtttaatttgtaattgggaagtattagtggaaagaggatgagatgctatcatctatgtactctgcaaatgcatctgacgttatatgagctgcttcatataatttgaattgctccattcttgccgacaatatattgcaaggtatatgcctagttccatcaaaagttctgttttttcattctaaaagcattttagtggcacacaatttttgtccatgagggaaagggaatctgttttggttactttgcttgaggtgcattcttcatatgtccagttttatggaagtaataaacttcagtttggtcataagatgtcatattaaagggcaaacatatattcaatgttcaattcatcgtaaatgttccctttttgtaaaagattgcatactcatttatttgagttgcaggtgtatctagtagttggaggag

SEQ ID NO: 106 - DNA, 1136 bp, Zea mays
aagcttgctactttctttccttaatgttgatttcccctttgttagatgttctttgtgttatatacactctgtatacaaggatgcgatacacacatcagctagtcctaatgatgccaccgactttacttgaggaaaaggaaacaaatatgatgtggccatcacattctcaataacaatgaccatgtgcgcaatgacataccatcatatttgatatcataaaaataaatttattatcaaagtaaacatatagttcatatatcagatattaaagtgataagaacaaatattacattttatcttatataaaatgacgaaaggtacgagttgaaaaggggtccaaccccttttttatagcttgttcggttgcttgttctccttcggctagcgaggtggtagaatgtgagagtgttgcgcgtggattcccgtcgtagtgttcttaggtgatttctcacggcccatctgtgatatagcgactcattatgtggtgtaatagcccattgggagaaggggagagatatagatctacgtgatttgcgcgtgatgcacgacgaacgaaactggtggtttaaagtagtagaggtttgtcattagtggtgtaagtggtacatatattatccgttcatattcgaatttgatccgtataagggggctaagatctaatccgtatacaagtccaagtattaagtatccgatccatatcggatctttatccgtatccgtatactcaaaatttgatgtttaagattttaatatatatttaaactttataggaactcgataatatttgtatctgatttgaattgtgaaaacaaatatggaacgattaatttcagtctatatccgttccgatatttgtcatgctttgctaaaaatacctttacaaggcatcttgtgcagattatatattaatctgaaatcagttagagaagcctacaaatttgaccaaatgccgagtcatccggcttatcccctttccaactttcagttctgcaagcgccagaaatcgtttttcatctacattgtctttgttgcctgcatacatctataaataggacctgctagatcaatcgcagtccatcggcctcagtcgcacatatctactatactatactctaggaagcaaggacaccaccgcc

SEQ ID NO: 107 - DNA, 856 bp, Zea mays
cgtagcaatgcacgggcatataactagtgcaacttaatacatgtgtgtattaagatgaataagagggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgaca

SEQ ID NO: 108 - DNA, 1401 bp, Zea mays
ccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgttttttctataaaaaaagggaaactgcccctcatttacagcaaattgtccgctgcctgtcgtccagatacaatgaacgtacctagtaggaactcttttacacgctcggtcgctcgccgcggatcggagtcccaggaacacgacaccactgtgtaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgctacctccgctttagttctgcatacagccaacccaa

SEQ ID NO: 109 - DNA, 1421 bp, Sugarcane bacilliform virus
gaagttgaagacaaagaaggtcttaaatcctggctagcaacactgaactatgccagaaaccacatcaaagatatgggcaagcttcttggcccattatatccaaagacctcagagaaaggtgagcgaaggctcaattcagaagattggaagctgatcaataggatcaagacaatggtgagaacgcttccaaatctcactattccaccagaagatgcatacattatcattgaaacagatgcatgtgcaactggatggggagcagtatgcaagtggaagaaaaacaaggcagacccaagaaatacagagcaaatctgtaggtatgccagtggaaaatttgataagccaaaaggaacctgtgatgcagaaatctatggggttatgaatggcttagaaaagatgagattgttctacttggacaaaagagagatcacagtcagaactgacagtagtgcaatcgaaaggttctacaacaagagtgctgaacacaagccttctgagatcagatggatcaggttcatggactacatcactggtgcaggaccagagatagtcattgaacacataaaagggaagagcaatggtttagctgacatcttgtccaggctcaaagccaaattagctcagaatgaaccaacggaagagatgatcctgcttacacaagccataagggaagtaattccttatccagatcatccatacactgagcaactcagagaatggggaaacaaaattctggatccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcacca

SEQ ID NO: 110 - DNA, 300 bp, Sugarcane bacilliform virus
agatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcacca

SEQ ID NO: 111 - DNA, 583 bp, Orzya sativa
gtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcag

SEQ ID NO: 112 - DNA, 331 bp, Oryza sativa
gtaagttctggctttcttgcttttggataaattttgcttcctttcttaacttgagcacaagcttgtgttatatgtggtgtggaatcttggttgccatgttgtgaggatttagctagagagtcaagaaagaggaatatatgctttatgtagataggagtaggatctctgggtctttaaacatcaccatgacaagcaaagataagaacaggagagcagttcttgattattatttttcttctcatcaagaaattaagccggagatagacatggcagctgcacgcagtgattcacttcttgatttcttgatttgggttgttgcgtttgtgtccag

SEQ ID NO: 113 - DNA, 153 bp, Silene pratensis
atggcttctacactctctaccctctcggtgagcgcatcgttgttgccaaagcaacaaccgatggtcgcctcatcgctaccaaccaacatgggccaagccttgtttggactgaaagccggttctcgtggcagagtgactgcaatggccacatac

SEQ ID NO: 114 - protein, 51 aa, Silene pratensis
MASTLSTLSVSASLLPKQQPMVASSLPTNMGQALFGLKAGSRGRVTAMATY

SEQ ID NO: 115 - DNA, 253 bp, Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 116 - DNA, 709 bp, Agrobacterium tumefaciens
tcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagca

SEQ ID NO: 117 - protein, 643 aa, Artificial sequence
  Consensus sequence of Figure 1
MAQVVATRSIXSSMLXPXSGXXSXXRXXXLXXPXSFAXKVLXXXXXRXXRXXXXXXXXXXXXXXXXXRXXXXXTEVIPVSPEDXPXXXXXXXXXXXXREEXLXXXXEMQQLGDTXXXXXXXSVGMWSKPTVRRKTKIVCTIGPSTNTREMIWKLAEAGMNVARLNMSHGDHASHQKVIDLVKEYNAQSKDNVIAIMLDTKXXXXGPEVRSGDLPXXQPIXLXXGQEFTFTIRRGVXXXTXDCVSVNYDDFVNDVEVGDMLLVDGGMMSLMVKSKTXDSVKCEVVDGGELKSRRHLNXXXXXXXXXXXVRGKSATLPSITEKDWEDIKXFGVDNKVDFYAVSFVKDAQVVHELKNYLKSCXXGADIHVIVKIESADSIPNLHSIITASDGAMVARGDLGAELPIEEVPLLQEEIIXLCRSMGKAVIVATNMLESMIVHPTPTRAEVSDIAIAVREGADAVMLSGETAHGKFPLKAVXVMHTVALRTEATIXXGXXXXXXMPPNLGQAFKNHMSEMFAYHATMMSNTLGTXSIVVFTRTGFMAILLSHYRPSGTIFAFTNXXXXXXXXXXXXXEKRIQQRLALYQGVCPIYMEFSDDAEETFAXALALLXKQGMVKXGEEVALVQSGXQPIWRXQSTHNIQVRKV

SEQ ID NO: 118 - protein, 578 aa, Artificial sequence
  Consensus sequence of Figure 2
AQVVATKXIXSSMXXPXXXXXSSRNXXLXLXXXXXXTLIGXXARXTXIXXRXXXLXXXVTSXKXXEDSXVIPVSPEDVPXXXEXLEXXXEMXRFGDTSVSMWSKPXVRRKTKIVCTIGPSTNTREMIWKLAEAGMNVARLNMSHGDHASHQKVIDLVKEYNAQSKDNXIAIMLDTKGPEVRSGDLPQPIXLEXGQEFTFTIKRGVSTXDCVSVNYDDFVNDVEXGDMLLVDGGMMSLMVKSKTXDSVKCEVVDGGELKSRRHLNVRGKSATLPSITDKDWEDIKFGVDNXVDFYAVSFVKDAXVVHELKNYLKSCGADIXVIVKIESADSIPNLHSIITASDGAMVARGDLGAELPIEEVPLLQEEIIRXCRSMGKAVIVATNMLESMIVHPTPTRAEVSDIAIAVREGADAVMLSGETAHGKFPLKAVXVMHTVALRTEASLTXGXTPANLGQAFKNHMSEMFAFHATIMSNTLGTSLVVFTRTGFMAVLLSHYRPSGTIFAFTNQKRIQQRLALYQGVCPIYMEFSDDAEETFAXALXLLXXXGMVKEGEEVALVQSGSQPIWRXQSTHNIQVRKV

SEQ ID NO: 119 - protein, 482 aa, Artificial sequence
  Consensus sequence of Figure 3
MSRRLRRTKIVTTLGPATDRDNNLEKIIAAGANVVRMNFSHGSPEDHXMRADKVREIAAKLGRHVAILGDLQGPKIRVSTFKEGXKVFLNIGDKFLLDANLGKGEGDKEKVGIDYKGLPADVVPGDILLLDDGRVQLKVLEVQGMKVFTEVTVGGPLSNNKGINKLGGGLSAEALTEKDKADIITAAKIGVDYLAVSFPRXGEDLNYARRLARDAGCDAKIVAKVERAEAVXSXEAMDDIILASDVVMVARGDLGVEIGDPELVGIQKKLIRRARQLNRXVITATQMMESMITNPMPTRAEVMDVANAVLDGTDAVMLSAETAAGQYPSETVAAMARVCLGAEKIPSINVSKHRLDXQFDXVEEAIAMSAMYAANHLKGVXAIIXMTESGRTALMMSRISSGLPIFAMSRHERTLNLTALYRGVTPVHFDSXTDGXVXAAXEAVNLLRDKGYLMSGDLVIVTQGDVMXTVGSTNTXRILXVE

SEQ ID NO: 120 - DNA, 153 bp, Silene pratensis
atggcttctacactctctaccctctcggtgagcgcatcgttgttgccaaagcaacaaccgatggtcgcctcatcgctaccaactaatatgggtcaagccttgtttggactgaaagccggttctcgtggcagagtgactgcaatggccacctac

SEQ ID NO: 121 - DNA, 519 bp, Arabidopsis thaliana
ttagatctcgtgccgtcgtgcgacgttgttttccggtacgtttattcctgttgattccttctctgtctctctcgattcactgctacttctgtttggattcctttcgcgcgatctctggatccgtgcgttattcattggctcgtcgttttcagatctgttgcgtttcttctgttttctgttatgagtggatgcgttttcttgtgattcgcttgtttgtaatgctggatctgtatctgcgtcgtgggaattcaaagtgatagtagttgatattttttccagatcaggcatgttctcgtataatcaggtctaatggttgatgattctgcggaattatagatctaagatcttgattgatttagatttgaggatatgaatgagattcgtaggtccacaaaggtcttgttatctctgctgctagatagatgattatccaattgcgtttcgtagttatttttatggattcaaggaattgcgtgtaattgagagttttactctgttttgtgaacaggcttgatcaaa

SEQ ID NO: 122 - DNA, 847 bp, Arabidopsis thaliana
tggtgcttaaacactctggtgagttctagtacttctgctatgatcgatctcattaccatttcttaaatttctctccctaaatattccgagttcttgatttttgataacttcaggttttctctttttgataaatctggtctttccatttttttttttttgtggttaatttagtttcctatgttcttcgattgtattatgcatgatctgtgtttggattctgttagattatgtattggtgaatatgtatgtgtttttgcatgtctggttttggtcttaaaaatgttcaaatctgatgatttgattgaagcttttttagtgttggtttgattcttctcaaaactactgttaatttactatcatgttttccaactttgattcatgatgacacttttgttctgctttgttataaaattttggttggtttgattttgtaattatagtgtaattttgttaggaatgaacatgttttaatactctgttttcgatttgtcacacattcgaattattaatcgataatttaactgaaaattcatggttctagatcttgttgtcatcagattatttgtttcgataattcatcaaatatgtagtccttttgctgatttgcgactgtttcattttttctcaaaattgttttttgttaagtttatctaacagttatcgttgtcaaaagtctctttcattttgcaaaatcttctttttttttttgtttgtaactttgttttttaagctacacatttagtctgtaaaatagcatcgaggaacagttgtcttagtagacttgcatgttcttgtaacttctatttgtttcagtttgttgatgactgctttgattttgtaggtcaaa

SEQ ID NO: 123 - DNA, 822 bp, Oryza sativa
ggttcttggtatatgccaacttttgtagcctgcaccagaaacaaaaatgaagacttttgctaaagatgtaaaagtggcatgatgtcctggatgaccaaataattcatgacaaatggattaaaagagcccaatatctgaaagagactggccagcagccactaatgtcaccaaccacatatgtaacacttggtgcataattcaagagggagcatctcctccagaatcaggattgaaaggtacaacctcatagtaaatcctcggaatatagcatgtgcagcataagaatatatcagtgttgtgctgggtaagaaaccacatgaaccaattaggaataaataatcatgctgaaattatagcaatgcttgcaatttgcaaacgataaagctagacgcgggttgctggaataacaatccatctccaacaaaatagtacagaatataactgaatggccagctcagaccctaacagaattgaaaagctggattcatcagcactccattgagcaatctagatcaggaaagagcatagatgcataatgaactgagatcccttcaaaatgactaactaatatttttttttcttataaaagagtttacaacagtacaaccacgaagatcagcactaccattactgattttgttaacatagagtgatttatcatgtgtgccagacaaacaacagatacattcatacatagcataacttacagcacatgatacagactacggagaacggttaatcttaaaataaaaacaaaaaaacaaggaggcaaagcttattttgcctgggattcatctaaatgcagttgtgtgcagaaggaga

SEQ ID NO: 124 - DNA, 99 bp, Citrullus lanatus
atgggatccatgaaagcatccattcttagatcagtccgctcagctgtctcacgctctagctcttctaatagactcctgtcccgtagttttgcaacacat

SEQ ID NO: 125 - protein, 33 aa, Citrullus lanatus
MGSMKASILRSVRSAVSRSSSSNRLLSRSFATH

SEQ ID NO: 126 - DNA, 946 bp, Zea mays
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatc

SEQ ID NO: 127 - DNA, 1012 bp, Zea mays
gtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 128 - DNA, 1198 bp, Zea mays
tcccgtgtccgtcaatgtgatactactagcatagtactagtaccatgcatacacacagcaggtcggccgcctggatggatcgatgatgatactacatcatcctgtcatccatccaggcgatctagaaggggcgtggctagctagcaaactgtgaccggtttttctacgccgataataatactttgtcatggtacagacgtacagtactggttatatatatctgtagatttcaactgaaaagctaggatagctagattaattcctgagaaacacagataaaattcgagcttggctatagatgacaaaacggaagacgcatgcattggacgacgtatgcaatgcgagcgcgtctcgtgtcgtcccgtccaagtctggcgatctcacgccacgtgctcaacagctcaaggactgttcgtcaccagcgttaaattcattgaagggatgacgcatttcggcatttgtcattgcttgtagctatatatatatatccaacagatttctctcaagcttttgtatgcgtgaatgtaaagtctagcttatacgacagcacgtgcagatatattaacgtcattattaggtggagagcaagatgcatgatctggtagaaattgtcgaaaacacaagagagagtgaagtgcacacttctggtataggagtgtatacgccgctggttggtgggcaatgcgcgccgcaatattggccaatgaaacctagcaacgcccactcgccacgccccatgaatggcccccgcacggcagcgagccagccagtgcccgcgcgcggcccagccggagtcggcggaacgcgccacgggggacgaggcgcccgagggccgaggcagcgcggcatggcaagcaagccgaagcgggcaagcgacctgcatgcagcccctgcccctcgccctcgtcagtcgtcccagcctcccactggaatccacccaacccgcccttcctctccaaagcacgcgccccgcgactcgcctccgcctacgtgtcggcagcgtccccgccggtcgcccacgtaccccgccccgttctcccacgtgcccctccctctgcgcgcgtccgattggctgacccgcccttcttaagccgcgccagcctcctgtccgggccccaacgccgtgctccgtcgtcgtctccgcccccagagtgatcgagcccactgacctggcccccgagcctcagctcgtgagtcc

(19) United States
(12) Patent Application Publication
(10) Pub. No.: US 2013/0045325 A1
(43) Pub. Date: Feb. 21, 2013

(54) METHODS OF INCREASING PROTEIN, OIL, AND/OR AMINO ACID CONTENT IN A PLANT

(71) Applicants: Heiko Härtel, Berlin (DE); Volker Haake, Berlin (DE); Alex Cernac, Raleigh, NC (US); Jamie Härtel, Berlin (DE); Christopher Kafer, Raleigh, NC (US)

(72) Inventors: Heiko Härtel, Berlin (DE); Volker Haake, Berlin (DE); Alex Cernac, Raleigh, NC (US); Jamie Härtel, Berlin (DE); Christopher Kafer, Raleigh, NC (US)

(73) Assignee: BASF Plant Science Company GmbH, Ludwigshafen (DE)

(21) Appl. No.: 13/587,095

(22) Filed: Aug. 16, 2012

Related U.S. Application Data
(60) Provisional application No. 61/525,232, filed on Aug. 19, 2011.

(51) Int. Cl. A01H 5/00; C12N 5/10; A01H 5/10; A23K 1/00; A01H 1/04; A23L 1/212; A23L 1/307; A23K 1/18; C12N 15/82; A01H 1/02

(52) U.S. Cl. 426/623; 435/320.1; 435/419; 800/298; 435/468; 800/281; 800/278; 800/275; 800/320.1; 800/264; 800/260; 426/615; 426/635; 426/665

(57) ABSTRACT

This invention relates generally to methods for preparing a plant, plant cell, or plant part with increased content in one or more of protein, oil, or one or more amino acids relative to a corresponding wild-type plant, plant cell, or plant part. Expression cassettes for achieving such gene expression manipulation, as well as recombinant constructs, vectors and plants, plant cells, or plant parts comprising the same, are also provided. Plants, plant cells, or plant parts with increased content in one or more of protein, oil, or one or more amino acids thus obtained may be useful in the preparation of foodstuffs and animal feeds. Plants, plant cells, or plant parts with increased content in one or more of protein, oil, or one or more amino acids thus obtained may also be useful in plant breeding programs for developing further hybrid or inbred lines.

### CROSS REFERENCE TO RELATED APPLICATIONS

This application claims the priority benefit of U.S. Provisional Application Ser. No. 61/525,232 filed Aug. 19, 2011, the entire contents of which is hereby incorporated by reference in its entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is Sequence_Listing_{—}17731_{—}00042_US. The size of the text file is 398 KB, and the text file was created on Jul. 13, 2012.

### FIELD OF THE INVENTION

This invention relates generally to methods for preparing a plant, plant cell, or plant part with increased content in one or more of protein, oil, or one or more amino acids relative to a corresponding wild-type plant, plant cell, or plant part by manipulating the expression level of a nucleic acid molecule encoding a polypeptide having pyruvate kinase activity in a plant, plant cell, or plant part. Expression cassettes for achieving such gene expression manipulation, as well as recombinant constructs, vectors and plants, plant cells, or plant parts comprising the same, are also provided. Plants, plant cells, or plant parts with increased content in one or more of protein, oil, or one or more amino acids thus obtained may be useful in the preparation of foodstuffs and animal feeds. Plants, plant cells, or plant parts with increased content in one or more of protein, oil, or one or more amino acids thus obtained may also be useful in plant breeding programs for developing further hybrid or inbred lines.

### BACKGROUND OF THE INVENTION

Crops such as rice, corn, soybean, sorghum, wheat, oats, rye, and barley are a major source of animal feed for many types of livestock and supply most of their dietary needs. These crops are also a primary source for human food and other industrial purposes. Corn tends to be the preferred feed grain because of its highly digestible carbohydrate content and relatively low fiber content, which is particularly important for swine and poultry (Hard, Proc. Southwest Nutr. Conf, 2005, 43-54). As a result, corn is the most widely produced feed grain globally, accounting for more than 90% of the grain used in feed. However, corn, as well as other crops commonly used as feed grain, have nutritional limitations such as protein and/or oil content, amino acid composition, minerals and vitamins for several types of livestock, especially swine, poultry, and cattle.

Because of the suboptimal protein and/or oil content and amino acid composition of plants, in comparison to the nutritional requirement of the animal, it is common practice to use feed additives and supplements, such as protein-rich feeds, amino acids, vitamins, minerals and fats in animal diets. The nutritional limitations of feed grain have become more critical as the demand for higher feeding efficiency has increased. The ratio of cereals to supplements in animal feed has changed through the years in an attempt to maximize feeding efficiency and minimize feeding costs. Major factors contributing to feed efficiency are the genetic potential of the animal and by the nutrients supplied to the animal. As the feed efficiency has improved due to genetic enhancements, the mineral and nutrient requirements for feed necessary to assure a complete and healthy diet have also risen. Since an animal's feed intake limits the amount of nutrients and calories it can consume, the feed industry has had to develop ways to make feeds that have improved protein quality, improved balance of essential amino acids, and metabolizable energy (oil).

Sources of feed protein, especially animal-derived protein, have come under global public scrutiny because of the bovine spongiform encephalopathy, or mad cow disease, crisis associated with the feeding of meat and bone meal as the primary protein source in animal diets in many parts of the world. Plant protein sources have become a dominant alternative protein supplement used in feed following bans on using meat and bone meal.

Plant protein sources, however, may lack sufficient levels of essential nutrients required for adequate animal health, growth and performance. Requirements vary depending on the species and age of the animal. For example, the order of the top three limiting amino acids in feed composed of corn and soybean meal is lysine, threonine, and tryptophan for swine, and methionine, lysine, and threonine for poultry. (FAO Animal Production and Health Proceedings, Protein Sources for the Animal Feed Industry, xi-xxv, 161-183 (2004)). These limiting amino acids must be available at specific minimum levels for the animals to use dietary protein efficiently. (Johnson et al. “Identification of Valuable Corn Quality Traits for Livestock Feed”, Report from the Center for Crops Utilization Research, Iowa State University, 1-22 (1999)). Furthermore, crude protein in feed ingredients is not totally digestible for any species. For example, corn protein is approximately 84% digestible by poultry and 82% digestible by swine (Johnson et al. (1999)). One method of increasing the nutritional quality of feed is to decrease crude protein in feed and supplement the feed with amino acids.

In addition to improving protein and amino acid composition, the feed industry has also had to develop ways to make feeds that are more calorie dense such as by adding fat to the feed, often in the form of a liquid such as oil. Fat has the advantage of supplying calories to each mouthful of feed. However, adding fat to feed has disadvantages such as increased cost, added labor, and technical difficulties associated with automatic feeding systems. Additionally, the fat is often of poor quality, thus reducing the overall quality of the feed. To reduce the use of liquid fat in feed, the industry has tried increasing the oil content of the grain used in feed. This extra oil in the grain reduces and may eliminate the need for the addition of liquid fat to the feed.

Each of the various ingredients necessary to produce the right combination of nutrients (i.e. protein, amino acids, enzymes, etc.) will need to be transported from site of production and/or processing to the site of the end-user. The availability, price, and transportation requirements and costs of each component of a particular feed will vary from year to year and in different geographical regions. Because of the variability of the supply and cost of nutrients and additives, livestock feeders and feed manufacturers would value plants with traits that decrease the need for more expensive feedstuffs and additives and can deliver increased nutrients in the same volume of grain.

Because feed is around 60% of animal production costs, any savings in feed costs can be considerable, especially in large operations. For example, nutritionally enhanced corn which can deliver higher levels of important nutrients and metabolizable energy, and/or enhanced digestibility and bioavailability of nutrients would provide the following benefits: reduced feed costs per unit weight gain or production of eggs or milk; reduced animal waste, particularly nitrogen and phosphorous; reduced veterinary costs and improved disease resistance; improved processing characteristics to make the feed; and improved quality (Johnson, et al. (1999)). Cost savings can be achieved by using nutritionally enhanced plants such as corn through, for example, reduced cost for needed supplements and synthetic additives, reduced transportation costs associated with the shipping of each additive and ingredients to produce the additives, reduced cost in mixing numerous additives during feed processing, and reduced costs associated with disposal of excess volume of manure.

Much effort has been instituted academically and industry-wide to improve the nutritional composition of feed grain. Both traditional plant breeding and biotechnology techniques have been used to develop plants with desirable traits. For example, U.S. Pat. No. 5,723,730 describes an inbred corn line used to produce a hybrid with elevated percent oil and protein in grain. U.S. Pat. No. 6,268,550 suggests that an increase in acetyl CoA carboxylase (ACCase) activity during the early to mid stages of soybean plant development leads to an increase in oil content. Zeh (Plant Physiol., 2001, 127: 792-802) describes increasing the methionine content in potato plants by inhibiting threonine synthase using antisense technology. U.S. Pat. No. 5,589,616 discloses producing higher amounts of amino acids in plants by overexpressing a monocot storage protein. Similar approaches have been used in U.S. Pat. No. 4,886,878, U.S. Pat. No. 5,082,993 and U.S. Pat. No. 5,670,635. Other methods for increasing amino acids are disclosed in WO 95/15392, WO 96/38574, WO 89/11789, and WO 93/19190. In these cases, specific enzymes in the amino acid biosynthetic pathway such as the dihydrodipicolinic acid synthase are deregulated leading to an increase in the production of lysine.

Examples of grain-based feed that provide improved animal nutrition and can reduce environmental impact of animal production are described by Chang et al. in U.S. Pat. Nos. 7,087,261 and 6,774,288 and in U.S. Publ. No. 2005/0246791.

Methods for producing plants having desirable high value traits are complex and involve particular difficulties or conditions. For example, high value traits are often associated with reduced plant vigor, yield, or seed viability.

There remains a need to develop plants with increased content in one or more of protein, oil, and/or one or more amino acids to reduce feed costs to supply improved quality food for both animals and humans. Crop plants, such as corn plants, having these desirable traits may be used as starting material for further breeding to develop additional inbred lines and hybrids with these traits.

### SUMMARY OF THE INVENTION

The present invention provides novel expression cassettes and methods for increasing content in one or more of protein, oil, or one or more amino acids in a plant, plant cell, or plant part. Recombinant constructs, vectors, and plant cells, plants or parts thereof, comprising the expression cassettes of the invention as well as methods for their production are also provided.

In one aspect, the invention provides an expression cassette conferring increased content in one or more of protein, oil, or one or more amino acids in a plant, plant cell, or plant part relative to a corresponding wild-type plant, plant cell, or plant part, comprising:

(a) a promoter that is functional in a plant;

(b) a nucleic acid molecule encoding a polypeptide having pyruvate kinase activity which is heterologous and operably linked to said promoter; and

(c) a rice intron,

wherein the nucleic acid molecule comprises:

(i) the nucleotide sequence of SEQ ID NO: 1, 3, 5, 7, 9, 11 or 13;

(ii) a nucleotide sequence encoding the amino acid sequence of SEQ ID NO: 2, 4, 6, 8, 10, 12 or 14;

(iii) a nucleotide sequence having at least 60% identity to the nucleotide sequence of SEQ ID NO: 1, 3, 5, 7, 9, 11 or 13 and encoding a polypeptide having a Pfam:PF00224 pyruvate kinase barrel domain and a Pfam:PF02887 pyruvate kinase alpha/beta domain;

(iv) a nucleotide sequence encoding an amino acid sequence having at least 60% identity to the amino acid sequence of SEQ ID NO: 2, 4, 6, 8, 10, 12 or 14 and having a Pfam:PF00224 pyruvate kinase barrel domain and a Pfam:PF02887 pyruvate kinase alpha/beta domain;

(v) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 109 to 449 of SEQ ID NO: 2 or the amino acid residues 98 to 439 of SEQ ID NO: 10, and wherein the Pfam:PF02887 pyruvate kinase alpha/beta domain has at least 80% identity to the amino acid residues 462 to 578 of SEQ ID NO: 2 or the amino acid residues 452 to 566 of SEQ ID NO: 10; or

(vi) a nucleotide sequence encoding an amino acid sequence having at least 60% identity to the amino acid sequence of SEQ ID NO: 2, 4, 6, 8, 10, 12 or 14, wherein said amino acid sequence further comprises the amino acid sequence of SEQ ID NO: 102 and 103.

In some embodiments, the promoter is a seed-specific or seed-preferential promoter. In other embodiments, the promoter is an endosperm-specific or endosperm-preferential promoter. In yet other embodiments, the promoter is an embryo-specific or embryo-preferential promoter.

In another aspect, the invention provides an expression cassette conferring increased content in one or more of protein, oil, or one or more amino acids in a plant, plant cell, or plant part relative to a corresponding wild-type plant, plant cell, or plant part, comprising:

(a) a promoter that is functional in a plant;

(b) a nucleic acid molecule encoding a polypeptide having pyruvate kinase activity which is heterologous and operably linked to said promoter; and

(c) an intron,

wherein the promoter is an endosperm-specific or endosperm-preferential promoter or an embryo-specific or embryo-preferential promoter,

and wherein the nucleic acid molecule comprises:

(i) the nucleotide sequence of SEQ ID NO: 1, 3, 5, 7, 9, 11 or 13;

(ii) a nucleotide sequence encoding the amino acid sequence of SEQ ID NO: 2, 4, 6, 8, 10, 12 or 14;

(iii) a nucleotide sequence having at least 60% identity to the nucleotide sequence of SEQ ID NO: 1, 3, 5, 7, 9, 11 or 13 and encoding a polypeptide having a Pfam:PF00224 pyruvate kinase barrel domain and a Pfam:PF02887 pyruvate kinase alpha/beta domain;

(iv) a nucleotide sequence encoding an amino acid sequence having at least 60% identity to the amino acid sequence of SEQ ID NO: 2, 4, 6, 8, 10, 12 or 14 and having a Pfam:PF00224 pyruvate kinase barrel domain and a Pfam:PF02887 pyruvate kinase alpha/beta domain;

(v) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 109 to 449 of SEQ ID NO: 2 or the amino acid residues 98 to 439 of SEQ ID NO: 10, and wherein the Pfam:PF02887 pyruvate kinase alpha/beta domain has at least 80% identity to the amino acid residues 462 to 578 of SEQ ID NO: 2 or the amino acid residues 452 to 566 of SEQ ID NO: 10; or

(vi) a nucleotide sequence encoding an amino acid sequence having at least 60% identity to the amino acid sequence of SEQ ID NO: 2, 4, 6, 8, 10, 12 or 14, wherein said amino acid sequence further comprises the amino acid sequence of SEQ ID NO: 102 and 103.

The intron may be a monocot intron in some embodiments. In other embodiments, the monocot intron may be a rice intron.

In embodiments where the promoter is a seed-specific or seed-preferential promoter, the promoter may comprise:

(a) the nucleotide sequence of SEQ ID NO: 104 or 105;

(b) a nucleotide sequence having at least 95% identity to the nucleotide sequence of SEQ ID NO: 104 or 105, wherein said nucleotide sequence has seed-specific or seed-preferential expression activity; or

(c) a fragment of the nucleotide sequence of SEQ ID NO: 104 or 105, wherein the fragment has seed-specific or seed-preferential expression activity.

In other embodiments where the promoter is an endosperm-specific or endosperm-preferential promoter, the promoter may comprise:

(a) the nucleotide sequence of SEQ ID NO: 106 or 107;

(b) a nucleotide sequence having at least 95% identity to the nucleotide sequence of SEQ ID NO: 106 or 107, wherein said nucleotide sequence has endosperm-specific or endosperm-preferential expression activity; or

(c) a fragment of the nucleotide sequence of SEQ ID NO: 106 or 107, wherein the fragment has endosperm-specific or endosperm-preferential expression activity.

In yet other embodiments where the promoter is an embryo-specific or embryo-preferential promoter, the promoter may comprise:

(a) the nucleotide sequence of SEQ ID NO: 108;

(b) a nucleotide sequence having at least 95% identity to the nucleotide sequence of SEQ ID NO: 108, wherein said nucleotide sequence has embryo-specific or embryo-preferential expression activity; or

(c) a fragment of the nucleotide sequence of SEQ ID NO: 108, wherein the fragment has embryo-specific or embryo-preferential expression activity.

In some specific embodiments, the nucleic acid molecule encoding a polypeptide having pyruvate kinase activity may comprise:

(a) a nucleotide sequence having at least 95% identity to the nucleotide sequence of SEQ ID NO: 1, 3, 5, 7, 9, 11 or 13; or

(b) a nucleotide sequence encoding an amino acid sequence having at least 95% identity to the amino acid sequence of SEQ ID NO: 2, 4, 6, 8, 10, 12 or 14.

In other specific embodiments, the nucleic acid molecule encoding a polypeptide having pyruvate kinase activity may comprise:

(a) the nucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, or 83; or

(b) a nucleotide sequence encoding the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, or 84.

In some embodiments where the intron is a rice intron, the rice intron may be an intron of the rice Metallothionin1 gene (Met1-1). In other embodiments, the rice intron may be an intron of the rice MADS3 gene (MADS3). In specific embodiments, the rice intron is the intron of the rice Met1-1 gene comprising the nucleotide sequence of SEQ ID NO: 111 or a nucleotide sequence having at least 90% identity to the nucleotide sequence of SEQ ID NO: 111. In other specific embodiments, the rice intron is the intron of the rice MADS3 gene comprising the nucleotide sequence of SEQ ID NO: 112 or a nucleotide sequence having at least 90% identity to the nucleotide sequence of SEQ ID NO: 112.

In a further specific embodiment, the expression cassette according to the present invention may comprise a promoter comprising the nucleotide sequence of SEQ ID NO: 106, a nucleic acid molecule encoding a polypeptide having pyruvate kinase activity comprising a nucleotide sequence encoding the amino acid sequence of SEQ ID NO: 2, and an intron of the rice Met1-1 gene comprising the nucleotide sequence of SEQ ID NO: 111. In a yet further specific embodiment, the aforementioned expression cassette may further comprise a nucleotide sequence encoding a plastid-targeting peptide comprising the amino acid sequence of SEQ ID NO: 114 and a terminator comprising the nucleotide sequence of SEQ ID NO: 115.

In another specific embodiment, the expression cassette according to the present invention may comprise a promoter comprising the nucleotide sequence of SEQ ID NO: 106, a nucleic acid molecule encoding a polypeptide having pyruvate kinase activity comprising a nucleotide sequence encoding the amino acid sequence of SEQ ID NO: 2, and an intron of the rice MADS3 gene comprising the nucleotide sequence of SEQ ID NO: 112. In a yet another specific embodiment, the aforementioned expression cassette may further comprise a nucleotide sequence encoding a plastid-targeting peptide comprising the amino acid sequence of SEQ ID NO: 114 and a terminator comprising the nucleotide sequence of SEQ ID NO: 115.

In a further aspect, the invention provides an expression cassette conferring increased content in one or more of protein, oil, or one or more amino acids in a plant, plant cell, or plant part relative to a corresponding wild-type plant, plant cell, or plant part, comprising:

(a) a promoter that is functional in a plant;

(b) a nucleic acid molecule encoding a polypeptide having pyruvate kinase activity which is heterologous and operably linked to said promoter; and

(c) the first intron of the rice Metallothionin1 gene (Met1-1),

wherein the nucleic acid molecule comprises:

(i) the nucleotide sequence of SEQ ID NO: 87 or 89;

(ii) a nucleotide sequence encoding the amino acid sequence of SEQ ID NO: 88 or 90;

(iii) a nucleotide sequence having at least 75% identity to the nucleotide sequence of SEQ ID NO: 87 or 89 and encoding a polypeptide having a Pfam:PF00224 pyruvate kinase barrel domain and a Pfam:PF02887 pyruvate kinase alpha/beta domain;

(iv) a nucleotide sequence encoding an amino acid sequence having at least 75% identity to the amino acid sequence of SEQ ID NO: 88 or 90 and having a Pfam:PF00224 pyruvate kinase barrel domain and a Pfam:PF02887 pyruvate kinase alpha/beta domain;

(v) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 5 to 350 of SEQ ID NO: 88, and wherein the Pfam:PF02887 pyruvate kinase alpha/beta domain has at least 80% identity to the amino acid residues 362 to 478 of SEQ ID NO: 88; or

(vi) a nucleotide sequence encoding an amino acid sequence having at least 75% identity to the amino acid sequence of SEQ ID NO: 88 or 90, wherein said amino acid sequence further comprises the amino acid sequence of SEQ ID NO: 102 and 103.

In some embodiments, the promoter is a constitutive promoter. In other embodiments, the promoter is a seed-specific or seed-preferential promoter. In yet other embodiments, the promoter is an endosperm-specific or endosperm-preferential promoter. In further yet other embodiments, the promoter is an embryo-specific or embryo-preferential promoter.

In specific embodiments, the first intron of the rice Met1-1 gene comprised in the aforementioned expression cassette may comprise the nucleotide sequence of SEQ ID NO: 111 or a nucleotide sequence having at least 90% identity to the nucleotide sequence of SEQ ID NO: 111.

In yet another aspect, the invention provides an expression cassette conferring increased content in one or more of protein, oil, or one or more amino acids in a plant, plant cell, or plant part relative to a corresponding wild-type plant, plant cell, or plant part, comprising:

(a) a constitutive promoter that is functional in a plant;

(b) a nucleic acid molecule encoding a polypeptide having pyruvate kinase activity which is heterologous and operably linked to said promoter; and

(c) an intron,

wherein the nucleic acid molecule comprises:

(i) the nucleotide sequence of SEQ ID NO: 87 or 89;

(ii) a nucleotide sequence encoding the amino acid sequence of SEQ ID NO: 88 or 90;

(iii) a nucleotide sequence having at least 75% identity to the nucleotide sequence of SEQ ID NO: 87 or 89 and encoding a polypeptide having a Pfam:PF00224 pyruvate kinase barrel domain and a Pfam:PF02887 pyruvate kinase alpha/beta domain;

(iv) a nucleotide sequence encoding an amino acid sequence having at least 75% identity to the amino acid sequence of SEQ ID NO: 88 or 90 and having a Pfam:PF00224 pyruvate kinase barrel domain and a Pfam:PF02887 pyruvate kinase alpha/beta domain;

(v) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 5 to 350 of SEQ ID NO: 88, and wherein the Pfam:PF02887 pyruvate kinase alpha/beta domain has at least 80% identity to the amino acid residues 362 to 478 of SEQ ID NO: 88; or

(vi) a nucleotide sequence encoding an amino acid sequence having at least 75% identity to the amino acid sequence of SEQ ID NO: 88 or 90, wherein said amino acid sequence further comprises the amino acid sequence of SEQ ID NO: 102 and 103,

and wherein the constitutive promoter comprises:

(a) the nucleotide sequence of SEQ ID NO: 109 or 110;

(b) a nucleotide sequence having at least 95% identity to the nucleotide sequence of SEQ ID NO: 109 or 110, wherein said nucleotide sequence has constitutive expression activity; or

(c) a fragment of the nucleotide sequence of SEQ ID NO: 109 or 110, wherein the fragment has constitutive expression activity.

In some embodiments, the intron is a monocot intron. In other embodiments, the intron is an intron of the rice Met1-1 gene. In yet other embodiments, the intron is an intron of the rice MADS3 gene. In specific embodiments, the rice intron is the intron of the rice Met1-1 gene comprising the nucleotide sequence of SEQ ID NO: 111 or a nucleotide sequence having at least 90% identity to the nucleotide sequence of SEQ ID NO: 111. In other specific embodiments, the rice intron is the intron of the rice MADS3 gene comprising the nucleotide sequence of SEQ ID NO: 112 or a nucleotide sequence having at least 90% identity to the nucleotide sequence of SEQ ID NO: 112.

In further embodiments, any of the aforementioned expression cassettes may further comprise a nucleotide sequence encoding a transit peptide targeting the polypeptide having pyruvate kinase activity to a plastid, preferably the nucleotide sequence is heterologous in relation to the nucleic acid molecule encoding the polypeptide having pyruvate kinase activity. In some specific embodiments, the transit peptide is a plastid-targeting peptide from a ferredoxin gene. In other specific embodiments, the nucleotide sequence encoding a transit peptide comprises:

(a) the nucleotide sequence of SEQ ID NO: 113;

(b) a nucleotide sequence encoding the amino acid sequence of SEQ ID NO: 114; or

(c) a nucleotide sequence encoding a peptide having at least 95% identity to the amino acid sequence of SEQ ID NO: 114.

In particular embodiments, the expression of the nucleic acid molecule encoding a polypeptide having pyruvate kinase activity comprised in any of the aforementioned expression cassettes in a plant, plant cell, or plant part confers increased content in one or more of protein, oil, or one or more amino acids in said plant, plant cell, or plant part relative to a corresponding wild-type plant, plant cell, or plant part. In other specific embodiments, the expression of the nucleic acid molecule encoding a polypeptide having pyruvate kinase activity comprised in any of the aforementioned expression cassettes in a plant, plant cell, or plant part confers increased content of protein, oil, and one or more amino acids in said plant, plant cell, or plant part relative to a corresponding wild-type plant, plant cell, or plant part.

In a yet further aspect, the invention provides an expression cassette conferring increased content of protein, oil, and one or more amino acids in a plant, plant cell, or plant part relative to a corresponding wild-type plant, plant cell, or plant part, comprising:

(a) a seed-specific or seed-preferential promoter; and

(b) a nucleic acid molecule encoding a polypeptide having pyruvate kinase activity which is heterologous and operably linked to said seed-specific or seed-preferential promoter,

wherein the nucleic acid molecule comprises:

(i) the nucleotide sequence of SEQ ID NO: 1, 3, 5, 7, 9, 11 or 13;

(ii) a nucleotide sequence encoding the amino acid sequence of SEQ ID NO: 2, 4, 6, 8, 10, 12 or 14;

(iii) a nucleotide sequence having at least 60% identity to the nucleotide sequence of SEQ ID NO: 1, 3, 5, 7, 9, 11 or 13 and encoding a polypeptide having a Pfam:PF00224 pyruvate kinase barrel domain and a Pfam:PF02887 pyruvate kinase alpha/beta domain;

(iv) a nucleotide sequence encoding an amino acid sequence having at least 60% identity to the amino acid sequence of SEQ ID NO: 2, 4, 6, 8, 10, 12 or 14 and having a Pfam:PF00224 pyruvate kinase barrel domain and a Pfam:PF02887 pyruvate kinase alpha/beta domain;

(v) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 109 to 449 of SEQ ID NO: 2 or the amino acid residues 98 to 439 of SEQ ID NO: 10, and wherein the Pfam:PF02887 pyruvate kinase alpha/beta domain has at least 80% identity to the amino acid residues 462 to 578 of SEQ ID NO: 2 or the amino acid residues 452 to 566 of SEQ ID NO: 10; or

(vi) a nucleotide sequence encoding an amino acid sequence having at least 60% identity to the amino acid sequence of SEQ ID NO: 2, 4, 6, 8, 10, 12 or 14, wherein said amino acid sequence further comprises the amino acid sequence of SEQ ID NO: 102 and 103,

and wherein expression of the nucleic acid molecule in a plant, plant cell, or plant part confers increased content of protein, oil, and one or more amino acids in said plant, plant cell, or plant part relative to a corresponding wild-type plant, plant cell, or plant part.

In some embodiments, the expression cassette may further comprise an intron. In other embodiments, the expression cassette may further comprise a nucleotide sequence encoding a transit peptide targeting the polypeptide having pyruvate kinase activity to a plastid, preferably the nucleotide sequence is heterologous in relation to the nucleic acid molecule encoding the polypeptide having pyruvate kinase activity. In yet other embodiments, the transit peptide is a plastid-targeting peptide from a ferredoxin gene.

In some specific embodiments, the seed-specific or seed-preferential promoter comprised in the aforementioned expression cassette is an endosperm-specific or endosperm-preferential promoter. In yet other specific embodiment, the seed-specific or seed-preferential promoter comprised in the aforementioned expression cassette is an embryo-specific or embryo-preferential promoter.

In other embodiments, any of the aforementioned expression cassettes may further comprise a terminator. In particular embodiments, the terminator comprises the nucleotide sequence of SEQ ID NO: 115 or 116, or a nucleotide sequence having at least 90% identity to the nucleotide sequence of SEQ ID NO: 115 or 116.

In a further aspect, the invention provides a recombinant construct comprising at least one of the aforementioned expression cassettes. The invention further provides vectors comprising any of the aforementioned recombinant constructs.

The invention also provides a microorganism comprising at least one of the aforementioned expression cassettes, a recombinant construct comprising at least one of the aforementioned expression cassettes, or a vector comprising at least one of the aforementioned expression cassettes or any of the aforementioned recombinant constructs. The invention further provides a plant cell, plant or part thereof comprising at least one of the aforementioned expression cassettes or a recombinant construct comprising at least one of the aforementioned expression cassettes, wherein the plant, plant cell, or plant part has increased content in one or more of protein, oil, or one or more amino acids relative to a corresponding wild-type plant, plant cell, or plant part. In some specific embodiments, the plant, plant cell, or plant part of the invention has increased content of protein, oil, and one or more amino acids relative to a corresponding wild-type plant, plant cell, or plant part.

In some embodiments, the plant is a monocotyledonous plant or the plant cell or plant part is from a monocotyledonous plant. In other embodiments, the plant is a maize plant or the plant cell or plant part is from a maize plant. In further specific embodiments, the plant part is a seed.

In yet another aspect, the invention provides a food or feed composition comprising any of the aforementioned plants, plant cells, or plant parts.

In some embodiments, the food or feed composition is not supplemented with additional protein, oil, or amino acids. In other embodiments, the food or feed composition has reduced supplementation with protein, oil, or amino acids relative to a food or feed composition comprising a corresponding wild-type plant, plant cell, or plant part. In specific embodiments, the feed composition may be formulated to meet the dietary requirements of swine, poultry, cattle, or companion animals.

Further, the invention provides a method for producing a transgenic plant, plant cell, or plant part having increased content in one or more of protein, oil, or one or more amino acids relative to a corresponding wild-type plant, plant cell, or plant part, comprising:

(a) transforming a plant, plant cell, or plant part with any of the aforementioned expression cassettes, a recombinant construct comprising at least one of the aforementioned expression cassettes, or a vector comprising at least one of the aforementioned expression cassettes or any of the aforementioned recombinant constructs, and

(b) optionally regenerating from the plant cell or plant part a transgenic plant,

wherein the transgenic plant, plant cell, or plant part has increased content in one or more of protein, oil, or one or more amino acids relative to a corresponding wild-type plant, plant cell, or plant part.

In still another aspect, the invention provides a method for increasing the content of one or more of protein, oil, or one or more amino acids in a plant, plant cell, or plant part relative to a corresponding wild-type plant, plant cell, or plant part, comprising:

(a) obtaining any of the aforementioned plants, plant cells, or plant parts; and

(b) selecting a plant, plant cell, or plant part with increased content in one or more of protein, oil, or one or more amino acids.

In some embodiments, the plant is a monocotyledonous plant or the plant cell or plant part is from a monocotyledonous plant. In other embodiments, the plant is a maize plant or the plant cell or plant part is from a maize plant.

In some embodiments, the content of one or more amino acids in the plant, plant cell, or plant part obtained from any of the aforementioned methods is increased relative to a corresponding wild-type plant, plant cell, or plant part. In other embodiments, the content of protein in the plant, plant cell, or plant part obtained from any of the aforementioned methods is increased relative to a corresponding wild-type plant, plant cell, or plant part. In yet further embodiments, the content of oil in the plant, plant cell, or plant part obtained from any of the aforementioned methods is increased relative to a corresponding wild-type plant, plant cell, or plant part. In another embodiment, the content of oil and one or more amino acids in a plant, plant cell, or plant part is increased relative to a corresponding wild-type plant, plant cell, or plant part. In a further embodiment, the content of protein and one or more amino acids in a plant, plant cell, or plant part is increased relative to a corresponding wild-type plant, plant cell, or plant part. In specific embodiments, the content of protein, oil and one or more amino acids in the plant, plant cell, or plant part obtained from any of the aforementioned methods is increased relative to a corresponding wild-type plant, plant cell, or plant part. In some particular embodiments, the plant, plant cell, or plant part obtained from any of the aforementioned methods has an increased content of one or more amino acids selected from the group consisting of arginine, cysteine, isoleucine, lysine, methionine, threonine, and valine. In other particular embodiments, the content of at least two amino acids in the plant, plant cell, or plant part is increased. In yet further embodiments, the content of two, three, four, five, six, or seven amino acids in the plant, plant cell, or plant part is increased relative to a corresponding wild-type plant, plant cell, or plant part.

In still a further aspect, the invention provides a method of producing a food or feed composition comprising:

(a) obtaining a plant, plant cell, or plant part having increased content in one or more of protein, oil, or one or more amino acids relative to a corresponding wild-type plant, plant cell, or plant part according to any of the aforementioned methods;

(b) producing a food or feed composition comprising said plant, plant cell, or plant part.

In still yet a further aspect, the invention provides a method for producing a hybrid maize plant or seed comprising:

(a) crossing a first inbred parent maize plant with a second inbred parent maize plant;

(b) harvesting a resultant hybrid maize seed; and

(c) optionally growing a hybrid maize plant from the resultant hybrid maize seed,

wherein said first inbred parent maize plant, and optionally said second inbred parent maize plant, comprises any of the aforementioned expression cassettes or a recombinant construct comprising any of the aforementioned expression cassettes.

The invention further provides a hybrid maize plant or seed produced by the aforementioned method. The invention additionally provides a plant produced by growing the aforementioned hybrid maize seed.

In still another further aspect, the invention provides a plant breeding program comprising utilizing any of the aforementioned plants, plant cells, or plant parts as a source of plant breeding material, wherein the plant, plant cell, or plant part has increased content in one or more of protein, oil, or one or more amino acids relative to a corresponding wild-type plant, plant cell, or plant part. The invention additionally provides a plant, plant cell, or plant part obtained from the aforementioned plant breeding program.

In yet another further aspect, the invention provides a method for developing a maize plant in a maize plant breeding program using plant breeding techniques comprising employing a maize plant, or its parts, as a source of plant breeding material, wherein the maize plant, or its parts, comprises any of the aforementioned expression cassettes or a recombinant construct comprising any of the aforementioned expression cassettes. The invention further provides a maize plant obtained from the aforemention method.

The invention additionally provides a method of plant breeding, comprising:

(a) obtaining any of the aforementioned hybrid maize plants;

(b) crossing said hybrid maize plant with a different maize plant; and

(c) selecting a resultant progeny with increased content in one or more of protein, oil, or one or more amino acids.

In a still further aspect, the invention provides a method for producing grain with increased content in one or more of protein, oil, or one or more amino acids, comprising:

(a) interplanting a first plant and at least one second plant, wherein the first plant comprises any of the aforementioned expression cassettes or a recombinant construct comprising any of the aforementioned expression cassettes;

(b) growing said first plant and said at least one second plant to obtain preferential inheritance of increased content in one or more of protein, oil, or one or more amino acids in a resultant progeny of said first plant and said at least one second plant; and

(c) harvesting grain from said resultant progeny.

In further embodiments, the invention provides grains produced by the aforementioned method, wherein the grain has increased content in one or more of protein, oil, or one or more amino acids relative to a corresponding wild-type grain. In other embodiments, the grains produced by the aforementioned method has increased content in one or more amino acids selected from the group consisting of arginine, cysteine, isoleucine, lysine, methionine, threonine, and valine. In specific embodiments, the grain is corn.

In still yet another aspect, the invention provides a method for producing a maize plant with increased content in one or more of protein, oil, or one or more amino acids, comprising:

(a) growing a progeny plant obtained from crossing a maize plant comprising any of the aforementioned expression cassettes or a recombinant construct comprising any of the aforementioned expression cassettes with a second maize plant;

(b) crossing said progeny plant with itself or a different maize plant to produce a resultant seed;

(c) growing said resultant seed to obtain a progeny plant of a subsequent generation; and

(d) crossing said progeny plant of a subsequent generation with itself or a different maize plant; and

(e) repeating steps (b) to (d) for additional 0-5 generations to produce a maize plant with increased content in one or more of protein, oil, or one or more amino acids.

In a further embodiment, the maize plant produced by the aforementioned method is an inbred maize plant. In another embodiment, the aforementioned method may further comprise crossing the inbred maize plant with a second, distinct inbred maize plant to produce an F1 hybrid maize plant.

### DESCRIPTION OF THE FIGURES

FIG. 1A-K shows the sequence alignment between pyruvate kinases having significant homology to SEQ ID NO: 2 with the conserved Pfam domains identified.

FIG. 2A-E shows the sequence alignment between pyruvate kinases having significant homology to SEQ ID NO: 10 with the conserved Pfam domains identified.

FIG. 3A-C shows the sequence alignment between pyruvate kinases having significant homology to SEQ ID NO: 88 with the conserved Pfam domains identified.

FIG. 4 shows the protein-protein identity for extracted domain Pfam:PF00224 pyruvate kinase barrel of pyruvate kinases having significant homology to SEQ ID NO: 2 (“SEQ ID NO: X_POS_Y_Z” indicates that the domain is located between amino acid residue Y and Z of the sequence of SEQ ID NO: X).

FIG. 5 shows the protein-protein identity for extracted domain Pfam:PF02887 pyruvate kinase alpha/beta domain of pyruvate kinases having significant homology to SEQ ID NO: 2 (“SEQ ID NO: X_POS_Y_Z” indicates that the domain is located between amino acid residue Y and Z of the sequence of SEQ ID NO: X).

FIG. 6 shows the protein-protein identity for extracted domain Pfam:PF00224 pyruvate kinase barrel domain of pyruvate kinases having significant homology to SEQ ID NO: 10 (“SEQ ID NO: X_POS_Y_Z” indicates that the domain is located between amino acid residue Y and Z of the sequence of SEQ ID NO: X).

FIG. 7 shows the protein-protein identity for extracted domain Pfam:PF02887 pyruvate kinase alpha/beta domain of pyruvate kinases having significant homology to SEQ ID NO: 10 (“SEQ ID NO: X_POS_Y_Z” indicates that the domain is located between amino acid residue Y and Z of the sequence of SEQ ID NO: X).

FIG. 8 shows the protein-protein identity for extracted domain Pfam:PF00224 pyruvate kinase barrel domain of pyruvate kinases having significant homology to SEQ ID NO: 88 (“SEQ ID NO: X_POS_Y_Z” indicates that the domain is located between amino acid residue Y and Z of the sequence of SEQ ID NO: X).

FIG. 9 shows the protein-protein identity for extracted domain Pfam:PF02887 pyruvate kinase alpha/beta domain of pyruvate kinases having significant homology to SEQ ID NO: 88 (“SEQ ID NO: X_POS_Y_Z” indicates that the domain is located between amino acid residue Y and Z of the sequence of SEQ ID NO: X).

### DESCRIPTION OF THE PREFERRED EMBODIMENTS

Throughout this application, various publications are referenced. The disclosures of all of these publications and those references cited within those publications are hereby incorporated by reference in their entireties into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used. The term “about” as used herein is to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20%, preferably 10% up or down (higher or lower). The word “comprise,” “comprising,” “include,” “including,” and “includes” as used herein and in the following claims is intended to specify the presence of one or more stated features, integers, components, or steps, but they do not preclude the presence or addition of one or more other features, integers, components, steps, or groups thereof.

In one aspect, the invention provides various novel expression cassettes conferring increased content in one or more of protein, oil, or one or more amino acids in a plant, plant cell, or plant part relative to a corresponding wild-type plant, plant cell, or plant part. In another aspect, the invention provides methods for overexpressing a pyruvate kinase in a plant, plant cell, or plant part which in turn confers increased content in one or more of protein, oil, or one or more amino acids in a plant, plant cell, or plant part relative to a corresponding wild-type plant, plant cell, or plant part, wherein various expression cassettes of the invention can be used.

The term “wild-type” as used herein refers to a plant, plant cell, seed, plant component, plant part, plant tissue, plant organ, or whole plant that has not been genetically modified with a polynucleotide in accordance with the invention.

The term “overexpressing” or “overexpression” as used herein means the level of expression of a nucleic acid molecule or a protein in a plant, plant cell, or plant part is higher or increased relative to its expression in a reference plant, plant cell, or plant part, such as a corresponding wild-type plant, plant cell, or plant part, grown under substantially identical conditions.

### 1. Expression Cassettes

1.1 Basic Components

The expression cassettes of the present invention generally comprise at least two components:

(a) a promoter that is functional in a plant, and

(b) a nucleic acid molecule encoding a polypeptide having pyruvate kinase activity which is heterologous and operably linked to said promoter,

wherein expression of the nucleic acid molecule in a plant, plant cell, or plant part confers an increase in one or more of protein, oil, or one or more amino acids in a plant, plant cell, or plant part relative to a corresponding wild-type plant, plant cell, or plant part.

As used herein, the term “nucleic acid,” “nucleic acid molecule,” “polynucleotide,” or “gene” is interchangeable and refers to naturally occurring or synthetic or artificial nucleic acid or polynucleotide. The term “nucleic acid,” “nucleic acid molecule,” “polynucleotide,” or “gene” comprises DNA or RNA or any nucleotide analogue and polymers or hybrids thereof in either linear or branched, single- or double-stranded, sense or antisense form. The term also encompasses RNA/DNA hybrids. Unless otherwise indicated, a particular nucleic acid molecule also implicitly encompasses conservatively modified variants thereof such as, but not limited to, degenerate codon substitutions and complementary sequences as well as the sequence explicitly indicated. A skilled worker will recognize that DNA sequence polymorphisms, which lead to changes in the encoded amino acid sequence, may exist within a population. These genetic polymorphisms in a gene may exist between individuals within a population owing to natural variation. These natural variants usually bring about a variance of 1 to 5% in the nucleotide sequence of a particular gene. Each and every one of these nucleotide variations and resulting amino acid polymorphisms in the encoded polypeptide which are the result of natural variation and do not modify the functional activity are also encompassed by the invention.

The terms “polypeptide” or “protein” are used interchangeably herein.

“Expression cassette” as used herein refers to a DNA molecule which includes sequences capable of directing expression of a particular nucleic acid molecule (e.g., which codes for a protein of interest) in an appropriate host cell, including regulatory sequences such as a promoter operably linked to a nucleic acid molecule of interest, optionally associated with transcription termination signals and/or other regulatory elements. An expression cassette may also comprise sequences required for proper translation of the nucleic acid molecule of interest. The expression cassette comprising the nucleic acid molecule of interest may be chimeric, meaning that at least one of its components is heterologous with respect to at least one of its other components. An expression cassette may be assembled entirely extracellularly (e.g., by recombinant cloning techniques). A nucleic acid molecule of interest according to the present invention may preferably encode a pyruvate kinase or a polypeptide having pyruvate kinase activity.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family. The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

The term “operably linked” or “operable linkage” encompasses, for example, an arrangement of the transcription regulating nucleotide sequence with the nucleic acid sequence to be expressed and, if appropriate, further regulatory elements, such as terminator or enhancers, in such a way that each of the regulatory elements can fulfill its intended function to allow, modify, facilitate or otherwise influence expression of the nucleic acid sequence under the appropriate conditions. Appropriate conditions relate to preferably the presence of the expression cassette in a plant cell. In a preferred arrangement, the nucleic acid sequence is placed down-stream (i.e. in 5′ to 3′-direction) of the transcription regulating nucleotide sequence. Optionally, additional sequences, such as a linker, multiple cloning site, intron, or nucleotide sequence encoding a protein targeting sequence may be inserted between the two sequences.

The term “heterologous” refers to material (nucleic acid or protein) which is obtained or derived from different source organisms, or, from different genes or proteins in the same source organism or a nucleic acid sequence to which it is not linked in nature or to which it is linked at a different location in nature. For example, a protein-coding nucleic acid sequence operably linked to a promoter which is not the native promoter of this protein-coding sequence is considered to be heterologous to the promoter.

All percentages of protein, oil, and amino acid content in a plant, plant cell, or plant part recited herein are percent dry weight. Methods for determining and calculating the protein, oil, and amino acid content in a plant, plant cell, or plant part are known in the art and routinely used by a skilled person.

In one embodiment, the content of one or more amino acids in the plant, plant cell, or plant part of the invention is increased by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, or 200% over the content of the corresponding one or more amino acids in a corresponding wild-type plant, plant cell, or plant part. Preferably, the amino acids, of which the content is increased in the plant, plant cell, or plant part of the invention, are selected from the group consisting of arginine, cysteine, isoleucine, lysine, methionine, threonine, and valine. More preferably, the plant, plant cell, or plant part of the invention demonstrates an increased content in one or more amino acids selected from the group consisting of arginine, cysteine, isoleucine, lysine, methionine, threonine, and valine by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, or 200% relative to a corresponding wild-type plant, plant cell, or plant part. In other embodiments, the increased content of one or more amino acids is an increase in two, three, four, five, six, or seven amino acids selected from the group consisting of arginine, cysteine, isoleucine, lysine, methionine, threonine, and valine.

In another embodiment, the oil content of the plant, plant cell, or plant part of the invention is increased by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, or 200% over the oil content of the corresponding wild-type plant, plant cell, or plant part.

In yet another embodiment, the protein content of the plant, plant cell, or plant part of the invention is increased by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, or 200% over the protein content of the corresponding wild-type plant, plant cell, or plant part.

In a further embodiment, the content of protein and one or more amino acids in the plant, plant cell, or plant part of the invention is increased by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, or 200% over the content of protein and one or more amino acids in a corresponding wild-type plant, plant cell, or plant part.

In yet a further embodiment, the content of protein, oil, and one or more amino acids in the plant, plant cell, or plant part of the invention is increased by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, or 200% over the content of protein, oil, and one or more amino acids in a corresponding wild-type plant, plant cell, or plant part.

1.1.1 Promoters

The term “promoter” as used herein is equivalent to the terms “promoter element,” “promoter sequence,” or “transcription regulating nucleotide sequence” and refers to a DNA sequence which, when linked to a nucleic acid molecule of interest, is capable of controlling the transcription of the nucleic acid molecule of interest into mRNA. A promoter is typically, though not necessarily, located 5′ (i.e. upstream) of a nucleic acid molecule of interest (e.g., proximal to the transcriptional start site of a structural gene) whose transcription into mRNA it controls, and provides a site for specific binding by RNA polymerase and other transcription factors for initiation of transcription.

For expressing a nucleic acid molecule of interest according to the present invention, the nucleic acid molecule of interest is operably linked to an appropriate promoter, preferably a promoter that is functional in a plant. Unless specifically provided otherwise, the promoter to be comprised in the expression cassettes of the invention is preferably a promoter that is functional in a plant. As used herein, “a promoter that is functional in a plant” means principally a promoter which is capable of driving the expression of a nucleic acid molecule operably linked thereto, in particular foreign nucleic acid sequences or genes, in plants or plant parts, plant cells, plant tissues, plant cultures. In this context, the expression specificity of said promoter functional in a plant can be, for example, constitutive, inducible, developmentally regulated, tissue-specific or tissue-preferential, organ-specific or organ-preferential, cell type-specific or cell type-preferential, spatial-specific or spatial-preferential, and/or temporal-specific or temporal-preferential.

Such promoters include, but not limited to, those that can be obtained from plants, plant viruses and bacteria that contain genes that are expressed in plants, such as Agrobacterium and Rhizobium.

Constitutive promoters are generally active under most environmental conditions and states of development or cell differentiation. Useful constitutive promoters for plants include those obtained from Ti- or Ri-plasmids, from plant cells, plant viruses or other organisms whose promoters are found to be functional in plants. Bacterial promoters that function in plants, and thus are suitable for use in the present invention include, but not limited to, the octopine synthetase promoter, the nopaline synthase promoter, and the mannopine synthetase promoter from the T-DNA of Agrobacterium. Likewise, viral promoters that function in plants can also be used in the present invention. Examples of viral promoters include, but are not limited to, the promoter isolated from sugarcane bacilliform virus (ScBV; U.S. Pat. No. 6,489,462; Nadiya et al., Biotechnology, 2010, published online), the cauliflower mosaic virus (CaMV) 35S transcription initiation region (Franck et al., Cell, 1980, 21: 285-294; Odell et al., Nature, 1985, 313: 810-812; Shewmaker et al., Virology, 1985, 140: 281-288; Gardner et al., Plant Mol. Biol., 1986, 6: 221-228), the cauliflower mosaic virus (CaMV) 19S transcription initiation region (U.S. Pat. No. 5,352,605 and WO 84/02913) and region VI promoters, and the full-length transcript promoter from Figwort mosaic virus. Other suitable constitutive promoters for use in plants include, but are not limited to, actin promoters such as the rice actin promoter (McElroy et al., Plant Cell, 1990, 2: 163-171) or the Arabidopsis actin promoter, histone promoters, tubulin promoters, or the mannopine synthase promoter (MAS), ubiquitin or poly-ubiquitin promoters (Sun and Callis, Plant J., 1997, 11(5): 1017-1027; Cristensen et al., Plant Mol. Biol., 1992, 18: 675-689; Christensen et al., Plant Mol. Biol., 1989 12: 619-632; Bruce et al., Proc. Natl. Acad. Sci. USA, 1989, 86: 9692-9696; Holtorf et al., Plant Mol. Biol., 1995, 29: 637-649; for example, the ubiquitin promoter from Zea mays (SEQ ID NO: 126)), the Mac or DoubleMac promoters (U.S. Pat. No. 5,106,739; Comai et al., Plant Mol. Biol., 1990, 15: 373-381), Rubisco small subunit (SSU) promoter (U.S. Pat. No. 4,962,028), the legumin B promoter (GenBank Acc. No. X03677), the TR dual promoter, the Smas promoter (Velten et al., EMBO J., 1984, 3: 2723-2730), the cinnamyl alcohol dehydrogenase promoter (U.S. Pat. No. 5,683,439), the promoters of the vacuolar ATPase subunits, the pEMU promoter (Last et al., Theor. Appl. Genet., 1991, 81: 581-588), the maize 113 histone promoter (Lepetit et al., Mol. Gen. Genet., 1992, 231: 276-285; Atanassova et al., Plant J., 1992, 2(3): 291-300), β-conglycinin promoter, the phaseolin promoter, the ADH promoter, and heat-shock promoters, the nitrilase promoter from Arabidopsis thaliana (WO 03/008596; GenBank Acc. No. U38846, nucleotides 3,862 to 5,325 or else 5,342), promoter of a proline-rich protein from wheat (WO 91/13991), the promoter of the Pisum sativum ptxA gene, and other promoters active in plant cells that are known to those of skill in the art.

In some embodiments, the expression cassettes of the invention comprise a constitutive promoter. Preferably, the constitutive promoter is isolated from sugarcane bacilliform virus (ScBV). More preferably, the constitutive promoter to be included in the expression cassettes of the invention comprises:

(a) the nucleotide sequence of SEQ ID NO: 109 or 110;

(b) a nucleotide sequence having at least 95%, preferably 96%, 97%, 98%, 99%, 99.1%, 99.2%, 99.3%, 99.4%, 99.5%, 99.6%, 99.7%, 99.8%, or 99.9% identity to the nucleotide sequence of SEQ ID NO: 109 or 110, wherein said nucleotide sequence has constitutive expression activity; or

(c) a fragment of the nucleotide sequence of SEQ ID NO: 109 or 110, wherein the fragment has constitutive expression activity.

Inducible promoters are active under certain environmental conditions, such as the presence or absence of a nutrient or metabolite, heat or cold, light, pathogen attack, anaerobic conditions, and the like. An inducible promoter can be induced in response to a chemical, environmental or physical stimulus, or may be “stress-inducible,” i.e. activated when a plant is exposed to various stress conditions, or “pathogen-inducible,” i.e. activated when a plant is exposed to exposure to various pathogens. Promoters responding to biotic or abiotic stress conditions are also suitable inducible promoters.

A cell-specific or cell-preferential, tissue-specific or tissue-preferential, or organ-specific or organ-preferential promoter is one that is capable of preferentially initiating transcription in certain types of cells, tissues, or organs, such as leaves, stems, roots, flowers, fruits, anthers, ovaries, pollen, seed tissue, green tissue, or meristem. A promoter is cell-, tissue- or organ-specific or preferential, if its activity, measured on the amount of RNA produced under control of the promoter, is at least 30%, 40%, 50%, preferably at least 60%, 70%, 80%, 90%, more preferably at least 100%, 200%, 300%, higher in a particular cell-type, tissue or organ, then in other cell-types or tissues of the same plant, preferably the other cell-types or tissues are cell types or tissues of the same plant organ, e.g., leaves or roots. In the case of organ specific or preferential promoters, the promoter activity has to be compared to the promoter activity in other plant organs, e.g., leaves, stems, flowers or seeds. For example, the tissue-specific ES promoter from tomato is particularly useful for directing expression in fruits (see, e.g., Lincoln et al., Proc. Natl. Acad. Sci. USA, 1988, 84: 2793-2797; Deikman et al., EMBO J., 1988, 7: 3315-3320; Deikman et al., Plant Physiol., 1992, 100: 2013-2017). Seed-specific or seed-preferential promoters are preferentially expressed during seed development and/or germination, which can be embryo-, endosperm-, and/or seed coat-specific or preferential. See Thompson et al., BioEs-says, 1989, 10: 108. Examples of seed-specific or preferential promoters include, but are not limited to, the pKG86 promoter from Zea maize (whole seed-specific or whole seed-preferential promoter), the promoters derived from the globulin 1 gene from maize (ZmGlb1) (Belanger et al., Genetics, 1991, 129: 863-872), the zein genes from maize, including 10 kDa zein, 19 kDa zein, and 27 kDa zein, the MAC1 gene from maize (Sheridan et al., Genetics, 1996, 142: 1009-1020), the Cat3 gene from maize (GenBank Accession No. L05934), the gene encoding oleosin 18 kD from maize (GenBank Accession No. J05212), viviparous-1 gene from Arabidopsis (Genbank Accession No. U93215), the gene encoding oleosin from Arabidopsis (Genbank Accession No. Z17657), the Atmyc1 gene from Arabidopsis (Urao et al., Plant Mol. Biol., 1996, 32: 571-576), the 2S seed storage protein gene family from Arabidopsis (Conceicao et al., Plant J., 1994, 5: 493-505), the gene encoding oleosin 20 kD from Brassica napus (GenBank Accession No. M63985), the napin gene from Brassica napus (GenBank Accession No. J02798; Joseffson et al., J. Biol. Chem., 1987, 262: 12196-12201), the napin gene family (e.g., from Brassica napus; Sjodahl et al., Planta, 1995, 197: 264-271, U.S. Pat. No. 5,608,152; Stalberg et al., Planta, 1996, 199: 515-519), the gene encoding the 2S storage protein from Brassica napus (Dasgupta et al., Gene, 1993, 133: 301-302), the genes encoding oleosin A (Genbank Accession No. U09118) and oleosin B (Genbank Accession No. U09119) from soybean, the gene encoding low molecular weight sulphur rich protein from soybean (Choi et al., Mol. Gen. Genet., 1995, 246: 266-268), the phaseolin gene (U.S. Pat. No. 5,504,200; Bustos et al., Plant Cell, 1989, 1(9): 839-853; Murai et al., Science, 1983, 23: 476-482; Sengupta-Gopalan et al., Proc. Natl. Acad. Sci. USA, 1985, 82: 3320-3324), the 2S albumin gene, the legumin gene (Shirsat et al., Mol. Gen. Genet., 1989, 215(2): 326-331), the USP (unknown seed protein) gene, the sucrose binding protein gene (WO 00/26388), the legumin B4 gene (LeB4; Fiedler et al., Biotechnology, 1995, 13(10): 1090-1093; Baumlein et al., Plant J., 1992, 2(2): 233-239; Baumlein et al., Mol. Gen. Genet., 1991, 225(3): 459-467; Baumlein et al., Mol. Gen. Genet., 1991, 225: 121-128), the Arabidopsis oleosin gene (WO 98/45461), the Brassica Bce4 gene (WO 91/13980), genes encoding the “high-molecular-weight glutenin” (HMWG), gliadin, branching enzyme, ADP-glucose pyrophosphatase (AGPase) or starch synthase. Further seed specific or preferential promoters include the KG86_{—}12a promoter (SEQ ID NO: 104) and the KG86 promoter (SEQ ID NO: 128).

Other suitable tissue- or organ-specific or preferential promoters include a leaf-specific and light-induced promoter such as that from cab or Rubisco (Timko et al., Nature, 1985, 318: 579-582; Simpson et al., EMBO J., 1985, 4: 2723-2729), an anther-specific promoter such as that from LAT52 (Twell et al., Mol. Gen. Genet., 1989, 217: 240-245), a pollen-specific promoter such as that from Zm13 (Guerrero et al., Mol. Gen. Genet., 1993, 224: 161-168), and a microspore-preferred promoter such as that from apg (Twell et al., Sex. Plant Reprod., 1983, 6: 217-224). Also suitable promoters are, for example, specific promoters for tubers, storage roots or roots such as, for example, the class I patatin promoter (B33), the potato cathepsin D inhibitor promoter, the starch synthase (GBSS1) promoter or the sporamin promoter, and fruit-specific promoters such as, for example, the tomato fruit-specific promoter (EP 0409625). Promoters which are furthermore suitable are those which ensure leaf-specific or leaf-preferential expression. Further examples of promoters which may be mentioned are the potato cytosolic FBPase promoter (WO 98/18940), the Rubisco (ribulose-1,5-bisphosphate carboxylase) SSU (small subunit) promoter or the potato ST-LSI promoter (Stockhaus et al., EMBO J., 1989, 8(9): 2445-2451). Other suitable promoters are those which govern expression in seeds and plant embryos. Further suitable promoters are, for example, fruit-maturation-specific promoters such as, for example, the tomato fruit-maturation-specific promoter (WO 94/21794), flower-specific promoters such as, for example, the phytoene synthase promoter (WO 92/16635) or the promoter of the P1-rr gene (WO 98/22593) or another node-specific promoter as described in EP 0249676 may be used advantageously. The promoter may also be a pith-specific promoter, such as the promoter isolated from a plant TrpA gene as described in WO 93/07278.

In some embodiments, the expression cassettes of the invention comprise a tissue-specific or tissue-preferential promoter. More preferably, the tissue-specific or tissue-preferential promoter is a seed-specific or seed-preferential promoter, such as a whole seed-specific or whole seed-preferential promoter, an endosperm-specific or endosperm-preferential promoter, or an embryo-specific or embryo-preferential promoter.

In some preferred embodiments, the promoter to be included in the expression cassettes of the invention is a seed-specific or seed-preferential promoter, preferably a whole seed-specific or whole seed-preferential promoter comprising:

(a) the nucleotide sequence of SEQ ID NO: 104 or 105 or 128;

(b) a nucleotide sequence having at least at least 95%, preferably 96%, 97%, 98%, 99%, 99.1%, 99.2%, 99.3%, 99.4%, 99.5%, 99.6%, 99.7%, 99.8%, or 99.9% identity to the nucleotide sequence of SEQ ID NO: 104 or 105 or 128, wherein said nucleotide sequence has seed-specific or seed-preferential expression activity; or

(c) a fragment of the nucleotide sequence of SEQ ID NO: 104 or 105 or 128, wherein the fragment has seed-specific or seed-preferential expression activity.

In other preferred embodiments, the promoter to be included in the expression cassettes of the invention is an endosperm-specific or endosperm-preferential promoter, preferably an endosperm-specific or endosperm-preferential promoter comprising:

(a) the nucleotide sequence of SEQ ID NO: 106 or 107;

(b) a nucleotide sequence having at least 95%, preferably 96%, 97%, 98%, 99%, 99.1%, 99.2%, 99.3%, 99.4%, 99.5%, 99.6%, 99.7%, 99.8%, or 99.9% identity to the nucleotide sequence of SEQ ID NO: 106 or 107, wherein said nucleotide sequence has endosperm-specific or endosperm-preferential expression activity; or

(c) a fragment of the nucleotide sequence of SEQ ID NO: 106 or 107, wherein the fragment has endosperm-specific or endosperm-preferential expression activity.

In yet other preferred embodiments, the promoter to be included in the expression cassettes of the invention is an embryo-specific or embryo-preferential promoter, preferably an embryo-specific or embryo-preferential promoter comprising:

(a) the nucleotide sequence of SEQ ID NO: 108;

(b) a nucleotide sequence having at least 95%, preferably 96%, 97%, 98%, 99%, 99.1%, 99.2%, 99.3%, 99.4%, 99.5%, 99.6%, 99.7%, 99.8%, or 99.9% identity to the nucleotide sequence of SEQ ID NO: 108, wherein said nucleotide sequence has embryo-specific or embryo-preferential expression activity; or

(c) a fragment of the nucleotide sequence of SEQ ID NO: 108, wherein the fragment has embryo-specific or embryo-preferential expression activity.

Developmentally regulated or developmental stage-preferential promoters are preferentially expressed at certain stages of development. Suitable developmental regulated promoters include, but not limited to, fruit-maturation-specific promoters, such as, for example, the fruit-maturation-specific promoter from tomato (WO 94/21794, EP 0409625). Developmental regulated promoters also include partly the tissue-specific or tissue-preferential promoters described above since individual tissues are, naturally, formed as a function of the development. An example of a development-regulated promoter is described in Baerson et al. (Plant Mol. Biol., 1993, 22(2): 255-267).

Other promoters or promoter elements suitable for the expression cassettes of the invention include, but not limited to, promoters or promoter elements capable of modifying the expression-governing characteristics. Thus, for example, the tissue-specific or tissue-preferential expression may take place in addition as a function of certain stress factors, owing to genetic control sequences. Such elements are, for example, described for water stress, abscisic acid (Lam and Chua, J. Biol. Chem., 1991, 266(26): 17131-17135) and heat stress (Schoffl et al., Molecular & General Genetics, 1989, 217(2-3): 246-253).

Unless specifically provided herein, the promoter to be included in the expression cassettes of the invention is a promoter that is functional in a plant.

1.1.2 Pyruvate Kinases

Pyruvate kinase (PK, EC 2.7.1.40) catalyses one of the key control points of glycolysis—the biochemical pathway central to energy metabolism and the production of precursors used in biosynthetic reactions in all living organisms. The enzyme requires magnesium and the majority of enzymes also require potassium ions for its activity and catalyses the transfer of a phosphoryl group from phosphoenolpyruvate (PEP) to adenosine diphosphate (ADP), generating the important biochemical intermediates pyruvate and adenosine triphosphate (ATP).

In vertebrates, there are four tissue-specific PK isozymes: L (liver), R (red cells), M1 (muscle, heart and brain), and M2 (early foetal tissue). In plants, PK exists as cytosolic (PKc) and plastidic (PKp) isoforms, while most bacteria and lower eukaryotes have one form of PK except in certain bacteria, such as Escherichia coli, that have two isozymes.

PK helps control the rate of glycolysis, along with phosphofructokinase and hexokinase. PK possesses allosteric sites for numerous effectors, yet the isozymes respond differently, in keeping with their different tissue distributions (Munoz et al., Comp. Biochem. Physiol. B. Biochem. Mol. Biol., 2003, 135(2): 197-218). For example, in vertebrates, the activity of L-type (liver) PK is increased by fructose-1,6-bisphosphate (F1, 6BP) and lowered by ATP and alanine (gluconeogenic precursor). As such, when glucose levels are high, glycolysis is promoted, whereas when glucose levels are low, gluconeogenesis is promoted. L-type PK is also hormonally regulated, being activated by insulin and inhibited by glucagon, which covalently modifies the PK enzyme. Conversely, M1-type (muscle and brain) PK is inhibited by ATP, but F1, 6BP and alanine have no effect, which correlates with the function of muscle and brain, as opposed to the liver. Similarly, the two forms of PK isolated from E. coli, PK type 1 (PK-I; b1676) and PK type 2 (PK-II; b1854), sharing approximately 37% sequence identity at the amino acid level, differ in the identity of their heterotropic allosteric effector. Although both PK-I (b1676) and PK-II (b1854) are homotropically activated by the substrate PEP, PK-II (b1854) is activated by AMP and monophosphorylated sugars, whereas PK-I (b1676) is activated by F1, 6BP (Lovell et al., J. Mol. Biol., 1998, 276: 839-851).

The structure of several PKs from various organisms have been determined (Valentini et al., J. Biol. Chem., 2002, 277: 23807-23814; Valentini et al., J. Biol. Chem., 2000, 275: 18145-18152). Plant PK proteins comprise the following domains: a small N-terminal helical domain (absent in bacterial PK), a beta/alpha-barrel domain, a beta-barrel domain (inserted within the beta/alpha-barrel domain), and a 3-layer alpha/beta/alpha sandwich domain. The beta/alpha-barrel domain and the beta-barrel domain inserted with it are also collectively identified as Pfam:PF00224 pyruvate kinase barrel domain (see website ebi.ac.uk/interpro/IEntry?ac=IPRO15793). It is predicted that this domain comprises the magnesium ion binding site, the potassium ion binding site and the PK active site. The S-layer alpha/beta/alpha sandwich domain is also identified as Pfam:PF02887 pyruvate kinase alpha/beta domain (see website ebi.ac.uk/interpro/ISearch?query=PF02887).

In plants, both cytosolic (PKc) and plastidic (PKp) PK isoforms differ markedly with respect to their physical, immunological and kinetic characteristics. Cytosolic forms of PK are homomeric, while plastidic forms of PK are generally thought to consist of α and β subunits (Munoz et al., Comp. Biochem. Physiol. B. Biochem. Mol. Biol., 2003, 135(2): 197-218). For instance, plastidic PKs purified from castor (Ricinus communis) endosperm and Brassica napus suspension cell cultures both consist of α- and β-subunits and appear to exist as 3α3β heterohexamers (Plaxton et al., Plant Physiol., 1990, 94: 1528-1534; Plaxton et al., Arch. Biochem. Biophys., 2002, 400: 54-62; Negm et al., Plant Physiol., 1995, 109: 1461-1469).

Plant PK activities arise from the expression of multiple isozymes with different biochemical properties that depend on the tissue and plant source. Arabidopsis, for example, has 14 annotated PK genes that likely exhibit a large degree of variation with respect to regulation of gene expression and enzyme activity. Among these 14 putative isoforms of PK, three are identified to be plastidic, two β-forms and one α-form (Andre et al., Plant Cell, 2007, 19: 2006-2022). Despite the potential variation in gene regulation and enzyme activity, all but one (encoded by At3g49160) of the predicted PKs contain a fully conserved PK active site of [LIVAC]-x-[LIVM-[LIVM]-[SAPCV]-K-[LIV]-E-[NKRST]-x-[DEQHS]-[GSTAHLIVM] (SEQ ID NO: 101) and are presumably active enzymes (Andre et al., Plant Cell, 2007, 19: 2006-2022). In addition to the conserved PK active site, plant PKs also contain the two conserved Pfam domains as the other PKs found in other organisms: the Pfam:PF00224 pyruvate kinase barrel domain and the Pfam:PF02887 pyruvate kinase alpha/beta domain.

It is found that, by expressing certain pyruvate kinases in a plant, plant cell, or plant part under control of some specific types of promoters, optionally in combination with other regulatory elements and/or targeting peptides, the content of one or more of protein, oil, and/or one or more amino acids in such a plant, plant cell, or plant part is surprisingly increased. Accordingly, in one aspect, the invention provides an expression cassette capable of expressing a nucleic acid molecule encoding a pyruvate kinase or a polypeptide having pyruvate kinase activity in a plant, plant cell, or plant part, wherein the expression of such a nucleic acid molecule confers increased content in one or more of protein, oil, or one or more amino acids in said plant, plant cell, or plant part relative to a corresponding wild-type plant, plant cell, or plant part. In another aspect, the expression of the nucleic acid molecule comprised in the expression cassettes of the invention confers increased content in protein and one or more amino acids in such a plant, plant cell, or plant part relative to a corresponding wild-type plant, plant cell, or plant part. In another embodiment, the expression of the nucleic acid molecule comprised in the expression cassette of the invention confers increased content in oil and one or more amino acids in a plant, plant cell, or plant part relative to a corresponding wild-type plant, plant cell, or plant part. In yet another aspect, the expression of the nucleic acid molecule comprised in the expression cassettes of the invention confers increased content in protein, oil and one or more amino acids in such a plant, plant cell, or plant part relative to a corresponding wild-type plant, plant cell, or plant part.

In some embodiments, the pyruvate kinases or the polypeptides having pyruvate kinase activity suitable for the present invention comprise the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100, or functional variants thereof, or encoded by a nucleic acid molecule comprising the polynucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, or 99, or functional variants thereof. In preferred embodiments, the pyruvate kinase suitable for the present invention comprises the amino acid sequence of SEQ ID NO: 2 (100% identical to SEQ ID NO: 4, 6, and 8), SEQ ID NO: 10 (100% identical to SEQ ID NO: 12 and 14), or SEQ ID NO: 88 (100% identical to SEQ ID NO: 90), or functional variants thereof, or encoded by a nucleic acid molecule comprising the polynucleotide sequence of SEQ ID NO: 1, 3, 5, 7, 9, 11, 13, 87, or 89, or functional variants thereof.

The pyruvate kinase comprising the amino acid sequence of SEQ ID NO: 2 corresponds to the pyruvate kinase PKp-β1 of Arabidopsis thaliana encoded by At5g52920 (PKpAt920). Other pyruvate kinases or the polypeptides having pyruvate kinase activity sharing significant sequence homology with the pyruvate kinase PKp-β1 (PKpAt920; SEQ ID NO: 2) include, but not limited to, the PKs provided in Table 1.

TABLE 1
Examples of PKs with significant sequence homology with the pyruvate
kinase PKp-β1 (PKpAt920; SEQ ID NO: 2).

| | | | Global amino acid |
|---|---|---|---|
| PK Gene SEQ ID NO | | | % identity to |
| Nucleic Acid | Amino Acid | Organism | SEQ ID NO: 2 |
| 3 | 4 | Arabidopsis thaliana | 89.1 |
| 5 | 6 | Arabidopsis thaliana | 88.7 |
| 7 | 8 | Synthetic | 100 |
| 9 | 10 | Arabidopsis thaliana | 64.2 |
| 11 | 12 | Arabidopsis thaliana | 61.3 |
| 13 | 14 | Synthetic | 64.2 |
| 15 | 16 | Linum usitatissimum | 80.6 |
| 17 | 18 | Synthetic | 80.6 |
| 19 | 20 | Arabidopsis thaliana | 99.8 |
| 21 | 22 | Arabidopsis thaliana | 99.8 |
| 23 | 24 | Arabidopsis thaliana | 99.7 |
| 25 | 26 | Synthetic | 96.4 |
| 27 | 28 | Brassica napus | 95.7 |
| 29 | 30 | Brassica napus | 95.5 |
| 31 | 32 | Ricinus communis | 84.8 |
| 33 | 34 | Vitis vinifera | 83.2 |
| 35 | 36 | Vitis vinifera | 83.1 |
| 37 | 38 | Populus trichocarpa | 82.9 |
| 39 | 40 | Vitis vinifera | 82.8 |
| 41 | 42 | Glycine max | 81.6 |
| 43 | 44 | Glycine max | 81.3 |
| 45 | 46 | Glycine max | 81.2 |
| 47 | 48 | Glycine max | 81.1 |
| 49 | 50 | Glycine max | 80.8 |
| 51 | 52 | Glycine max | 80.7 |
| 53 | 54 | Glycine max | 80.2 |
| 59 | 60 | Brassica napus | 64.4 |
| 67 | 68 | Helianthus annuus | 77.1 |
| 69 | 70 | Synthetic | 77.1 |

The pyruvate kinase comprising the amino acid sequence of SEQ ID NO: 10 corresponds to the pyruvate kinase PKp-β2 of Arabidopsis thaliana encoded by At1g32440 (PKpAt440). Other pyruvate kinases or the polypeptides having pyruvate kinase activity sharing significant sequence homology with the pyruvate kinase PKp-β2 (PKpAt440; SEQ ID NO: 10) include, but not limited to, the PKs provided in Table 2.

TABLE 2
Examples of PKs with significant sequence homology with the pyruvate
kinase PKp-β2 (PKpAt440; SEQ ID NO: 10).

| | | | Global amino |
|---|---|---|---|
| PK Gene SEQ ID NO | | | acid % identity to |
| Nucleic Acid | Amino Acid | Organism | SEQ ID NO: 10 |
| 1 | 2 | Arabidopsis thaliana | 64.2 |
| 3 | 4 | Arabidopsis thaliana | 62.9 |
| 5 | 6 | Arabidopsis thaliana | 57 |
| 7 | 8 | Synthetic | 64.2 |
| 11 | 12 | Arabidopsis thaliana | 90.4 |
| 13 | 14 | Synthetic | 100 |
| 15 | 16 | Linum usitatissimum | 66.7 |
| 17 | 18 | Synthetic | 66.7 |
| 55 | 56 | Arabidopsis thaliana | 99.1 |
| 57 | 58 | Arabidopsis lyrata subsp. | 96.7 |
| | | lyrata | |
| 59 | 60 | Brassica napus | 91.6 |
| 67 | 68 | Helianthus annuus | 65.5 |
| 69 | 70 | Synthetic | 65.5 |

Both PKp-β1 (PKpAt920; SEQ ID NO: 2) and PKp-β2 (PKpAt440; SEQ ID NO: 10) are identified as being plastid localized with a chloroplast targeting signal of 63 and 55 amino acids, respectively (Andre et al., Plant Cell, 2007, 19: 2006-2022). Other pyruvate kinases or polypeptides having pyruvate kinase activity that are identified as being plastid localized (i.e. plastidic PKs) include, but not limited to, the PKs provided in Table 3.

TABLE 3
Examples of plastidic pyruvate kinases.

| PK Gene SEQ ID NO | | |
|---|---|---|
| Nucleic acid SEQ ID NO | Amino acid SEQ ID NO | Organism |
| 5 | 6 | Arabidopsis |
| | | thaliana |
| 7 | 8 | Synthetic |
| 13 | 14 | Synthetic |
| 15 | 16 | Linum |
| | | usitatissimum |
| 17 | 18 | Synthetic |
| 59 | 60 | Brassica napus |
| 61 | 62 | Zea mays |
| 63 | 64 | Helianthus annuus |
| 65 | 66 | Synthetic |
| 67 | 68 | Helianthus annuus |
| 69 | 70 | Synthetic |
| 83 | 84 | Arabidopsis |
| | | thaliana |

The pyruvate kinase comprising the amino acid sequence of SEQ ID NO: 88 corresponds to the pyruvate kinase II of Escherichia coli (b1854). Other pyruvate kinases or the polypeptides having pyruvate kinase activity sharing significant sequence homology with the pyruvate kinase b1854 (SEQ ID NO: 88) include, but not limited to, the PKs provided in Table 4.

TABLE 4
Examples of PKs with significant sequence homology with the pyruvate
kinase II of Escherichia coli (b1854; SEQ ID NO: 88).

| | | | Global amino |
|---|---|---|---|
| PK Gene SEQ ID NO | | | acid % identity to |
| Nucleic Acid | Amino Acid | Organism | SEQ ID NO: 88 |
| 75 | 76 | Pectobacterium wasabiae | 91 |
| 89 | 90 | Synthetic | 100 |
| 91 | 92 | Escherichia coli B185 | 99.8 |
| 93 | 94 | Escherichia coli 2362-75 | 99.6 |
| 95 | 96 | Photorhabdus | 86.9 |
| | | luminescens subsp. | |
| | | laumondii TTO1 | |
| 97 | 98 | Photorhabdus | 85.6 |
| | | asymbiotica subsp. | |
| | | asymbiotica ATCC | |
| | | 43949 | |
| 99 | 100 | Actinobacillus | 80.2 |
| | | succinogenes 130Z | |

Contrary to PKp-β1 and PKp-β2, the pyruvate kinase II of Escherichia coli (b1854; SEQ ID NO: 88) is a cytosolic pyruvate kinase which does not contain any targeting signal. Other pyruvate kinases or polypeptides having pyruvate kinase activity that are identified as being cytosolic form of PK (i.e. cytosolic PKs) include, but not limited to, the PKs provided in Table 5.

TABLE 5
Examples of cytosolic pyruvate kinases.

| PK Gene SEQ ID NO | | |
|---|---|---|
| Nucleic acid SEQ ID NO | Amino acid SEQ ID NO | Organism |
| 71 | 72 | Helianthus annuus |
| 73 | 74 | Synthetic |
| 75 | 76 | Pectobacterium |
| | | wasabiae |
| 77 | 78 | Zymomonas mobilis |
| 79 | 80 | Photobacterium |
| | | profundum |
| 81 | 82 | Arabidopsis |
| | | thaliana |
| 85 | 86 | Escherichia coli |
| 89 | 90 | Synthetic |

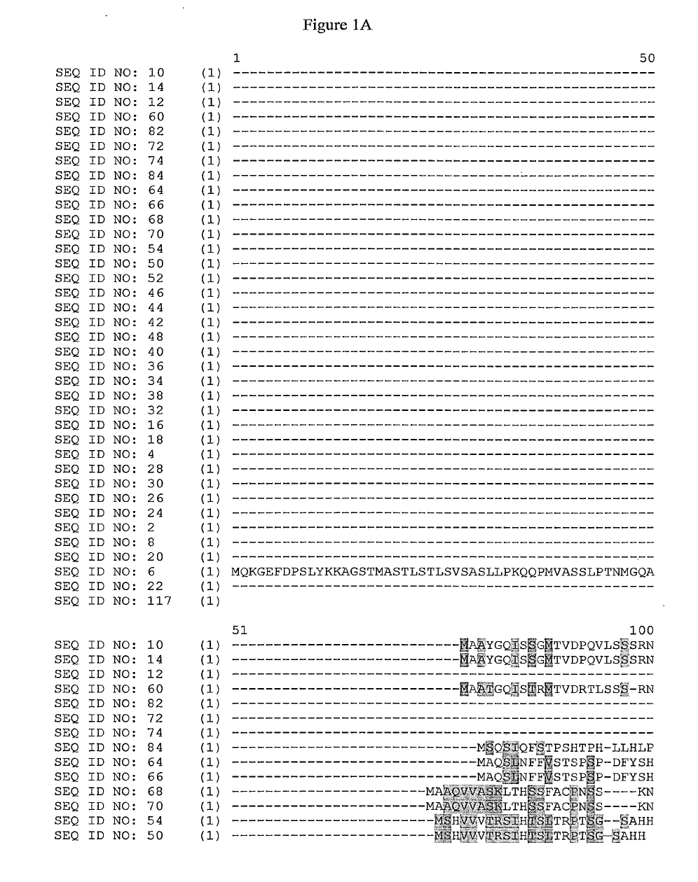

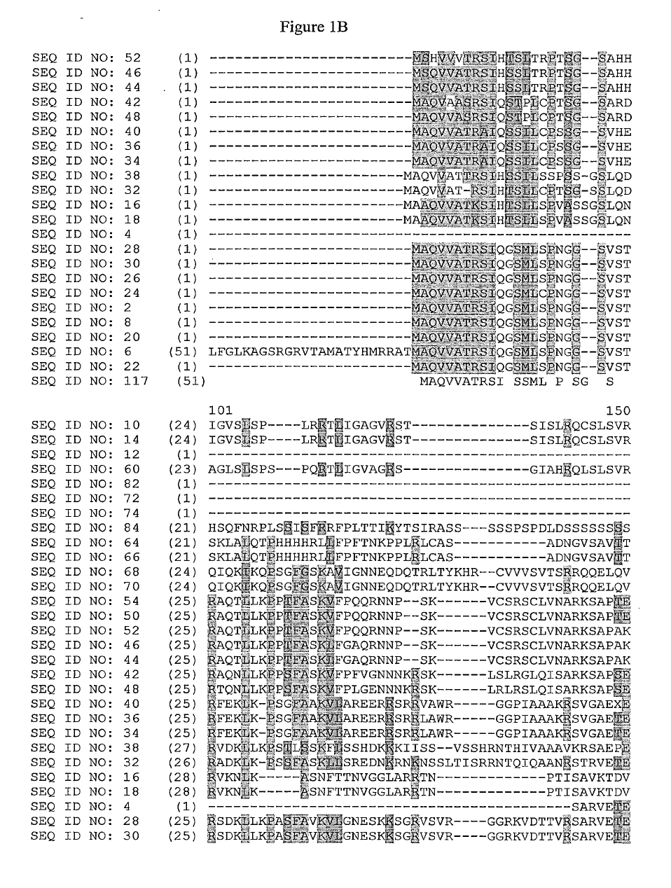

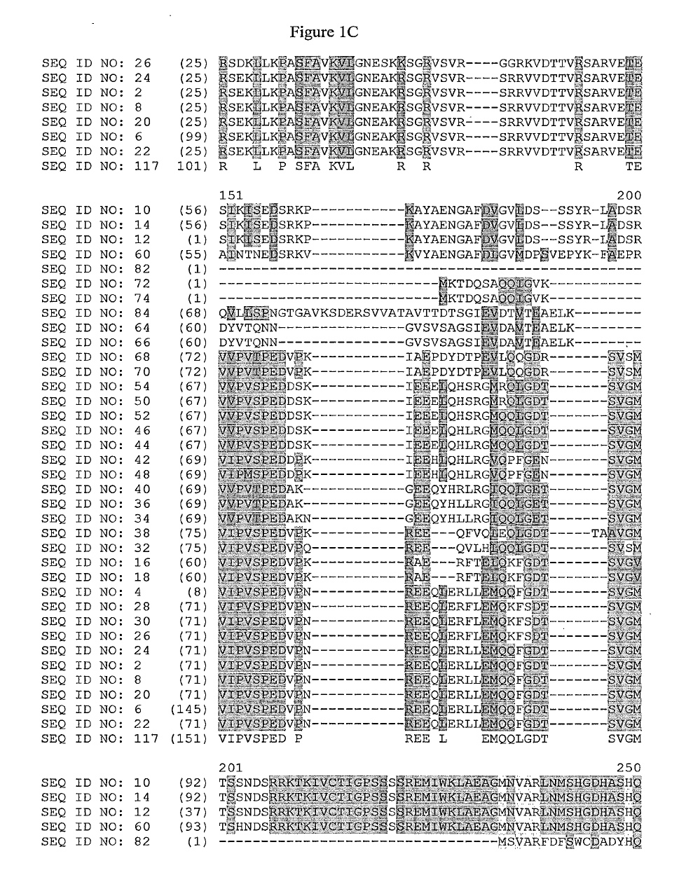

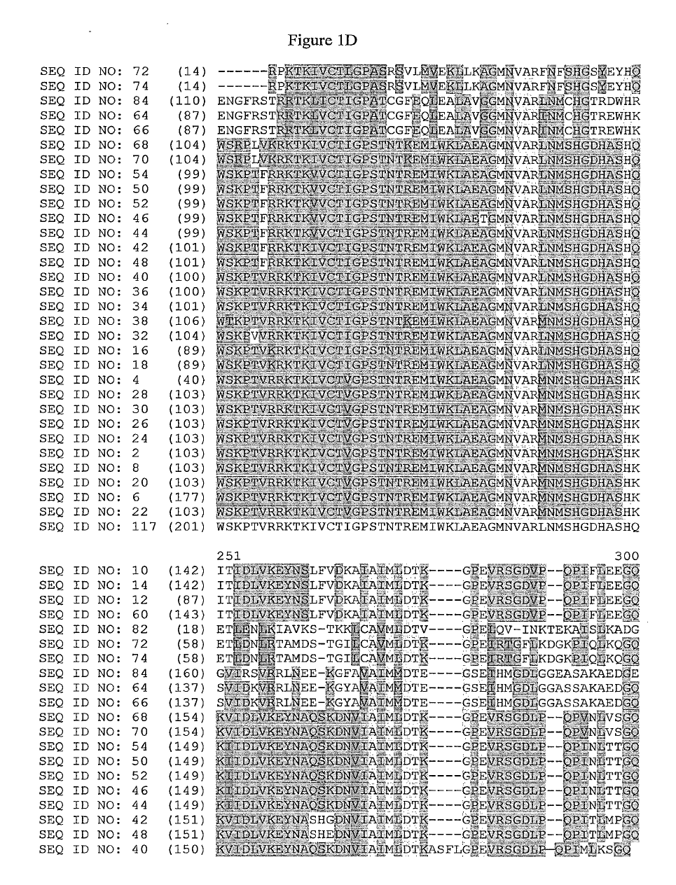

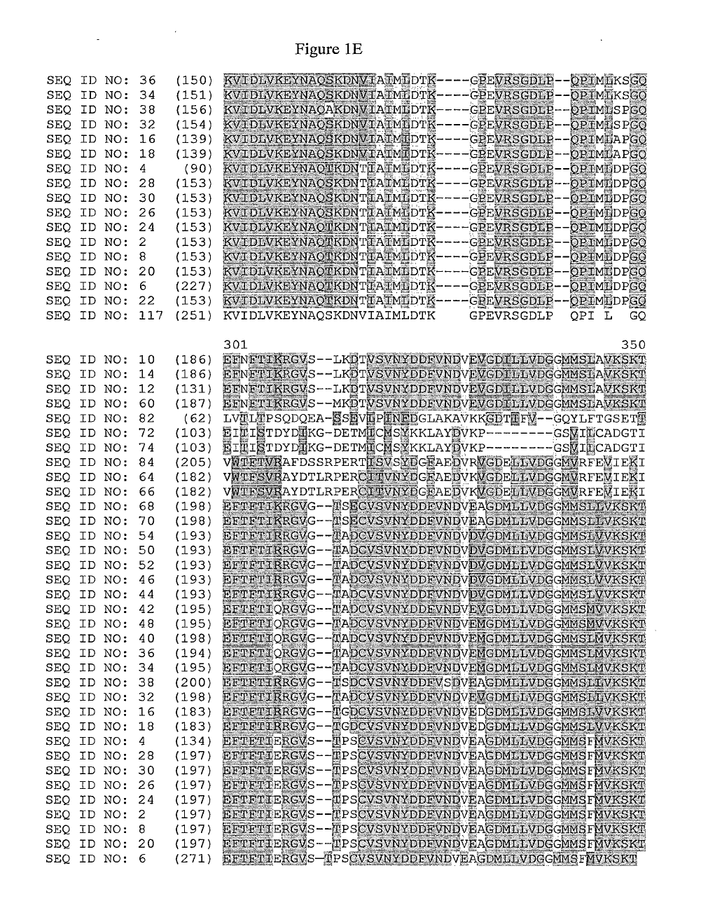

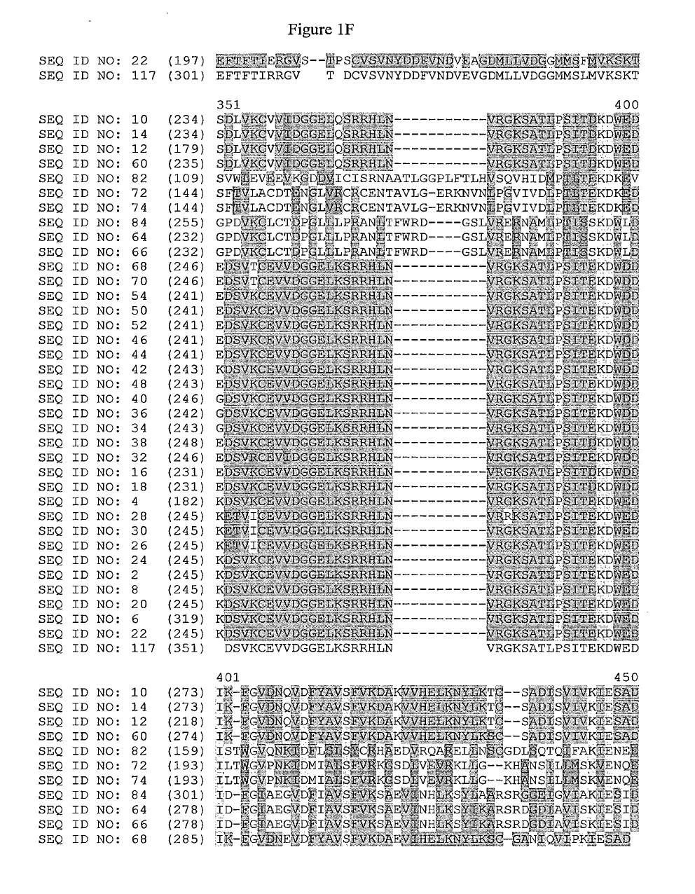

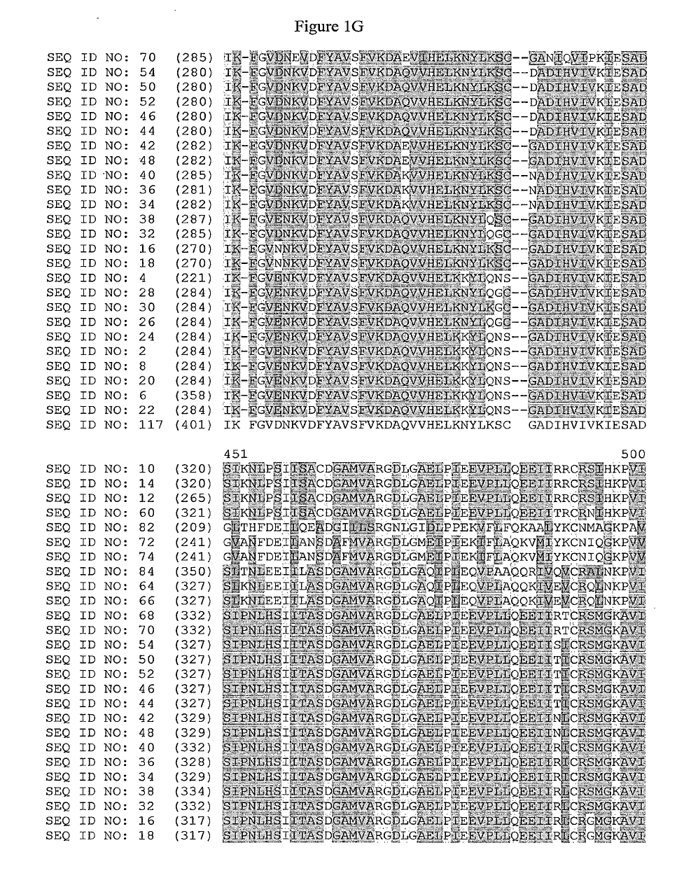

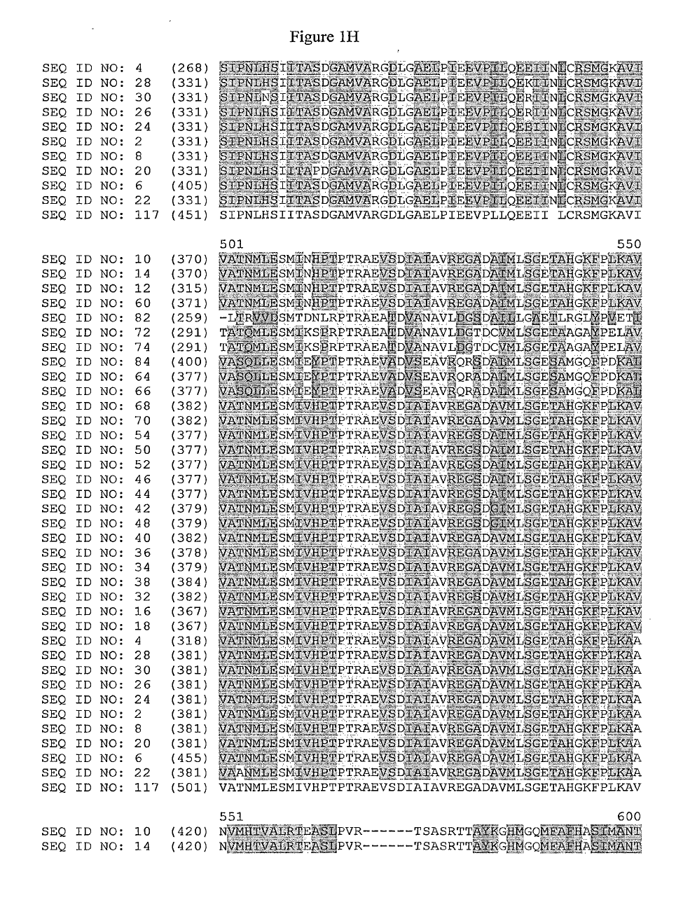

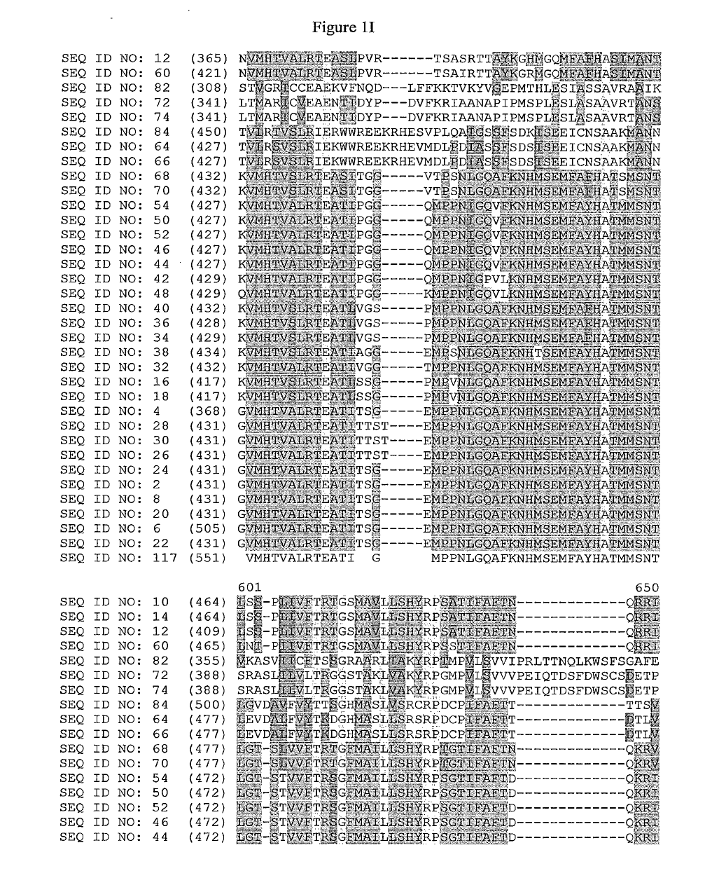

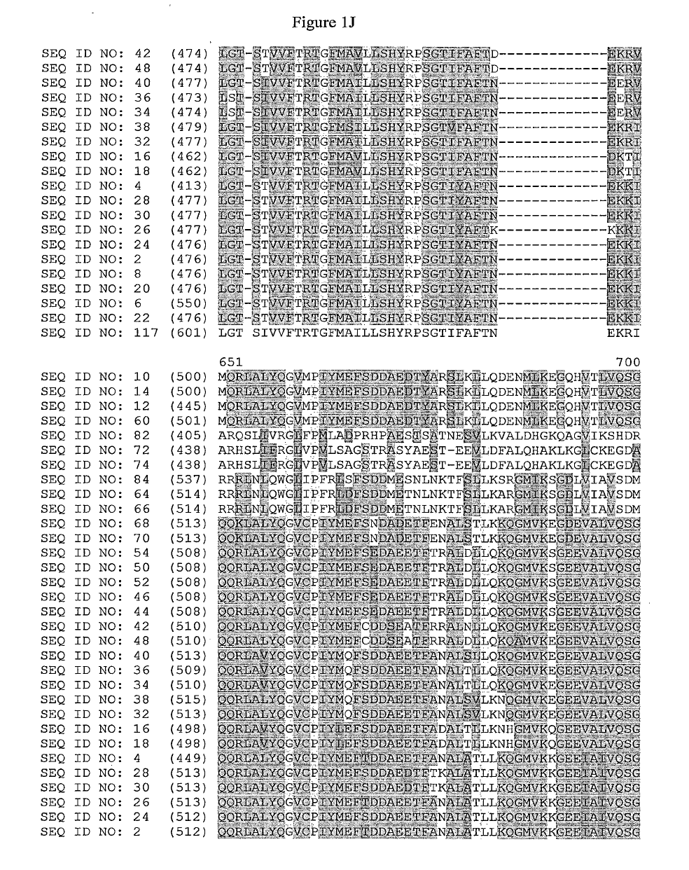

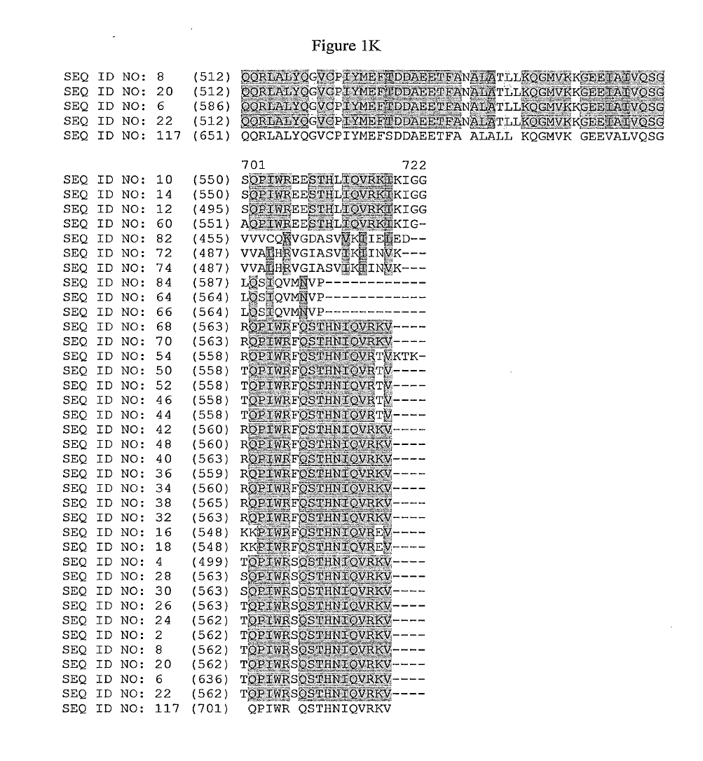

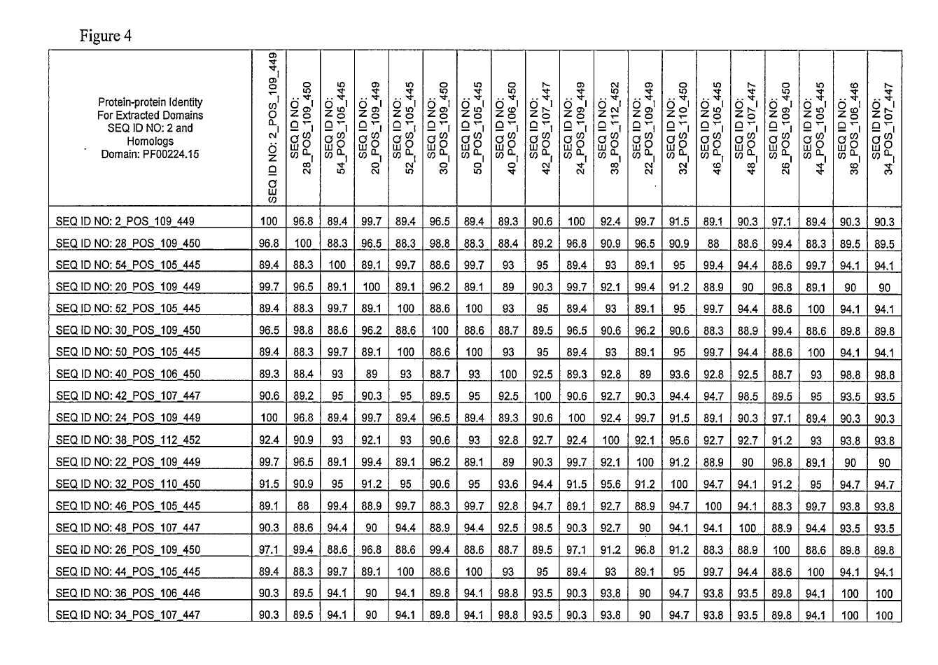

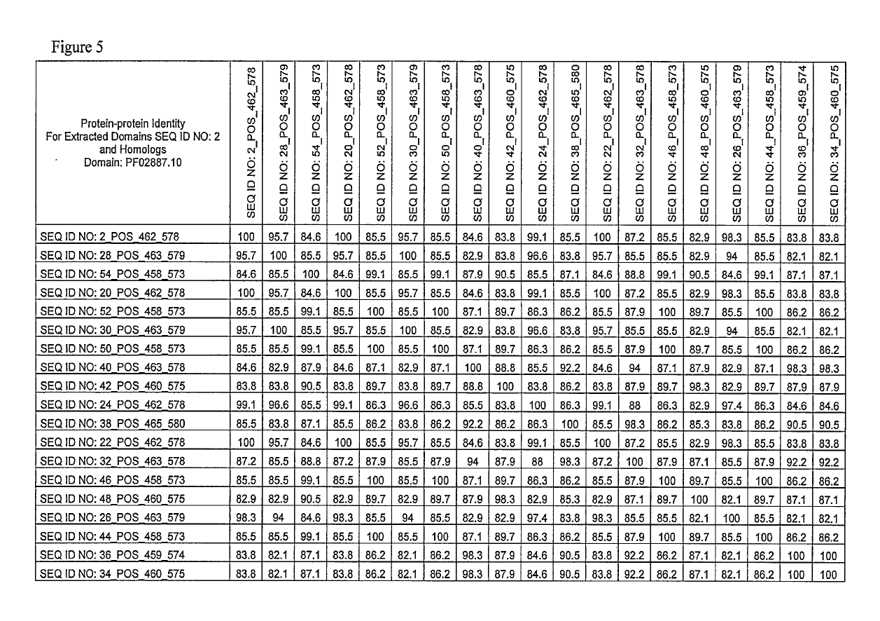

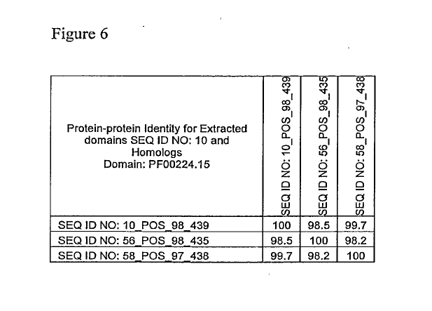

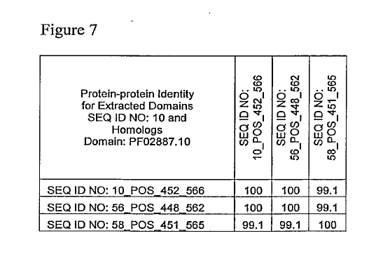

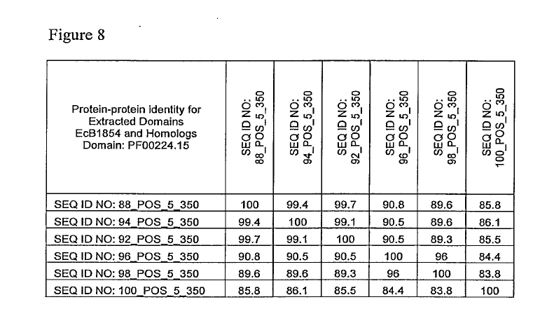

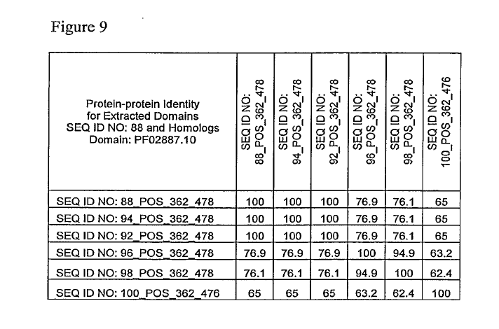

In other embodiments, the pyruvate kinases or the polypeptides having pyruvate kinase activity suitable for the present invention comprise a Pfam:PF00224 pyruvate kinase barrel domain and a Pfam:PF02887 pyruvate kinase alpha/beta domain. Examples of such pyruvate kinases or such polypeptides having pyruvate kinase activity and the location of their corresponding Pfam domains are provided in Table 6. Sequence alignments between various pyruvate kinases are also provided in FIGS. 1 to 3 with the conserved Pfam domains and PK active site identified. The level of homology between various pyruvate kinases within the region of the conserved Pfam domains are also provides in FIGS. 4 to 9.

TABLE 6
Pfam: PF00224 pyruvate kinase barrel domain and Pfam: PF02887 pyruvate kinase alpha/beta domain in the amino acid
sequences of pyruvate kinases. % Identity = the percent amino acid sequence identity of the Pfam domain of
each pyruvate kinase to the corresponding Pfam domain of the pyruvate kinases as shown in SEQ ID NO: 2, 10, and 88.

| | | | Pfam: PF00224 pyruvate kinase | | | | | Pfam: PF02887 pyruvate kinase | | | | |
|---|---|---|---|---|---|---|---|---|---|---|---|---|
| | | | barrel domain | | | | | alpha/beta domain | | | | |
| | | | | | % | % | % | | | % | % | % |
| PK Gene | | | | | identity | identity | identity | | | identity | identity | identity |
| SEQ ID NO | | | | | to SEQ | to SEQ | to SEQ | | | to SEQ | to SEQ | to SEQ |
| Nucleic | Amino | | Pfam | Pfam | ID NO: | ID NO: | ID NO: | Pfam | Pfam | ID NO: | ID NO: | ID NO: |
| Acid | Acid | Organism | Start | End | 2 | 10 | 88 | Start | End | 2 | 10 | 88 |
| 1 | 2 | Arabidopsis | 109 | 449 | 100 | 81.1 | 37.4 | 462 | 578 | 100 | 64.1 | 22.7 |
| | | thaliana | | | | | | | | | | |
| 3 | 4 | Arabidopsis | 46 | 386 | 100 | 81.1 | 37.4 | 399 | 515 | 100 | 64.1 | 22.7 |
| | | thaliana | | | | | | | | | | |
| 5 | 6 | Arabidopsis | 183 | 523 | 100 | 81.1 | 37.4 | 536 | 652 | 100 | 64.1 | 22.7 |
| | | thaliana | | | | | | | | | | |
| 7 | 8 | Synthetic | 109 | 449 | 100 | 81.1 | 37.4 | 462 | 578 | 100 | 64.1 | 22.7 |
| 9 | 10 | Arabidopsis | 98 | 439 | 81.1 | 100 | 36.2 | 452 | 566 | 64.1 | 100 | 24.2 |
| | | thaliana | | | | | | | | | | |
| 11 | 12 | Arabidopsis | 43 | 384 | 81.1 | 100 | 36.2 | 397 | 511 | 64.1 | 100 | 24.2 |
| | | thaliana | | | | | | | | | | |
| 13 | 14 | Synthetic | 98 | 439 | 81.1 | 100 | 36.2 | 452 | 566 | 64.1 | 100 | 24.2 |
| 15 | 16 | Linum | 95 | 435 | 90.6 | 82.6 | 38.3 | 448 | 563 | 82.1 | 65.8 | 24.6 |
| | | usitatissimum | | | | | | | | | | |
| 17 | 18 | Synthetic | 95 | 435 | 90.6 | 82.6 | 38.3 | 448 | 563 | 82.1 | 65.8 | 24.6 |
| 19 | 20 | Arabidopsis | 109 | 449 | 99.7 | 81.1 | 37.1 | 462 | 578 | 100 | 64.1 | 22.7 |
| | | thaliana | | | | | | | | | | |
| 21 | 22 | Arabidopsis | 109 | 449 | 99.7 | 80.8 | 37.1 | 462 | 578 | 100 | 64.1 | 22.7 |
| | | thaliana | | | | | | | | | | |
| 23 | 24 | Arabidopsis | 109 | 449 | 100 | 81.1 | 37.4 | 462 | 578 | 99.1 | 65 | 22.7 |
| | | thaliana | | | | | | | | | | |
| 25 | 26 | Synthetic | 109 | 450 | 97.1 | 81 | 37.1 | 463 | 579 | 98.3 | 63.2 | 22.7 |
| 27 | 28 | Brassica napus | 109 | 450 | 96.8 | 80.7 | 36.9 | 463 | 579 | 95.7 | 65.8 | 22.7 |
| 29 | 30 | Brassica napus | 109 | 450 | 96.5 | 81.3 | 37.1 | 463 | 579 | 95.7 | 65.8 | 22.7 |
| 31 | 32 | Ricinus | 110 | 450 | 91.5 | 83.9 | 37.8 | 463 | 578 | 87.2 | 67.5 | 25.4 |
| | | communis | | | | | | | | | | |
| 33 | 34 | Vitis vinifera | 107 | 447 | 90.3 | 83.8 | 37.6 | 460 | 575 | 83.8 | 69.2 | 26.3 |
| 35 | 36 | Vitis vinifera | 106 | 446 | 90.3 | 83.8 | 37.6 | 459 | 574 | 83.8 | 69.2 | 26.3 |
| 37 | 38 | Populus | 112 | 452 | 92.4 | 82.5 | 37.8 | 465 | 580 | 85.5 | 65.8 | 23.7 |
| | | trichocarpa | | | | | | | | | | |
| 39 | 40 | Vitis vinifera | 106 | 450 | 89.3 | 82.8 | 37.2 | 463 | 578 | 84.6 | 68.4 | 26.3 |
| 41 | 42 | Glycine max | 107 | 447 | 90.6 | 83.6 | 37.6 | 460 | 575 | 83.8 | 67.5 | 27.1 |
| 43 | 44 | Glycine max | 105 | 445 | 89.4 | 83.6 | 37.1 | 458 | 573 | 85.5 | 67.5 | 27.1 |
| 45 | 46 | Glycine max | 105 | 445 | 89.1 | 83.3 | 36.8 | 458 | 573 | 85.5 | 67.5 | 27.1 |
| 47 | 48 | Glycine max | 107 | 447 | 90.3 | 83.3 | 37.1 | 460 | 575 | 82.9 | 67.5 | 25.4 |
| 49 | 50 | Glycine max | 105 | 445 | 89.4 | 83.6 | 37.1 | 458 | 573 | 85.5 | 67.5 | 27.1 |
| 51 | 52 | Glycine max | 105 | 445 | 89.4 | 83.6 | 37.1 | 458 | 573 | 85.5 | 67.5 | 27.1 |
| 53 | 54 | Glycine max | 105 | 445 | 89.4 | 83.6 | 37.1 | 458 | 573 | 84.6 | 67.5 | 27.1 |
| 55 | 56 | Arabidopsis | 98 | 435 | 80.8 | 98.5 | 35.9 | 448 | 562 | 64.1 | 100 | 24.2 |
| | | thaliana | | | | | | | | | | |
| 57 | 58 | Arabidopsis | 97 | 438 | 81.4 | 99.7 | 36.2 | 451 | 565 | 64.1 | 99.1 | 24.2 |
| | | lyrata subsp. | | | | | | | | | | |
| | | lyrata | | | | | | | | | | |
| 59 | 60 | Brassica napus | 99 | 440 | 80.8 | 98.8 | 35.9 | 452 | 567 | 65 | 94.8 | 25.8 |
| 61 | 62 | Zea mays | 68 | 420 | 48.3 | 47 | 34.6 | 438 | 547 | 30.5 | 30.5 | 28.2 |
| 63 | 64 | Helianthus | 93 | 446 | 50.3 | 49.9 | 35.5 | 463 | 572 | 31.4 | 33.9 | 29.1 |
| | | annuus | | | | | | | | | | |
| 65 | 66 | Synthetic | 93 | 446 | 50.3 | 49.9 | 35.5 | 463 | 572 | 31.4 | 33.9 | 29.1 |
| 67 | 68 | Helianthus | 110 | 449 | 88 | 81.9 | 38.6 | 463 | 578 | 81.2 | 65.8 | 26.3 |
| | | annuus | | | | | | | | | | |
| 69 | 70 | Synthetic | 110 | 449 | 88 | 81.9 | 38.6 | 463 | 578 | 81.2 | 65.8 | 26.3 |
| 71 | 72 | Helianthus | 14 | 360 | 41.7 | 40.4 | 40.8 | 374 | 503 | 19.4 | 18.8 | 21.3 |
| | | annuus | | | | | | | | | | |
| 73 | 74 | Synthetic | 14 | 360 | 41.7 | 40.4 | 40.8 | 374 | 503 | 19.4 | 18.8 | 21.3 |
| 75 | 76 | Pectobacterium | 5 | 350 | 36.5 | 35.9 | 93.1 | 362 | 478 | 21 | 23.5 | 84.6 |
| | | wasabiae | | | | | | | | | | |
| 77 | 78 | Zymomonas | 9 | 347 | 38.4 | 38.6 | 39.8 | 359 | 474 | 26.3 | 24.6 | 26.5 |
| | | mobilis | | | | | | | | | | |
| 79 | 80 | Photobacterium | 5 | 350 | 36.9 | 36.2 | 82.7 | 362 | 479 | 25 | 19.8 | 67.8 |
| | | profundum | | | | | | | | | | |
| 81 | 82 | Arabidopsis | 1 | 327 | 27.1 | 26.1 | 28.4 | 341 | 471 | 22.4 | 19.3 | 24.8 |
| | | thaliana | | | | | | | | | | |
| 83 | 84 | Arabidopsis | 116 | 469 | 49.3 | 48.7 | 33.9 | 486 | 595 | 32.2 | 32.2 | 29.9 |
| | | thaliana | | | | | | | | | | |
| 85 | 86 | Escherichia | 1 | 345 | 44.5 | 43.4 | 41.3 | 355 | 469 | 23.7 | 25.8 | 25 |
| | | coli | | | | | | | | | | |
| 87 | 88 | Escherichia | 5 | 350 | 37.4 | 36.2 | 100 | 362 | 478 | 22.7 | 24.2 | 100 |
| | | coli | | | | | | | | | | |
| 89 | 90 | Synthetic | 5 | 350 | 37.4 | 36.2 | 100 | 362 | 478 | 22.7 | 24.2 | 100 |
| 91 | 92 | Escherichia | 5 | 350 | 37.4 | 36.2 | 99.7 | 362 | 478 | 22.7 | 24.2 | 100 |
| | | coli B185 | | | | | | | | | | |
| 93 | 94 | Escherichia | 5 | 350 | 37.4 | 36.5 | 99.4 | 362 | 478 | 22.7 | 24.2 | 100 |
| | | coli 2362-75 | | | | | | | | | | |
| 95 | 96 | Photorhabdus | 5 | 350 | 35.7 | 36.5 | 90.8 | 362 | 478 | 22.7 | 22.5 | 76.9 |
| | | luminescens | | | | | | | | | | |
| | | subsp. | | | | | | | | | | |
| | | laumondii | | | | | | | | | | |
| | | TTO1 | | | | | | | | | | |
| 97 | 98 | Photorhabdus | 5 | 350 | 35.4 | 35.9 | 89.6 | 362 | 478 | 22.7 | 23.5 | 76.1 |
| | | asymbiotica | | | | | | | | | | |
| | | subsp. | | | | | | | | | | |
| | | asymbiotica | | | | | | | | | | |
| | | ATCC 43949 | | | | | | | | | | |
| 99 | 100 | Actinobacillus | 5 | 350 | 37.2 | 37.1 | 85.8 | 362 | 476 | 24.2 | 24.4 | 65 |
| | | succinogenes | | | | | | | | | | |
| | | 130Z | | | | | | | | | | |

As provided in Table 6, some pyruvate kinases or polypeptides having pyruvate kinase activity comprise both Pfam:PF00224 pyruvate kinase barrel domain and Pfam:PF02887 pyruvate kinase alpha/beta domain having significant sequence identity to those domains found in the pyruvate kinases as shown in SEQ ID NO: 2, 4, 6, 8, 10, 12, 14, 88, or 90. Accordingly, in other embodiments, the pyruvate kinases or the polypeptides having pyruvate kinase activity suitable for the present invention may comprise a Pfam:PF00224 pyruvate kinase barrel domain and a Pfam:PF02887 pyruvate kinase alpha/beta domain, wherein the Pfam:PF00224 pyruvate kinase barrel domain has at least 80%, preferably 85%, 90%, 91%, 92%, 93%, 94%, 95%, 96%, 97%, 98%, or 99% identity to the amino acid residues 109 to 449 of SEQ ID NO: 2, the amino acid residues 98 to 439 of SEQ ID NO: 10, or the amino acid residues 5 to 350 of SEQ ID NO: 88, and wherein the Pfam:PF02887 pyruvate kinase alpha/beta domain has at least 80%, preferably 85%, 90%, 91%, 92%, 93%, 94%, 95%, 96%, 97%, 98%, or 99% identity to the amino acid residues 462 to 578 of SEQ ID NO: 2, the amino acid residues 452 to 566 of SEQ ID NO: 10, or the amino acid residues 362 to 478 of SEQ ID NO: 88.

The pyruvate kinases or the polypeptides having pyruvate kinase activity suitable for the present invention may comprise consensus sequence(s) in the Pfam:PF00224 pyruvate kinase barrel domain and the Pfam:PF02887 pyruvate kinase alpha/beta domain. For example, as shown in FIGS. 1-3, the pyruvate kinases or the polypeptides having pyruvate kinase activity disclosed herein all comprise a consensus sequence having the amino acid sequence of G-x(2)-G-x-[DEQ]-x-[GLP]-x-[EP]-x-[ILV]-x(3)-Q-x(21, 22)—S-M-x(3)-[LP]-x-P-T-R-A-E-[AV]-x-D-[IV]-[AS]-x-A-[IV]-x-[DEQ]-x-[AST]-D-[ACG]-[ILV]-[LM]-L-[GS]-[AG]-E-[ST]-[AL]-x-G-x-[FWY]-P-x(2)-[AT]-[AILV]-x(2)-[LMV]-x(2)-[IV]-[ACS]-x(3)-[DE] (SEQ ID NO: 102) in the Pfam:PF00224 pyruvate kinase barrel domain and a consensus sequence having the amino acid sequence of T-x-[DGST]-G-x(6, 9)-R-x(3)-[GPT]-x(3)-[FILV] (SEQ ID NO: 103) in the Pfam:PF02887 pyruvate kinase alpha/beta domain. Likewise, as shown in Table 7 below, the pyruvate kinases or the polypeptides having pyruvate kinase activity disclosed herein also comprise a conserved PK active site having the amino acid sequence of [LIVAC]-x-[LIVM]-[LIVM]-[SAPCV]-K-[LIV]-E-[NKRST]-x-[DEQHS]-[GSTA]-[LIVM] (SEQ ID NO: 101). Accordingly, in further embodiments, the pyruvate kinases or the polypeptides having pyruvate kinase activity suitable for the present invention may comprise an amino acid sequence having at least 60%, preferably, 65%, 70%, 75%, 80%, 85%, 90%, 91%, 92%, 93%, 94%, 95%, 96%, 97%, 98%, or 99% identity to the amino acid sequence of SEQ ID NO: 2, 4, 6, 8, 10, 12, 14, 88, or 90, wherein the amino acid sequence further comprises the amino acid sequence of SEQ ID NO: 102 and 103. In yet further embodiments, the pyruvate kinases or the polypeptides having pyruvate kinase activity suitable for the present invention may further comprise the conserved PK active site.

TABLE 7
Identification of PK active site in the amino acid sequences of pyruvate kinases.

| PK Gene | | | PK Active | | |
|---|---|---|---|---|---|
| SEQ ID NO | | | Domain | | Best Matches |
| Nucleic | | | Domain | Domain | (Fuzzpro, max 4 |
| Acid | Amino Acid | Organism | Start | End | mismatches allowed) |
| 1 | 2 | Arabidopsis | 320 | 332 | Fits with 0 mismatches |
| | | thaliana | | | |
| 3 | 4 | Arabidopsis | 257 | 269 | Fits with 0 mismatches |
| | | thaliana | | | |
| 5 | 6 | Arabidopsis | 394 | 406 | Fits with 0 mismatches |
| | | thaliana | | | |
| 7 | 8 | Synthetic | 320 | 332 | Fits with 0 mismatches |
| 9 | 10 | Arabidopsis | 309 | 321 | Fits with 0 mismatches |
| | | thaliana | | | |
| 11 | 12 | Arabidopsis | 254 | 266 | Fits with 0 mismatches |
| | | thaliana | | | |
| 13 | 14 | Synthetic | 309 | 321 | Fits with 0 mismatches |
| 15 | 16 | Linum | 306 | 318 | Fits with 0 mismatches |
| | | usitatissimum | | | |
| 17 | 18 | Synthetic | 306 | 318 | Fits with 0 mismatches |
| 19 | 20 | Arabidopsis | 320 | 332 | Fits with 0 mismatches |
| | | thaliana | | | |
| 21 | 22 | Arabidopsis | 320 | 332 | Fits with 0 mismatches |
| | | thaliana | | | |
| 23 | 24 | Arabidopsis | 320 | 332 | Fits with 0 mismatches |
| | | thaliana | | | |
| 25 | 26 | Synthetic | 320 | 332 | Fits with 0 mismatches |
| 27 | 28 | Brassica napus | 320 | 332 | Fits with 0 mismatches |
| 29 | 30 | Brassica napus | 320 | 332 | Fits with 0 mismatches |
| 31 | 32 | Ricinus communis | 321 | 333 | Fits with 0 mismatches |
| 33 | 34 | Vitis vinifera | 318 | 330 | Fits with 0 mismatches |
| 35 | 36 | Vitis vinifera | 317 | 329 | Fits with 0 mismatches |
| 37 | 38 | Populus | 323 | 335 | Fits with 0 mismatches |
| | | trichocarpa | | | |
| 39 | 40 | Vitis vinifera | 321 | 333 | Fits with 0 mismatches |
| 41 | 42 | Glycine max | 318 | 330 | Fits with 0 mismatches |
| 43 | 44 | Glycine max | 316 | 328 | Fits with 0 mismatches |
| 45 | 46 | Glycine max | 316 | 328 | Fits with 0 mismatches |
| 47 | 48 | Glycine max | 318 | 330 | Fits with 0 mismatches |
| 49 | 50 | Glycine max | 316 | 328 | Fits with 0 mismatches |
| 51 | 52 | Glycine max | 316 | 328 | Fits with 0 mismatches |
| 53 | 54 | Glycine max | 316 | 328 | Fits with 0 mismatches |
| 55 | 56 | Arabidopsis | 305 | 317 | Fits with 0 mismatches |
| | | thaliana | | | |
| 57 | 58 | Arabidopsis lyrata | 308 | 320 | Fits with 0 mismatches |
| | | subsp. lyrata | | | |
| 59 | 60 | Brassica napus | 310 | 322 | Fits with 0 mismatches |
| 61 | 62 | Zea mays | 291 | 303 | Fits with 0 mismatches |
| 63 | 64 | Helianthus annuus | 316 | 328 | Fits with 0 mismatches |
| 65 | 66 | Synthetic | 316 | 328 | Fits with 0 mismatches |
| 67 | 68 | Helianthus annuus | 321 | 333 | Fits with 0 mismatches |
| 69 | 70 | Synthetic | 321 | 333 | Fits with 0 mismatches |
| 71 | 72 | Helianthus annuus | 230 | 242 | Fits with 0 mismatches |
| 73 | 74 | Synthetic | 230 | 242 | Fits with 0 mismatches |
| 75 | 76 | Pectobacterium | 218 | 230 | Fits with 0 mismatches |
| | | wasabiae | | | |
| 77 | 78 | Zymomonas mobilis | 217 | 229 | Fits with 1 mismatches |
| 79 | 80 | Photobacterium | 218 | 230 | Fits with 0 mismatches |
| | | profundum | | | |
| 81 | 82 | Arabidopsis | 198 | 210 | Fits with 2 mismatches |
| | | thaliana | | | |
| 83 | 84 | Arabidopsis | 339 | 351 | Fits with 0 mismatches |
| | | thaliana | | | |
| 85 | 86 | Escherichia coli | 215 | 227 | Fits with 0 mismatches |
| 87 | 88 | Escherichia coli | 218 | 230 | Fits with 0 mismatches |
| 89 | 90 | Synthetic | 218 | 230 | Fits with 0 mismatches |
| 91 | 92 | Escherichia coli | 218 | 230 | Fits with 0 mismatches |
| | | B185 | | | |
| 93 | 94 | Escherichia coli | 218 | 230 | Fits with 0 mismatches |
| | | 2362-75 | | | |
| 95 | 96 | Photorhabdus | 218 | 230 | Fits with 1 mismatches |
| | | luminescens subsp. | | | |
| | | laumondii TTO1 | | | |
| 97 | 98 | Photorhabdus | 218 | 230 | Fits with 1 mismatches |
| | | asymbiotica subsp. | | | |
| | | asymbiotica ATCC | | | |
| | | 43949 | | | |
| 99 | 100 | Actinobacillus | 218 | 230 | Fits with 0 mismatches |
| | | succinogenes 130Z | | | |

As used herein, “functional variants” or “functional equivalent” of a molecule (e.g., a polypeptide or nucleic acid molecule) is intended to mean a molecule having substantially similar sequence as compared to the non-variant molecule while retaining the activity of the non-variant molecule in whole or in part.

For nucleotide sequences comprising an open reading frame, functional variants include those sequences that, because of the degeneracy of the genetic code, encode the identical amino acid sequence of the native protein. Naturally occurring allelic variants can be identified with the use of well-known molecular biology techniques, such as, for example, with polymerase chain reaction (PCR) and hybridization techniques. Functional variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. A variant nucleotide sequence may also contain insertions, deletions, or substitutions of one or more nucleotides relative to the nucleotide sequence found in nature. Accordingly, a variant protein may contain insertions, deletions, or substitutions of one or more amino acid residues relative the amino acid sequence found in nature. Generally, variants of the polynucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, or 99, or the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100, will have at least 60%, preferably 70%, 75%, 80%, 85%, 90%, 95%, 96%, 97%, 98%, 99%, 99.1%, 99.2%, 99.3%, 99.4%, 99.5%, 99.6%, 99.7%, 99.8%, or 99.9% sequence identity to the corresponding nucleotide or amino acid sequence. The functional variants of the polynucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, or 99 may be variants of the corresponding wild-type polynucleotide sequence, provided that they encode a polypeptide retaining the activity of the polypeptide encoded by the polynucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, or 99 in conferring increased content in one or more of protein, oil, or one or more amino acids in a plant, plant cell, or plant part relative to a corresponding wild-type plant, plant cell, or plant part. In some embodiments, such functional variants are capable of conferring increased content in protein and one or more amino acids in a plant, plant cell, or plant part relative to a corresponding wild-type plant, plant cell, or plant part. In another embodiment, such functional variants are capable of conferring increased content in oil and one or more amino acids in a plant, plant cell, or plant part relative to a corresponding wild-type plant, plant cell, or plant part. In other embodiment, such functional variants are capable of conferring increased content in protein, oil and one or more amino acids in a plant, plant cell, or plant part relative to a corresponding wild-type plant, plant cell, or plant part.

Likewise, the functional variants of the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 may be variants of the corresponding wild-type amino acid sequence, provided that they retain the activity of the protein having the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 in conferring increased content in one or more of protein, oil, or one or more amino acids in a plant, plant cell, or plant part relative to a corresponding wild-type plant, plant cell, or plant part. In some embodiments, such functional variants are capable of conferring increased content in protein and one or more amino acids in a plant, plant cell, or plant part relative to a corresponding wild-type plant, plant cell, or plant part. In another embodiment, such functional variants are capable of conferring increased content in oil and one or more amino acids in a plant, plant cell, or plant part relative to a corresponding wild-type plant, plant cell, or plant part. In other embodiment, such functional variants are capable of conferring increased content in protein, oil and one or more amino acids in a plant, plant cell, or plant part relative to a corresponding wild-type plant, plant cell, or plant part. Preferably, the functional variants of the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 also comprise one or more of the aforementioned conserved PK active site, the Pfam:PF00224 pyruvate kinase barrel domain (and/or the consensus sequence of SEQ ID NO: 102), and/or the Pfam:PF02887 pyruvate kinase alpha/beta domain (and/or the consensus sequence of SEQ ID NO: 103). Methods for analyzing an amino acid sequence and identifying functional domain(s) contained therein are known in the art. For example, HMMER algorithm (Durbin et al., “Biological sequence analysis: probabilistic models of proteins and nucleic acids,” Cambridge University Press, 1998; Eddy S., Bioinformatics, 1998, 14(9): 755-763; Schultz et al., Proc. Natl. Acad. Sci. USA, 1998, 95(11): 5857-5864) against the PFAM (comprehensive database of conserved protein family) database may be used to predict domain profile of a particular amino acid sequence. The PFAM database (Finn et al. Nucleic Acids Research, 2006, Database Issue 34: D247-D251) compiles a large collection of multiple sequence alignments and hidden Markov models (HMM) covering many common protein domains and families and is available through the Sanger Institute in the United Kingdom (Bateman et al., Nucleic Acids Research, 2002, 30(1): 276-280). Tools useful in searching such databases are known in the art, for example INTERPRO (European Bioinformatics institute, UK) which allows searching several protein domain databases simultaneously. The amino acid positions of two Pfam domains in the sequences of various pyruvate kinases are provided in Table 1 above.

Moreover, in addition to the polypeptide having pyruvate kinases activity shown in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100, which is encoded by the polynucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, or 99, respectively, the skilled worker will recognize that DNA sequence polymorphisms which lead to changes in the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 may exist naturally within a population. These genetic polymorphisms in the polynucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, or 99 may exist between individuals within a population owing to natural variation. These natural variants usually bring about a variance of 1 to 5% in the polynucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, or 99. Each and every one of these nucleotide variations and resulting amino acid polymorphisms in the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100, which are the result of natural variation and do not modify the functional activity, are also encompassed by the invention.

As used herein, “sequence identity” or “identity” refers to a relationship between two or more polynucleotide or polypeptide sequences, as determined by aligning the sequences for maximum correspondence over a specified comparison window. As used in the art, “identity” also means the degree of sequence relatedness between polynucleotide or polypeptide sequences as determined by the match between strings of such sequences.

“Percent identity” (% identity) or “percent sequence identity” (% sequence identity) as used herein refers to the value determined by comparing two optimally aligned sequences over a specified comparison window.

The percent identity of protein sequences as shown in Tables 1, 2, and 4 was determined by pairwise alignment of the sequences over in each case the entire sequence length, using the algorithm of Needleman and Wunsch, as implemented in the European Molecular Biology Open Software Suite (EMBOSS), version 6.3.1.2 (Trends in Genetics, 2000, 16(6): 276). Parameters used were Matrix=EBLOSUM62; gapopen=10.0; gapextend=2.0.

Multiple protein alignments as shown in the Figures and derived dendograms were produced by using the clustal algorithm as implemented in AlignX (version 31 Jul. 2006), a component of the Vector NTI Advance 10.3.0 software package of the Invitrogen Corporation. Parameters used for multiple alignments were default parameters, using gap opening penalty=10; gap extension penalty=0.05; gap separation penalty range=8; matrix=blosum62. The clustal algorithm is publicly available from various sources, e.g., from the ftp server of the European Bioinformatics Institute (EBI) (see website at ebi.ac.uk/pub/software).

For identification of domains in the sequences of this application, as shown in Table 6, the PFAM-A database release 25.0 was used, which is publicly available (e.g., see website at pfam.sanger.ac.uk). Domains were identified by using the hmmscan algorithm. This algorithm is part of the HMMER3 software package and is publicly available (e.g., from the Howard Hughes Medical Institute, Janelia Farm Research Campus, see website at hmmer.org). Parameters for the hmmscan algorithm were default parameters as implemented in hmmscan (HMMER release 3.0). Domains were scored to be present in a given sequence when the reported E-value was 0.1 or lower and if at least 80% of the length of the PFAM domain model was covered in the algorithm-produced alignment.

Sequence alignments and calculation of percent sequence identity may also be performed with CLUSTAL (see website at ebi.ac.uk/Tools/clustalw2/index.html), the program PileUp (Feng et al., J. Mol. Evolution., 1987, 25: 351-360; Higgins et al., CABIOS, 1989, 5: 151-153), or the programs Gap and BestFit (Needleman and Wunsch, J. Mol. Biol., 1970, 48: 443-453; Smith and Waterman, Adv. Appl. Math., 1981, 2: 482-489), which are part of the GCG software packet (Gentics Computer Group, 575 Science Drive, Madison, Wis.).

Other methods of sequence alignment for comparison and calculation of percent sequence identity are well known in the art. For example, the percent sequence identity may be determined with the Vector NTI Advance 10.3.0 (PC) software package (Invitrogen, 1600 Faraday Ave., Carlsbad, Calif. 92008). For percent identity calculated with Vector NTI, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (e.g., Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide. Sequence alignments and calculation of percent sequence identity may also be performed with CLUSTAL (see website at ebi.ac.uk/Tools/clustalw2/index.html), the program PileUp (Feng et al., J. Mol. Evolution., 1987, 25: 351-360; Higgins et al., CABIOS, 1989, 5: 151-153), or the programs Gap and BestFit (Needleman and Wunsch, J. Mol. Biol., 1970, 48: 443-453; Smith and Waterman, Adv. Appl. Math., 1981, 2: 482-489), which are part of the GCG software packet (Gentles Computer Group, 575 Science Drive, Madison, Wis.).

Methods of identifying homologous sequences with sequence similarity to a reference sequence are known in the art. For example, software for performing BLAST analyses for identification of homologous sequences is publicly available through the National Center for Biotechnology Information (see website at ncbi.nlm.nih.gov). PSI-BLAST (in BLAST 2.0) can also be used to perform an iterated search that detects distant relationships between molecules. When utilizing BLAST or PSI-BLAST, the default parameters of the respective programs (e.g., BLASTN for nucleotide sequences, BLASTX for proteins) can be used (see ncbi.nlm.nih.gov website). Alignment may also be performed manually by inspection. These methods may be used, for example, to identify homologs or variants of the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100, and/or the corresponding coding nucleotide sequences for the use in the expression cassettes of the invention.

Nucleic acid molecules encoding functional variants, homologs, analogs, and orthologs of polypeptides can be isolated. The polynucleotides encoding the respective polypeptides or primers based thereon can be used as hybridization probes according to standard hybridization techniques under stringent hybridization conditions. As used herein with regard to hybridization for DNA to a DNA blot, the term “stringent conditions” refers to hybridization overnight at 60° C. in 10×Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10×Denhart's solution, 6×SSC, 0.5% SDS and 100 mg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for performing nucleic acid hybridizations are well known in the art.

The term “homolog(s)” is a generic term used in the art to indicate a polynucleotide or polypeptide sequence possessing a high degree of sequence relatedness to a reference sequence. Such relatedness may be quantified by determining the degree of identity and/or similarity between the two sequences. Falling within this generic term are the terms “ortholog(s)” and “paralog(s).” The term “ortholog(s)” refers to a homologous polynucleotide or polypeptide in different organisms due to ancestral relationship of these genes. The term “paralog(s)” refers to a homologous polynucleotide or polypeptide that results from one or more gene duplications within the genome of a species. The orthologs, paralogs or homologs of the protein having the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 may be identified or isolated from the genome of any desired organism, preferably from another plant, according to well known techniques based on their sequence similarity to the open reading frame having the polynucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, or 99, respectively, e.g., hybridization, PCR, or computer generated sequence comparisons. For example, all or a portion of a particular open reading frame can be used as a probe that selectively hybridizes to other gene sequences present in a population of cloned genomic DNA fragments (i.e. genomic libraries) from a chosen source organism. Further, suitable genomic libraries may be prepared from any cell or tissue of an organism. Such techniques include hybridization screening of plated DNA libraries (either plaques or colonies; see, e.g., Sambrook, 1989, Molecular Cloning: A Laboratory Manual, 2^{nd} ed., Cold Spring Harbor Laboratory, Cold Spring Harbor Laboratory Press, Cold Spring Harbor, N.Y.) and amplification by PCR using oligonucleotide primers preferably corresponding to sequence domains conserved among related polypeptides or subsequences of the nucleotide sequences provided herein. These methods are known and particularly well suited to the isolation of gene sequences from organisms closely related to the organism from which the probe sequence is derived. The application of these methods using all or a portion of an open reading frame encoding a polypeptide having pyruvate kinase activity as probes is well suited for the isolation of gene sequences from any source organism, preferably other plant species. In a PCR approach, oligonucleotide primers can be designed for use in PCR reactions to amplify corresponding DNA sequences from cDNA or genomic DNA extracted from any plant of interest. Methods for designing PCR primers and PCR cloning are known in the art.

Suitable oligonucleotides for use as primers in probing or amplification reactions as the PCR reaction described above, may be about 30 or fewer nucleotides in length (e.g., 9, 12, 15, 18, 20, 21, 22, 23, or 24, or any number between 9 and 30). Generally, specific primers are upwards of 14 nucleotides in length. For optimum specificity and cost effectiveness, primers of 16 to 24 nucleotides in length are preferred. Those skilled in the art are well versed in the design of primers for use in processes such as PCR. If required, probing can be done with entire restriction fragments of the genes disclosed herein which may be 100's or even 1000's of nucleotides in length.

Accordingly, in some preferred embodiments, the nucleic acid molecule encoding a polypeptide having pyruvate kinase activity to be included in the expression cassettes of the invention comprises a polynucleotide sequence selected from the group consisting of:

(a) the polynucleotide sequence of SEQ ID NO: 1, 3, 5, 7, 9, 11 or 13;

(b) a nucleotide sequence encoding the amino acid sequence of SEQ ID NO: 2, 4, 6, 8, 10, 12 or 14;

(c) a nucleotide sequence having at least 60% identity to the nucleotide sequence of SEQ ID NO: 1, 3, 5, 7, 9, 11 or 13 and encoding a polypeptide having a Pfam:PF00224 pyruvate kinase barrel domain and a Pfam:PF02887 pyruvate kinase alpha/beta domain;

(d) a nucleotide sequence encoding an amino acid sequence having at least 60% identity to the amino acid sequence of SEQ ID NO: 2, 4, 6, 8, 10, 12 or 14 and having a Pfam:PF00224 pyruvate kinase barrel domain and a Pfam:PF02887 pyruvate kinase alpha/beta domain;

(e) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 109 to 449 of SEQ ID NO: 2 or the amino acid residues 98 to 439 of SEQ ID NO: 10, and wherein the Pfam:PF02887 pyruvate kinase alpha/beta domain has at least 80% identity to the amino acid residues 462 to 578 of SEQ ID NO: 2 or the amino acid residues 452 to 566 of SEQ ID NO: 10; and

(f) a nucleotide sequence encoding an amino acid sequence having at least 60% identity to the amino acid sequence of SEQ ID NO: 2, 4, 6, 8, 10, 12 or 14, wherein said amino acid sequence further comprises the amino acid sequence of SEQ ID NO: 102 and 103,

wherein the expression of the nucleic acid molecule in a plant, plant cell, or plant part confers increased content in one or more of protein, oil, or one or more amino acids in a plant, plant cell, or plant part relative to a corresponding wild-type plant, plant cell, or plant part.

In other preferred embodiments, the nucleic acid molecule encoding a polypeptide having pyruvate kinase activity to be included in the expression cassettes of the invention comprises a polynucleotide sequence selected from the group consisting of:

(a) the nucleotide sequence of SEQ ID NO: 87 or 89;

(b) a nucleotide sequence encoding the amino acid sequence of SEQ ID NO: 88 or 90;

(c) a nucleotide sequence having at least 75% identity to the nucleotide sequence of SEQ ID NO: 87 or 89 and encoding a polypeptide having a Pfam:PF00224 pyruvate kinase barrel domain and a Pfam:PF02887 pyruvate kinase alpha/beta domain;

(d) a nucleotide sequence encoding an amino acid sequence having at least 75% identity to the amino acid sequence of SEQ ID NO: 88 or 90 and having a Pfam:PF00224 pyruvate kinase barrel domain and a Pfam:PF02887 pyruvate kinase alpha/beta domain,

(e) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 5 to 350 of SEQ ID NO: 88, and wherein the Pfam:PF02887 pyruvate kinase alpha/beta domain has at least 80% identity to the amino acid residues 362 to 478 of SEQ ID NO: 88; and

(f) a nucleotide sequence encoding an amino acid sequence having at least 75% identity to the amino acid sequence of SEQ ID NO: 88 or 90, wherein said amino acid sequence further comprises the amino acid sequence of SEQ ID NO: 102 and 103,

wherein the expression of the nucleic acid molecule in a plant, plant cell, or plant part confers increased content in one or more of protein, oil, or one or more amino acids in a plant, plant cell, or plant part relative to a corresponding wild-type plant, plant cell, or plant part.

Nucleotide sequences may be codon optimized to improve expression in heterologous host cells. Nucleotide sequences from a heterologous source are codon optimized to match the codon bias of the host. A codon consists of a set of three nucleotides, referred to as a triplet, which encodes a specific amino acid in a polypeptide chain or for the termination of translation (stop codons). The genetic code is redundant in that multiple codons specify the same amino acid, i.e., 61 codons encoding for 20 amino acids. Organisms exhibit preference for one of the several codons encoding the same amino acid, which is known as codon usage bias. The frequency of codon usage for different species has been determined and recorded in codon usage tables. Codon optimization replaces infrequently used codons present in a DNA sequence of a heterologous gene with preferred codons of the host, based on a codon usage tables. The amino acid sequence is not altered during the process. Codon optimization can be performed using gene optimization software, such as Leto 1.0 from Entelechon. Protein sequences for the genes to be codon optimized are back-translated in the program and the codon usage is selected from a list of organisms. Leto 1.0 replaces codons from the original sequence with codons that are preferred by the organism into which the sequence will be transformed. The DNA sequence output is translated and aligned to the original protein sequence to ensure that no unwanted amino acid changes were introduced. For example, the nucleotide sequence of SEQ ID NO: 7 is the codon optimized version of the nucleotide sequence of SEQ ID NO: 1 for expression of the amino acid sequence of SEQ ID NO: 8 and 2, respectively, in maize. Similarly, the nucleotide sequence of SEQ ID NO: 13 is the codon optimized version of the nucleotide sequence of SEQ ID NO: 9 for expression of the amino acid sequence of SEQ ID NO: 14 and 10, respectively, in maize. Likewise, the nucleotide sequence of SEQ ID NO: 89 is the codon optimized version of the nucleotide sequence of SEQ ID NO: 87 for expression of the amino acid sequence of SEQ ID NO: 90 and 88, respectively, in maize.

In addition to codon optimization of a sequence from a heterologous source, gene optimization entails further modifications to the DNA sequence to optimize the gene sequence for expression without altering the protein sequence. The Leto 1.0 program can also be used to remove sequences that might negatively impact gene expression, transcript stability, protein expression or protein stability, including but not limited to, transcription splice sites, DNA instability motifs, plant polyadenylation sites, secondary structure, AU-rich RNA elements, secondary ORFs, codon tandem repeats, long range repeats. This can also be done to optimize gene sequences originating from the host organism. Another component of gene optimization is to adjust the G/C content of a heterologous sequence to match the average G/C content of endogenous genes of the host.

For example, to provide plant optimized nucleic acids, the DNA sequence of a gene can be modified to: 1) comprise codons preferred by highly expressed plant genes; 2) comprise an A+T content in nucleotide base composition to that substantially found in plants; 3) form a plant initiation sequence; 4) eliminate sequences that cause destabilization, inappropriate polyadenylation, degradation and termination of RNA, or that form secondary structure hairpins or RNA splice sites; or 5) eliminate antisense open reading frames. Increased expression of nucleic acids in plants can be achieved by utilizing the distribution frequency of codon usage in plants in general or in a particular plant. Methods for optimizing nucleic acid expression in plants can be found in EP 0359472, EP 0385962, WO 91/16432, U.S. Pat. No. 5,380,831, U.S. Pat. No. 5,436,391, Perlack et al. (Proc. Natl. Acad. Sci. USA, 1991, 88: 3324-3328), and Murray et al. (Nucleic Acids Res., 1989, 17: 477-498).

Accordingly, in some other embodiments of the invention, the nucleic acid molecule encoded by the transgene is codon optimized to improve expression of the transgene in host cells. The nucleic acid sequence may be codon optimized for any host cell in which it is expressed. In one embodiment, the nucleic acid sequence is codon optimized for maize. In further embodiments, the nucleic acid sequence may also be codon optimized for other plant species including, but not limited to rice, wheat, barley, soybean, canola, rapeseed, cotton, sugarcane, or alfalfa.

1.2 Other Regulatory Elements

In addition to the promoter and the nucleic acid molecule encoding a polypeptide having pyruvate kinase activity, the expression cassettes of the present invention may further comprise other regulatory elements. The term “regulatory elements” encompasses all sequences which may influence construction or function of the expression cassette. Regulatory elements may, for example, modify transcription and/or translation of a gene in a prokaryotic or eukaryotic organism. Thus, the expression profile of the nucleic acid molecule included in the expression cassettes of the invention may be modulated depending on the combination of the transcription regulating nucleotide sequence and the other regulatory element(s) comprised in the expression cassette.

Accordingly, in one embodiment, the expression cassettes of the invention may further comprise at least one additional regulatory element selected from the group consisting of:

(a) 5′-untranslated sequences (or 5′-untranslated regions or 5′-UTR),

(b) intron sequences,

(c) transcription termination sequences (or terminators).

A variety of 5′ and 3′ transcriptional regulatory sequences are available for use in the expression cassettes of the present invention. As the DNA sequence between the transcription initiation site and the start codon of the coding sequence, i.e., the 5′-untranslated sequence, can influence gene expression, one may wish to include a particular 5′-untranslated sequence in the expression cassettes of the invention. Preferred 5′-untranslated sequences include those sequences predicted to direct optimum expression of the attached gene, i.e., consensus 5′-untranslated sequences which may increase or maintain mRNA stability and prevent inappropriate initiation of translation. The choice of such sequences will be known to those of skill in the art. Sequences obtained from genes that are highly expressed in plants will be most preferred. Also preferred is the 5′-untranslated region obtained from the same gene as the transcription regulating sequence to be included in the expression cassette of the invention.

Additionally, it is known in the art that a number of non-translated leader sequences are capable of enhancing expression, for example, leader sequences derived from viruses. For example, leader sequences from Tobacco Mosaic Virus (TMV), Maize Chlorotic Mottle Virus (MCMV), and Alfalfa Mosaic Virus (AMV) have been shown to be effective in enhancing expression (e.g., Gallie 1987; Skuzeski 1990). Other viral leader sequences known in the art include, but not limited to, Picornavirus leaders, for example, EMCV leader (Encephalomyocarditis 5′ noncoding region) (Elroy-Stein 1989), Potyvirus leaders, for example, TEV leader (Tobacco Etch Virus), MDMV leader (Maize Dwarf Mosaic Virus), Human immunoglobulin heavy-chain binding protein (BiP) leader (Macejak 1991), and untranslated leader from the coat protein mRNA of alfalfa mosaic virus (AMV RNA 4) (Jobling 1987).

The 3′ regulatory sequence preferably includes from about 50 to about 1,000, more preferably about 100 to about 1,000, base pairs and contains plant transcriptional and translational termination sequences. Transcription termination sequences, or terminators, are responsible for the termination of transcription and correct mRNA polyadenylation. Thus, the terminators preferably comprise a sequence inducing polyadenylation. The terminator may be heterologous with respect to the transcription regulating nucleotide sequence and/or the nucleic acid sequence to be expressed, but may also be the natural terminator of the gene from which the transcription regulating nucleotide sequence and/or the nucleic acid sequence to be expressed is obtained. In one embodiment, the terminator is heterologous to the transcription regulating nucleotide sequence and/or the nucleic acid sequence to be expressed. In another embodiment, the terminator is the natural terminator of the gene of the transcription regulating nucleotide sequence.

Appropriate terminators and those which are known to function in plants include, but are not limited to, CaMV 35S terminator, the tml terminator, the nopaline synthase (NOS) terminator (t-NOS) (SEQ ID NO: 115), the pea rbcS E9 terminator, the terminator for the T7 transcript from the octopine synthase (OCS) gene of Agrobacterium tumefaciens (t-OCS3) (SEQ ID NO: 116), the 3′ end of the protease inhibitor I or II genes from potato or tomato, and the TOI3357 terminator from Oryza sativa (SEQ ID NO: 123). Alternatively, one also could use a gamma coixin, oleosin 3 or other terminator from the genus Coix. Preferred 3′ regulatory elements include, but are not limited to, those from the nopaline synthase (NOS) gene of Agrobacterium tumefaciens (Bevan 1983) (SEQ ID NO: 115), the terminator for the T7 transcript from the octopine synthase gene of Agrobacterium tumefaciens (SEQ ID NO: 116), and the 3′ end of the protease inhibitor I or II genes from potato or tomato. Non-limiting examples of terminators to be included in the expression cassettes of the invention may comprise the nucleotide sequence of SEQ ID NO: 115 or 116.

Accordingly, in some preferred embodiments, the expression cassettes of the invention may further comprise a terminator selected from the group consisting of:

(a) a terminator comprising the nucleotide sequence of SEQ ID NO: 115 or 116; and

(b) a terminator comprising a nucleotide sequence having at least 90%, preferably 95%, 96%, 97%, 98%, 99%, 99.1%, 99.2%, 99.3%, 99.4%, 99.5%, 99.6%, 99.7%, 99.8%, or 99.9% identity to the nucleotide sequence of SEQ ID NO: 115 or 116.

Transcription regulatory elements can also include intron sequences that have been shown to enhance gene expression in transgenic plants, particularly in monocotyledonous plants. The intron sequence is preferably inserted in the expression cassettes of the invention between the promoter and the nucleic acid molecule to be expressed. In some preferred embodiments, such expression enhancing intron sequences are from monocotyledonous plants. In other preferred embodiments, such expression enhancing intron sequences are obtained from rice. Preferred intron sequences include, but are not limited to, intron sequences from Adh1 (Callis 1987), bronze1, actin1, actin2 (WO 00/760067), Met1 (US 2009/0144863), and MADS3 genes, or the sucrose synthase intron (Vasil 1989), see The Maize Handbook, Chapter 116 (Freeling and Walbot, Eds., Springer, New York, 1994); the Atc17 intron from the ADP-ribosylation factor 1 (ARF1) gene NEENAc17 intron from Arabidopsis thaliana (SEQ ID NO: 121), and the Atss1 intron from the aspartyl protease family protein related NEENA gene intron from Arabidopsis thaliana (SEQ ID NO: 122) More preferably, the intron sequences are:

(a) the introns of the rice Metallothionin1 (Met1) gene as described in, for example, US 2009/0144863 (hereby incorporated by reference in its entirety), preferably the first intron (intron I) thereof, most preferably an intron comprising the nucleotide sequence of SEQ ID NO: 111,

(b) the introns of the rice MADS3 gene, preferably the first intron (intron I) thereof, most preferably an intron comprising the nucleotide sequence of SEQ ID NO: 112,

(c) the introns of the Zea mays ubiquitin gene, preferably the first intron (intron I) thereof, as one embodiment an intron comprising the sequence of SEQ ID NO: 127,

(d) the introns of the rice actin gene, preferably the first intron (intron I) thereof, most preferably an intron comprising the nucleotides 121 to 568 of the sequence described by GenBank Accession No. X63830, and

(e) the introns of the Zea mays alcohol dehydrogenase (adh) gene, preferably the sixth intron (intron 6) thereof, most preferably an intron comprising the nucleotides 3,135 to 3,476 of the sequence described by GenBank Accession No. X04049.

Accordingly, in some preferred embodiments, the expression cassettes of the invention may further comprise an intron selected from the group consisting of:

(a) an intron of the rice Metallothionin1 gene comprises the nucleotide sequence of SEQ ID NO: 111 or a nucleotide sequence having at least 90%, preferably 95%, 96%, 97%, 98%, 99%, 99.1%, 99.2%, 99.3%, 99.4%, 99.5%, 99.6%, 99.7%, 99.8%, or 99.9% identity to the nucleotide sequence of SEQ ID NO: 111; and

(b) an intron of the rice MADS3 gene comprises the nucleotide sequence of SEQ ID NO: 112 or a nucleotide sequence having at least 90%, preferably 95%, 96%, 97%, 98%, 99%, 99.1%, 99.2%, 99.3%, 99.4%, 99.5%, 99.6%, 99.7%, 99.8%, or 99.9% identity to the nucleotide sequence of SEQ ID NO: 112.

Isolation of rice Metallothionein1 introns and functional variants thereof are described for example in US 2009/0144863 (hereby incorporated by reference in its entirety). Additional intron sequences with expression enhancing properties in plants may also be identified and isolated according to the disclosure of US 2006/0094976 (hereby incorporated by reference in its entirety).

1.3 Protein Targeting Sequences

In addition to the aforementioned components, the expression cassettes of the present invention may further comprise protein targeting sequences. The term “protein targeting sequences” as used herein encompasses all nucleotide sequences encoding transit peptides for directing a protein to a particular cell compartment such as vacuole, nucleus, all types of plastids like amyloplasts, chloroplasts, or chromoplasts, extracellular space, mitochondria, endoplasmic reticulum, oil bodies, peroxisomes and other compartments of plant cells (for review see Kermode 1996, Crit. Rev. Plant Sci. 15: 285-423 and references cited therein).

In some embodiments, it may be advantageous to direct the pyruvate kinase or the polypeptide having pyruvate kinase activity that are encoded by the nucleic acid molecule comprised in the expression cassettes of the invention to a particular cell compartment or organelle, such as plastids or mitochondria. To do so, a plastid transit peptide or a mitochondrial peptide may be used. Nucleotide sequences encoding plastid transit peptides are known in the art, for example, as disclosed in U.S. Pat. No. 5,717,084, U.S. Pat. No. 5,728,925, U.S. Pat. No. 6,063,601, U.S. Pat. No. 6,130,366 and the like. Plastid-targeting transit peptides include, but are not limited to, the ferredoxin transit peptide and the starch branching enzyme 2b transit peptide. In one embodiment, the transit peptide is a plastid-targeting peptide from a ferredoxin gene. In another embodiment, the plastid-targeting peptide is from the ferredoxin gene of Silene pratensins (SpFdx) (for example, SEQ ID NO: 113 or SEQ ID NO: 120, each encoding SEQ ID NO: 114). SpFdx and several of its variants have been shown to effectively target polypeptides to the stroma (Pilon, et al., 1995, J Biol Chem. 270(8): 3882-93; Rensink, et al., 1998, Plant Physiol. 118(2): 691-699). In one embodiment, a mitochondria-targeting peptide from Citrullus lanatus can be used for targeting to the mitochondria (for example, SEQ ID NO: 124 encoding SEQ ID NO: 125).

Accordingly, in some preferred embodiments, the expression cassettes of the invention may further comprise at least one heterologous nucleotide sequence encoding a transit peptide to target the polypeptide having pyruvate kinase activity to a plastid, wherein the nucleotide sequence encoding the plastid-targeting transit peptide comprises:

(a) the nucleotide sequence of SEQ ID NO: 113 or 120;

(b) a nucleotide sequence having at least 95%, preferably 96%, 97%, 98%, 99%, 99.1%, 99.2%, 99.3%, 99.4%, 99.5%, 99.6%, 99.7%, 99.8%, or 99.9% identity to the sequence of SEQ ID NO: 113 or 120;

(c) a nucleotide sequence encoding the amino acid sequence of SEQ ID NO: 114; or

(d) a nucleotide sequence encoding a peptide having at least 95%, preferably 96%, 97%, 98%, 99%, 99.1%, 99.2%, 99.3%, 99.4%, 99.5%, 99.6%, 99.7%, 99.8%, or 99.9% identity to the amino acid sequence of SEQ ID NO: 114.

In other preferred embodiments, the expression cassettes of the invention may further comprise at least one heterologous nucleotide sequence encoding a transit peptide to target the polypeptide having pyruvate kinase activity to a mitochondria, wherein the nucleotide sequence encoding the mitochondrial targeting peptide comprises:

(a) the nucleotide sequence of SEQ ID NO: 124;

(b) a nucleotide sequence having at least 95%, preferably 96%, 97%, 98%, 99%, 99.1%, 99.2%, 99.3%, 99.4%, 99.5%, 99.6%, 99.7%, 99.8%, or 99.9% identity to the sequence of SEQ ID NO: 124;

(c) a nucleotide sequence encoding the amino acid sequence of SEQ ID NO: 125; or

(d) a nucleotide sequence encoding a peptide having at least 95%, preferably 96%, 97%, 98%, 99%, 99.1%, 99.2%, 99.3%, 99.4%, 99.5%, 99.6%, 99.7%, 99.8%, or 99.9% identity to the amino acid sequence of SEQ ID NO: 125.

1.4 Preferred Embodiments of Expression Cassettes

It is found that, by expressing certain pyruvate kinases in a plant, plant cell, or plant part under control of some specific types of promoters, optionally in combination with other specific types of regulatory elements and/or targeting peptides, the content of one or more of protein, oil, or one or more amino acids in such a plant, plant cell, or plant part is surprisingly increased. In some situations, it is found that the expression of a pyruvate kinase under the control of such a combination of regulatory sequences may result in an unexpected increase in the content of protein and one or more amino acids in such a plant, plant cell, or plant part. In further situations, it is found that such expression surprisingly confers increased content in oil and one or more amino acids in a plant, plant cell, or plant part relative to a corresponding wild-type plant, plant cell, or plant part. In other situations, it is found that such expression surprisingly confers an increased content in protein, oil and one or more amino acids in such a plant, plant cell, or plant part relative to a corresponding wild-type plant, plant cell, or plant part. This section exemplifies some of such preferred expression cassettes of the invention.

In one aspect, the present invention provides expression cassette (I) comprising:

(a) a promoter that is functional in a plant as disclosed in Section 1.1.1;

(b) a nucleic acid molecule encoding a polypeptide having pyruvate kinase activity as disclosed in Section 1.1.2, wherein the nucleic acid molecule is heterologous and operably linked to the promoter; and

(c) a rice intron as disclosed in Section 1.2.

In another aspect, the present invention provides expression cassette (II) comprising:

(a) an endosperm-specific or endosperm-preferential promoter or an embryo-specific or embryo-preferential promoter as disclosed in Section 1.1.1;

(b) a nucleic acid molecule encoding a polypeptide having pyruvate kinase activity as disclosed in Section 1.1.2, wherein the nucleic acid molecule is heterologous and operably linked to the promoter; and

(c) as disclosed in Section 1.2.

In yet another aspect, the present invention provides expression cassette (III) comprising:

(a) a seed-specific or seed-preferential promoter as disclosed in Section 1.1.1; and

(b) a nucleic acid molecule encoding a polypeptide having pyruvate kinase activity as disclosed in Section 1.1.2, wherein the nucleic acid molecule is heterologous and operably linked to the promoter,

wherein expression of the nucleic acid molecule in a plant, plant cell, or plant part confers increased content of protein, oil, and one or more amino acids in said plant, plant cell, or plant part relative to a corresponding wild-type plant, plant cell, or plant part.

Preferably, the nucleic acid molecule encoding a polypeptide having pyruvate kinase activity to be included in the aforementioned expression cassettes (I), (II) and (III) of the invention comprises:

(i) the nucleotide sequence of SEQ ID NO: 1, 3, 5, 7, 9, 11 or 13;

(ii) a nucleotide sequence encoding the amino acid sequence of SEQ ID NO: 2, 4, 6, 8, 10, 12 or 14;

(iii) a nucleotide sequence having at least 60% identity to the nucleotide sequence of SEQ ID NO: 1, 3, 5, 7, 9, 11 or 13 and encoding a polypeptide having a Pfam:PF00224 pyruvate kinase barrel domain and a Pfam:PF02887 pyruvate kinase alpha/beta domain;

(iv) a nucleotide sequence encoding an amino acid sequence having at least 60% identity to the amino acid sequence of SEQ ID NO: 2, 4, 6, 8, 10, 12 or 14 and having a Pfam:PF00224 pyruvate kinase barrel domain and a Pfam:PF02887 pyruvate kinase alpha/beta domain;

(v) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 109 to 449 of SEQ ID NO: 2 or the amino acid residues 98 to 439 of SEQ ID NO: 10, and wherein the Pfam:PF02887 pyruvate kinase alpha/beta domain has at least 80% identity to the amino acid residues 462 to 578 of SEQ ID NO: 2 or the amino acid residues 452 to 566 of SEQ ID NO: 10; or

(vi) a nucleotide sequence encoding an amino acid sequence having at least 60% identity to the amino acid sequence of SEQ ID NO: 2, 4, 6, 8, 10, 12 or 14, wherein said amino acid sequence further comprises the amino acid sequence of SEQ ID NO: 102 and 103.

More preferably, the nucleic acid molecule encoding a polypeptide having pyruvate kinase activity to be included in the aforementioned expression cassettes (I) and (II) of the invention comprises:

(a) the nucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, or 83;

(b) a nucleotide sequence encoding the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, or 84;

(c) a nucleotide sequence having at least 95% identity to the nucleotide sequence of SEQ ID NO: 1, 3, 5, 7, 9, 11 or 13; or

(d) a nucleotide sequence encoding an amino acid sequence having at least 95% identity to the amino acid sequence of SEQ ID NO: 2, 4, 6, 8, 10, 12 or 14.

In another aspect, the present invention provides expression cassette (IV) comprising:

(a) a promoter that is functional in a plant as disclosed in Section 1.1.1;

(b) a nucleic acid molecule encoding a polypeptide having pyruvate kinase activity as disclosed in Section 1.1.2, wherein the nucleic acid molecule is heterologous and operably linked to the promoter; and

(c) the first intron of the rice Metallothionin1 gene as disclosed in Section 1.2.

In a further aspect, the present invention provides expression cassette (V) comprising:

(a) a constitutive promoter that is functional in a plant as disclosed in Section 1.1.1;

(b) a nucleic acid molecule encoding a polypeptide having pyruvate kinase activity as disclosed in Section 1.1.2, wherein the nucleic acid molecule is heterologous and operably linked to the promoter; and

(c) an intron,

wherein the constitutive promoter comprises:

(i) the nucleotide sequence of SEQ ID NO: 109 or 110;

(ii) a nucleotide sequence having at least 95% identity to the nucleotide sequence of SEQ ID NO: 109 or 110, wherein said nucleotide sequence has constitutive expression activity; or

(iii) a fragment of the nucleotide sequence of SEQ ID NO: 109 or 110, wherein the fragment has constitutive expression activity.

Preferably, the nucleic acid molecule encoding a polypeptide having pyruvate kinase activity to be included in the aforementioned expression cassettes (IV) and (V) of the invention comprises:

(a) the nucleotide sequence of SEQ ID NO: 87 or 89;

(b) a nucleotide sequence encoding the amino acid sequence of SEQ ID NO: 88 or 90;

(c) a nucleotide sequence having at least 75% identity to the nucleotide sequence of SEQ ID NO: 87 or 89 and encoding a polypeptide having a Pfam:PF00224 pyruvate kinase barrel domain and a Pfam:PF02887 pyruvate kinase alpha/beta domain;

(d) a nucleotide sequence encoding an amino acid sequence having at least 75% identity to the amino acid sequence of SEQ ID NO: 88 or 90 and having a Pfam:PF00224 pyruvate kinase barrel domain and a Pfam:PF02887 pyruvate kinase alpha/beta domain;

(e) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 5 to 350 of SEQ ID NO: 88, and wherein the Pfam:PF02887 pyruvate kinase alpha/beta domain has at least 80% identity to the amino acid residues 362 to 478 of SEQ ID NO: 88; or

(f) a nucleotide sequence encoding an amino acid sequence having at least 75% identity to the amino acid sequence of SEQ ID NO: 88 or 90, wherein said amino acid sequence further comprises the amino acid sequence of SEQ ID NO: 102 and 103.

In some embodiments, the intron to be included in the aforementioned expression cassettes (I)-(V) of the invention is selected from the group consisting of:

(a) an intron of the rice Metallothionin1 gene, preferably, comprising the nucleotide sequence of SEQ ID NO: 111 or a nucleotide sequence having at least 90% identity to the nucleotide sequence of SEQ ID NO: 111; and

(b) an intron of the rice MADS3 gene, preferably, comprising the nucleotide sequence of SEQ ID NO: 112 or a nucleotide sequence having at least 90% identity to the nucleotide sequence of SEQ ID NO: 112.

Optionally, the aforementioned expression cassettes of the invention further comprise a heterologous nucleotide sequence encoding a transit peptide targeting the pyruvate kinase or the polypeptide having pyruvate kinase activity to a plastid as disclosed in Section 1.3.

The aforementioned expression cassettes of the invention may also optionally comprise a terminator as disclosed in Section 1.2.

Accordingly, examples of the expression cassettes of the invention may include, but not limited to, the various combinations of the nucleotide components as exemplified in Table 8 below.

TABLE 8
Examples of the expression cassettes of the invention.

| | | Targeting | | |
|---|---|---|---|---|
| Promoter | Intron | peptide | Gene | Terminator |
| Whole-seed | An intron of rice | Organelle- | Plant pyruvate | t-NOS (e.g. SEQ |
| specific or | Met1 (e.g. SEQ | targeting peptide | kinase (SEQ ID | ID NO: 115) or t- |
| preferential (e.g. | ID NO: 111) or | (e.g. SEQ ID | NO: 1, 3, 5, 7, 9, | OCS3 (e.g. SEQ |
| SEQ ID NO: 104 | an intron of rice | NO: 113, 120, or | 11, or 13) | ID NO: 116) |
| or 105 or 128) | MADS3 (e.g. | 124) | | |
| | SEQ ID NO: | | | |
| | 112) | | | |
| Endosperm | An intron of rice | Organelle- | Plant pyruvate | t-NOS (e.g. SEQ |
| specific or | Met1 (e.g. SEQ | targeting peptide | kinase (SEQ ID | ID NO: 115) or t- |
| preferential (e.g. | ID NO: 111) or | (e.g. SEQ ID | NO: 1, 3, 5, 7, 9, | OCS3 (e.g. SEQ |
| SEQ ID NO: 106 | an intron of rice | NO: 113, 120, or | 11, or 13) | ID NO: 116) |
| or 107) | MADS3 (e.g. | 124) | | |
| | SEQ ID NO: | | | |
| | 112) | | | |
| Embryo specific | An intron of rice | Organelle- | Plant pyruvate | t-NOS (e.g. SEQ |
| or preferential | Met1 (e.g. SEQ | targeting peptide | kinase (SEQ ID | ID NO: 115) or t- |
| (SEQ ID NO: | ID NO: 111) or | (e.g. SEQ ID | NO: 1, 3, 5, 7, 9, | OCS3 (e.g. SEQ |
| 108) | an intron of rice | NO: 113, 120, or | 11, or 13) | ID NO: 116) |
| | MADS3 (e.g. | 124) | | |
| | SEQ ID NO: | | | |
| | 112) | | | |
| Constitutive (e.g. | An intron of rice | Organelle- | E. coli pyruvate | t-NOS (e.g. SEQ |
| SEQ ID NO: 109 | Met1 (e.g. SEQ | targeting peptide | kinase (SEQ ID | ID NO: 115) or t- |
| or 110) | ID NO: 111) or | (e.g. SEQ ID | NO: 87 or 89) | OCS3 (e.g. SEQ |
| | an intron of rice | NO: 113, 120, or | | ID NO: 116) |
| | MADS3 (e.g. | 124) | | |
| | SEQ ID NO: | | | |
| | 112) | | | |
| Constitutive (e.g. | An intron of rice | None | E. coli pyruvate | t-NOS (e.g. SEQ |
| SEQ ID NO: 109 | Met1 (e.g. SEQ | | kinase (SEQ ID | ID NO: 115) or t- |
| or 110) | ID NO: 111) or | | NO: 87 or 89) | OCS3 (e.g. SEQ |
| | an intron of rice | | | ID NO: 116) |
| | MADS3 (e.g. | | | |
| | SEQ ID NO: | | | |
| | 112) | | | |
| Whole-seed | An intron of rice | Organelle- | E. coli pyruvate | t-NOS (e.g. SEQ |
| specific or | Met1 (e.g. SEQ | targeting peptide | kinase (SEQ ID | ID NO: 115) or t- |
| preferential (e.g. | ID NO: 111) or | (e.g. SEQ ID | NO: 87 or 89) | OCS3 (e.g. SEQ |
| SEQ ID NO: 104 | an intron of rice | NO: 113, 120, or | | ID NO: 116) |
| or 105 or 128) | MADS3 (e.g. | 124) | | |
| | SEQ ID NO: | | | |
| | 112) | | | |
| Whole-seed | An intron of rice | None | E. coli pyruvate | t-NOS (e.g. SEQ |
| specific or | Met1 (e.g. SEQ | | kinase (SEQ ID | ID NO: 115) or t- |
| preferential (e.g. | ID NO: 111) or | | NO: 87 or 89) | OCS3 (e.g. SEQ |
| SEQ ID NO: 104 | an intron of rice | | | ID NO: 116) |
| or 105 or 128) | MADS3 (e.g. | | | |
| | SEQ ID NO: | | | |
| | 112) | | | |
| Endosperm | An intron of rice | Organelle- | E. coli pyruvate | t-NOS (e.g. SEQ |
| specific or | Met1 (e.g. SEQ | targeting peptide | kinase (SEQ ID | ID NO: 115) or t- |
| preferential (e.g. | ID NO: 111) or | (e.g. SEQ ID | NO: 87 or 89) | OCS3 (e.g. SEQ |
| SEQ ID NO: 106 | an intron of rice | NO: 113, 120, or | | ID NO: 116) |
| or 107) | MADS3 (e.g. | 124) | | |
| | SEQ ID NO: | | | |
| | 112) | | | |
| Endosperm | An intron of rice | None | E. coli pyruvate | t-NOS (e.g. SEQ |
| specific or | Met1 (e.g. SEQ | | kinase (SEQ ID | ID NO: 115) or t- |
| preferential (e.g. | ID NO: 111) or | | NO: 87 or 89) | OCS3 (e.g. SEQ |
| SEQ ID NO: 106 | an intron of rice | | | ID NO: 116) |
| or 107) | MADS3 (e.g. | | | |
| | SEQ ID NO: | | | |
| | 112) | | | |
| Embryo specific | An intron of rice | Organelle- | E. coli pyruvate | t-NOS (e.g. SEQ |
| or preferential | Met1 (e.g. SEQ | targeting peptide | kinase (SEQ ID | ID NO: 115) or t- |
| (SEQ ID NO: | ID NO: 111) or | (e.g. SEQ ID | NO: 87 or 89) | OCS3 (e.g. SEQ |
| 108) | an intron of rice | NO: 113, 120, or | | ID NO: 116) |
| | MADS3 (e.g. | 124) | | |
| | SEQ ID NO: | | | |
| | 112) | | | |
| Embryo specific | An intron of rice | None | E. coli pyruvate | t-NOS (e.g. SEQ |
| or preferential | Met1 (e.g. SEQ | | kinase (SEQ ID | ID NO: 115) or t- |
| (SEQ ID NO: | ID NO: 111) or | | NO: 87 or 89) | OCS3 (e.g. SEQ |
| 108) | an intron of rice | | | ID NO: 116) |
| | MADS3 (e.g. | | | |
| | SEQ ID NO: | | | |
| | 112) | | | |

In some embodiments, the expression of the nucleic acid molecule encoding a polypeptide having pyruvate kinase activity included in the expression cassettes of the invention in a plant, plant cell, or plant part confers increased content in one or more of protein, oil, or one or more amino acids in said plant, plant cell, or plant part relative to a corresponding wild-type plant, plant cell, or plant part. In other embodiments, the expression of the nucleic acid molecule encoding a polypeptide having pyruvate kinase activity included in the expression cassettes of the invention in a plant, plant cell, or plant part confers increased content in protein and one or more amino acids in said plant, plant cell, or plant part relative to a corresponding wild-type plant, plant cell, or plant part. In further embodiments, the expression of the nucleic acid molecule encoding a polypeptide having pyruvate kinase activity included in the expression cassettes of the invention in a plant, plant cell, or plant part confers increased content in protein, oil, and one or more amino acids in said plant, plant cell, or plant part relative to a corresponding wild-type plant, plant cell, or plant part.

### 2. Recombinant Constructs and Vectors

The aforementioned expression cassettes are preferably comprised in a recombinant construct and/or a vector, preferably a plant transformation vector. Numerous vectors for recombinant DNA manipulation or plant transformation are known to the person skilled in the pertinent art. The selection of vector will depend upon the host cell employed. Similarly, the selection of plant transformation vector will depend upon the preferred transformation technique and the target species for transformation.

2.1 Recombinant Constructs

Another aspect of the invention refers to a recombinant construct comprising at least one of the aforementioned expression cassettes. Preferably, the recombinant construct comprises at least one aforementioned expression cassette comprising other regulatory elements described herein for directing the expression of the nucleic acid molecule comprised in the aforementioned expression cassette in an appropriate host cell. More preferably, the recombinant construct comprises at least one aforementioned expression cassette with at least one terminator. Optionally, or in another embodiment, the recombinant construct may comprise at least one aforementioned expression cassette further comprising at least one expression enhancing sequence such as an intron sequence as exemplified herein, for example, in Section 1.2.

It is further within the scope of the invention that a recombinant construct may comprise more than one aforementioned expression cassette. It is also to be understood that each expression cassette to be included in the recombinant construct may further comprise at least one regulatory element of the same or different type as described herein.

2.2 Vectors

Another aspect of the invention refers to a vector comprising the aforementioned expression cassette or a recombinant construct derived therefrom. The term “vector,” preferably, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination. The vector encompassing the expression cassettes or recombinant constructs of the invention, preferably, further comprises selectable markers as described below for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion.

Vectors can be introduced into prokaryotic or eukaryotic cells via conventional transformation or transfection techniques. The terms “transformation” and “transfection,” conjugation and transduction, as used in the present context, are intended to comprise a multiplicity of processes known in the art for introducing foreign nucleic acid (e.g., DNA) into a host cell, including, but not limited to, calcium phosphate, rubidium chloride or calcium chloride co-precipitation, DEAE-dextran-mediated transfection, lipofection, natural competence, carbon-based clusters, chemically mediated transfer, electroporation or particle bombardment (e.g., “gene-gun”). Suitable methods for the transformation or transfection of host cells, including plant cells, can be found in Sambrook et al. (Molecular Cloning: A Laboratory Manual, 2^{nd} ed., 1989, Cold Spring Harbor Laboratory, Cold Spring Harbor Laboratory Press, Cold Spring Harbor, N.Y.) and other laboratory manuals, such as Methods in Molecular Biology (Gartland and Davey eds., 1995, Vol. 44, Agrobacterium Protocols, Humana Press, Totowa, N.J.). Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host or host cells. Preferably, the vector referred to herein is suitable as a cloning vector, i.e. replicable in microbial systems. Such vectors ensure efficient cloning in bacteria and, preferably, yeasts or fungi and make possible the stable transformation of plants. Examples of suitable vectors include, but not limited to, various binary and co-integrated vector systems which are suitable for the T-DNA-mediated transformation as described herein. These vector systems, preferably, also comprise further cis-regulatory elements as described herein, such as selection markers or reporter genes.

2.3 Vector Elements

Recombinant constructs and the vectors derived therefrom may comprise further functional elements. The term “functional element” is to be understood in the broad sense and means all those elements which have an effect on the generation, multiplication or function of the recombinant constructs, vectors or transgenic organisms according to the invention. Examples of such function elements include, but not limited to, selection marker genes, reporter genes, origins of replication, elements necessary for Agrobacterium-mediated transformation, and multiple cloning sites (MCS).

Selection marker genes are useful to select and separate successfully transformed cells. Preferably, within the method of the invention one marker may be employed for selection in a prokaryotic host, while another marker may be employed for selection in a eukaryotic host, particularly the plant species host. The marker may confer resistance against a biocide, such as antibiotics, toxins, heavy metals, or the like, or may function by complementation, imparting prototrophy to an auxotrophic host. Preferred selection marker genes for plants may include, but not limited to, negative selection markers, positive selection markers, and counter selection markers.

Negative selection markers include markers which confer a resistance to a biocidal compound such as a metabolic inhibitor (e.g., 2-deoxyglucose-6-phosphate, WO 98/45456), antibiotics (e.g., kanamycin, G418, bleomycin or hygromycin) or herbicides (e.g., phosphinothricin or glyphosate). Especially preferred negative selection markers are those which confer resistance to herbicides. These markers can be used, beside their function as a selection marker, to confer a herbicide resistance trait to the resulting transgenic plant. Examples of negative selection markers include, but not limited to
Phosphinothricin acetyltransferases (PAT; also named Bialophos resistance; bar; de Block et al., EMBO J., 1987, 6: 2513-2518; EP 0333033; U.S. Pat. No. 4,975,374);
5-enolpyruvylshikimate-3-phosphate synthase (EPSPS; U.S. Pat. No. 5,633,435) or glyphosate oxidoreductase gene (U.S. Pat. No. 5,463,175) conferring resistance to Glyphosate (N-phosphonomethyl glycine) (Shah et al., Science, 1986, 233: 478);
Glyphosate degrading enzymes (Glyphosate oxidoreductase; gox);
Dalapon inactivating dehalogenases (deh);
Sulfonylurea- and imidazolinone-inactivating acetolactate synthases (for example mutated ALS variants with, for example, the S4 and/or Hra mutation);
Bromoxynil degrading nitrilases (bxn);
Kanamycin- or G418-resistance genes (NPTII or NPTI) coding for neomycin phosphotransferases (Fraley et al., Proc. Natl. Acad. Sci. USA, 1983, 80: 4803), which expresses an enzyme conferring resistance to the antibiotic kanamycin and the related antibiotics neomycin, paromomycin, gentamicin, and G418;
2-Deoxyglucose-6-phosphate phosphatase (DOGR1-Gene product; WO 98/45456; EP 0807836) conferring resistance against 2-desoxyglucose (Randez-Gil et al., Yeast, 1995, 11: 1233-1240);
Hygromycin phosphotransferase (HPT), which mediates resistance to hygromycin (Vanden Elzen et al., Plant Mol. Biol., 1985, 5: 299); and
Dihydrofolate reductase (Eichholtz et al., Somatic Cell and Molecular Genetics, 1987, 13: 67-76).

Additional negative selection marker genes of bacterial origin that confer resistance to antibiotics include the aadA gene, which confers resistance to the antibiotic spectinomycin, gentamycin acetyl transferase, streptomycin phosphotransferase (SPT), aminoglycoside-3-adenyl transferase and the bleomycin resistance determinant (Svab et al., Plant Mol. Biol., 1990, 14: 197; Jones et al., Mol. Gen. Genet., 1987, 210: 86; Hille et al., Plant Mol. Biol., 1986, 7: 171; Hayford et al., Plant Physiol., 1988, 86: 1216). Other negative selection markers include those confer resistance against the toxic effects imposed by D-amino acids like e.g., D-alanine and D-serine (WO 03/060133; Erikson et al., Nat Biotechnol., 2004, 22(4): 455-458), the daol gene encoding a D-amino acid oxidase (EC 1.4.3.3; GenBank Accession No. U60066) from Rhodotorula gracilis (Rhodosporidium toruloides), and the dsdA gene encoding a D-serine deaminase (EC 4.3.1.18; GenBank Accession No. J01603) from E. coli. Depending on the employed D-amino acid, the D-amino acid oxidase markers can be employed as dual function marker offering negative selection (e.g., when combined with for example D-alanine or D-serine) or counter selection (e.g., when combined with D-leucine or D-isoleucine).

Positive selection markers include markers which confer a growth advantage to a transformed plant in comparison with a non-transformed one. Genes like isopentenyltransferase from Agrobacterium tumefaciens (strain PO22; Genbank Accession No. AB025109) may, as a key enzyme of the cytokinin biosynthesis, facilitate regeneration of transformed plants (e.g., by selection on cytokinin-free medium). Corresponding selection methods are described in Ebinuma et al. (Proc. Natl. Acad. Sci. USA, 2000, 94: 2117-2121) and Ebinuma et al. (“Selection of marker-free transgenic plants using the oncogenes (ipt, rol A, B, C) of Agrobacterium as selectable markers,” 2000, in Molecular Biology of Woody Plants, Kluwer Academic Publishers). Additional positive selection markers, which confer a growth advantage to a transformed plant in comparison with a non-transformed one, are described in, for example, EP 0601092. Growth stimulation selection markers may include, but not limited to, β-glucuronidase (in combination with, for example, cytokinin glucuronide), mannose-6-phosphate isomerase (in combination with mannose), UDP-galactose-4-epimerase (in combination with, for example, galactose), wherein mannose-6-phosphate isomerase in combination with mannose is especially preferred.

Counter selection markers are especially suitable to select organisms with defined deleted sequences comprising said marker (Koprek et al., Plant J., 1999, 19(6): 719-726). Examples for counter selection marker include, but not limited to, thymidine kinases (TK), cytosine deaminases (Gleave et al., Plant Mol. Biol., 1999, 40(2): 223-35; Perera et al., Plant Mol. Biol., 1993, 23(4): 793-799; Stougaard, Plant J., 1993, 3: 755-761), cytochrom P450 proteins (Koprek et al., Plant J., 1999, 19(6): 719-726), haloalkan dehalogenases (Naested, Plant J., 1999, 18: 571-576), iaaH gene products (Sundaresan et al., Gene Develop., 1995, 9: 1797-1810), cytosine deaminase codA (Schlaman and Hooykaas, Plant J., 1997, 11: 1377-1385), and tms2 gene products (Fedoroff and Smith, Plant J., 1993, 3: 273-289).

Reporter genes encode readily quantifiable proteins and, via their color or enzyme activity, make possible an assessment of the transformation efficacy, the site of expression or the time of expression. Very especially preferred in this context are genes encoding reporter proteins (Schenborn and Groskreutz, Mol. Biotechnol., 1999, 13(1): 29-44) such as the green fluorescent protein (GFP) (Haseloff et al., Proc. Natl. Acad. Sci. USA, 1997, 94(6): 2122-2127; Sheen et al., Plant J., 1995, 8(5): 777-784; Reichel et al., Proc. Natl. Acad. Sci. USA, 1996, 93(12): 5888-5893; Chui et al., Curr. Biol., 1996, 6: 325-330; Leffel et al., Biotechniques, 1997, 23(5): 912-918; Tian et al., Plant Cell Rep., 1997, 16: 267-271; WO 97/41228), chloramphenicol transferase, a luciferase (Millar et al., Plant Mol. Biol. Rep., 1992, 10: 324-414; Ow et al., Science, 1986, 234: 856-859), the aequorin gene (Prasher et al., Biochem. Biophys. Res. Commun., 1985, 126(3): 1259-1268), β-galactosidase, R locus gene (encoding a protein which regulates the production of anthocyanin pigments (red coloring) in plant tissue and thus makes possible the direct analysis of the promoter activity without addition of further auxiliary substances or chromogenic substrates; see Dellaporta et al., 1988, In: Chromosome Structure and Function: Impact of New Concepts, 18th Stadler Genetics Symposium, 11: 263-282; Ludwig et al., Science, 1990, 247: 449), with β-glucuronidase (GUS) being very especially preferred (Jefferson, Plant Mol. Bio. Rep., 1987, 5: 387-405; Jefferson et al., EMBO J., 1987, 6: 3901-3907). β-glucuronidase (GUS) expression is detected by a blue color on incubation of the tissue with 5-bromo-4-chloro-3-indolyl-β-D-glucuronic acid, bacterial luciferase (LUX) expression is detected by light emission, firefly luciferase (LUC) expression is detected by light emission after incubation with luciferin, and galactosidase expression is detected by a bright blue color after the tissue was stained with 5-bromo-4-chloro-3-indolyl-β-D-galactopyranoside. Reporter genes may also be used as scorable markers as alternatives to antibiotic resistance markers. Such markers can be used to detect the presence or to measure the level of expression of the transferred gene. The use of scorable markers in plants to identify or tag genetically modified cells works well when efficiency of modification of the cell is high. Origins of replication which ensure amplification of the recombinant constructs or vectors according to the invention in, for example, E. coli. Examples of suitable origins of replication include, but not limited to, ORI (origin of DNA replication), the pBR322 ori or the P15A ori (Sambrook et al., Molecular Cloning: A Laboratory Manual, 2^{nd} ed., Cold Spring Harbor Laboratory, Cold Spring Harbor Laboratory Press, Cold Spring Harbor, N.Y., 1989). Additional examples for replication systems functional in E. coli, are ColE1, pSC101, pACYC184, or the like. In addition to or in place of the E. coli replication system, a broad host range replication system may be employed, such as the replication systems of the P-1 Incompatibility plasmids, e.g., pRK290. These plasmids are particularly effective with aimed and disarmed Ti-plasmids for transfer of T-DNA to the plant host.

Other functional elements may be included in the recombinant constructs and the vector derived therefrom of the invention include, but not limited to, other genetic control elements for excision of the inserted sequences from the genome, elements necessary for Agrobacterium-mediated transformation, and multiple cloning sites (MCS).

Other genetic control elements for excision permit removal of the inserted sequences from the genome. Methods based on the ere/lox (Dale and Ow, Proc. Natl. Acad. Sci. USA, 1991, 88: 10558-10562; Sauer, Methods, 1998, 14(4): 381-392; Odell et al., Mol. Gen. Genet., 1990, 223: 369-378), FLP/FRT (Lysnik et al., Nucleic Acid Research, 1993, 21: 969-975), or Ac/Ds system (Lawson et al., Mol. Gen. Genet., 1994, 245: 608-615; Wader et al., in Tomato Technology (Alan R. Liss, Inc.), 1987, pp. 189-198; U.S. Pat. No. 5,225,341; Baker et al., EMBO J., 1987, 6: 1547-1554) permit removal of a specific DNA sequence from the genome of the host organism, if appropriate, in a tissue-specific and/or inducible manner. In this context, the control sequences may mean the specific flanking sequences (e.g., lox sequences) which later allow removal (e.g., by means of cre recombinase) of a specific DNA sequence.

Elements necessary for Agrobacterium-mediated transformation may include, but not limited to, the right and/or, optionally, left border of the T-DNA or the vir region.

Multiple cloning sites (MCS) can be included in the recombinant construct or the vector of the invention to enable and facilitate the insertion of one or more nucleic acid sequences.

2.4 Vectors for Plant Transformation

If Agrobacteria are used for plant transformation, the recombinant construct is to be integrated into specific plasmid vectors, either into a shuttle or intermediate vector, or into a binary vector. If a Ti or Ri plasmid is to be used for the transformation, at least the right border, but in most cases the right and the left border, of the Ti or Ri plasmid T-DNA is flanking the region with the recombinant construct to be introduced into the plant genome. Preferably, binary vectors for the Agrobacterium transformation can be used. Binary vectors are capable of replicating both in E. coli and in Agrobacterium. They preferably comprise a selection marker gene and a linker or polylinker flanked by the right and, optionally, left T-DNA border sequence. They can be transformed directly into Agrobacterium (Holsters et al., Mol. Gen. Genet., 1978, 163: 181-187). A selection marker gene may be included in the vector which permits a selection of transformed Agrobacteria (e.g., the nptIII gene). The Agrobacterium, which acts as host organism in this case, may already comprise a disarmed (i.e. non-oncogenic) plasmid with the vir region for transferring the T-DNA to the plant cell. The use of T-DNA for the transformation of plant cells has been studied and described extensively (e.g., EP 0120516; Hoekema, In: The Binary Plant Vector System, Offsetdrukkerij Kanters B. V., Alblasserdam, Chapter V; An et al., EMBO J., 1985, 4: 277-287). A variety of binary vectors are known and available for transformation using Agrobacterium, such as, for example, pBI101.2 or pBIN19 (Clontech Laboratories, Inc. USA; Bevan et al., Nucl. Acids Res., 1984, 12: 8711), pBinAR, pPZP200 or pPTV.

Transformation can also be realized without the use of Agrobacterium. Non-Agrobacterium transformation circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include, but not limited to, transformation via particle bombardment, protoplast uptake (e.g., PEG and electroporation) and microinjection, all are well known in the art. The choice of vector depends largely on the preferred selection for the species being transformed. Typical vectors suitable for non-Agrobacterium transformation include pCIB3064, pSOG19, and pSOG35 (see e.g., U.S. Pat. No. 5,639,949).

3. Introduction of Expression Cassette into Cells and Organisms

The aforementioned expression cassettes, or the recombinant constructs or vectors derived therefrom, can be introduced into a cell or an organism in various ways known to the skilled worker. “To introduce” is to be understood in the broad sense and comprises, for example, all those methods suitable for directly or indirectly introducing a DNA or RNA molecule into an organism or a cell, compartment, tissue, organ or seed of same, or generating it therein. The introduction can bring about either a transient presence or a stable presence of such a DNA or RNA molecule in the cell or organism.

Thus, a further aspect of the invention relates to cells and organisms (e.g., plants, plant cells, microorganisms, bacteria, etc.), which comprise at least one expression cassette of the invention, or a recombinant construct or a vector derived therefrom. In certain embodiments, the cell is suspended in culture, while in other embodiments the cell is in, or in part of, a whole organism, such as a microorganism or a plant. The cell can be prokaryotic or of eukaryotic nature. For plants and plant cells, preferably, the expression cassette or recombinant construct is integrated into the genomic DNA, more preferably within the chromosomal or plastidic DNA, most preferably in the chromosomal DNA of the cell. For microorganisms, the expression cassette or recombinant construct is preferably incorporated into a plasmid, which is then introduced into the microorganism. Accordingly, in one embodiment, the present invention relates to a transformed plant cell, plant or part thereof, comprising in its genome at least one stably incorporated expression cassette of the present invention, or a recombinant construct or a vector derived therefrom. In another embodiment, the present invention relates to a transformed microorganism comprising a plasmid containing the expression cassette or recombinant construct of the present invention.

Preferred prokaryotic cells include mainly bacteria such as bacteria of the genus Escherichia, Corynebacterium, Bacillus, Clostridium, Proionibacterium, Butyrivibrio, Eubacterium, Lactobacillus, Erwinia, Agrobacterium, Flavobacterium, Alcaligenes, Phaeodactylum, Colpidium, Mortierella, Entomophthora, Mucor, Crypthecodinium or Cyanobacteria, for example of the genus Synechocystis. Microorganisms which are preferred are mainly those which are capable of infecting plants and thus of transferring the expression cassette or construct of the invention. Preferred microorganisms are those of the genus Agrobacterium and in particular the species Agrobacterium tumefaciens and Agrobacterium rhizogenes.

Eukaryotic cells and organisms comprise plant and animal (preferably non-human) organisms and/or cells and eukaryotic microorganisms such as, for example, yeasts, algae or fungi. Preferred fungi include Aspergillus, Trichoderma, Ashbya, Neurospora, Fusarium, Beauveria or those described in Indian Chem Engr., Section B., 1995, 37(1, 2): 15, Table 6. Especially preferred is the filamentous Hemiascomycete Ashbya gossypii. Preferred yeasts include Candida, Saccharomyces, Hansenula or Pichia, especially preferred are Saccharomyces cerevisiae or Pichia pastoris (ATCC Accession No. 201178). Preferred eukaryotic cells or organisms comprise plant cells and/or organisms, or eukaryotic microorganisms. A corresponding transgenic organism can be generated for example by introducing a desired expression system into a cell derived from such an organism by ways and methods known in the art.

The “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” may also include parts of plants, such as pollen, flowers, kernels, ears, cobs, leaves, husks, stalks, and the like. The term “plant” also encompasses plant cells, plant protoplasts, plant cell tissue cultures, callus tissue, embryos, meristematic regions, gametophytes, sporophytes, pollen and microspores, gamete producing cells, and a cell that regenerates into a whole plant, again wherein each of the aforementioned comprises the gene/nucleic acid of interest.

Plants that are particularly useful in the present invention include all plants which belong to the superfamily Viridiplantae, in particular monocotyledonous and dicotyledonous plants including fodder or forage legumes, ornamental plants, food crops, trees, shrubs, or algae selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Jatropha curcas, Juglans spp., Lactuca sativa, Lathyrus spp., Lens culinaris, Lesquerella fendleri (Gray) Wats, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Sorghum halepense, Spinacia spp., Syzygium spp., Tagetes spp., Tamarindus indica, Theobroma cacao, Trifolium spp., Triticosecale rimpaui, Triticum spp. (e.g. Triticum aestivum, Triticum durum, Triticum turgidum, Triticum hybernum, Triticum macha, Triticum sativum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., Cyclotella cryptica, Navicula saprophila, Synechococcus 7002 and Anabaena 7120, Chlorella protothecoides, Dunaliella salina, Chlorella spp, Dunaliella tertiolecta, Gracilaria, Sargassum, Pleurochrisis carterae, Laminaria 3840 hyperbore, Laminaria saccharina, Gracialliaria, Sargassum, Botryccoccus braunii, Arthospira platensis, amongst others. Especially preferred are rice, oilseed rape, canola, soybean, corn (maize), cotton, sugarcane, micro algae, alfalfa, sorghum, and wheat.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

Preferably, the organisms are plant organisms. Preferred plants are selected in particular from among crop plants. More preferred plants include, but not limited to, maize, soybean, barley, alfalfa, sunflower, flax, linseed, oilseed rape, canola, sesame, safflower (Carthamus tinctorius), olive tree, peanut, castor-oil plant, oil palm, cacao shrub, or various nut species such as, for example, walnut, coconut or almond, soybean, cotton, peanut, sorghum, tobacco, sugarbeet, sugarcane, rice, wheat, rye, turfgrass, millet, sugarcane, tomato, or potato.

It is noted that a plant need not be considered a “plant variety” simply because it contains stably within its genome a transgene, introduced into a cell of the plant or an ancestor thereof. In addition to a plant, the present invention provides any clone of such a plant, seed, selfed or hybrid progeny and descendants, and any part or propagule of any of these, such as cuttings and seed, which may be used in reproduction or propagation, sexual or asexual. Also encompassed by the invention is a plant which is a sexually or asexually propagated offspring, progeny, clone or descendant of such a plant, or any part or propagule of said plant, offspring, clone or descendant. Genetically modified plants according to the invention, which can be consumed by humans or animals, can also be used as food or feedstuffs, for example directly or following processing known in the art, or be used in biofuel production. The present invention also provides for parts of the organism especially plants, particularly reproductive or storage parts. Plant parts, without limitation, include seed, endosperm, ovule, pollen, roots, tubers, stems, leaves, stalks, fruit, berries, nuts, bark, pods, seeds and flowers.

The expression cassette of the invention, or a recombinant construct or vector derived therefrom, is typically introduced or administered in an amount that allows delivery of at least one copy per cell. Higher amounts (for example at least 5, 10, 100, 500 or 1000 copies per cell) can, if appropriate, result in a more efficient phenotype (e.g., higher expression or higher suppression of the target gene). The amount of the expression cassette, recombinant construct, or vector administered to a cell, tissue, or organism depends on the nature of the cell, tissue, or organism, the nature of the target gene, and the nature of the expression cassette, recombinant construct, or vector, and can readily be optimized to obtain the desired level of expression or inhibition.

Preferably at least about 100 molecules, preferably at least about 1000, more preferably at least about 10,000 of the expression cassette, recombinant construct, or vector, most preferably at least about 100,000 of the expression cassette, recombinant construct, or vector are introduced. In the case of administration of the expression cassette, recombinant construct, or vector to a cell culture or to cells in tissue, by methods other than injection, for example by soaking, electroporation, or lipid-mediated transfection, the cells are preferably exposed to similar levels of the expression cassette, recombinant construct, or vector in the medium.

For example, the expression cassette, recombinant construct, or vector of the invention may be introduced into cells via transformation, transfection, injection, projection, conjugation, endocytosis, and phagocytosis, all are well known in the art. Preferred methods for introduction include, but not limited to:

(a) methods of direct or physical introduction of the expression cassette, recombinant construct, or vector of the invention into the target cell or organism, and

(b) methods of indirect introduction of the expression cassette, recombinant construct, or vector of the invention into the target cell or organism by, for example, a first introduction of a recombinant construct and a subsequent intracellular expression.

### 4. Plant Transformation Techniques

In a further embodiment, the invention provides a method of producing a transgenic plant, plant cell, or plant part comprising:

(a) transforming a plant, plant cell, or plant part with at least one aforementioned expression cassettes, or a recombinant construct or vector derived therefrom, and

(b) optionally regenerating from the plant cell or plant part a transgenic plant.

A variety of methods for introducing nucleic acid sequences (e.g., vectors) into the genome of plants and for the regeneration of plants from plant tissues or plant cells are known in the art (Plant Molecular Biology and Biotechnology, Chapter 6-7, pp. 71-119, CRC Press, Boca Raton, Fla., 1993; White F. F., “Vectors for Gene Transfer in Higher Plants,” in Transgenic Plants, Vol. 1, Engineering and Utilization, Kung and Wu, eds., Academic Press, pp. 15-38, 1993; Jenes et al., “Techniques for Gene Transfer,” in Transgenic Plants, Vol. 1, Engineering and Utilization, Kung and Wu, eds., Academic Press, pp. 128-143, 1993; Potrykus, Anna. Rev. Plant Physiol. Plant Mol. Biol., 1991, 42:205-225; Halford et al., Br. Med. Bull., 2000, 56(1): 62-73).

4.1 Non-Agrobacterium Transformation

Transformation methods may include direct and indirect methods of transformation. Suitable direct methods include, but not limited to, polyethylene glycol induced DNA uptake, liposome-mediated transformation (U.S. Pat. No. 4,536,475), biolistic methods using the gene gun (Fromm et al., Bio/Technology, 1990, 8(9): 833-839; Gordon-Kamm et al., Plant Cell, 1990, 2: 603), electroporation, incubation of dry embryos in DNA-comprising solution, and microinjection. In the case of these direct transformation methods, the plasmid used need not meet any particular requirements. Simple plasmids, such as those of the pUC series, pBR322, M13mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

4.2 Agrobacterium Transformation

Transformation can also be carried out by bacterial infection by means of Agrobacterium (for example EP 0116718), viral infection by means of viral vectors (EP 0067553; U.S. Pat. No. 4,407,956; WO 95/34668; WO 93/03161) or by means of pollen (EP 0270356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch et al., Science, 1985, 227: 1229-1231. The Agrobacterium-mediated transformation is best suited to dicotyledonous plants but has also been adopted to monocotyledonous plants. The transformation of plants by Agrobacteria is described in, for example, White F. F., “Vectors for Gene Transfer in Higher Plants,” in Transgenic Plants, Vol. 1, Engineering and Utilization, Kung and Wu, eds., Academic Press, pp. 15-38, 1993; Jenes et al., “Techniques for Gene Transfer,” in Transgenic Plants, Vol. 1, Engineering and Utilization, Kung and Wu, eds., Academic Press, pp. 128-143, 1993; Potrykus, Annu. Rev. Plant Physiol. Plant Mol. Biol., 1991, 42: 205-225.

Transformation may result in transient or stable transformation and expression. Although an expression cassette of the present invention can be inserted into any plant and plant cell falling within these broad classes, it is particularly useful in crop plant cells.

Various tissues are suitable as starting material (explant) for the Agrobacterium-mediated transformation process including, but not limited to, callus (U.S. Pat. No. 5,591,616; EP 604662), immature embryos (EP 672752), pollen (U.S. Pat. No. 5,929,300), shoot apex (U.S. Pat. No. 5,164,310), or in planta transformation (U.S. Pat. No. 5,994,624). The method and material described herein can be combined with Agrobacterium mediated transformation methods known in the art.

4.3 Plastid Transformation

In another embodiment, the expression cassette or recombinant construct is directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit high expression levels. In one embodiment, the nucleotide sequence is inserted into a plastid targeting vector and transformed into the plastid genome of a desired plant host. Plants homoplasmic for plastid genomes containing the nucleotide sequence are obtained, and are preferentially capable of high expression of the nucleotide sequence.

Plastid transformation technology is extensively described in, for example, U.S. Pat. No. 5,451,513, U.S. Pat. No. 5,545,817, U.S. Pat. No. 5,545,818, U.S. Pat. No. 5,877,462, WO 95/16783, WO 97/32977, and in McBride et al., Proc. Natl. Acad. Sci. USA, 1994, 91: 7301-7305. The basic technique for plastid transformation involves introducing regions of cloned plastid DNA flanking a selectable marker together with the nucleotide sequence into a suitable target tissue, e.g., using biolistic or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al., Proc. Natl. Acad. Sci. USA, 1990, 87: 8526-8530; Staub et al., Plant Cell, 1992, 4: 39-45). The presence of cloning sites between these markers allowed creation of a plastid targeting vector for introduction of foreign genes (Staub et al., EMBO J., 1993, 12: 601-606). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et al., Proc. Natl. Acad. Sci. USA, 1993, 90: 913-917). Other selectable markers useful for plastid transformation are known in the art and encompassed within the scope of the invention.

### 5. Selection and Regeneration Techniques

To select cells which have successfully undergone transformation, it is preferred to introduce a selectable marker which confers, to the cells which have successfully undergone transformation, a resistance to a biocide (for example a herbicide), a metabolism inhibitor such as 2-deoxyglucose-6-phosphate (WO 98/45456) or an antibiotic. The selection marker permits the transformed cells to be selected from untransformed cells (McCormick et al., Plant Cell Reports, 1986, 5: 81-84). Suitable selection markers are described above.

Transgenic plants can be regenerated in the known manner from the transformed cells. The resulting plantlets can be planted and grown in the customary manner. Preferably, two or more generations should be cultured to ensure that the genomic integration is stable and hereditary. Suitable methods are described in, for example, Fennell et al., Plant Cell Rep., 1992, 11:567-570; Stoeger et al., Plant Cell Rep., 1995, 14: 273-278; and Jahne et al., Theor. Appl. Genet., 1994, 89: 525-533.

### 6. Biotechnological Applications

The expression cassettes, and recombinant constructs and vectors derived therefrom, can be used to manipulate the production of protein, oils, and/or amino acids and the like in a plant, plant cell, or plant part. The invention, in one embodiment, provides a method for increasing the content of one or more of protein, oil or one or more amino acids in a plant, plant cell, or plant part relative to a corresponding wild-type plant, plant cell, or plant part, comprising:

(a) obtaining a plant, plant cell, or plant part comprising at least one aforementioned expression cassette, or at least one recombinant construct or vector derived therefrom, and

(b) selecting a plant, plant cell, or plant part with increased content in one or more of protein, oil, or one or more amino acids.

Preferably, expression of the nucleic acid molecule comprised in the aforementioned expression cassettes in the transformed and/or regenerated transgenic plant, plant cell, or plant part increases the protein, oil, and/or amino acid content of the transgenic plant, plant cell, or plant part, as compared to a corresponding wild-type plant, plant cell, or plant part. Methods of transforming a plant, plant cell, or plant part, selecting a transformed plant, plant cell, or plant part, and regenerating a plant from a plant cell or plant part are well known to one skilled in the art in view of the disclosure herein above.

Increases in protein, oil and/or amino acid content can be assessed by various methods known to one skilled in the art.

Plants suitable for the use in the methods of the invention can be monocotyledonous or dicotyledonous plants. In a preferred embodiment, the plant is a monocotyledonous plant, and more preferably, a maize plant, or the plant cell or plant part is from a monocotyledonous plant, preferably a maize plant.

The plant, plant cell, or plant part that is obtained from the aforementioned methods can be used for production of food, feed, a food supplement, or a feed supplement. Accordingly, in a further embodiment, the present invention relates to the use of the plant, plant cell, or plant part obtained according to the aforementioned methods for the preparation of a food or feed composition or a composition intended for use as a food or feed supplement. The invention further relates to a method of producing a food or feed composition intended for animal or livestock feed comprising the plant, plant cell, or plant part obtained according to the aforementioned methods, and to the composition intended for animal or livestock feed thus obtained. In a preferred embodiment, said plant is a monocotyledonous plant, and more preferably, a maize plant, or the plant cell or plant part is from a monocotyledonous plant, preferably a maize plant.

In one embodiment, the plants, seed, or grain of the invention are used for production of human food, animal or livestock feed, as raw material in industry, pet foods, and food products. Such products can provide increased nutrition because of the increased nutrient value. In a further embodiment, the present invention also relates to animal feed which is formulated for a specific animal type, for example, as in U.S. Pat. No. 6,774,288, which is hereby incorporated by reference in its entirety. The seed or grain with increased content in one or more of protein, oil, or one or more amino acids may be seed or grain from any crop species including a high protein maize, for example, as in U.S. Pat. No. 6,774,288, which is hereby incorporated by reference in its entirety. The animal feed may be used for feeding non ruminant animals, such as swine, poultry, horses, or sheep, small companion animals such as cats or dogs, and fish such as tilapia or salmon. For example, maize is used extensively as livestock feed, primarily for beef cattle, dairy cattle, hogs, and poultry. See, for example, U.S. Pat. No. 7,087,261, U.S. Pat. No. 6,774,288, and US 2005/0246791.

### 7. Plant Breeding

7.1 Traditional Breeding Methods

The plants and plant parts obtained from the aforementioned methods can also be used in a plant breeding program. In one embodiment, the invention relates to methods for producing a maize plant by crossing a first parent maize plant with a second parent maize plant wherein either the first or second parent maize plant comprises an expression cassette or recombinant construct described herein. The other parent may be any other maize plant, such as another inbred line or a plant that is part of a cultivated or natural population. Any plant breeding method may be used, including but not limited to selfing, sibbing, backcrossing, recurrent selection, mass selection, pedigree breeding, double haploids, bulk selection, hybrid production, crosses to populations, and the like. These methods are well known in the art.

For example, pedigree breeding is used commonly for the improvement of self-pollinating crops or inbred lines of cross-pollinating crops. Pedigree breeding starts with the crossing of two genotypes, such as a first inbred line comprising an expression cassette or recombinant construct described herein and a second elite inbred line having one or more desirable characteristics that is lacking or which complements the first inbred line. If the two original parents do not provide all the desired characteristics, other sources can be included in the breeding population. In the pedigree method, superior plants are selfed and selected in successive filial generations. In the succeeding filial generations the heterozygous condition gives way to homogeneous lines as a result of self-pollination and selection.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or inbred line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

Several different physiological and morphological characteristics can be selected for as attributes of the recurrent parent in a backcross breeding program, including days to maturity (e.g. days from emergence to 50% of plants in silk or 50% of plants in pollen), plant height, ear height, average length of top ear internode, average number of tillers, average number of ears per stalk, anthocyanin content of brace roots, width of ear node leaf, length of ear node leaf, number of leaves above top ear, leaf angle from second leaf above ear at anthesis to stalk above leaf, leaf color, leaf sheath pubescence, leaf marginal waves, leaf longitudinal creases, number of lateral branches on tassel, branch angle from central spike of tassel, tassel length, pollen shed, anther color, glume color, bar glumes, ear silk color, fresh husk color, dry husk color, position of ear, husk tightness, husk extension, ear length, ear diameter at mid-point, ear weight, number of kernel rows, kernel rows, row alignment, shank length, ear taper, kernel length, kernel width, kernel thickness, kernel shape, aleurone color pattern, aleurone color, hard endosperm color, endosperm type, weight per 100 kernels, cob diameter at mid-point, cob color, and agronomic traits such as stay green (late season plant health), dropped ears (percentage of plants that dropped an ear prior to harvest), pre-anthesis brittle snapping (stalk breaking near the time of pollination), pre-anthesis root lodging (lean from the vertical axis at an approximate 30° angle or greater near the time of pollination), and post-anthesis root lodging.

7.2 Breeding with Molecular Markers

Molecular markers, which includes markers identified through the use of techniques such as Isozyme Electrophoresis, Restriction Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs), and Single Nucleotide Polymorphisms (SNPs), may be used in plant breeding methods utilizing the inbred of the present invention. Molecular markers can be used to identify the unique genetic composition of the invention and progeny lines retaining that unique genetic composition. Various molecular marker techniques may be used in combination to enhance overall resolution.

One use of molecular markers is Quantitative Trait Loci (QTL) mapping. QTL mapping is the use of markers, which are known to be closely linked to alleles that have measurable effects on a quantitative trait. Selection in the breeding process is based upon the accumulation of markers linked to the positive effecting alleles and/or elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select for the genome of the recurrent parent and can minimize the amount of genome from the donor parent that remains in the selected plants. It can also be used to reduce the number of crosses back to the recurrent parent needed in a backcrossing program. The use of molecular markers in the selection process is often called genetic marker enhanced selection.

Descriptions of breeding methods can also be found in one of several reference books (e.g., Allard, Principles of Plant Breeding, 1960; Simmonds, Principles of Crop Improvement, 1979; Fehr, “Breeding Methods for Cultivar Development”, Production and Uses, 2nd ed., Wilcox editor, 1987). See also U.S. Pat. No. 7,183,470 and U.S. Pat. No. 7,339,097, the disclosures of which are expressly incorporated herein by reference.

7.3 Maize Hybrids

A single cross maize hybrid results from the cross of two inbred lines, each of which has a genotype that complements the genotype of the other. The hybrid progeny of the first generation is designated F1. In the development of commercial hybrids in a maize plant breeding program, only the F1 hybrid plants are sought. F1 hybrids are more vigorous than their inbred parents. This hybrid vigor, or heterosis, can be manifested in many polygenic traits, including increased vegetative growth and increased yield.

An inbred maize line comprising an expression cassette or recombinant construct described herein may be used to produce hybrid maize. One such embodiment is the method of crossing the inbred maize line comprising an expression cassette or recombinant construct of the invention with another maize plant, such as a different maize inbred line, to form a first generation F1 hybrid seed. The first generation F1 hybrid seed, plant and plant part produced by this method is an embodiment of the invention. The first generation F1 seed, plant and plant part will comprise an essentially complete set of the alleles of the inbred line comprising an expression cassette or recombinant construct described herein. One of ordinary skill in the art can utilize either breeder books or molecular methods to identify a particular F1 hybrid plant produced using the inbred line comprising an expression cassette or recombinant construct described herein. Further, one of ordinary skill in the art may also produce F1 hybrids with transgenic, male sterile and/or backcross conversions of the inbred line comprising an expression cassette or recombinant construct described herein.

The development of a maize hybrid in a maize plant breeding program involves three steps: (1) the selection of plants from various germplasm pools for initial breeding crosses; (2) the selling of the selected plants from the breeding crosses for several generations to produce a series of inbred lines, such as an inbred line comprising an expression cassette or recombinant construct described herein, which, although different from each other, breed true and are highly uniform; and (3) crossing the selected inbred lines with different inbred lines to produce the hybrids. During the inbreeding process in maize, the vigor of the lines decreases, and so one would not be likely to use an inbred line comprising an expression cassette or recombinant construct described herein directly to produce grain. However, vigor can be restored by crossing the inbred line comprising an expression cassette or recombinant construct described herein with a different inbred line to produce a commercial F1 hybrid. An important consequence of the homozygosity and homogeneity of the inbred line is that the hybrid between a defined pair of inbreds may be reproduced indefinitely as long as the homogeneity of the inbred parents is maintained.

The inbred line comprising an expression cassette or recombinant construct described herein may be used to produce a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B)×(C×D). A three-way cross hybrid is produced from three inbred lines where two of the inbred lines are crossed (A×B) and then the resulting F1 hybrid is crossed with the third inbred (A×B)×C.

One or more genetic traits which have been engineered into the genome of a particular maize plant or plants using transformation techniques could be moved into the genome of another line using traditional breeding techniques that are well known in the plant breeding arts. For example, a backcrossing approach is commonly used to move a transgene from a transformed maize plant to an elite inbred line, and the resulting progeny would then comprise the transgene(s). In a single gene converted plant, the plant would have essentially all the desired morphological and physiological characteristics of the inbred in addition to the single gene transferred via backcrossing or via genetic engineering. Also, if an inbred line was used for the transformation then the transgenic plants could be crossed to a different inbred in order to produce a transgenic hybrid maize plant. In the same manner, more than one transgene can be transferred into the inbred.

Hybrid plants produced by the plant breeding methods described above may be used for producing grain with increased content in one or more of protein, oil, or one or more amino acids by interplanting at least two hybrid plant populations. For example, hybrid seed comprising an expression cassette or recombinant construct described herein may be interplanted with another hybrid seed with high yield to obtain grain with increased content in one or more of protein, oil, or one or more amino acids at competitive yields. The invention includes methods for producing grain by planting a first hybrid seed comprising an expression cassette or recombinant construct described herein, and at least a second hybrid seed; growing the seeds under conditions that allow for cross pollination between the plant produced from, the seed of the first hybrid and the plant produced by the seed of the second hybrid; and harvesting the grain. Conditions that allow for cross pollination between the hybrid plants include interplanting the hybrid populations in close enough proximity to allow for pollen transfer between the hybrid populations, and timing the planting of the hybrids such that pollen is released from one of the hybrids when the other hybrid is receptive to pollination. Methods of producing grain with increased value through interplanting of two or more hybrids are described, for example, in WO 2010/025213.

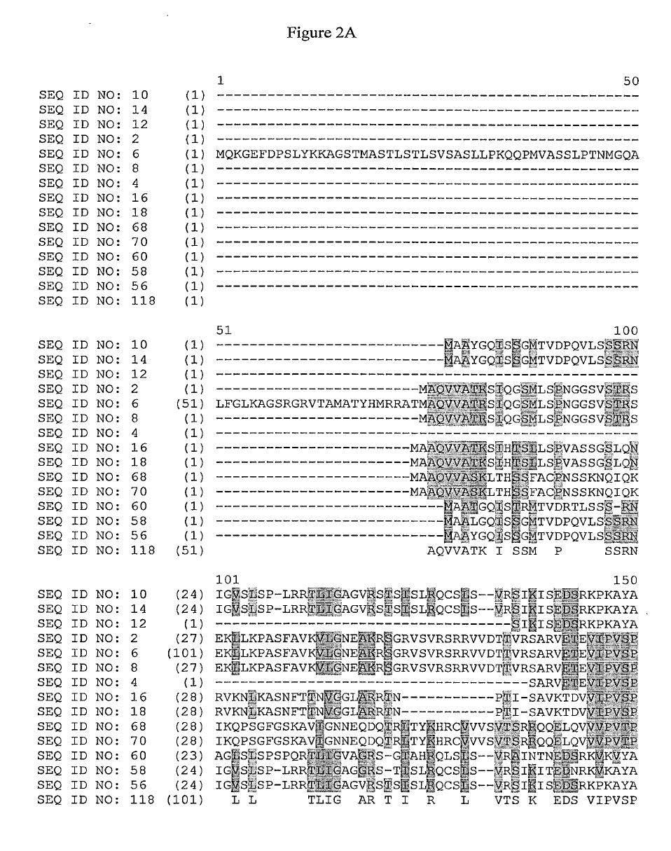

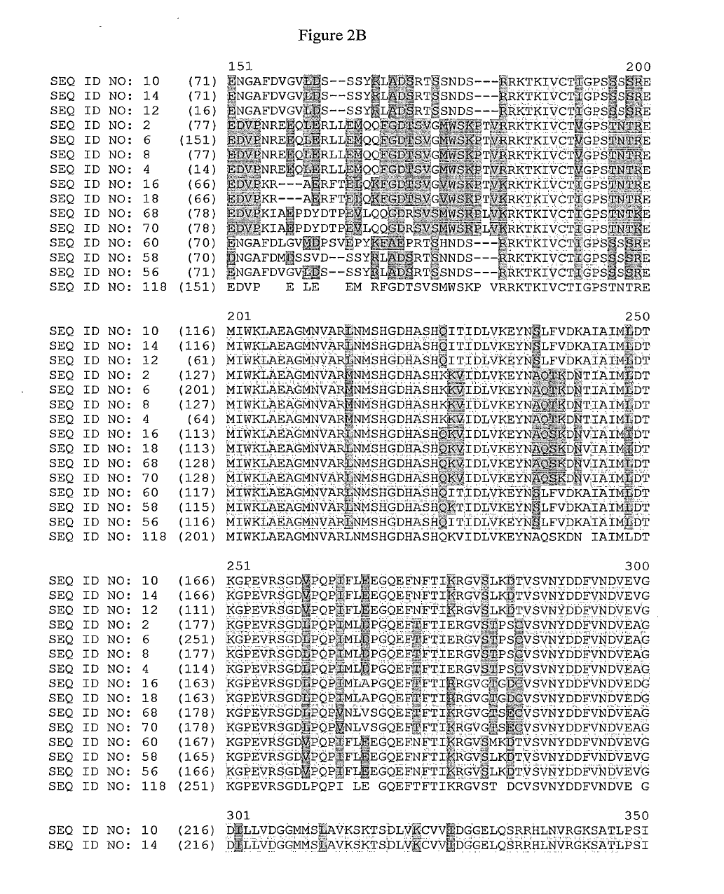

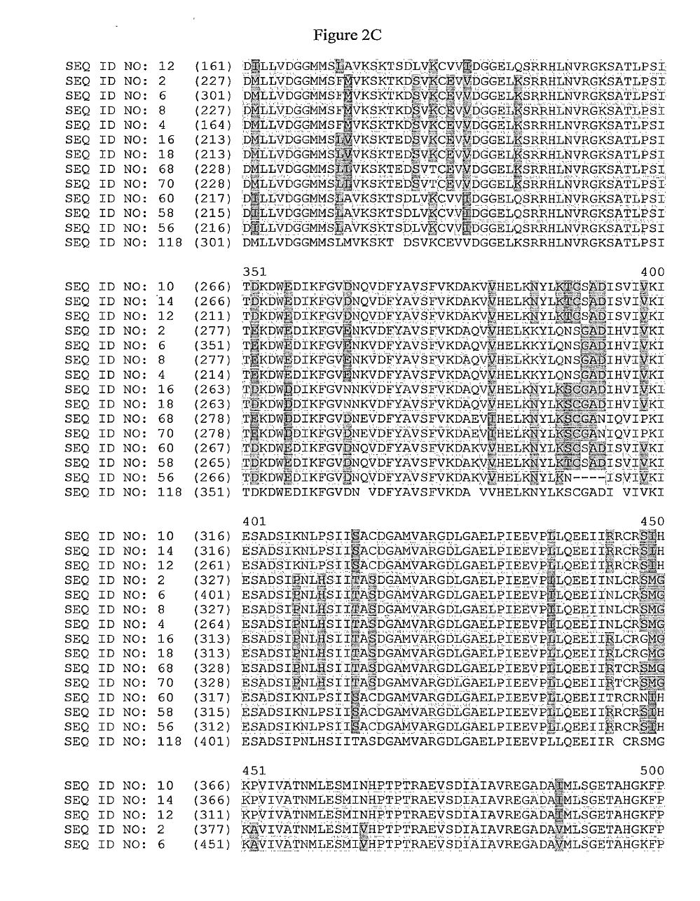

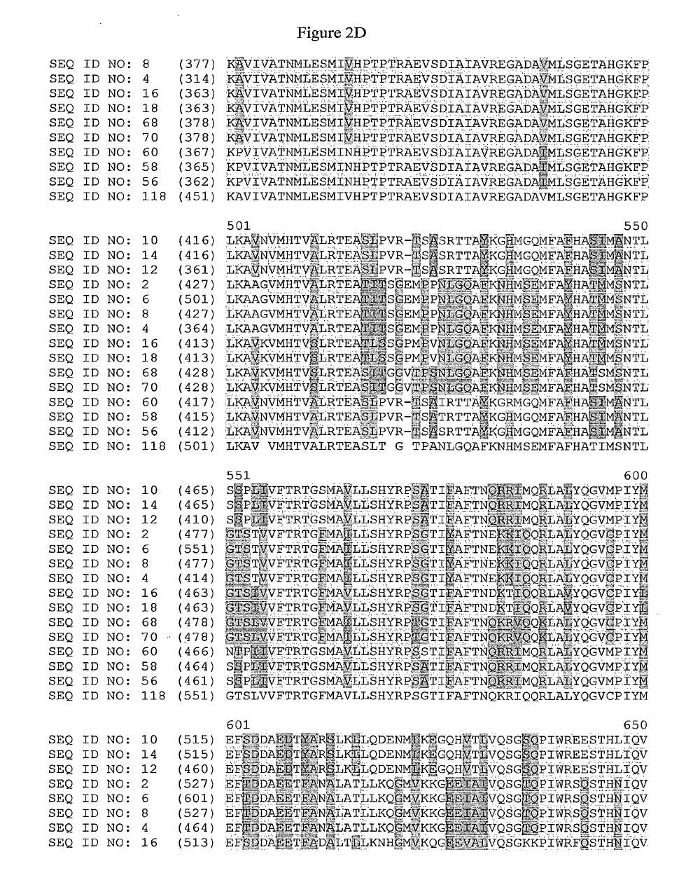

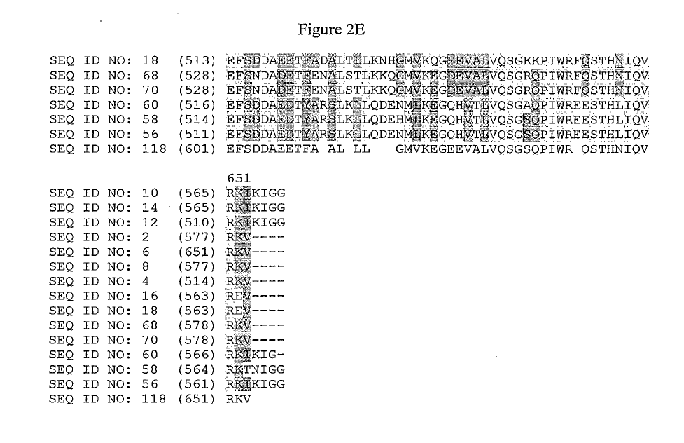

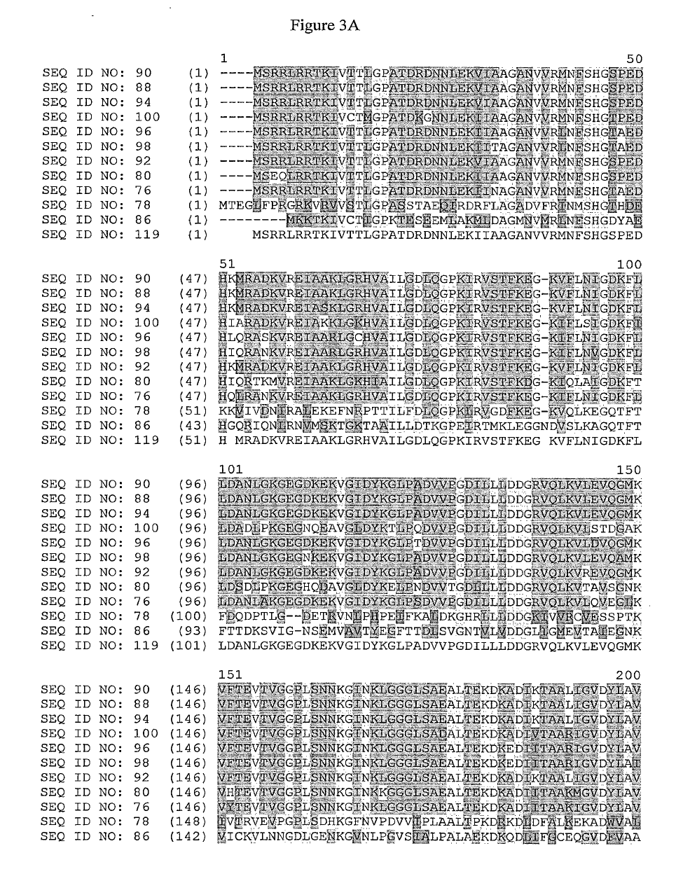

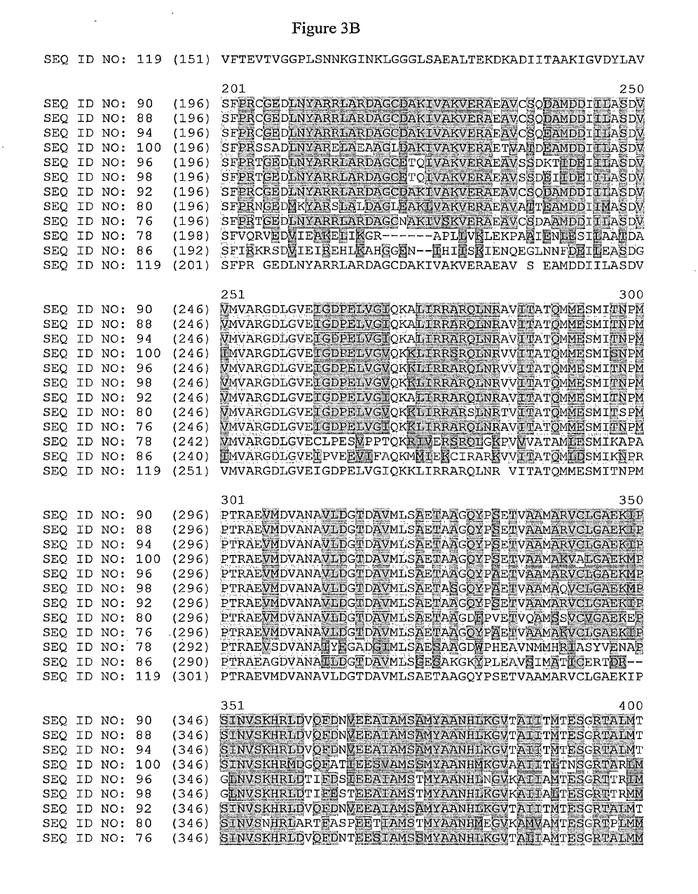

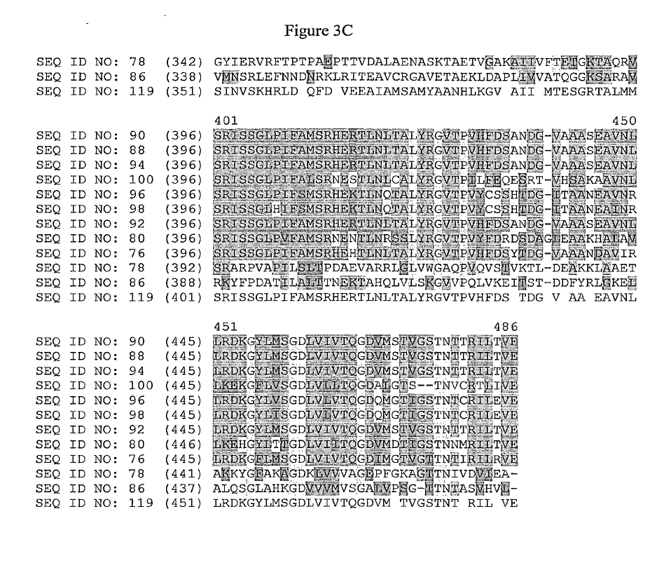

### Description of Sequences.

| | Nucleotide | Amino Acid |
|---|---|---|
| Sequence Description | SEQ ID NO | SEQ ID NO |
| PKpAt920 (with native peptide) | 1 | 2 |
| PKpAt920 (w/o native peptide) | 3 | 4 |
| PKpAt920 (synthetic) | 5 | 6 |
| PKpAt920.Zm, codon optimized for Z. mays | 7 | 8 |
| PKpAt440 (with native peptide) | 9 | 10 |
| PKpAt440 (w/o native peptide) | 11 | 12 |
| PKpAt440.Zm (codon optimized for Z. mays) | 13 | 14 |
| PK homolog from L. usitatissimum | 15 | 16 |
| PK homolog from L. usitatissimum codon optimized for Z. mays | 17 | 18 |
| PK homolog from A. thaliana | 19 | 20 |
| PK homolog from A. thaliana | 21 | 22 |
| PK homolog from A. thaliana | 23 | 24 |
| PK homolog, Synthetic | 25 | 26 |
| PK homolog from B. napus | 27 | 28 |
| PK homolog from B. napus | 29 | 30 |
| PK homolog from R. communis | 31 | 32 |
| PK homolog from V. vinifera | 33 | 34 |
| PK homolog from V. vinifera | 35 | 36 |
| PK homolog from P. trichocarpa | 37 | 38 |
| PK homolog from V. vinifera | 39 | 40 |
| PK homolog from G. max | 41 | 42 |
| PK homolog from G. max | 43 | 44 |
| PK homolog from G. max | 45 | 46 |
| PK homolog from G. max | 47 | 48 |
| PK homolog from G. max | 49 | 50 |
| PK homolog from G. max | 51 | 52 |
| PK homolog from G. max | 53 | 54 |
| PK homolog from A. thaliana | 55 | 56 |
| PK homolog from A. lyrata subsp. lyrata | 57 | 58 |
| PK homolog from B. napus | 59 | 60 |
| PK homolog from Z. mays | 61 | 62 |
| PK homolog from H. annuus | 63 | 64 |
| PK homolog from H. annuus codon optimized for Z. mays | 65 | 66 |
| PK homolog from H. annuus | 67 | 68 |
| PK homolog from H. annuus codon optimized for Z. mays | 69 | 70 |
| PK homolog from H. annuus | 71 | 72 |
| PK homolog from H. annuus codon optimized for Z. mays | 73 | 74 |
| PK homolog from P. wasabiae | 75 | 76 |
| PK homolog from Z. mobilis | 77 | 78 |
| PK homolog from P. profundum | 79 | 80 |
| PK homolog from A. thaliana | 81 | 82 |
| PKpAt960 | 83 | 84 |
| B1676, PK homolog from E. coli | 85 | 86 |
| B1854, PK homolog from E. coli | 87 | 88 |
| B1854.Zm, codon optimized for Z. mays | 89 | 90 |
| B1854, PK homolog from E. coli | 91 | 92 |
| PK homolog from E. coli | 93 | 94 |
| PK homolog from P. luminescens subsp. laumondii TTO1 | 95 | 96 |
| PK homolog from P. asymbiotica subsp. asymbiotica | 97 | 98 |
| ATCC 43949 | | |
| PK homolog from A. succinogenes 130Z | 99 | 100 |
| PK active site | — | 101 |
| Pfam:PF00224 consensus sequence | — | 102 |
| Pfam:PF02887 consensus sequence | — | 103 |
| KG86_12a promoter (whole-seed specific) | 104 | — |
| Sh2 promoter from Z. mays (endosperm specific) | 105 | — |
| 10 kDaZein promoter (endosperm specific) | 106 | — |
| 27 kDaZein promoter (endosperm specific) | 107 | — |
| ZmGlb1 promoter (embryo specific) | 108 | — |
| ScBV promoter (constitutive, longer version) | 109 | — |
| ScBV254 promoter (constitutive, shorter version) | 110 | — |
| Met1-1 intron (O. sativa) | 111 | — |
| MADS3 intron (O. sativa) | 112 | — |
| SpFdx transit peptide from S. pratensis | 113 | 114 |
| NOS terminator | 115 | — |
| OCS3 terminator | 116 | — |
| Consensus sequence in FIG. 1 | — | 117 |
| Consensus sequence in FIG. 2 | — | 118 |
| Consensus sequence in FIG. 3 | — | 119 |
| Modified transit peptide SpFdx | 120 | 114 |
| Atc17 intron | 121 | — |
| Atss1 intron | 122 | — |
| TOI3357 terminator | 123 | — |
| ClmMD mitochondrial transit peptide from C. lanatus | 124 | 125 |
| Ubiquitin promoter from Z. mays (constitutive) | 126 | |
| Ubiquitin intron from Z. mays | 127 | |
| KG86 promoter (whole-seed specific) | 128 | |

The following examples serve to illustrate certain embodiments and aspects of the present invention and are not to be construed as limiting the scope thereof.

### EXAMPLES

### Example 1

### Construction of Expression Cassettes

General cloning processes such as, for example, restriction digests, agarose gel electrophoresis, purification of DNA fragments, PCR amplification, transformation of E. coli cells, growth of bacteria and sequence analysis of recombinant DNA were carried out as described in Sambrook and Russell. (2001, Molecular Cloning: A Laboratory Manual, Third Edition, Cold Spring Harbor Laboratory Press: ISBN 0-87969-577-3), Kaiser et al. (1994, “Methods in Yeast Genetics,” Cold Spring Harbor Laboratory Press: ISBN 0-87969-451-3), or “Gateway® Technology,” Version E, (Invitrogen, (Carlsbad, Calif.), 2010, see webpage at tools.invitrogen.com/content/sfs/manuals/gatewayman.pdf). Specific cloning methods include ligation of DNA fragments, ligation independent cloning (LIC), and/or Gateway cloning as described in Sambrook and Russell. (2001, Molecular Cloning: A Laboratory Manual, Third Edition, Cold Spring Harbor Laboratory Press: ISBN 0-87969-577-3), or “Gateway® Technology,” Version E, (Invitrogen, (Carlsbad, Calif.), 2010, see webpage at tools.invitrogen.com/content/sfs/manuals/gatewayman.pdf).

The nucleic acid molecules encoding the pyruvate kinases from Arabidopsis (At1g32440, At2g36580, or At5g52920), Brassica napus, and E. coli (b1676 or b1854) can be generated through reverse translation of the protein sequence, codon optimization of the resulting nucleotide sequence for expression in maize, and DNA synthesis. Specifically, the nucleic acid molecules encoding the pyruvate kinases used in constructs 1-10 of Table 10 below were PCR amplified and cloned. The nucleic acid molecules encoding the pyruvate kinases may also be synthesized and cloned into a construct.

DNA synthesis is performed by a range of commercial vendors including Epoch Life Science (Missouri City, Tex.), Invitrogen, (Carlsbad, Calif.), Blue Heron Biotechnology (Bothell, Wash.) and DNA 2.0 (Menlo Park, Calif.). After synthesis, the nucleic acid sequence encoding the Arabidopsis pyruvate kinase (At1g32440, At2g36580, or At5g52920), Brassica napus pyruvate kinase, and E. coli pyruvate kinase (b1676 or b1854) were cloned into standard cloning vectors and sequenced.

The expression cassettes were then assembled in a vector by cloning the synthesized or cloned DNA encoding the pyruvate kinase from Arabidopsis, Brassica napus, or E. coli downstream of a promoter and upstream of a terminator. An intron from the rice Met1 gene was also cloned in between of the promoter and the pyruvate kinase coding sequence. Instead of an intron from the rice Met1 gene, an intron from the rice MADS3 gene can also be used. In some constructs where targeting the pyruvate kinase into a plastid was desired, a nucleotide sequence encoding the SpFdx transit peptide was also cloned between the intron sequence and the pyruvate kinase coding sequence. Based on various combinations of different components, examples of the expression cassettes of the invention may include, but not limited to, the expression cassettes exemplified in Tables 9 and 10 below. As further examples of expression constructs, the expression cassettes as illustrated in Table 9 optionally do not have a terminator.

TABLE 9
Examples of the expression cassettes of the invention.

| Promoter | Intron | PK Gene | Transit Peptide | Terminator |
|---|---|---|---|---|
| SEQ ID NO | SEQ ID NO | SEQ ID NO | SEQ ID NO | SEQ ID NO |
| 104 | 111 | 1 | 113 or 120 | 115 |
| 104 | 111 | 3 | 113 or 120 | 115 |
| 104 | 111 | 5 | 113 or 120 | 115 |
| 104 | 111 | 7 | 113 or 120 | 115 |
| 104 | 111 | 9 | 113 or 120 | 115 |
| 104 | 111 | 11 | 113 or 120 | 115 |
| 104 | 111 | 13 | 113 or 120 | 115 |
| 105 | 111 | 1 | 113 or 120 | 115 |
| 105 | 111 | 3 | 113 or 120 | 115 |
| 105 | 111 | 5 | 113 or 120 | 115 |
| 105 | 111 | 7 | 113 or 120 | 115 |
| 105 | 111 | 9 | 113 or 120 | 115 |
| 105 | 111 | 11 | 113 or 120 | 115 |
| 105 | 111 | 13 | 113 or 120 | 115 |
| 106 | 111 | 1 | 113 or 120 | 115 |
| 106 | 111 | 3 | 113 or 120 | 115 |
| 106 | 111 | 5 | 113 or 120 | 115 |
| 106 | 111 | 7 | 113 or 120 | 115 |
| 106 | 111 | 9 | 113 or 120 | 115 |
| 106 | 111 | 11 | 113 or 120 | 115 |
| 106 | 111 | 13 | 113 or 120 | 115 |
| 107 | 111 | 1 | 113 or 120 | 115 |
| 107 | 111 | 3 | 113 or 120 | 115 |
| 107 | 111 | 5 | 113 or 120 | 115 |
| 107 | 111 | 7 | 113 or 120 | 115 |
| 107 | 111 | 9 | 113 or 120 | 115 |
| 107 | 111 | 11 | 113 or 120 | 115 |
| 107 | 111 | 13 | 113 or 120 | 115 |
| 108 | 111 | 1 | 113 or 120 | 115 |
| 108 | 111 | 3 | 113 or 120 | 115 |
| 108 | 111 | 5 | 113 or 120 | 115 |
| 108 | 111 | 7 | 113 or 120 | 115 |
| 108 | 111 | 9 | 113 or 120 | 115 |
| 108 | 111 | 11 | 113 or 120 | 115 |
| 108 | 111 | 13 | 113 or 120 | 115 |
| 104 | 112 | 1 | 113 or 120 | 115 |
| 104 | 112 | 3 | 113 or 120 | 115 |
| 104 | 112 | 5 | 113 or 120 | 115 |
| 104 | 112 | 7 | 113 or 120 | 115 |
| 104 | 112 | 9 | 113 or 120 | 115 |
| 104 | 112 | 11 | 113 or 120 | 115 |
| 104 | 112 | 13 | 113 or 120 | 115 |
| 105 | 112 | 1 | 113 or 120 | 115 |
| 105 | 112 | 3 | 113 or 120 | 115 |
| 105 | 112 | 5 | 113 or 120 | 115 |
| 105 | 112 | 7 | 113 or 120 | 115 |
| 105 | 112 | 9 | 113 or 120 | 115 |
| 105 | 112 | 11 | 113 or 120 | 115 |
| 105 | 112 | 13 | 113 or 120 | 115 |
| 106 | 112 | 1 | 113 or 120 | 115 |
| 106 | 112 | 3 | 113 or 120 | 115 |
| 106 | 112 | 5 | 113 or 120 | 115 |
| 106 | 112 | 7 | 113 or 120 | 115 |
| 106 | 112 | 9 | 113 or 120 | 115 |
| 106 | 112 | 11 | 113 or 120 | 115 |
| 106 | 112 | 13 | 113 or 120 | 115 |
| 107 | 112 | 1 | 113 or 120 | 115 |
| 107 | 112 | 3 | 113 or 120 | 115 |
| 107 | 112 | 5 | 113 or 120 | 115 |
| 107 | 112 | 7 | 113 or 120 | 115 |
| 107 | 112 | 9 | 113 or 120 | 115 |
| 107 | 112 | 11 | 113 or 120 | 115 |
| 107 | 112 | 13 | 113 or 120 | 115 |
| 108 | 112 | 1 | 113 or 120 | 115 |
| 108 | 112 | 3 | 113 or 120 | 115 |
| 108 | 112 | 5 | 113 or 120 | 115 |
| 108 | 112 | 7 | 113 or 120 | 115 |
| 108 | 112 | 9 | 113 or 120 | 115 |
| 108 | 112 | 11 | 113 or 120 | 115 |
| 108 | 112 | 13 | 113 or 120 | 115 |
| 108 | 112 | 59 | — | 115 |
| 108 | 112 | 81 | — | 115 |
| 108 | 111 | 85 | 113 or 120 | 115 |
| 108 | 111 | 87 | 113 or 120 | 115 |
| 108 | 111 | 89 | 113 or 120 | 115 |
| 109 | 111 | 85 | — | 115 |
| 109 | 111 | 87 | — | 115 |
| 109 | 111 | 89 | — | 115 |
| 109 | 112 | 85 | — | 115 |
| 109 | 112 | 87 | — | 115 |
| 109 | 112 | 89 | — | 115 |

Table 10 below exemplifies expression cassettes that are used for overexpressing the Arabidopsis, Brassica, and E. coli pyruvate kinases in maize. Maize plants containing Construct 1, 2, 3, 4, 5, 6, 7, 8, 9, 10, or 13 were evaluated in field trials for yield and protein, oil, and amino acid content (see Examples 4 and 5).

TABLE 10
Examples of expression cassettes for overexpressing the Arabidopsis,
Brassica, and E. coli pyruvate kinases in maize.

| Construct | Cassette component | SEQ ID NO |
|---|---|---|
| 1 | p10kDaZein::i-Met1-1::SpFdx::PKpAt920::t-NOS | 106, 111, 113, 1, 115 |
| 2 | pZmGlb1::i-Met1-1::SpFdx::PKpAt920::t-NOS | 108, 111, 113, 1, 115 |
| 3 | p10kDaZein::i-Met1-1::SpFdx::PKpAt440::t-NOS | 106, 111, 113, 9, 115 |
| 4 | pZmGlb1::i-Met1-1::SpFdx::PKpAt440::t-NOS | 108, 111, 113, 9, 115 |
| 5 | pScBV::i-Met1-1::b1854::t-NOS | 109, 111, 87, 115 |
| 6 | pSh2::i-Met1-1::SpFdx::b1854::t-NOS | 105, 111, 113, 87, 115 |
| 7 | pZmGlb1::i-Met1-1::SpFdx::b1854::t-NOS | 108, 111, 113, 87, 115 |
| 8 | pZmGlb1::i-Met1-1::At2g36580::t-NOS | 108, 111, 81, 115 |
| 9 | pZmGlb1::i-Met1-1::PKpBn::t-NOS | 108, 111, 59, 115 |
| 10 | pZmGlb1::i-Met1-1::SpFdx::b1676::t-NOS | 108, 111, 113, 85, 115 |
| 11 | p10kDaZein::i-MADS3::SpFdx::PKpAt920::t-NOS | 106, 112, 120, 1, 115 |
| 12 | p10kDaZein::i-Met1-1::SpFdx::PKpAt920::t-NOS | 106, 111, 120, 1, 115 |
| 13 | pScBV::i-Met1-1::SpFdx::b1854::t-NOS | 109, 111, 113, 87, 115 |
| 14 | pUBI::i-Ubi:: PKpAt920::t-NOS | 126, 127, 1, 115 |
| 15 | pScBV254::i-Met1-1::SpFdx::PKpAt920.Zm::t-NOS | 110, 111, 113, 7, 115 |
| 16 | pScBV254::i-Met1-1:: PKpAt920::t-NOS | 110, 111, 3, 115 |
| 17 | p10kDaZein::i-Met1-1::SpFdx:: PKpAt920::t-NOS | 106, 111, 113, 3, 115 |
| 18 | p10kDaZein::i-Met1-1:: PKpAt920::t-NOS | 106, 111, 3, 115 |
| 19 | p10kDaZein::i-Met1-1::SpFdx::b1854.Zm::t-NOS | 106, 111, 113, 89, 115 |
| 20 | p10kDaZein::i-Atc17:: b1854.Zm::t-TOI3357 | 106, 121, 89, 123 |
| 21 | pUBI::i-Ubi:: PkpAt440::t-OCS3 | 126, 127, 9, 116 |
| 22 | pUBI::i-Ubi::PKpAt920.Zm::t-NOS | 126, 127, 7, 115 |
| 23 | p10kDaZein::i-Met1-1::PKpAt920.Zm::t-NOS | 106, 111, 7, 115 |
| 24 | pUBI::i-Ubi:: PkpAt440.Zm::t-OCS3 | 126, 127, 13, 116 |
| 25 | p10kDaZein::PKpAt920::t-OCS3 | 106, 1, 116 |

### Example 2

### Construction of Plant Transformation Vectors

Plant transformation binary vectors such as pBi-nAR are used (Höfgen & Willmitzer 1990, Plant Sci. 66:221-230). Construction of the binary vectors was performed by ligation of the expression cassette into the binary vector. Further examples for plant binary vectors are the pSUN300 or pSUN2-GW vectors and the pPZP vectors (Hajdukiewicz et al., Plant Molecular Biology 25: 989-994, 1994). These binary vectors contain an antibiotic resistance gene under the control of the NOS promoter. Expression cassettes are cloned into the multiple cloning site of the pEntry vector using standard cloning procedures. pEntry vectors are combined with a pSUN destination vector to form a binary vector by the use of the GATEWAY technology (Invitrogen, webpage at invitrogen.com) following the manufacturer's instructions. The recombinant vector containing the expression cassette was transformed into Top10 cells (Invitrogen) using standard conditions. Transformed cells were selected on LB agar containing 50 μg/ml kanamycin grown overnight at 37° C. Plasmid DNA was extracted using the QIAprep Spin Miniprep Kit (Qiagen) following manufacturer's instructions. Analysis of subsequent clones and restriction mapping was performed according to standard molecular biology techniques (Sambrook et al., 1989, “Molecular Cloning: A Laboratory Manual,” 2nd Edition, Cold Spring Harbor Laboratory Press, Cold Spring Harbor, N.Y.).

### Example 3

### Plant Transformation

### Maize

Agrobacterium cells harboring a plasmid containing the gene of interest and the mutated maize AHAS gene were grown in YP medium supplemented with appropriate antibiotics for 1-2 days. One loop of Agrobacterium cells was collected and suspended in 1.8 ml M-LS-002 medium (LS-inf). The cultures were incubated while shaking at 1,200 rpm for 5 min-3 hrs. Corn cobs were harvested at 8-11 days after pollination. The cobs were sterilized in 20% Clorox solution for 5 min, followed by spraying with 70% Ethanol and then thoroughly rinsed with sterile water. Immature embryos 0.8-2.0 mm in size were dissected into the tube containing Agrobacterium cells in LS-inf solution.

The constructs were transformed into immature embryos by a protocol modified from Japan Tobacco Agrobacterium mediated plant transformation method (U.S. Pat. Nos. 5,591,616; 5,731,179; 6,653,529; and U.S. Patent Application Publication No. 2009/0249514). Two types of plasmid vectors were used for transformation. One type had only one T-DNA border on each of left and right side of the border, and selectable marker gene and gene of interest were between the left and right T-DNA borders. The other type was so called “two T-DNA constructs” as described in Japan Tobacco U.S. Pat. No. 5,731,179. In the two DNA constructs, the selectable marker gene was located between one set of T-DNA borders and the gene of interest was included in between the second set of T-DNA borders. Either plasmid vector can be used. The plasmid vector was electroporated into Agrobacterium.

Agrobacterium infection of the embryos was carried out by inverting the tube several times. The mixture was poured onto a filter paper disk on the surface of a plate containing co-cultivation medium (M-LS-011). The liquid agro-solution was removed and the embryos were checked under a microscope and placed scutellum side up. Embryos were cultured in the dark at 22° C. for 2-4 days, and transferred to M-MS-101 medium without selection and incubated for four to seven days. Embryos were then transferred to M-LS-202 medium containing 0.75 μM imazethapyr and grown for three weeks at 27° C. to select for transformed callus cells.

Plant regeneration was initiated by transferring resistant calli to M-LS-504 medium supplemented with 0.75 μM imazethapyr and growing under light at 26° C. for two to three weeks. Regenerated shoots were then transferred to a rooting box with M-MS-618 medium (0.5 μM imazethapyr). Plantlets with roots were transferred to soil-less potting mixture and grown in a growth chamber for a week, then transplanted to larger pots and maintained in a greenhouse until maturity.

Transgenic maize plant production is also described, for example, in U.S. Pat. Nos. 5,591,616 and 6,653,529; U.S. Patent Application Publication No. 2009/0249514; and WO/2006136596, each of which are hereby incorporated by reference in their entirety. Transformation of maize may be made using Agrobacterium transformation, as described in U.S. Pat. Nos. 5,591,616; 5,731,179; U.S. Patent Application Publication No. 2002/0104132, and the like. Transformation of maize (Zea mays L.) can also be performed with a modification of the method described by Ishida et al. (Nature Biotech., 1996, 14:745-750). The inbred line A188 (University of Minnesota) or hybrids with A188 as a parent are good sources of donor material for transformation (Fromm et al., Biotech, 1990, 8:833), but other genotypes can be used successfully as well. Ears are harvested from corn plants at approximately 11 days after pollination (DAP) when the length of immature embryos is about 1 to 1.2 mm. Immature embryos are co-cultivated with Agrobacterium tumefaciens that carry “super binary” vectors and transgenic plants are recovered through organogenesis. The super binary vector system is described in WO 94/00977 and WO 95/06722. Vectors are constructed as described. Various selection marker genes are used including the maize gene encoding a mutated acetohydroxy acid synthase (AHAS) enzyme (U.S. Pat. No. 6,025,541). Similarly, various promoters are used to regulate the trait gene to provide constitutive, developmental, inducible, tissue or environmental regulation of gene transcription.

Excised embryos can be used and can be grown on callus induction medium, then maize regeneration medium, containing imidazolinone as a selection agent. The Petri dishes are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

### Wheat

A specific example of wheat transformation can be found in WO 93/07256. Transformation of wheat can also be performed with the method described by Ishida et al. (Nature Biotech., 1996, 14: 745-750). The cultivar Bobwhite (available from CYMMIT, Mexico) is commonly used in transformation. Immature embryos are co-cultivated with Agrobacterium tumefaciens that carry “super binary” vectors, and transgenic plants are recovered through organogenesis. The super binary vector system is described in WO 94/00977 and WO 95/06722, which are hereby incorporated by reference in its entirety. Vectors are constructed as described. Various selection marker genes can be used including the maize gene encoding a mutated acetohydroxy acid synthase (AHAS) enzyme (U.S. Pat. No. 6,025,541). Similarly, various promoters can be used to regulate the trait gene to provide constitutive, inducible, developmental, tissue or environmental regulation of gene transcription.

After incubation with Agrobacterium, the embryos are grown on callus induction medium, then regeneration medium, containing imidazolinone as a selection agent. The Petri dishes are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

### Rice

Rice may be transformed using methods disclosed in U.S. Pat. No. 4,666,844, U.S. Pat. No. 5,350,688, U.S. Pat. No. 6,153,813, U.S. Pat. No. 6,333,449, U.S. Pat. No. 6,288,312, U.S. Pat. No. 6,365,807, U.S. Pat. No. 6,329,571, and the like.

### Soybean

Transformation of soybean can be performed using, for example, a technique described in EP 0424047, U.S. Pat. No. 5,322,783, EP 0397687, U.S. Pat. No. 5,376,543, U.S. Pat. No. 5,169,770, or by any of a number of other transformation procedures known in the art. Soybean seeds are surface sterilized with 70% ethanol for 4 minutes at room temperature with continuous shaking, followed by 20% (v/v) bleach supplemented with 0.05% (v/v) TWEEN for 20 minutes with continuous shaking. Then the seeds are rinsed 4 times with distilled water and placed on moistened sterile filter paper in a Petri dish at room temperature for 6 to 39 hours. The seed coats are peeled off, and cotyledons are detached from the embryo axis. The embryo axis is examined to make sure that the meristematic region is not damaged. The excised embryo axes are collected in a half-open sterile Petri dish and air-dried to a moisture content less than 20% (fresh weight) in a sealed Petri dish until further use.

Brassica napus

Canola may be transformed, for example, using methods such as those disclosed in U.S. Pat. No. 5,188,958, U.S. Pat. No. 5,463,174, U.S. Pat. No. 5,750,871, EP1566443, WO02/00900, and the like.

For example, seeds of canola are surface sterilized with 70% ethanol for 4 minutes at room temperature with continuous shaking, followed by 20% (v/v) CLOROX supplemented with 0.05% (v/v) TWEEN for 20 minutes, at room temperature with continuous shaking. Then, the seeds are rinsed four times with distilled water and placed on moistened sterile filter paper in a Petri dish at room temperature for 18 hours. The seed coats are removed and the seeds are air dried overnight in a half-open sterile Petri dish. During this period, the seeds lose approximately 85% of their water content. The seeds are then stored at room temperature in a sealed Petri dish until further use.

Agrobacterium tumefaciens culture is prepared from a single colony in LB solid medium plus appropriate antibiotics (e.g. 100 mg/l streptomycin, 50 mg/l kanamycin) followed by growth of the single colony in liquid LB medium to an optical density at 600 nm of 0.8. Then, the bacteria culture is pelleted at 7000 rpm for 7 minutes at room temperature, and resuspended in MS (Murashige et al., 1962, Physiol. Plant. 15: 473-497) medium supplemented with 100 mM acetosyringone. Bacteria cultures are incubated in this pre-induction medium for 2 hours at room temperature before use. The axis of canola zygotic seed embryos at approximately 44% moisture content are imbibed for 2 hours at room temperature with the pre-induced Agrobacterium suspension culture. (The imbibition of dry embryos with a culture of Agrobacterium is also applicable to maize and soybean embryo axes). The embryos are removed from the imbibition culture and are transferred to Petri dishes containing solid MS medium supplemented with 2% sucrose and incubated for 2 days, in the dark at room temperature. Alternatively, the embryos are placed on top of moistened (liquid MS medium) sterile filter paper in a Petri dish and incubated under the same conditions described above. After this period, the embryos are transferred to either solid or liquid MS medium supplemented with 500 mg/l carbenicillin or 300 mg/l cefotaxime to kill the Agrobacteria. The liquid medium is used to moisten the sterile filter paper. The embryos are incubated during 4 weeks at 25° C., under 440 μmol m²s¹ and a 12 hour photoperiod. Once the seedlings have produced roots, they are transferred to sterile soil. The medium of the in vitro plants is washed off before transferring the plants to soil. The plants are kept under a plastic cover for 1 week to favor the acclimatization process. Then the plants are transferred to a growth room where they are incubated at 25° C., under 440 μmol m²s¹ light intensity and 12-hour photoperiod for about 80 days.

Samples of the primary transgenic plants (T0) are analyzed by PCR to confirm the presence of T-DNA. These results can be confirmed by Southern hybridization wherein DNA is electrophoresed on a 1% agarose gel and transferred to a positively charged nylon membrane (Roche Diagnostics). The PCR DIG Probe Synthesis Kit (Roche Diagnostics) is used to prepare a digoxigenin labeled probe by PCR as recommended by the manufacturer.

### Example 4

### Yield and Grain Composition of F1 Hybrid Maize Plants

Transgenic events were produced by transformation of a maize inbred line with Constructs 1, 2, 3, 4, 5, 6, 7, 8, 9, 10, or 13. Homozygous events were planted in an isolated crossing block, detasseled, and open pollinated with a male tester to produce hybrid seed (F1 generation). The hybrid seed was used in field trials for grain yield and composition and were planted in three to twelve locations with two to four replications per location. Separate field trials were conducted for yield and analysis of grain composition. Field trials for yield were allowed to open pollinate. Field trials for composition were hand pollinated. However, either pollination method may be used for yield or composition trials. Trials were planted in a randomized complete block design, with all events per construct and corresponding isogenic non-transgenic hybrid controls. Data were collected from the composition trials for grain protein, oil and six or seven amino acids (arginine, cysteine, isoleucine, lysine, methionine, threonine, and valine) on a percent dry weight basis. Data were generated for one to four hybrid combinations over one or two years. Data was subjected to ANOVA by using JMP, where locations were treated as blocks and means were separated at the 0.05 level of significance

### Example 5

### Analysis of Protein, Oil, and Amino Acid Content

Protein content and content of one or more amino acids of transgenic and corresponding wild-type plants, plant parts, or seeds can be evaluated by methods known in the art, for example, as described for corn in US 2005/0241020, which is hereby incorporated by reference in its entirety.

Protein content and content of one or more amino acids of transgenic and corresponding wild-type plants and seeds can be evaluated by methods known in the art, for example, as described for corn in US 2005/0241020 which is hereby incorporated by reference in its entirety.

Protein and oil content was determined on a dry matter basis. Protein and oil content was measured by near-infrared (NIR) spectroscopy using a Perten DA7200 NIR analyzer and Partial Least Squares (PLS) calibration models developed based on nitrogen combustion and supercritical fluid extraction reference methods for measurement of total protein and total oil, respectively (Williams, P., Norris, K., Eds. Near-Infrared Technology in the Agricultural and Food Industries, 2nd ed.; American Association of Cereal Chemists, Inc., St. Paul, Minn., 2001; AACC, Approved Methods, 10th ed., AACC Method 39-00, Near-Infrared Methods—Guidelines for Model Development and Maintenance; American Association of Cereal Chemists, Inc.; St. Paul, Minn., 2000). Samples may also be analyzed for crude protein (2000, Combustion Analysis (LECO) AOAC Official Method 990.03), crude fat (2000, Ether Extraction, AOAC Official Method 920.39 (A)), and moisture (2000, vacuum oven, AOAC Official Method 934.01).

An example of amino acid analysis of transgenic seed can be found for corn in US 2005/0241020. For example, mature seed samples were ground with an IKA A11 basic analytical mill. Samples were analyzed for amino acids using a modified Association of Official Analytical Chemists (AOAC) official method 982.30 E (a, b, c), CHP 45.3.05, 2000, with four repetitions, modified by using the Waters AccuTag system on the Acquity HPLC platform (reference paper accepted for publication). Samples may also analyzed for complete amino acid profile (AAP) using the Association of Official Analytical Chemists (AOAC) official method 982.30 E (a, b, c), CHP 45.3.05, 2000.

Protein, oil, and amino acid content will vary widely from one location to another due to environmental effects such as weather conditions, nutrient availability, and soil moisture, as well as variation in agronomic conditions such as planting density. Thus, it is important to consider the relative difference between the transgenic hybrid and the isogenic hybrid control at each location to determine transgene effects.

As shown in Tables 11-14 (Construct 1) and 17-21 (Construct 3), overexpression of two Arabidopsis pyruvate kinases, At5g52920 and At1g32440, in maize endosperm that are targeted to the plastid significantly increased the content of oil and one or more of the following amino acids: arginine, valine, methionine, lysine, cysteine and threonine. In some events, the overexpression also increased the content of protein as shown in Table 11-14 (Construct 1). Embryo-specific expression of the Arabidopsis pyruvate kinase, At5g52920, via the ZmG1b1 promoter also resulted in significant increase in oil content (Tables 15-16; Construct 2). Plastid-targeted overexpression of the E. coli pyruvate kinase II (b1854) in maize endosperm via the pSh2 promoter (a medium strength promoter compared to the stronger 10 kDa zein promoter) did not result in a significant effect in kernel composition (Tables 30-31; Construct 6). When constitutively overexpressing the E. coli pyruvate kinase II (b1854) via the ScBV promoter without plastid targeting (Tables 25-29; Construct 5) and with plastid targeting (Tables 40-41; Construct 13), a high increase in the content of protein, oil, and one or more of the amino acids arginine, valine, methionine, lysine, cysteine, isoleucine, and threonine was observed.

TABLE 11
Summary of field data for Construct 1. Numbers shown in bold are significantly different from the control
at the p-value shown, bu/a is bushels per acre. “All” indicates the average across all events.
(T/C) % is the value for the transgenic hybrid combination (T) expressed as a percent of the control (C).

| | | Yield | Oil | Protein | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p < 0.1 | p < 0.05 | p < 0.05 | p < 0.05 | p < 0.05 | p < 0.05 | p < 0.05 | p < 0.05 | p < 0.05 |
| All | (T/C) % | 98 | 100 | 103 | 114 | 110 | 110 | 110 | 107 | 110 |
| 1A | (T/C) % | 96 | 99 | 104 | 110 | 107 | 106 | 109 | 106 | 109 |
| 1B | (T/C) % | 97 | 101 | 104 | 117 | 113 | 111 | 110 | 108 | 114 |
| 1C | (T/C) % | 96 | 98 | 108 | 113 | 114 | 108 | 112 | 107 | 112 |
| 1D | (T/C) % | 96 | 101 | 103 | 117 | 113 | 114 | 108 | 107 | 111 |
| 1E | (T/C) % | 102 | 99 | 100 | 114 | 107 | 113 | 110 | 107 | 111 |
| 1F | (T/C) % | 98 | 99 | 100 | 113 | 107 | 111 | 108 | 106 | 109 |
| 1G | (T/C) % | 100 | 102 | 102 | 113 | 110 | 109 | 112 | 106 | 110 |

TABLE 12
Field data for Construct 1. Numbers shown in bold are significantly different from the control at the p-value shown,
bu/a is bushels per acre. “All” indicates the average across all events or all testers. (T/C) % is the value
for the transgenic hybrid combination (T) expressed as a percent of the control (C). T − C is the transgenic
hybrid combination minus the control. Oil, protein, and amino acid content are shown as percent of seed dry weight.

| | | Yield | Oil | Protein | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p < 0.1 | p < 0.05 | p < 0.05 | p < 0.05 | p < 0.05 | p < 0.05 | p < 0.05 | p < 0.05 | p < 0.05 |
| All | Construct (T) | 159.5 | 5.3 | 9.9 | 0.373 | 0.184 | 0.386 | 0.186 | 0.336 | 0.514 |
| All | Control (C) | 162.8 | 5.3 | 9.6 | 0.329 | 0.167 | 0.349 | 0.169 | 0.315 | 0.465 |
| All | T − C | −3.2 | 0.0 | 0.3 | 0.044 | 0.017 | 0.036 | 0.017 | 0.021 | 0.048 |
| All | (T/C) % | 98 | 100 | 103 | 114 | 110 | 110 | 110 | 107 | 110 |
| All | p-value | 0.16 | 0.85 | 0.03 | 0.00 | 0.00 | 0.00 | 0.00 | 0.00 | 0.00 |
| 1A | Event (T) | 156.9 | 5.2 | 10.0 | 0.361 | 0.179 | 0.372 | 0.184 | 0.334 | 0.507 |
| 1A | Control (C) | 162.7 | 5.3 | 9.6 | 0.329 | 0.167 | 0.350 | 0.169 | 0.315 | 0.465 |
| 1A | T − C | −5.7 | −0.1 | 0.4 | 0.032 | 0.011 | 0.023 | 0.015 | 0.020 | 0.041 |
| 1A | (T/C) % | 96 | 99 | 104 | 110 | 107 | 106 | 109 | 108 | 109 |
| 1A | p-value | 0.15 | 0.45 | 0.05 | 0.00 | 0.08 | 0.01 | 0.00 | 0.00 | 0.00 |
| 1B | Event (T) | 158.2 | 5.3 | 9.9 | 0.383 | 0.188 | 0.389 | 0.187 | 0.341 | 0.529 |
| 1B | Control (C) | 162.7 | 5.3 | 9.6 | 0.329 | 0.167 | 0.350 | 0.169 | 0.314 | 0.465 |
| 1B | T − C | −4.5 | 0.0 | 0.4 | 0.054 | 0.021 | 0.039 | 0.018 | 0.025 | 0.064 |
| 1B | (T/C) % | 97 | 101 | 104 | 117 | 113 | 111 | 110 | 108 | 114 |
| 1B | p-value | 0.28 | 0.70 | 0.06 | 0.00 | 0.00 | 0.00 | 0.00 | 0.00 | 0.00 |
| 1C | Event (T) | 156.5 | 5.2 | 10.4 | 0.371 | 0.190 | 0.379 | 0.189 | 0.338 | 0.520 |
| 1C | Control (C) | 162.7 | 5.3 | 9.6 | 0.329 | 0.167 | 0.350 | 0.169 | 0.315 | 0.466 |
| 1C | T − C | −6.2 | −0.1 | 0.8 | 0.042 | 0.023 | 0.029 | 0.020 | 0.023 | 0.054 |
| 1C | (T/C) % | 96 | 98 | 108 | 113 | 114 | 108 | 112 | 107 | 112 |
| 1C | p-value | 0.18 | 0.21 | 0.00 | 0.00 | 0.00 | 0.00 | 0.00 | 0.00 | 0.00 |
| 1D | Event (T) | 156.8 | 5.4 | 9.9 | 0.384 | 0.189 | 0.397 | 0.182 | 0.335 | 0.516 |
| 1D | Control (C) | 162.7 | 5.3 | 9.6 | 0.329 | 0.167 | 0.349 | 0.169 | 0.315 | 0.465 |
| 1D | T − C | −5.9 | 0.0 | 0.3 | 0.055 | 0.022 | 0.048 | 0.013 | 0.021 | 0.051 |
| 1D | (T/C) % | 96 | 101 | 103 | 117 | 113 | 114 | 108 | 107 | 111 |
| 1D | p-value | 0.16 | 0.70 | 0.09 | 0.00 | 0.00 | 0.00 | 0.01 | 0.00 | 0.00 |
| 1E | Event (T) | 166.6 | 5.3 | 9.6 | 0.375 | 0.179 | 0.396 | 0.185 | 0.336 | 0.515 |
| 1E | Control (C) | 162.7 | 5.3 | 9.6 | 0.329 | 0.167 | 0.350 | 0.169 | 0.315 | 0.465 |
| 1E | T − C | 3.9 | 0.0 | 0.0 | 0.046 | 0.012 | 0.046 | 0.016 | 0.021 | 0.049 |
| 1E | (T/C) % | 102 | 99 | 100 | 114 | 107 | 113 | 110 | 107 | 111 |
| 1E | p-value | 0.32 | 0.68 | 0.96 | 0.00 | 0.05 | 0.00 | 0.00 | 0.00 | 0.00 |
| 1F | Event (T) | 159.9 | 5.2 | 9.6 | 0.370 | 0.179 | 0.389 | 0.183 | 0.333 | 0.506 |
| 1F | Control (C) | 162.7 | 5.3 | 9.6 | 0.329 | 0.167 | 0.350 | 0.169 | 0.315 | 0.465 |
| 1F | T − C | −2.8 | −0.1 | 0.0 | 0.041 | 0.012 | 0.040 | 0.014 | 0.019 | 0.040 |
| 1F | (T/C) % | 98 | 99 | 100 | 113 | 107 | 111 | 108 | 106 | 109 |
| 1F | p-value | 0.44 | 0.47 | 0.79 | 0.00 | 0.05 | 0.00 | 0.00 | 0.00 | 0.00 |
| 1G | Event (T) | 162.7 | 5.4 | 9.8 | 0.370 | 0.184 | 0.382 | 0.189 | 0.334 | 0.511 |
| 1G | Control (C) | 152.7 | 5.3 | 9.6 | 0.328 | 0.167 | 0.349 | 0.169 | 0.315 | 0.465 |
| 1G | T − C | 0.0 | 0.1 | 0.2 | 0.042 | 0.017 | 0.033 | 0.020 | 0.020 | 0.046 |
| 1G | (T/C) % | 100 | 102 | 102 | 113 | 110 | 109 | 112 | 106 | 110 |
| 1G | p-value | 1.00 | 0.17 | 0.26 | 0.00 | 0.01 | 0.00 | 0.00 | 0.00 | 0.00 |

TABLE 13
Summary of field data for Construct 1 by year. Numbers shown in bold are significantly different from the
control at the p-value shown, bu/a is bushels per acre. “All” indicates the average across both years.
(T/C) % is the value for the transgenic hybrid combination (T) expressed as a percent of the control (C).

| | | Yield | Oil | Protein | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Year | Description | p < 0.1 | p < 0.05 | p < 0.05 | p < 0.05 | p < 0.05 | p < 0.05 | p < 0.05 | p < 0.05 | p < 0.05 |
| All | (T/C) % | 96 | 104 | 105 | 114 | 107 | 115 | 110 | 106 | 109 |
| 2 | (T/C) % | 97 | 102 | 106 | 111 | 110 | 105 | 109 | 105 | 109 |
| 1 | (T/C) % | 98 | 104 | 104 | 114 | 104 | 117 | 109 | 105 | 108 |

TABLE 14
Field data for Construct 1 by year. Numbers shown in bold are significantly different
from the control at the p-value shown. bu/a is bushels per acre. “All” indicates
the average across all events or all years. (T/C) % is the value for the transgenic hybrid
combination (T) expressed as a percent of the control (C). T − C is the transgenic
hybrid combination minus the control. Oil, protein, and amino acid content are
shown as percent of seed dry weight.

| | | | Yield | Oil | Protein | Arg | Cys | Lys | Met | Thr | Val |
|---|---|---|---|---|---|---|---|---|---|---|---|
| | | | (bu/a) | (%) | (%) | (%) | (%) | (%) | (%) | (%) | (%) |
| Event | Year | Description | p < 0.1 | p < 0.05 | p < 0.05 | p < 0.05 | p < 0.05 | p < 0.05 | p < 0.05 | p < 0.05 | p < 0.05 |
| All | All | Construct (T) | 173.2 | 4.8 | 9.9 | 0.350 | 0.163 | 0.366 | 0.176 | 0.317 | 0.505 |
| All | All | Control (C) | 180.2 | 4.6 | 9.4 | 0.308 | 0.152 | 0.319 | 0.160 | 0.298 | 0.462 |
| All | All | T − C | −7.0 | 0.2 | 0.4 | 0.042 | 0.011 | 0.047 | 0.016 | 0.019 | 0.043 |
| All | All | (T/C) % | 96 | 104 | 105 | 114 | 107 | 115 | 110 | 106 | 109 |
| All | All | p-value | 0.01 | 0.00 | 0.00 | 0.00 | 0.02 | 0.00 | 0.00 | 0.00 | 0.00 |
| All | 2 | Construct (T) | 166.4 | 4.9 | 10.0 | 0.363 | 0.172 | 0.377 | 0.186 | 0.333 | 0.521 |
| All | 2 | Control (C) | 172.2 | 4.8 | 9.4 | 0.327 | 0.156 | 0.360 | 0.170 | 0.316 | 0.478 |
| All | 2 | T − C | −5.8 | 0.1 | 0.6 | 0.036 | 0.016 | 0.016 | 0.016 | 0.017 | 0.043 |
| All | 2 | (T/C) % | 97 | 102 | 106 | 111 | 110 | 105 | 109 | 105 | 109 |
| All | 2 | p-value | 0.08 | 0.18 | 0.03 | 0.01 | 0.12 | 0.19 | 0.06 | 0.03 | 0.00 |
| All | 1 | Construct (T) | 183.0 | 4.8 | 9.7 | 0.344 | 0.157 | 0.360 | 0.168 | 0.307 | 0.496 |
| All | 1 | Control (C) | 187.5 | 4.6 | 9.4 | 0.303 | 0.152 | 0.307 | 0.155 | 0.292 | 0.457 |
| All | 1 | T − C | −4.5 | 0.2 | 0.3 | 0.041 | 0.005 | 0.053 | 0.013 | 0.015 | 0.039 |
| All | 1 | (T/C) % | 98 | 104 | 104 | 114 | 104 | 117 | 109 | 105 | 108 |
| All | 1 | p-value | 0.07 | 0.00 | 0.02 | 0.00 | 0.21 | 0.00 | 0.00 | 0.00 | 0.00 |
| 1A | All | Event (T) | 169.8 | 4.7 | 9.9 | 0.345 | 0.154 | 0.357 | 0.175 | 0.313 | 0.500 |
| 1A | All | Control (C) | 180.8 | 4.6 | 9.4 | 0.309 | 0.153 | 0.320 | 0.160 | 0.298 | 0.462 |
| 1A | All | T − C | −11.0 | 0.1 | 0.4 | 0.036 | 0.001 | 0.037 | 0.015 | 0.015 | 0.038 |
| 1A | All | (T/C) % | 94 | 102 | 105 | 112 | 101 | 112 | 109 | 105 | 108 |
| 1A | All | p-value | 0.03 | 0.27 | 0.04 | 0.00 | 0.81 | 0.00 | 0.01 | 0.05 | 0.00 |
| 1A | 2 | Event (T) | 161.6 | 4.8 | 10.1 | 0.356 | 0.158 | 0.358 | 0.179 | 0.328 | 0.517 |
| 1A | 2 | Control (C) | 172.2 | 4.8 | 9.5 | 0.327 | 0.158 | 0.359 | 0.171 | 0.317 | 0.479 |
| 1A | 2 | T − C | −10.6 | 0.0 | 0.6 | 0.028 | −0.001 | −0.001 | 0.008 | 0.012 | 0.038 |
| 1A | 2 | (T/C) % | 94 | 100 | 106 | 109 | 100 | 100 | 105 | 104 | 108 |
| 1A | 2 | p-value | 0.08 | 1.00 | 0.15 | 0.20 | 0.97 | 0.98 | 0.45 | 0.38 | 0.10 |
| 1A | 1 | Event (T) | 181.0 | 4.7 | 9.7 | 0.339 | 0.153 | 0.357 | 0.170 | 0.303 | 0.491 |
| 1A | 1 | Control (C) | 187.5 | 4.6 | 9.4 | 0.303 | 0.152 | 0.307 | 0.154 | 0.293 | 0.459 |
| 1A | 1 | T − C | −6.5 | 0.1 | 0.3 | 0.036 | 0.002 | 0.050 | 0.016 | 0.010 | 0.032 |
| 1A | 1 | (T/C) % | 97 | 102 | 103 | 112 | 101 | 116 | 110 | 103 | 107 |
| 1A | 1 | p-value | 0.19 | 0.20 | 0.28 | 0.00 | 0.83 | 0.00 | 0.01 | 0.17 | 0.01 |
| 1B | All | Event (T) | 179.1 | 4.9 | 9.8 | 0.348 | 0.164 | 0.356 | 0.176 | 0.318 | 0.505 |
| 1B | All | Control (C) | 181.2 | 4.6 | 9.4 | 0.309 | 0.152 | 0.320 | 0.160 | 0.298 | 0.462 |
| 1B | All | T − C | −2.1 | 0.3 | 0.4 | 0.039 | 0.012 | 0.036 | 0.016 | 0.020 | 0.043 |
| 1B | All | (T/C) % | 99 | 107 | 104 | 113 | 108 | 111 | 110 | 107 | 109 |
| 1B | All | p-value | 0.68 | 0.00 | 0.06 | 0.00 | 0.15 | 0.00 | 0.01 | 0.01 | 0.00 |
| 1B | 2 | Event (T) | 167.7 | 4.9 | 10.1 | 0.365 | 0.177 | 0.360 | 0.190 | 0.340 | 0.530 |
| 1B | 2 | Control (C) | 172.2 | 4.8 | 9.5 | 0.330 | 0.158 | 0.361 | 0.171 | 0.317 | 0.481 |
| 1B | 2 | T − C | −4.5 | 0.1 | 0.6 | 0.035 | 0.019 | −0.001 | 0.019 | 0.022 | 0.049 |
| 1B | 2 | (T/C) % | 97 | 103 | 107 | 111 | 112 | 100 | 111 | 107 | 110 |
| 1B | 2 | p-value | 0.44 | 0.29 | 0.10 | 0.12 | 0.26 | 0.96 | 0.13 | 0.09 | 0.02 |
| 1B | 1 | Event (T) | 189.2 | 4.9 | 9.5 | 0.331 | 0.151 | 0.353 | 0.162 | 0.298 | 0.480 |
| 1B | 1 | Control (C) | 187.5 | 4.6 | 9.4 | 0.303 | 0.152 | 0.306 | 0.154 | 0.293 | 0.458 |
| 1B | 1 | T − C | 1.7 | 0.3 | 0.1 | 0.028 | −0.001 | 0.048 | 0.007 | 0.006 | 0.021 |
| 1B | 1 | (T/C) % | 101 | 107 | 101 | 109 | 100 | 116 | 105 | 102 | 105 |
| 1B | 1 | p-value | 0.71 | 0.00 | 0.71 | 0.02 | 0.93 | 0.00 | 0.17 | 0.45 | 0.09 |
| 1C | All | Event (T) | 172.7 | 4.7 | 10.4 | 0.354 | 0.170 | 0.355 | 0.180 | 0.321 | 0.514 |
| 1C | All | Control (C) | 181.4 | 4.6 | 9.4 | 0.309 | 0.152 | 0.320 | 0.160 | 0.298 | 0.462 |
| 1C | All | T − C | −8.7 | 0.1 | 0.9 | 0.045 | 0.018 | 0.035 | 0.020 | 0.023 | 0.052 |
| 1C | All | (T/C) % | 95 | 102 | 110 | 115 | 112 | 111 | 112 | 108 | 111 |
| 1C | All | p-value | 0.08 | 0.35 | 0.00 | 0.00 | 0.02 | 0.00 | 0.00 | 0.00 | 0.00 |
| 1C | 2 | Event (T) | 167.3 | 4.7 | 11.1 | 0.357 | 0.190 | 0.344 | 0.195 | 0.332 | 0.524 |
| 1C | 2 | Control (C) | 172.2 | 4.7 | 9.7 | 0.330 | 0.161 | 0.355 | 0.173 | 0.319 | 0.484 |
| 1C | 2 | T − C | −4.9 | −0.1 | 1.4 | 0.027 | 0.030 | −0.011 | 0.022 | 0.013 | 0.040 |
| 1C | 2 | (T/C) % | 97 | 98 | 115 | 108 | 119 | 97 | 113 | 104 | 108 |
| 1C | 2 | p-value | 0.40 | 0.53 | 0.01 | 0.30 | 0.15 | 0.60 | 0.16 | 0.42 | 0.17 |
| 1C | 1 | Event (T) | 177.3 | 4.8 | 10.1 | 0.359 | 0.162 | 0.364 | 0.170 | 0.317 | 0.516 |
| 1C | 1 | Control (C) | 187.5 | 4.6 | 9.4 | 0.304 | 0.152 | 0.307 | 0.154 | 0.293 | 0.459 |
| 1C | 1 | T − C | −10.2 | 0.2 | 0.7 | 0.055 | 0.010 | 0.057 | 0.016 | 0.024 | 0.057 |
| 1C | 1 | (T/C) % | 95 | 104 | 107 | 118 | 106 | 119 | 110 | 108 | 112 |
| 1C | 1 | p-value | 0.03 | 0.01 | 0.01 | 0.00 | 0.17 | 0.00 | 0.00 | 0.00 | 0.00 |
| 1D | All | Event (T) | 169.6 | 4.7 | 9.8 | 0.343 | 0.158 | 0.364 | 0.172 | 0.311 | 0.495 |
| 1D | All | Control (C) | 180.5 | 4.6 | 9.4 | 0.309 | 0.152 | 0.320 | 0.160 | 0.298 | 0.462 |
| 1D | All | T − C | −10.9 | 0.1 | 0.4 | 0.034 | 0.006 | 0.044 | 0.012 | 0.013 | 0.033 |
| 1D | All | (T/C) % | 94 | 102 | 104 | 111 | 104 | 114 | 108 | 104 | 107 |
| 1D | All | p-value | 0.04 | 0.05 | 0.08 | 0.00 | 0.43 | 0.00 | 0.03 | 0.09 | 0.00 |
| 1D | 2 | Event (T) | 160.4 | 4.9 | 10.6 | 0.367 | 0.178 | 0.378 | 0.185 | 0.333 | 0.523 |
| 1D | 2 | Control (C) | 172.2 | 4.7 | 9.7 | 0.330 | 0.161 | 0.355 | 0.173 | 0.319 | 0.484 |
| 1D | 2 | T − C | −11.8 | 0.2 | 0.9 | 0.037 | 0.017 | 0.023 | 0.012 | 0.014 | 0.039 |
| 1D | 2 | (T/C) % | 93 | 104 | 109 | 111 | 111 | 107 | 107 | 104 | 108 |
| 1D | 2 | p-value | 0.08 | 0.05 | 0.01 | 0.12 | 0.27 | 0.38 | 0.36 | 0.29 | 0.10 |
| 1D | 1 | Event (T) | 182.9 | 4.7 | 9.5 | 0.333 | 0.149 | 0.355 | 0.164 | 0.301 | 0.485 |
| 1D | 1 | Control (C) | 187.5 | 4.6 | 9.4 | 0.304 | 0.152 | 0.308 | 0.154 | 0.293 | 0.459 |
| 1D | 1 | T − C | −4.5 | 0.1 | 0.0 | 0.029 | −0.003 | 0.048 | 0.010 | 0.008 | 0.026 |
| 1D | 1 | (T/C) % | 98 | 103 | 100 | 110 | 98 | 116 | 106 | 103 | 106 |
| 1D | 1 | p-value | 0.33 | 0.11 | 0.88 | 0.01 | 0.68 | 0.00 | 0.05 | 0.26 | 0.02 |
| 1E | All | Event (T) | 178.2 | 4.8 | 9.6 | 0.353 | 0.158 | 0.376 | 0.172 | 0.316 | 0.505 |
| 1E | All | Control (C) | 182.1 | 4.6 | 9.4 | 0.309 | 0.153 | 0.320 | 0.160 | 0.298 | 0.462 |
| 1E | All | T − C | −3.9 | 0.2 | 0.2 | 0.044 | 0.005 | 0.056 | 0.012 | 0.018 | 0.043 |
| 1E | All | (T/C) % | 98 | 104 | 102 | 114 | 103 | 118 | 108 | 106 | 109 |
| 1E | All | p-value | 0.41 | 0.05 | 0.47 | 0.00 | 0.39 | 0.00 | 0.03 | 0.02 | 0.00 |
| 1E | 2 | Event (T) | 174.2 | 4.8 | 9.8 | 0.376 | 0.177 | 0.389 | 0.182 | 0.341 | 0.535 |
| 1E | 2 | Control (C) | 172.2 | 4.7 | 9.7 | 0.330 | 0.161 | 0.355 | 0.173 | 0.319 | 0.484 |
| 1E | 2 | T − C | 2.0 | 0.1 | 0.1 | 0.047 | 0.016 | 0.034 | 0.009 | 0.022 | 0.051 |
| 1E | 2 | (T/C) % | 101 | 102 | 101 | 114 | 110 | 110 | 105 | 107 | 111 |
| 1E | 2 | p-value | 0.66 | 0.42 | 0.80 | 0.04 | 0.25 | 0.10 | 0.42 | 0.11 | 0.02 |
| 1E | 1 | Event (T) | 180.7 | 4.8 | 9.6 | 0.342 | 0.149 | 0.368 | 0.163 | 0.302 | 0.490 |
| 1E | 1 | Control (C) | 187.5 | 4.6 | 9.4 | 0.302 | 0.151 | 0.306 | 0.154 | 0.292 | 0.458 |
| 1E | 1 | T − C | −6.8 | 0.2 | 0.2 | 0.039 | −0.002 | 0.062 | 0.010 | 0.010 | 0.032 |
| 1E | 1 | (T/C) % | 96 | 105 | 102 | 113 | 99 | 120 | 106 | 103 | 107 |
| 1E | 1 | p-value | 0.19 | 0.01 | 0.56 | 0.00 | 0.75 | 0.00 | 0.06 | 0.18 | 0.01 |
| 1F | All | Event (T) | 174.6 | 4.7 | 9.8 | 0.347 | 0.168 | 0.362 | 0.180 | 0.316 | 0.502 |
| 1F | All | Control (C) | 181.3 | 4.6 | 9.4 | 0.309 | 0.152 | 0.320 | 0.160 | 0.298 | 0.462 |
| 1F | All | T − C | −6.7 | 0.1 | 0.4 | 0.038 | 0.016 | 0.042 | 0.020 | 0.018 | 0.040 |
| 1F | All | (T/C) % | 96 | 102 | 104 | 112 | 111 | 113 | 112 | 106 | 109 |
| 1F | All | p-value | 0.16 | 0.14 | 0.06 | 0.00 | 0.03 | 0.00 | 0.00 | 0.02 | 0.00 |
| 1F | 2 | Event (T) | 165.9 | 4.8 | 10.0 | 0.357 | 0.179 | 0.378 | 0.192 | 0.334 | 0.523 |
| 1F | 2 | Control (C) | 172.2 | 4.7 | 9.7 | 0.327 | 0.159 | 0.355 | 0.173 | 0.317 | 0.481 |
| 1F | 2 | T − C | −6.3 | 0.1 | 0.3 | 0.030 | 0.020 | 0.022 | 0.020 | 0.017 | 0.042 |
| 1F | 2 | (T/C) % | 96 | 102 | 103 | 109 | 113 | 106 | 112 | 105 | 109 |
| 1F | 2 | p-value | 0.16 | 0.43 | 0.32 | 0.19 | 0.18 | 0.29 | 0.13 | 0.22 | 0.09 |
| 1F | 1 | Event (T) | 188.6 | 4.7 | 9.8 | 0.342 | 0.162 | 0.353 | 0.170 | 0.306 | 0.492 |
| 1F | 1 | Control (C) | 187.5 | 4.6 | 9.4 | 0.303 | 0.151 | 0.307 | 0.154 | 0.292 | 0.457 |
| 1F | 1 | T − C | 1.1 | 0.1 | 0.3 | 0.040 | 0.011 | 0.046 | 0.016 | 0.014 | 0.035 |
| 1F | 1 | (T/C) % | 101 | 102 | 104 | 113 | 107 | 115 | 110 | 105 | 108 |
| 1F | 1 | p-value | 0.83 | 0.20 | 0.16 | 0.00 | 0.16 | 0.00 | 0.00 | 0.07 | 0.01 |
| 1G | All | Event (T) | 173.8 | 4.9 | 9.9 | 0.365 | 0.171 | 0.380 | 0.184 | 0.325 | 0.521 |
| 1G | All | Control (C) | 181.0 | 4.6 | 9.4 | 0.309 | 0.153 | 0.320 | 0.160 | 0.298 | 0.462 |
| 1G | All | T − C | −7.2 | 0.3 | 0.5 | 0.056 | 0.018 | 0.060 | 0.024 | 0.027 | 0.059 |
| 1G | All | (T/C) % | 96 | 107 | 105 | 118 | 112 | 119 | 115 | 109 | 113 |
| 1G | All | p-value | 0.14 | 0.00 | 0.03 | 0.00 | 0.01 | 0.00 | 0.00 | 0.00 | 0.00 |
| 1G | 2 | Event (T) | 165.5 | 5.0 | 10.0 | 0.381 | 0.176 | 0.402 | 0.196 | 0.337 | 0.537 |
| 1G | 2 | Control (C) | 172.2 | 4.7 | 9.7 | 0.330 | 0.161 | 0.355 | 0.173 | 0.319 | 0.484 |
| 1G | 2 | T − C | −6.7 | 0.3 | 0.3 | 0.052 | 0.015 | 0.047 | 0.023 | 0.019 | 0.053 |
| 1G | 2 | (T/C) % | 96 | 106 | 103 | 116 | 110 | 113 | 113 | 106 | 111 |
| 1G | 2 | p-value | 0.19 | 0.02 | 0.38 | 0.04 | 0.36 | 0.04 | 0.05 | 0.21 | 0.03 |
| 1G | 1 | Event (T) | 181.0 | 4.9 | 10.0 | 0.361 | 0.171 | 0.368 | 0.175 | 0.320 | 0.518 |
| 1G | 1 | Control (C) | 187.5 | 4.6 | 9.4 | 0.304 | 0.151 | 0.308 | 0.154 | 0.293 | 0.459 |
| 1G | 1 | T − C | −6.4 | 0.3 | 0.6 | 0.057 | 0.020 | 0.061 | 0.020 | 0.028 | 0.060 |
| 1G | 1 | (T/C) % | 97 | 106 | 106 | 119 | 113 | 120 | 113 | 109 | 113 |
| 1G | 1 | p-value | 0.19 | 0.00 | 0.03 | 0.00 | 0.01 | 0.00 | 0.00 | 0.00 | 0.00 |

TABLE 15
Summary of field data for Construct 2. Numbers shown in bold are significantly different from the control at the p-value
shown. bu/a is bushels per acre. “All” indicates the average across all events or testers. (T/C) % is the value for
the transgenic hybrid combination (T) expressed as a percent of the control (C).

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | (p <= 0.10) | (p <= 0.15) | (p <= 0.15) | (p <= 0.15) | (p <= 0.15) | (p <= 0.15) | (p <= 0.15) | (p <= 0.15) | (p <= 0.15) |
| All | (T/C) % | 100 | 102 | 101 | 99 | 99 | 100 | 99 | 100 | 100 |
| 2H | (T/C) % | 98 | 108 | 93 | 95 | 91 | 100 | 95 | 95 | 94 |
| 2I | (T/C) % | 97 | 104 | 99 | 99 | 104 | 100 | 101 | 100 | 99 |
| 2J | (T/C) % | 100 | 103 | 102 | 101 | 99 | 103 | 100 | 102 | 101 |
| 2K | (T/C) % | 102 | 105 | 97 | 96 | 92 | 100 | 98 | 99 | 97 |
| 2L | (T/C) % | 100 | 96 | 105 | 102 | 105 | 101 | 104 | 103 | 103 |
| 2M | (T/C) % | 104 | 107 | 108 | 105 | 107 | 102 | 108 | 106 | 106 |
| 2N | (T/C) % | 100 | 98 | 101 | 99 | 103 | 101 | 99 | 98 | 100 |
| 2O | (T/C) % | 98 | 99 | 101 | 100 | 107 | 98 | 101 | 102 | 102 |
| 2P | (T/C) % | 102 | 106 | 100 | 100 | 91 | 105 | 91 | 99 | 99 |

TABLE 16
Field data for Construct 2. Numbers shown in bold are significantly different from the control at the p-value shown. bu/a is bushels per
acre. “All” indicates the average across all events or testers. (T/C) % is the value for the transgenic hybrid combination (T)
expressed as a percent of the control (C). T − C is the transgenic hybrid combination minus the control. Oil, protein, and
amino acid content are shown as percent of seed dry weight.

| | | | | | | | | | | Val |
|---|---|---|---|---|---|---|---|---|---|---|
| | | Yield | Oil | Prot | Arg | Cys | Lys | Met | Thr | (%) |
| | | (bu/a) | (%) | (%) | (%) | (%) | (%) | (%) | (%) | (p <= |
| Event | Description | (p <= 0.10) | (p <= 0.15) | (p <= 0.15) | (p <= 0.15) | (p <= 0.15) | (p <= 0.15) | (p <= 0.15) | (p <= 0.15) | 0.15) |
| All | Construct (T) | 188.1 | 4.3 | 9.7 | 0.321 | 0.159 | 0.322 | 0.168 | 0.302 | 0.472 |
| All | Control (C) | 187.8 | 4.2 | 9.6 | 0.325 | 0.161 | 0.323 | 0.170 | 0.301 | 0.473 |
| All | T − C | 0.3 | 0.1 | 0.1 | −0.004 | −0.002 | 0.000 | −0.002 | 0.001 | −0.001 |
| All | (T/C) % | 100 | 102 | 101 | 99 | 99 | 100 | 99 | 100 | 100 |
| All | p-value | 0.76 | 0.23 | 0.70 | 0.48 | 0.62 | 0.94 | 0.21 | 0.83 | 0.87 |
| 2H | Event (T) | 186.0 | 4.5 | 9.1 | 0.308 | 0.146 | 0.315 | 0.161 | 0.285 | 0.449 |
| 2H | Control (C) | 189.3 | 4.2 | 9.8 | 0.323 | 0.159 | 0.314 | 0.169 | 0.299 | 0.475 |
| 2H | T − C | −3.2 | 0.3 | −0.7 | −0.015 | −0.014 | 0.001 | −0.008 | −0.014 | −0.026 |
| 2H | (T/C) % | 98 | 108 | 93 | 95 | 91 | 100 | 95 | 95 | 94 |
| 2H | p-value | 0.75 | 0.03 | 0.24 | 0.37 | 0.12 | 0.92 | 0.11 | 0.27 | 0.24 |
| 2I | Event (T) | 183.8 | 4.4 | 9.5 | 0.320 | 0.168 | 0.320 | 0.171 | 0.300 | 0.469 |
| 2I | Control (C) | 188.9 | 4.2 | 9.6 | 0.323 | 0.160 | 0.320 | 0.170 | 0.301 | 0.473 |
| 2I | T − C | −5.1 | 0.2 | −0.1 | −0.003 | 0.007 | 0.000 | 0.001 | 0.000 | −0.004 |
| 2I | (T/C) % | 97 | 104 | 99 | 99 | 104 | 100 | 101 | 100 | 99 |
| 2I | p-value | 0.31 | 0.23 | 0.49 | 0.83 | 0.47 | 0.96 | 0.72 | 0.95 | 0.74 |
| 2J | Event (T) | 188.4 | 4.3 | 9.8 | 0.329 | 0.160 | 0.331 | 0.169 | 0.308 | 0.479 |
| 2J | Control (C) | 187.8 | 4.2 | 9.6 | 0.325 | 0.161 | 0.323 | 0.170 | 0.301 | 0.473 |
| 2J | T − C | 0.6 | 0.1 | 0.2 | 0.004 | −0.002 | 0.009 | −0.001 | 0.007 | 0.006 |
| 2J | (T/C) % | 100 | 103 | 102 | 101 | 99 | 103 | 100 | 102 | 101 |
| 2J | p-value | 0.87 | 0.26 | 0.47 | 0.60 | 0.86 | 0.26 | 0.91 | 0.49 | 0.74 |
| 2K | Event (T) | 191.0 | 4.4 | 9.5 | 0.314 | 0.149 | 0.323 | 0.167 | 0.300 | 0.462 |
| 2K | Control (C) | 187.8 | 4.2 | 9.7 | 0.328 | 0.162 | 0.324 | 0.170 | 0.302 | 0.477 |
| 2K | T − C | 3.2 | 0.2 | −0.3 | −0.014 | −0.013 | −0.001 | −0.003 | −0.003 | −0.015 |
| 2K | (T/C) % | 102 | 105 | 97 | 96 | 92 | 100 | 98 | 99 | 97 |
| 2K | p-value | 0.52 | 0.09 | 0.48 | 0.30 | 0.08 | 0.87 | 0.11 | 0.77 | 0.41 |
| 2L | Event (T) | 187.2 | 4.1 | 10.1 | 0.329 | 0.166 | 0.319 | 0.174 | 0.307 | 0.484 |
| 2L | Control (C) | 187.7 | 4.2 | 9.6 | 0.321 | 0.158 | 0.315 | 0.167 | 0.298 | 0.470 |
| 2L | T − C | −0.5 | −0.2 | 0.5 | 0.008 | 0.008 | 0.004 | 0.006 | 0.009 | 0.014 |
| 2L | (T/C) % | 100 | 96 | 105 | 102 | 105 | 101 | 104 | 103 | 103 |
| 2L | p-value | 0.96 | 0.20 | 0.16 | 0.32 | 0.08 | 0.87 | 0.02 | 0.16 | 0.02 |
| 2M | Event (T) | 194.7 | 4.5 | 10.2 | 0.340 | 0.173 | 0.331 | 0.183 | 0.318 | 0.497 |
| 2M | Control (C) | 187.8 | 4.2 | 9.5 | 0.324 | 0.162 | 0.324 | 0.170 | 0.299 | 0.470 |
| 2M | T − C | 6.9 | 0.3 | 0.7 | 0.016 | 0.011 | 0.006 | 0.013 | 0.019 | 0.028 |
| 2M | (T/C) % | 104 | 107 | 108 | 105 | 107 | 102 | 108 | 106 | 106 |
| 2M | p-value | 0.39 | 0.04 | 0.07 | 0.50 | 0.41 | 0.69 | 0.11 | 0.27 | 0.30 |
| 2N | Event (T) | 187.6 | 4.2 | 9.6 | 0.316 | 0.163 | 0.319 | 0.165 | 0.291 | 0.468 |
| 2N | Control (C) | 187.8 | 4.2 | 9.5 | 0.319 | 0.158 | 0.317 | 0.168 | 0.297 | 0.467 |
| 2N | T − C | −0.3 | −0.1 | 0.1 | −0.003 | 0.004 | 0.002 | −0.002 | −0.005 | 0.001 |
| 2N | (T/C) % | 100 | 98 | 101 | 99 | 103 | 101 | 99 | 98 | 100 |
| 2N | p-value | 0.96 | 0.41 | 0.77 | 0.85 | 0.71 | 0.82 | 0.75 | 0.61 | 0.92 |
| 2O | Event (T) | 184.7 | 4.2 | 9.8 | 0.328 | 0.173 | 0.316 | 0.172 | 0.307 | 0.486 |
| 2O | Control (C) | 187.8 | 4.2 | 9.8 | 0.328 | 0.161 | 0.322 | 0.171 | 0.300 | 0.479 |
| 2O | T − C | −3.1 | −0.1 | 0.1 | 0.000 | 0.012 | −0.006 | 0.002 | 0.007 | 0.007 |
| 2O | (T/C) % | 98 | 99 | 101 | 100 | 107 | 98 | 101 | 102 | 102 |
| 2O | p-value | 0.39 | 0.50 | 0.82 | 1.00 | 0.15 | 0.48 | 0.70 | 0.55 | 0.57 |
| 2P | Event (T) | 190.6 | 4.4 | 9.7 | 0.322 | 0.145 | 0.335 | 0.155 | 0.296 | 0.466 |
| 2P | Control (C) | 186.8 | 4.2 | 9.6 | 0.322 | 0.158 | 0.318 | 0.170 | 0.299 | 0.473 |
| 2P | T − C | 3.9 | 0.3 | 0.0 | 0.000 | −0.014 | 0.017 | −0.015 | −0.003 | −0.007 |
| 2P | (T/C) % | 102 | 106 | 100 | 100 | 91 | 105 | 91 | 99 | 99 |
| 2P | p-value | 0.43 | 0.08 | 0.94 | 0.99 | 0.19 | 0.17 | 0.05 | 0.76 | 0.65 |

TABLE 17
Summary of field data for Construct 3 for Year 1. Numbers shown in bold are significantly
different from the control at the p-value shown. bu/a is bushels per acre. “All” indicates
the average across all events or testers. (T/C) % is the value for the transgenic hybrid
combination (T) expressed as a percent of the control (C).

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | (T/C) % | 100 | 104 | 99 | 114 | 104 | 119 | 106 | 105 | 109 |
| 3Q | (T/C) % | 102 | 104 | 98 | 118 | 107 | 123 | 109 | 106 | 111 |
| 3R | (T/C) % | 105 | 104 | 97 | 107 | 99 | 113 | 99 | 104 | 104 |
| 3S | (T/C) % | 94 | 106 | 101 | 124 | 117 | 123 | 122 | 110 | 116 |
| 3T | (T/C) % | 100 | 106 | 95 | 106 | 98 | 118 | 98 | 102 | 103 |
| 3U | (T/C) % | 97 | 96 | 100 | 99 | 94 | 100 | 90 | 98 | 99 |

TABLE 18
Field data for Construct 3 for Year 1. Numbers shown in bold are significantly
different from the control at the p-value shown. bu/a is bushels per acre. “All”
indicates the average across all events or testers. (T/C) % is the value for the transgenic
hybrid combination (T) expressed as a percent of the control (C). T − C is the
transgenic hybrid combination minus the control. Oil, protein, and amino acid
content are shown as percent of seed dry weight.

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | Construct (T) | 155.1 | 5.0 | 8.8 | 0.336 | 0.156 | 0.407 | 0.162 | 0.318 | 0.471 |
| All | Control (C) | 155.2 | 4.8 | 8.9 | 0.296 | 0.150 | 0.343 | 0.153 | 0.302 | 0.434 |
| All | T − C | −0.1 | 0.2 | −0.1 | 0.040 | 0.006 | 0.064 | 0.009 | 0.016 | 0.037 |
| All | (T/C) % | 100 | 104 | 99 | 114 | 104 | 119 | 106 | 105 | 109 |
| All | p-value | 0.98 | 0.11 | 0.59 | 0.00 | 0.31 | 0.00 | 0.19 | 0.03 | 0.01 |
| 3Q | Event (T) | 159.0 | 5.0 | 8.7 | 0.348 | 0.161 | 0.423 | 0.167 | 0.320 | 0.483 |
| 3Q | Control (C) | 155.2 | 4.8 | 8.9 | 0.296 | 0.150 | 0.343 | 0.153 | 0.302 | 0.434 |
| 3Q | T − C | 3.8 | 0.2 | −0.2 | 0.052 | 0.011 | 0.080 | 0.014 | 0.018 | 0.049 |
| 3Q | (T/C) % | 102 | 104 | 98 | 118 | 107 | 123 | 109 | 106 | 111 |
| 3Q | p-value | 0.69 | 0.40 | 0.50 | 0.00 | 0.14 | 0.00 | 0.08 | 0.03 | 0.00 |
| 3R | Event (T) | 163.4 | 5.0 | 8.6 | 0.320 | 0.148 | 0.391 | 0.151 | 0.314 | 0.454 |
| 3R | Control (C) | 155.2 | 4.8 | 8.9 | 0.298 | 0.150 | 0.345 | 0.152 | 0.303 | 0.436 |
| 3R | T − C | 8.2 | 0.2 | −0.3 | 0.022 | −0.002 | 0.046 | −0.001 | 0.011 | 0.018 |
| 3R | (T/C) % | 105 | 104 | 97 | 107 | 99 | 113 | 99 | 104 | 104 |
| 3R | p-value | 0.40 | 0.37 | 0.24 | 0.05 | 0.66 | 0.01 | 0.93 | 0.17 | 0.10 |
| 3S | Event (T) | 145.5 | 5.1 | 9.0 | 0.368 | 0.175 | 0.422 | 0.186 | 0.331 | 0.504 |
| 3S | Control (C) | 155.2 | 4.8 | 8.9 | 0.296 | 0.150 | 0.343 | 0.153 | 0.302 | 0.434 |
| 3S | T − C | −9.7 | 0.3 | 0.1 | 0.072 | 0.025 | 0.079 | 0.033 | 0.029 | 0.070 |
| 3S | (T/C) % | 94 | 106 | 101 | 124 | 117 | 123 | 122 | 110 | 116 |
| 3S | p-value | 0.35 | 0.18 | 0.69 | 0.00 | 0.01 | 0.00 | 0.00 | 0.02 | 0.00 |
| 3T | Event (T) | 155.8 | 5.0 | 8.4 | 0.313 | 0.146 | 0.401 | 0.148 | 0.308 | 0.446 |
| 3T | Control (C) | 155.2 | 4.7 | 8.8 | 0.296 | 0.149 | 0.340 | 0.151 | 0.301 | 0.433 |
| 3T | T − C | 0.6 | 0.3 | −0.4 | 0.017 | −0.003 | 0.061 | −0.003 | 0.007 | 0.013 |
| 3T | (T/C) % | 100 | 106 | 95 | 106 | 98 | 118 | 98 | 102 | 103 |
| 3T | p-value | 0.95 | 0.26 | 0.04 | 0.12 | 0.54 | 0.00 | 0.56 | 0.30 | 0.21 |
| 3U | Event (T) | 145.9 | 5.2 | 9.2 | 0.305 | 0.138 | 0.373 | 0.138 | 0.305 | 0.438 |
| 3U | Control (C) | 149.7 | 5.4 | 9.2 | 0.309 | 0.147 | 0.373 | 0.153 | 0.311 | 0.443 |
| 3U | T − C | −3.8 | −0.2 | 0.0 | −0.004 | −0.009 | 0.000 | −0.015 | −0.006 | −0.005 |
| 3U | (T/C) % | 97 | 96 | 100 | 99 | 94 | 100 | 90 | 98 | 99 |
| 3U | p-value | 0.79 | 0.22 | 0.95 | 0.81 | 0.38 | 1.00 | 0.13 | 0.62 | 0.80 |

TABLE 19
Summary of field data for Construct 3 for Year 2. Numbers shown in bold are significantly different from the control at the p-value shown.
bu/a is bushels per acre. The events are an average over three testers. “All” indicates the average across all events or testers.
(T/C) % is the value for the transgenic hybrid combination (T) expressed as a percent of the control (C).

| | | Yield | Oil | Prot | Arg | Cys | Ile | Lys | Met | Thr | Val |
|---|---|---|---|---|---|---|---|---|---|---|---|
| | | (bu/a) | (%) | (%) | (%) | (%) | (%) | (%) | (%) | (%) | (%) |
| Event | Description | p <= 0.10 | p <= 0.05 | p <= 0.05 | p <= 0.05 | p <= 0.05 | P <= 0.05 | p <= 0.05 | p <= 0.05 | p <= 0.05 | p <= 0.05 |
| All | (T/C) % | 95 | 100 | 98 | 110 | 104 | 102 | 115 | 106 | 104 | 106 |
| 3Q | (T/C) % | 99 | 100 | 98 | 111 | 105 | 102 | 117 | 109 | 104 | 107 |
| 3R | (T/C) % | 95 | 100 | 96 | 111 | 101 | 100 | 117 | 102 | 103 | 106 |
| 3S | (T/C) % | 92 | 98 | 101 | 110 | 103 | 103 | 114 | 104 | 104 | 107 |
| 3T | (T/C) % | 95 | 102 | 99 | 109 | 107 | 101 | 110 | 109 | 103 | 105 |

TABLE 20
Field data for Construct 3 for Year 2. Numbers shown in bold are significantly different from the control
at the p-value shown. bu/a is bushels per acre. The events are an average over three testers. “All”
indicates the average across all events or testers. (T/C) % is the value for the transgenic hybrid
combination (T) expressed as a percent of the control (C). T − C is the transgenic hybrid combination
minus the control. Oil, protein, and amino acid content are shown as percent of seed dry weight.

| | | Yield | Oil | Prot | Arg | Cys | Ile | Lys | Met | Thr | Val |
|---|---|---|---|---|---|---|---|---|---|---|---|
| | | (bu/a) | (%) | (%) | (%) | (%) | (%) | (%) | (%) | (%) | (%) |
| Event | Description | p <= 0.10 | p <= 0.15 | p <= 0.15 | p <= 0.15 | p <= 0.15 | P <= 0.05 | p <= 0.15 | p <= 0.15 | p <= 0.15 | p <= 0.15 |
| All | Construct (T) | 168.3 | 4.0 | 9.7 | 0.506 | 0.232 | 0.374 | 0.402 | 0.235 | 0.485 | 0.573 |
| All | Control (C) | 176.7 | 4.0 | 9.9 | 0.458 | 0.223 | 0.368 | 0.351 | 0.222 | 0.468 | 0.539 |
| All | T − C | −8.4 | 0.0 | −0.2 | 0.048 | 0.009 | 0.006 | 0.051 | 0.013 | 0.017 | 0.034 |
| All | (T/C) % | 95 | 100 | 98 | 110 | 104 | 102 | 115 | 106 | 104 | 106 |
| All | p-value | 0.06 | 0.72 | 0.61 | 0.01 | 0.14 | 0.20 | 0.01 | 0.02 | 0.04 | 0.04 |
| 3Q | Event (T) | 175.5 | 4.0 | 9.7 | 0.510 | 0.234 | 0.375 | 0.411 | 0.241 | 0.488 | 0.575 |
| 3Q | Control (C) | 176.7 | 4.0 | 9.9 | 0.458 | 0.223 | 0.368 | 0.351 | 0.222 | 0.468 | 0.539 |
| 3Q | T − C | −1.2 | 0.0 | −0.2 | 0.052 | 0.011 | 0.007 | 0.060 | 0.019 | 0.020 | 0.036 |
| 3Q | (T/C) % | 99 | 100 | 98 | 111 | 105 | 102 | 117 | 109 | 104 | 107 |
| 3Q | p-value | 0.48 | 0.55 | 0.57 | 0.05 | 0.53 | 0.48 | 0.00 | 0.06 | 0.19 | 0.14 |
| 3R | Event (T) | 167.9 | 4.0 | 9.5 | 0.509 | 0.226 | 0.369 | 0.411 | 0.226 | 0.483 | 0.572 |
| 3R | Control (C) | 176.7 | 4.0 | 9.9 | 0.458 | 0.223 | 0.368 | 0.351 | 0.222 | 0.468 | 0.539 |
| 3R | T − C | −8.8 | 0.0 | −0.4 | 0.051 | 0.003 | 0.001 | 0.060 | 0.004 | 0.015 | 0.033 |
| 3R | (T/C) % | 95 | 100 | 96 | 111 | 101 | 100 | 117 | 102 | 103 | 106 |
| 3R | p-value | 0.18 | 0.61 | 0.28 | 0.11 | 0.52 | 1.00 | 0.03 | 0.56 | 0.33 | 0.19 |
| 3S | Event (T) | 162.3 | 3.9 | 10.0 | 0.505 | 0.230 | 0.378 | 0.399 | 0.231 | 0.486 | 0.577 |
| 3S | Control (C) | 176.7 | 4.0 | 9.9 | 0.458 | 0.223 | 0.368 | 0.351 | 0.222 | 0.468 | 0.539 |
| 3S | T − C | −14.4 | −0.1 | 0.1 | 0.047 | 0.007 | 0.010 | 0.048 | 0.009 | 0.018 | 0.038 |
| 3S | (T/C) % | 92 | 98 | 101 | 110 | 103 | 103 | 114 | 104 | 104 | 107 |
| 3S | p-value | 0.00 | 0.08 | 0.76 | 0.02 | 0.12 | 0.12 | 0.00 | 0.01 | 0.09 | 0.06 |
| 3T | Event (T) | 167.9 | 4.1 | 9.8 | 0.498 | 0.238 | 0.373 | 0.387 | 0.241 | 0.481 | 0.566 |
| 3T | Control (C) | 176.7 | 4.0 | 9.9 | 0.458 | 0.223 | 0.368 | 0.351 | 0.222 | 0.468 | 0.539 |
| 3T | T − C | −8.8 | 0.1 | −0.1 | 0.040 | 0.015 | 0.005 | 0.036 | 0.019 | 0.013 | 0.027 |
| 3T | (T/C) % | 95 | 102 | 99 | 109 | 107 | 101 | 110 | 109 | 103 | 105 |
| 3T | p-value | 0.05 | 0.48 | 0.51 | 0.19 | 0.20 | 0.48 | 0.07 | 0.19 | 0.30 | 0.12 |

TABLE 21
Field data for Construct 3 by Year by Tester. Numbers shown in bold are significantly different from the control at the p-value shown.
bu/a is bushels per acre. “All” indicates the average across all events or years. (T/C) % is the value for the transgenic hybrid
combination (T) expressed as a percent of the control (C). T − C is the transgenic hybrid combination minus the control. Oil,
protein, and amino acid content are shown as percent of seed dry weight.

| | | | | Yield | Oil | Prot | Arg | Cys | Lys | Met | Thr | Val |
|---|---|---|---|---|---|---|---|---|---|---|---|---|
| | | | | (bu/a) | (%) | (%) | (%) | (%) | (%) | (%) | (%) | (%) |
| Event | Year | Tester | Description | p <= 0.10 | p <= 0.15 | p <= 0.15 | p <= 0.15 | p <= 0.15 | p <= 0.15 | p <= 0.15 | p <= 0.15 | p <= 0.15 |
| All | All | 1 | Construct (T) | 150.6 | 4.8 | 9.5 | 0.446 | 0.203 | 0.427 | 0.205 | 0.427 | 0.552 |
| All | All | 1 | Control (C) | 159.6 | 4.8 | 10.1 | 0.402 | 0.198 | 0.362 | 0.200 | 0.415 | 0.522 |
| All | All | 1 | T − C | −9.0 | 0.0 | −0.6 | 0.044 | 0.005 | 0.065 | 0.005 | 0.012 | 0.030 |
| All | All | 1 | (T/C) % | 94 | 100 | 94 | 111 | 103 | 118 | 102 | 103 | 106 |
| All | All | 1 | p-value | 0.16 | 0.98 | 0.05 | 0.02 | 0.57 | 0.00 | 0.60 | 0.28 | 0.11 |
| All | 1 | 1 | Construct (T) | 150.4 | 5.6 | 8.9 | 0.361 | 0.167 | 0.437 | 0.177 | 0.335 | 0.504 |
| All | 1 | 1 | Control (C) | 149.7 | 5.6 | 9.2 | 0.297 | 0.158 | 0.369 | 0.165 | 0.306 | 0.441 |
| All | 1 | 1 | T − C | 0.7 | 0.0 | −0.2 | 0.064 | 0.009 | 0.068 | 0.012 | 0.029 | 0.063 |
| All | 1 | 1 | (T/C) % | 100 | 100 | 97 | 122 | 106 | 118 | 107 | 109 | 114 |
| All | 1 | 1 | p-value | 0.87 | 0.70 | 0.49 | 0.07 | 0.44 | 0.01 | 0.29 | 0.06 | 0.06 |
| All | 2 | 1 | Construct (T) | 166.5 | 4.1 | 10.0 | 0.525 | 0.235 | 0.419 | 0.230 | 0.514 | 0.595 |
| All | 2 | 1 | Control (C) | 186.8 | 4.2 | 10.4 | 0.480 | 0.230 | 0.357 | 0.225 | 0.506 | 0.578 |
| All | 2 | 1 | T − C | −20.3 | −0.1 | −0.5 | 0.045 | 0.005 | 0.062 | 0.005 | 0.008 | 0.017 |
| All | 2 | 1 | (T/C) % | 89 | 98 | 95 | 109 | 102 | 117 | 102 | 102 | 103 |
| All | 2 | 1 | p-value | 0.00 | 0.59 | 0.03 | 0.02 | 0.52 | 0.00 | 0.74 | 0.48 | 0.32 |
| All | All | 2 | Construct (T) | 160.2 | 4.1 | 9.2 | 0.426 | 0.208 | 0.400 | 0.215 | 0.402 | 0.518 |
| All | All | 2 | Control (C) | 168.3 | 3.9 | 9.5 | 0.395 | 0.205 | 0.345 | 0.208 | 0.399 | 0.498 |
| All | All | 2 | T − C | −8.1 | 0.2 | −0.3 | 0.031 | 0.003 | 0.055 | 0.007 | 0.003 | 0.020 |
| All | All | 2 | (T/C) % | 95 | 105 | 97 | 108 | 101 | 116 | 103 | 101 | 104 |
| All | All | 2 | p-value | 0.28 | 0.01 | 0.21 | 0.02 | 0.75 | 0.00 | 0.38 | 0.69 | 0.13 |
| All | 1 | 2 | Construct (T) | 158.0 | 4.7 | 8.7 | 0.337 | 0.158 | 0.393 | 0.158 | 0.315 | 0.466 |
| All | 1 | 2 | Control (C) | 156.4 | 4.4 | 8.6 | 0.287 | 0.149 | 0.321 | 0.149 | 0.301 | 0.431 |
| All | 1 | 2 | T − C | 1.6 | 0.3 | 0.1 | 0.050 | 0.009 | 0.072 | 0.009 | 0.014 | 0.035 |
| All | 1 | 2 | (T/C) % | 101 | 107 | 101 | 117 | 106 | 122 | 106 | 105 | 108 |
| All | 1 | 2 | p-value | 0.76 | 0.01 | 0.75 | 0.01 | 0.30 | 0.00 | 0.36 | 0.20 | 0.09 |
| All | 2 | 2 | Construct (T) | 164.4 | 3.5 | 9.6 | 0.492 | 0.251 | 0.391 | 0.257 | 0.472 | 0.558 |
| All | 2 | 2 | Control (C) | 176.6 | 3.6 | 9.5 | 0.447 | 0.239 | 0.347 | 0.242 | 0.453 | 0.519 |
| All | 2 | 2 | T − C | −12.2 | −0.1 | 0.1 | 0.045 | 0.012 | 0.044 | 0.015 | 0.019 | 0.039 |
| All | 2 | 2 | (T/C) % | 93 | 97 | 101 | 110 | 105 | 113 | 106 | 104 | 108 |
| All | 2 | 2 | p-value | 0.02 | 0.58 | 0.64 | 0.01 | 0.22 | 0.00 | 0.08 | 0.04 | 0.01 |
| 3Q | All | 1 | Event (T) | 154.0 | 4.8 | 9.4 | 0.445 | 0.201 | 0.439 | 0.203 | 0.427 | 0.554 |
| 3Q | All | 1 | Control (C) | 159.6 | 4.9 | 9.6 | 0.381 | 0.186 | 0.365 | 0.187 | 0.401 | 0.500 |
| 3Q | All | 1 | T − C | −5.6 | −0.1 | −0.2 | 0.064 | 0.015 | 0.074 | 0.016 | 0.026 | 0.054 |
| 3Q | All | 1 | (T/C) % | 96 | 99 | 98 | 117 | 108 | 120 | 109 | 106 | 111 |
| 3Q | All | 1 | p-value | 0.46 | 0.57 | 0.41 | 0.00 | 0.04 | 0.00 | 0.15 | 0.02 | 0.00 |
| 3Q | 1 | 1 | Event (T) | 152.3 | 5.4 | 9.1 | 0.371 | 0.174 | 0.452 | 0.177 | 0.342 | 0.514 |
| 3Q | 1 | 1 | Control (C) | 149.7 | 5.4 | 9.1 | 0.301 | 0.148 | 0.366 | 0.157 | 0.306 | 0.436 |
| 3Q | 1 | 1 | T − C | 2.6 | 0.0 | 0.0 | 0.070 | 0.026 | 0.086 | 0.020 | 0.036 | 0.078 |
| 3Q | 1 | 1 | (T/C) % | 102 | 100 | 100 | 123 | 118 | 123 | 113 | 112 | 118 |
| 3Q | 1 | 1 | p-value | 0.86 | 0.94 | 0.91 | 0.00 | 0.06 | 0.01 | 0.16 | 0.01 | 0.00 |
| 3Q | 2 | 1 | Event (T) | 174.2 | 4.1 | 9.8 | 0.524 | 0.235 | 0.424 | 0.237 | 0.514 | 0.596 |
| 3Q | 2 | 1 | Control (C) | 186.8 | 4.2 | 10.4 | 0.476 | 0.227 | 0.359 | 0.223 | 0.505 | 0.576 |
| 3Q | 2 | 1 | T − C | −12.6 | −0.1 | −0.6 | 0.048 | 0.008 | 0.065 | 0.014 | 0.009 | 0.020 |
| 3Q | 2 | 1 | (T/C) % | 93 | 98 | 94 | 110 | 104 | 118 | 106 | 102 | 103 |
| 3Q | 2 | 1 | p-value | 0.01 | 0.53 | 0.33 | 0.19 | 0.58 | 0.00 | 0.20 | 0.70 | 0.61 |
| 3Q | All | 2 | Event (T) | 169.1 | 4.0 | 8.8 | 0.426 | 0.203 | 0.409 | 0.215 | 0.386 | 0.509 |
| 3Q | All | 2 | Control (C) | 168.3 | 4.0 | 9.3 | 0.380 | 0.204 | 0.336 | 0.207 | 0.387 | 0.487 |
| 3Q | All | 2 | T − C | 0.8 | 0.0 | −0.5 | 0.046 | −0.001 | 0.073 | 0.008 | −0.001 | 0.022 |
| 3Q | All | 2 | (T/C) % | 100 | 101 | 95 | 112 | 100 | 122 | 104 | 100 | 105 |
| 3Q | All | 2 | p-value | 0.94 | 0.66 | 0.11 | 0.03 | 0.96 | 0.00 | 0.37 | 0.94 | 0.24 |
| 3Q | 1 | 2 | Event (T) | 166.4 | 4.6 | 8.3 | 0.326 | 0.147 | 0.394 | 0.157 | 0.298 | 0.452 |
| 3Q | 1 | 2 | Control (C) | 159.0 | 4.4 | 8.7 | 0.292 | 0.152 | 0.328 | 0.151 | 0.299 | 0.432 |
| 3Q | 1 | 2 | T − C | 7.4 | 0.2 | −0.4 | 0.034 | −0.005 | 0.066 | 0.006 | −0.001 | 0.020 |
| 3Q | 1 | 2 | (T/C) % | 105 | 105 | 95 | 112 | 97 | 120 | 104 | 100 | 105 |
| 3Q | 1 | 2 | p-value | 0.59 | 0.14 | 0.05 | 0.01 | 0.45 | 0.00 | 0.45 | 0.90 | 0.18 |
| 3Q | 2 | 2 | Event (T) | 172.3 | 3.4 | 9.4 | 0.503 | 0.252 | 0.408 | 0.256 | 0.471 | 0.562 |
| 3Q | 2 | 2 | Control (C) | 179.5 | 3.6 | 9.2 | 0.434 | 0.233 | 0.342 | 0.234 | 0.442 | 0.507 |
| 3Q | 2 | 2 | T − C | −7.2 | −0.2 | 0.2 | 0.069 | 0.019 | 0.066 | 0.022 | 0.029 | 0.055 |
| 3Q | 2 | 2 | (T/C) % | 96 | 94 | 102 | 116 | 108 | 119 | 109 | 107 | 111 |
| 3Q | 2 | 2 | p-value | 0.32 | 0.53 | 1.00 | 0.04 | 0.47 | 0.01 | 0.24 | 0.43 | 0.22 |
| 3R | All | 1 | Event (T) | 154.7 | 4.8 | 9.2 | 0.411 | 0.182 | 0.421 | 0.180 | 0.418 | 0.528 |
| 3R | All | 1 | Control (C) | 159.6 | 4.9 | 9.6 | 0.381 | 0.186 | 0.365 | 0.187 | 0.401 | 0.500 |
| 3R | All | 1 | T − C | −4.9 | −0.1 | −0.4 | 0.030 | −0.004 | 0.056 | −0.007 | 0.017 | 0.028 |
| 3R | All | 1 | (T/C) % | 97 | 98 | 96 | 108 | 98 | 115 | 96 | 104 | 106 |
| 3R | All | 1 | p-value | 0.55 | 0.33 | 0.26 | 0.16 | 0.47 | 0.01 | 0.54 | 0.23 | 0.24 |
| 3R | 1 | 1 | Event (T) | 157.2 | 5.4 | 8.4 | 0.309 | 0.141 | 0.409 | 0.150 | 0.312 | 0.455 |
| 3R | 1 | 1 | Control (C) | 149.7 | 5.4 | 9.2 | 0.309 | 0.147 | 0.373 | 0.153 | 0.311 | 0.443 |
| 3R | 1 | 1 | T − C | 7.5 | 0.0 | −0.8 | 0.000 | −0.006 | 0.036 | −0.003 | 0.001 | 0.012 |
| 3R | 1 | 1 | (T/C) % | 105 | 100 | 91 | 100 | 96 | 110 | 98 | 100 | 103 |
| 3R | 1 | 1 | p-value | 0.60 | 0.75 | 0.06 | 0.99 | 0.52 | 0.17 | 0.74 | 0.86 | 0.48 |
| 3R | 2 | 1 | Event (T) | 164.3 | 4.0 | 9.5 | 0.512 | 0.220 | 0.426 | 0.213 | 0.504 | 0.579 |
| 3R | 2 | 1 | Control (C) | 186.8 | 4.2 | 10.4 | 0.476 | 0.227 | 0.359 | 0.223 | 0.505 | 0.576 |
| 3R | 2 | 1 | T − C | −22.5 | −0.2 | −0.9 | 0.036 | −0.007 | 0.067 | −0.010 | −0.001 | 0.003 |
| 3R | 2 | 1 | (T/C) % | 88 | 95 | 91 | 108 | 97 | 119 | 96 | 100 | 101 |
| 3R | 2 | 1 | p-value | 0.09 | 0.12 | 0.39 | 0.00 | 0.70 | 0.04 | 0.31 | 0.56 | 0.52 |
| 3R | All | 2 | Event (T) | 170.4 | 4.1 | 8.9 | 0.407 | 0.197 | 0.386 | 0.204 | 0.388 | 0.496 |
| 3R | All | 2 | Control (C) | 168.3 | 4.0 | 9.3 | 0.380 | 0.204 | 0.336 | 0.207 | 0.387 | 0.487 |
| 3R | All | 2 | T − C | 2.1 | 0.1 | −0.4 | 0.027 | −0.007 | 0.050 | −0.003 | 0.001 | 0.009 |
| 3R | All | 2 | (T/C) % | 101 | 102 | 95 | 107 | 97 | 115 | 99 | 100 | 102 |
| 3R | All | 2 | p-value | 0.83 | 0.49 | 0.17 | 0.13 | 0.60 | 0.00 | 0.76 | 0.91 | 0.60 |
| 3R | 1 | 2 | Event (T) | 170.3 | 4.6 | 8.8 | 0.329 | 0.154 | 0.376 | 0.152 | 0.315 | 0.454 |
| 3R | 1 | 2 | Control (C) | 159.0 | 4.4 | 8.7 | 0.292 | 0.152 | 0.328 | 0.151 | 0.299 | 0.432 |
| 3R | 1 | 2 | T − C | 11.3 | 0.2 | 0.1 | 0.037 | 0.002 | 0.048 | 0.001 | 0.016 | 0.022 |
| 3R | 1 | 2 | (T/C) % | 107 | 105 | 101 | 113 | 101 | 115 | 101 | 105 | 105 |
| 3R | 1 | 2 | p-value | 0.42 | 0.04 | 0.61 | 0.02 | 0.75 | 0.01 | 0.88 | 0.17 | 0.17 |
| 3R | 2 | 2 | Event (T) | 167.6 | 3.5 | 9.4 | 0.481 | 0.246 | 0.381 | 0.253 | 0.469 | 0.555 |
| 3R | 2 | 2 | Control (C) | 179.5 | 3.6 | 9.2 | 0.434 | 0.233 | 0.342 | 0.234 | 0.442 | 0.507 |
| 3R | 2 | 2 | T − C | −11.9 | −0.1 | 0.2 | 0.047 | 0.013 | 0.039 | 0.019 | 0.027 | 0.048 |
| 3R | 2 | 2 | (T/C) % | 93 | 97 | 102 | 111 | 106 | 111 | 108 | 106 | 109 |
| 3R | 2 | 2 | p-value | 0.42 | 0.82 | 0.83 | 0.50 | 0.89 | 0.00 | 0.72 | 0.77 | 0.63 |
| 3S | All | 1 | Event (T) | 153.1 | 4.1 | 9.6 | 0.426 | 0.212 | 0.400 | 0.227 | 0.397 | 0.527 |
| 3S | All | 1 | Control (C) | 168.3 | 4.0 | 9.3 | 0.380 | 0.204 | 0.336 | 0.207 | 0.387 | 0.487 |
| 3S | All | 1 | T − C | −15.2 | 0.1 | 0.3 | 0.046 | 0.008 | 0.064 | 0.020 | 0.010 | 0.040 |
| 3S | All | 1 | (T/C) % | 91 | 102 | 103 | 112 | 104 | 119 | 110 | 103 | 108 |
| 3S | All | 1 | p-value | 0.02 | 0.95 | 0.46 | 0.00 | 0.06 | 0.00 | 0.34 | 0.10 | 0.03 |
| 3S | 1 | 1 | Event (T) | 139.1 | 5.5 | 8.8 | 0.360 | 0.169 | 0.423 | 0.187 | 0.331 | 0.502 |
| 3S | 1 | 1 | Control (C) | 149.7 | 5.4 | 9.1 | 0.301 | 0.148 | 0.366 | 0.157 | 0.306 | 0.436 |
| 3S | 1 | 1 | T − C | −10.6 | 0.1 | −0.3 | 0.059 | 0.021 | 0.057 | 0.030 | 0.025 | 0.066 |
| 3S | 1 | 1 | (T/C) % | 93 | 102 | 97 | 120 | 114 | 116 | 119 | 108 | 115 |
| 3S | 1 | 1 | p-value | 0.46 | 0.53 | 0.50 | 0.11 | 0.08 | 0.03 | 0.01 | 0.17 | 0.09 |
| 3S | 2 | 1 | Event (T) | 160.4 | 4.2 | 9.9 | 0.525 | 0.233 | 0.422 | 0.215 | 0.515 | 0.599 |
| 3S | 2 | 1 | Control (C) | 186.8 | 4.2 | 10.4 | 0.476 | 0.227 | 0.359 | 0.223 | 0.505 | 0.576 |
| 3S | 2 | 1 | T − C | −26.4 | 0.0 | −0.5 | 0.049 | 0.006 | 0.063 | −0.008 | 0.010 | 0.023 |
| 3S | 2 | 1 | (T/C) % | 86 | 100 | 95 | 110 | 103 | 118 | 96 | 102 | 104 |
| 3S | 2 | 1 | p-value | 0.01 | 0.69 | 0.53 | 0.00 | 0.41 | 0.09 | 0.58 | 0.38 | 0.22 |
| 3S | All | 2 | Event (T) | 137.9 | 4.9 | 9.4 | 0.445 | 0.201 | 0.423 | 0.201 | 0.424 | 0.553 |
| 3S | All | 2 | Control (C) | 159.6 | 4.9 | 9.6 | 0.381 | 0.186 | 0.365 | 0.187 | 0.401 | 0.500 |
| 3S | All | 2 | T − C | −21.7 | 0.0 | −0.2 | 0.064 | 0.015 | 0.058 | 0.014 | 0.023 | 0.053 |
| 3S | All | 2 | (T/C) % | 86 | 100 | 98 | 117 | 108 | 116 | 107 | 106 | 111 |
| 3S | All | 2 | p-value | 0.08 | 0.17 | 0.30 | 0.01 | 0.35 | 0.00 | 0.04 | 0.43 | 0.03 |
| 3S | 1 | 2 | Event (T) | 153.3 | 4.7 | 9.2 | 0.376 | 0.180 | 0.422 | 0.184 | 0.330 | 0.506 |
| 3S | 1 | 2 | Control (C) | 159.0 | 4.4 | 8.7 | 0.292 | 0.152 | 0.328 | 0.151 | 0.299 | 0.432 |
| 3S | 1 | 2 | T − C | −5.7 | 0.3 | 0.5 | 0.084 | 0.028 | 0.094 | 0.033 | 0.031 | 0.074 |
| 3S | 1 | 2 | (T/C) % | 96 | 107 | 106 | 129 | 118 | 129 | 122 | 110 | 117 |
| 3S | 1 | 2 | p-value | 0.71 | 0.02 | 0.26 | 0.00 | 0.04 | 0.00 | 0.02 | 0.08 | 0.03 |
| 3S | 2 | 2 | Event (T) | 155.6 | 3.5 | 9.7 | 0.459 | 0.239 | 0.367 | 0.240 | 0.450 | 0.538 |
| 3S | 2 | 2 | Control (C) | 179.5 | 3.6 | 9.2 | 0.434 | 0.233 | 0.342 | 0.234 | 0.442 | 0.507 |
| 3S | 2 | 2 | T − C | −23.9 | −0.1 | 0.5 | 0.025 | 0.006 | 0.025 | 0.006 | 0.008 | 0.031 |
| 3S | 2 | 2 | (T/C) % | 87 | 97 | 105 | 106 | 103 | 107 | 103 | 102 | 106 |
| 3S | 2 | 2 | p-value | 0.03 | 0.95 | 0.61 | 0.57 | 0.84 | 0.19 | 0.39 | 0.71 | 0.55 |
| 3T | All | 1 | Event (T) | 155.8 | 4.5 | 9.6 | 0.448 | 0.206 | 0.398 | 0.211 | 0.449 | 0.541 |
| 3T | All | 1 | Control (C) | 159.6 | 4.8 | 9.7 | 0.389 | 0.188 | 0.363 | 0.190 | 0.405 | 0.506 |
| 3T | All | 1 | T − C | −3.8 | −0.3 | −0.1 | 0.059 | 0.018 | 0.035 | 0.021 | 0.044 | 0.035 |
| 3T | All | 1 | (T/C) % | 98 | 94 | 99 | 115 | 110 | 110 | 111 | 111 | 107 |
| 3T | All | 1 | p-value | 0.95 | 1.00 | 0.22 | 0.23 | 0.60 | 0.05 | 0.40 | 0.79 | 0.59 |
| 3T | 1 | 1 | Event (T) | 158.9 | 5.3 | 8.7 | 0.316 | 0.145 | 0.429 | 0.151 | 0.316 | 0.453 |
| 3T | 1 | 1 | Control (C) | 149.7 | 5.4 | 9.0 | 0.304 | 0.143 | 0.364 | 0.152 | 0.306 | 0.433 |
| 3T | 1 | 1 | T − C | 9.2 | −0.1 | −0.3 | 0.012 | 0.002 | 0.065 | −0.001 | 0.010 | 0.020 |
| 3T | 1 | 1 | (T/C) % | 106 | 98 | 97 | 104 | 101 | 118 | 99 | 103 | 105 |
| 3T | 1 | 1 | p-value | 0.52 | 0.67 | 0.27 | 0.49 | 0.82 | 0.06 | 0.88 | 0.30 | 0.15 |
| 3T | 2 | 1 | Event (T) | 167.1 | 4.2 | 10.0 | 0.498 | 0.229 | 0.386 | 0.234 | 0.499 | 0.574 |
| 3T | 2 | 1 | Control (C) | 186.8 | 4.2 | 10.4 | 0.476 | 0.227 | 0.359 | 0.223 | 0.505 | 0.576 |
| 3T | 2 | 1 | T − C | −19.7 | 0.0 | −0.4 | 0.022 | 0.002 | 0.027 | 0.011 | −0.006 | −0.002 |
| 3T | 2 | 1 | (T/C) % | 89 | 100 | 96 | 105 | 101 | 108 | 105 | 99 | 100 |
| 3T | 2 | 1 | p-value | 0.02 | 0.60 | 0.54 | 0.51 | 0.79 | 0.14 | 0.60 | 0.94 | 0.76 |
| 3T | All | 2 | Event (T) | 157.4 | 4.2 | 8.9 | 0.398 | 0.201 | 0.389 | 0.198 | 0.386 | 0.492 |
| 3T | All | 2 | Control (C) | 168.3 | 4.0 | 9.3 | 0.380 | 0.204 | 0.336 | 0.207 | 0.387 | 0.487 |
| 3T | All | 2 | T − C | −10.9 | 0.2 | −0.5 | 0.018 | −0.003 | 0.053 | −0.009 | −0.001 | 0.005 |
| 3T | All | 2 | (T/C) % | 94 | 105 | 95 | 105 | 99 | 116 | 96 | 100 | 101 |
| 3T | All | 2 | p-value | 0.18 | 0.12 | 0.04 | 0.25 | 0.81 | 0.00 | 0.26 | 0.88 | 0.72 |
| 3T | 1 | 2 | Event (T) | 152.2 | 4.7 | 8.3 | 0.311 | 0.146 | 0.379 | 0.145 | 0.302 | 0.440 |
| 3T | 1 | 2 | Control (C) | 159.0 | 4.4 | 8.7 | 0.292 | 0.152 | 0.328 | 0.151 | 0.299 | 0.432 |
| 3T | 1 | 2 | T − C | −6.8 | 0.3 | −0.4 | 0.019 | −0.006 | 0.051 | −0.006 | 0.003 | 0.008 |
| 3T | 1 | 2 | (T/C) % | 96 | 107 | 95 | 107 | 96 | 116 | 96 | 101 | 102 |
| 3T | 1 | 2 | p-value | 0.62 | 0.03 | 0.04 | 0.22 | 0.37 | 0.01 | 0.55 | 0.72 | 0.62 |
| 3T | 2 | 2 | Event (T) | 168.7 | 3.5 | 9.5 | 0.492 | 0.258 | 0.388 | 0.257 | 0.470 | 0.558 |
| 3T | 2 | 2 | Control (C) | 179.5 | 3.6 | 9.2 | 0.434 | 0.233 | 0.342 | 0.234 | 0.442 | 0.507 |
| 3T | 2 | 2 | T − C | −10.8 | −0.1 | 0.3 | 0.058 | 0.025 | 0.046 | 0.023 | 0.028 | 0.051 |
| 3T | 2 | 2 | (T/C) % | 94 | 97 | 103 | 113 | 111 | 113 | 110 | 106 | 110 |
| 3T | 2 | 2 | p-value | 0.07 | 0.94 | 0.81 | 0.21 | 0.38 | 0.02 | 0.50 | 0.47 | 0.34 |

TABLE 22
Summary of field data for Construct 4. Numbers shown in bold are significantly different from the control at the
p-value shown. bu/a is bushels per acre. “All” indicates the average across all events or testers. (T/C) %
is the value for the transgenic hybrid combination (T) expressed as a percent of the control (C).

| | | Yield | Oil | Protein | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p < 0.1 | p < 0.05 | p < 0.05 | p < 0.05 | p < 0.05 | p < 0.05 | p < 0.05 | p < 0.05 | p < 0.05 |
| All | (T/C) % | 101 | 102 | 102 | 103 | 102 | 101 | 98 | 101 | 102 |
| 4V | (T/C) % | 103 | 102 | 101 | 101 | 99 | 100 | 96 | 100 | 101 |
| 4W | (T/C) % | 100 | 102 | 103 | 105 | 104 | 102 | 100 | 102 | 104 |

TABLE 23
Field data for Construct 4. Numbers shown in bold are significantly different from the control at the p-value shown.
bu/a is bushels per acre. “All” indicates the average across all events or testers. (T/C) % is the value for the
transgenic hybrid combination expressed as a percent of the control. T − C is the transgenic hybrid combination
minus the control. Oil, protein, and amino acid content are shown as percent of seed dry weight.

| | | Yield | Oil | Protein | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p < 0.1 | p < 0.05 | p < 0.05 | p < 0.05 | p < 0.05 | p < 0.05 | p < 0.05 | p < 0.05 | p < 0.05 |
| All | Construct (T) | 160.1 | 5.3 | 9.7 | 0.323 | 0.166 | 0.333 | 0.172 | 0.319 | 0.473 |
| All | Control (C) | 157.8 | 5.2 | 9.6 | 0.314 | 0.163 | 0.331 | 0.176 | 0.315 | 0.462 |
| All | T − C | 2.3 | 0.1 | 0.2 | 0.009 | 0.003 | 0.002 | −0.004 | 0.004 | 0.011 |
| All | (T/C) % | 101 | 102 | 102 | 103 | 102 | 101 | 98 | 101 | 102 |
| All | p-value | 0.51 | 0.34 | 0.44 | 0.32 | 0.66 | 0.81 | 0.46 | 0.57 | 0.29 |
| 4V | Event (T) | 162.2 | 5.2 | 9.6 | 0.317 | 0.162 | 0.330 | 0.168 | 0.315 | 0.466 |
| 4V | Control (C) | 157.9 | 5.2 | 9.6 | 0.314 | 0.163 | 0.331 | 0.176 | 0.315 | 0.462 |
| 4V | T − C | 4.4 | 0.1 | 0.1 | 0.003 | −0.002 | −0.001 | −0.008 | 0.000 | 0.004 |
| 4V | (T/C) % | 103 | 102 | 101 | 101 | 99 | 100 | 96 | 100 | 101 |
| 4V | p-value | 0.30 | 0.48 | 0.79 | 0.76 | 0.81 | 0.92 | 0.20 | 0.96 | 0.71 |
| 4W | Event (T) | 158.1 | 5.3 | 9.8 | 0.329 | 0.171 | 0.337 | 0.177 | 0.322 | 0.480 |
| 4W | Control (C) | 157.8 | 5.2 | 9.6 | 0.314 | 0.163 | 0.331 | 0.176 | 0.315 | 0.462 |
| 4W | T − C | 0.3 | 0.1 | 0.3 | 0.015 | 0.007 | 0.005 | 0.000 | 0.007 | 0.018 |
| 4W | (T/C) % | 100 | 102 | 103 | 105 | 104 | 102 | 100 | 102 | 104 |
| 4W | p-value | 0.95 | 0.35 | 0.32 | 0.15 | 0.34 | 0.59 | 0.98 | 0.36 | 0.15 |

TABLE 24
Summary of field data for Construct 4 by year. Numbers shown in bold are significantly different from the
control at the p-value shown. bu/a is bushels per acre. “All” indicates the average across both years.
(T/C) % is the value for the transgenic hybrid combination (T) expressed as a percent of the control (C).

| | | Yield | Oil | Protein | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Year | Description | p < 0.1 | p < 0.05 | p < 0.05 | p < 0.05 | p < 0.05 | p < 0.05 | p < 0.05 | p < 0.05 | p < 0.05 |
| All | (T/C) % | 98 | 104 | 104 | 104 | 113 | 101 | 106 | 105 | 105 |
| 2 | (T/C) % | 98 | 103 | 97 | 101 | 102 | 100 | 99 | 100 | 102 |
| 1 | (T/C) % | 102 | 102 | 108 | 105 | 115 | 101 | 106 | 106 | 106 |

TABLE 25
Summary of field data for Construct 5 for Year 1. Numbers shown in bold are significantly different from the control
at the p-value shown. bu/a is bushels per acre. “All” indicates the average across all events or testers. (T/C) %
is the value for the transgenic hybrid combination (T) expressed as a percent of the control (C).

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | (T/C) % | 93 | 102 | 112 | 113 | 108 | 111 | 109 | 106 | 109 |
| 5X | (T/C) % | 94 | 106 | 112 | 117 | 113 | 112 | 115 | 108 | 111 |
| 5Y | (T/C) % | 98 | 109 | 112 | 119 | 111 | 120 | 112 | 108 | 112 |
| 5Z | (T/C) % | 98 | 109 | 112 | 119 | 111 | 120 | 112 | 108 | 112 |
| 5A1 | (T/C) % | 94 | 104 | 112 | 110 | 103 | 112 | 102 | 107 | 108 |
| 5B1 | (T/C) % | 83 | 106 | 111 | 115 | 113 | 112 | 110 | 107 | 110 |
| 5C1 | (T/C) % | 102 | 106 | 115 | 117 | 124 | 106 | 118 | 110 | 112 |

TABLE 26
Field data for Construct 5 for Year 1. Numbers shown in bold are significantly different from the control at the p-value
shown. bu/a is bushels per acre. “All” indicates the average across all events or testers. (T/C) % is the value
for the transgenic hybrid combination (T) expressed as a percent of the control (C). T − C is the transgenic hybrid
combination minus the control. Oil, protein, and amino acid content are shown as percent of seed dry weight.

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | Construct (T) | 134.9 | 5.0 | 9.9 | 0.335 | 0.171 | 0.355 | 0.182 | 0.309 | 0.476 |
| All | Control (C) | 144.8 | 4.9 | 8.8 | 0.297 | 0.158 | 0.321 | 0.167 | 0.292 | 0.435 |
| All | T − C | −9.9 | 0.1 | 1.1 | 0.038 | 0.013 | 0.034 | 0.015 | 0.017 | 0.041 |
| All | (T/C) % | 93 | 102 | 112 | 113 | 108 | 111 | 109 | 106 | 109 |
| All | p-value | 0.09 | 0.39 | 0.00 | 0.00 | 0.06 | 0.00 | 0.01 | 0.01 | 0.00 |
| 5X | Event (T) | 136.4 | 5.0 | 9.9 | 0.342 | 0.171 | 0.359 | 0.187 | 0.309 | 0.481 |
| 5X | Control (C) | 144.8 | 4.7 | 8.8 | 0.293 | 0.151 | 0.320 | 0.163 | 0.286 | 0.432 |
| 5X | T − C | −8.4 | 0.3 | 1.1 | 0.049 | 0.020 | 0.039 | 0.024 | 0.023 | 0.049 |
| 5X | (T/C) % | 94 | 106 | 112 | 117 | 113 | 112 | 115 | 108 | 111 |
| 5X | p-value | 0.32 | 0.23 | 0.00 | 0.00 | 0.02 | 0.06 | 0.01 | 0.01 | 0.00 |
| 5Y | Event (T) | 141.8 | 5.1 | 9.9 | 0.347 | 0.168 | 0.383 | 0.182 | 0.311 | 0.483 |
| 5Y | Control (C) | 144.8 | 4.7 | 8.8 | 0.292 | 0.151 | 0.318 | 0.163 | 0.287 | 0.433 |
| 5Y | T − C | −3.0 | 0.4 | 1.1 | 0.055 | 0.017 | 0.065 | 0.019 | 0.024 | 0.050 |
| 5Y | (T/C) % | 98 | 109 | 112 | 119 | 111 | 120 | 112 | 108 | 112 |
| 5Y | p-value | 0.73 | 0.13 | 0.00 | 0.00 | 0.04 | 0.00 | 0.01 | 0.01 | 0.00 |
| 5Z | Event (T) | 141.8 | 5.1 | 9.9 | 0.347 | 0.168 | 0.383 | 0.182 | 0.311 | 0.483 |
| 5Z | Control (C) | 144.8 | 4.7 | 8.8 | 0.292 | 0.151 | 0.318 | 0.163 | 0.287 | 0.433 |
| 5Z | T − C | −3.0 | 0.4 | 1.1 | 0.055 | 0.017 | 0.065 | 0.019 | 0.024 | 0.050 |
| 5Z | (T/C) % | 98 | 109 | 112 | 119 | 111 | 120 | 112 | 108 | 112 |
| 5Z | p-value | 0.73 | 0.13 | 0.00 | 0.00 | 0.04 | 0.00 | 0.01 | 0.01 | 0.00 |
| 5A1 | Event (T) | 135.4 | 4.9 | 9.9 | 0.321 | 0.155 | 0.358 | 0.166 | 0.305 | 0.466 |
| 5A1 | Control (C) | 144.8 | 4.7 | 8.8 | 0.293 | 0.151 | 0.320 | 0.163 | 0.286 | 0.432 |
| 5A1 | T − C | −9.4 | 0.2 | 1.1 | 0.028 | 0.004 | 0.038 | 0.003 | 0.019 | 0.034 |
| 5A1 | (T/C) % | 94 | 104 | 112 | 110 | 103 | 112 | 102 | 107 | 108 |
| 5A1 | p-value | 0.27 | 0.42 | 0.00 | 0.04 | 0.70 | 0.04 | 0.72 | 0.04 | 0.03 |
| 5B1 | Event (T) | 120.4 | 5.2 | 9.8 | 0.343 | 0.179 | 0.359 | 0.183 | 0.312 | 0.477 |
| 5B1 | Control (C) | 144.8 | 4.9 | 8.8 | 0.297 | 0.158 | 0.321 | 0.167 | 0.292 | 0.435 |
| 5B1 | T − C | −24.4 | 0.3 | 1.0 | 0.046 | 0.021 | 0.038 | 0.016 | 0.020 | 0.042 |
| 5B1 | (T/C) % | 83 | 106 | 111 | 115 | 113 | 112 | 110 | 107 | 110 |
| 5B1 | p-value | 0.01 | 0.18 | 0.00 | 0.00 | 0.03 | 0.02 | 0.02 | 0.03 | 0.00 |
| 5C1 | Event (T) | 147.1 | 5.0 | 10.1 | 0.341 | 0.187 | 0.336 | 0.192 | 0.317 | 0.487 |
| 5C1 | Control (C) | 144.8 | 4.7 | 8.8 | 0.292 | 0.151 | 0.318 | 0.163 | 0.287 | 0.433 |
| 5C1 | T − C | 2.3 | 0.3 | 1.3 | 0.049 | 0.036 | 0.018 | 0.029 | 0.030 | 0.054 |
| 5C1 | (T/C) % | 102 | 106 | 115 | 117 | 124 | 106 | 118 | 110 | 112 |
| 5C1 | p-value | 0.80 | 0.19 | 0.00 | 0.00 | 0.00 | 0.28 | 0.00 | 0.00 | 0.00 |

TABLE 27
Summary of field data for Construct 5 for Year 2. Numbers shown in bold are significantly different from the control at the
p-value shown. bu/a is bushels per acre. The events are an average over four testers. “All” indicates the average across
all events or testers. (T/C) % is the value for the transgenic hybrid combination (T) expressed as a percent of the control (C).

| | | Yield | Oil | Prot | Arg | Cys | Ile | Lys | Met | Thr | Val |
|---|---|---|---|---|---|---|---|---|---|---|---|
| | | (bu/a) | (%) | (%) | (%) | (%) | (%) | (%) | (%) | (%) | (%) |
| Event | Description | p <= 0.10 | p <= 0.15 | p <= 0.15 | p <= 0.15 | p <= 0.15 | P <= 0.05 | p <= 0.15 | p <= 0.15 | p <= 0.15 | p <= 0.15 |
| All | (T/C) % | 92 | 103 | 113 | 116 | 110 | 106 | 114 | 109 | 108 | 111 |
| 5X | (T/C) % | 90 | 103 | 117 | 121 | 111 | 109 | 116 | 112 | 110 | 114 |
| 5Y | (T/C) % | 90 | 103 | 112 | 116 | 108 | 106 | 115 | 109 | 109 | 111 |
| 5Z | (T/C) % | 94 | 105 | 111 | 112 | 105 | 104 | 114 | 105 | 106 | 109 |
| 5A1 | (T/C) % | 88 | 108 | 112 | 119 | 113 | 106 | 117 | 111 | 108 | 111 |
| 5B1 | (T/C) % | 94 | 100 | 110 | 113 | 108 | 106 | 110 | 108 | 107 | 110 |
| 5C1 | (T/C) % | 97 | 103 | 112 | 117 | 113 | 107 | 113 | 112 | 109 | 112 |

TABLE 28
Field data for Construct 5 for Year 2. Numbers shown in bold are significantly different from the control at the p-value shown. bu/a
is bushels per acre. The events are an average over four testers. “All” indicates the average across all events or testers. (T/C) % is the
value for the transgenic hybrid combination (T) expressed as a percent of the control (C). T − C is the transgenic hybrid combination
minus the control. Oil, protein, and amino acid content are shown as percent of seed dry weight.

| | | Yield | Oil | Prot | Arg | Cys | Ile | Lys | Met | Thr | Val |
|---|---|---|---|---|---|---|---|---|---|---|---|
| | | (bu/a) | (%) | (%) | (%) | (%) | (%) | (%) | (%) | (%) | (%) |
| Event | Description | p <= 0.10 | p <= 0.15 | p <= 0.15 | p <= 0.15 | p <= 0.15 | P <= 0.05 | p <= 0.15 | p <= 0.15 | p <= 0.15 | p <= 0.15 |
| All | Construct (T) | 160.4 | 4.0 | 11.3 | 0.541 | 0.251 | 0.396 | 0.407 | 0.256 | 0.515 | 0.604 |
| All | Control (C) | 174.3 | 3.9 | 10.0 | 0.465 | 0.229 | 0.372 | 0.356 | 0.234 | 0.476 | 0.543 |
| All | T − C | −13.9 | 0.1 | 1.3 | 0.076 | 0.022 | 0.024 | 0.051 | 0.022 | 0.039 | 0.061 |
| All | (T/C) % | 92 | 103 | 113 | 116 | 110 | 106 | 114 | 109 | 108 | 111 |
| All | p-value | 0.00 | 0.14 | 0.01 | 0.01 | 0.12 | 0.06 | 0.00 | 0.06 | 0.02 | 0.01 |
| 5X | Event (T) | 156.4 | 4.0 | 11.7 | 0.563 | 0.255 | 0.405 | 0.413 | 0.262 | 0.523 | 0.620 |
| 5X | Control (C) | 174.3 | 3.9 | 10.0 | 0.465 | 0.229 | 0.372 | 0.356 | 0.234 | 0.476 | 0.543 |
| 5X | T − C | −17.9 | 0.1 | 1.7 | 0.098 | 0.026 | 0.033 | 0.057 | 0.028 | 0.047 | 0.077 |
| 5X | (T/C) % | 90 | 103 | 117 | 121 | 111 | 109 | 116 | 112 | 110 | 114 |
| 5X | p-value | 0.04 | 0.51 | 0.01 | 0.04 | 0.14 | 0.18 | 0.01 | 0.08 | 0.07 | 0.05 |
| 5Y | Event (T) | 156.5 | 4.0 | 11.2 | 0.538 | 0.247 | 0.395 | 0.411 | 0.254 | 0.517 | 0.605 |
| 5Y | Control (C) | 174.3 | 3.9 | 10.0 | 0.465 | 0.229 | 0.372 | 0.356 | 0.234 | 0.476 | 0.543 |
| 5Y | T − C | −17.8 | 0.1 | 1.2 | 0.073 | 0.018 | 0.023 | 0.055 | 0.020 | 0.041 | 0.062 |
| 5Y | (T/C) % | 90 | 103 | 112 | 116 | 108 | 106 | 115 | 109 | 109 | 111 |
| 5Y | p-value | 0.01 | 0.40 | 0.00 | 0.02 | 0.15 | 0.05 | 0.00 | 0.07 | 0.01 | 0.01 |
| 5Z | Event (T) | 163.9 | 4.1 | 11.1 | 0.523 | 0.241 | 0.386 | 0.405 | 0.246 | 0.504 | 0.590 |
| 5Z | Control (C) | 174.3 | 3.9 | 10.0 | 0.465 | 0.229 | 0.372 | 0.356 | 0.234 | 0.476 | 0.543 |
| 5Z | T − C | −10.4 | 0.2 | 1.1 | 0.058 | 0.012 | 0.014 | 0.049 | 0.012 | 0.028 | 0.047 |
| 5Z | (T/C) % | 94 | 105 | 111 | 112 | 105 | 104 | 114 | 105 | 106 | 109 |
| 5Z | p-value | 0.02 | 0.00 | 0.01 | 0.00 | 0.20 | 0.09 | 0.01 | 0.14 | 0.00 | 0.00 |
| 5A1 | Event (T) | 154.2 | 4.2 | 11.2 | 0.552 | 0.258 | 0.393 | 0.416 | 0.260 | 0.515 | 0.605 |
| 5A1 | Control (C) | 174.3 | 3.9 | 10.0 | 0.465 | 0.229 | 0.372 | 0.356 | 0.234 | 0.476 | 0.543 |
| 5A1 | T − C | −20.1 | 0.3 | 1.2 | 0.087 | 0.029 | 0.021 | 0.060 | 0.026 | 0.039 | 0.062 |
| 5A1 | (T/C) % | 88 | 108 | 112 | 119 | 113 | 106 | 117 | 111 | 108 | 111 |
| 5A1 | p-value | 0.01 | 0.05 | 0.05 | 0.01 | 0.04 | 0.16 | 0.01 | 0.02 | 0.04 | 0.03 |
| 5B1 | Event (T) | 163.2 | 4.0 | 11.1 | 0.529 | 0.247 | 0.398 | 0.392 | 0.253 | 0.514 | 0.600 |
| 5B1 | Control (C) | 174.3 | 4.0 | 10.1 | 0.468 | 0.229 | 0.374 | 0.357 | 0.235 | 0.479 | 0.546 |
| 5B1 | T − C | −11.2 | 0.0 | 1.0 | 0.061 | 0.018 | 0.024 | 0.035 | 0.018 | 0.035 | 0.054 |
| 5B1 | (T/C) % | 94 | 100 | 110 | 113 | 108 | 106 | 110 | 108 | 107 | 110 |
| 5B1 | p-value | 0.05 | 0.97 | 0.02 | 0.01 | 0.10 | 0.09 | 0.00 | 0.12 | 0.03 | 0.02 |
| 5C1 | Event (T) | 168.9 | 4.0 | 11.2 | 0.544 | 0.259 | 0.399 | 0.402 | 0.261 | 0.519 | 0.606 |
| 5C1 | Control (C) | 174.3 | 3.9 | 10.0 | 0.465 | 0.229 | 0.372 | 0.356 | 0.234 | 0.476 | 0.543 |
| 5C1 | T − C | −5.4 | 0.1 | 1.2 | 0.079 | 0.030 | 0.027 | 0.046 | 0.027 | 0.043 | 0.063 |
| 5C1 | (T/C) % | 97 | 103 | 112 | 117 | 113 | 107 | 113 | 112 | 109 | 112 |
| 5C1 | p-value | 0.23 | 0.40 | 0.00 | 0.01 | 0.13 | 0.06 | 0.02 | 0.09 | 0.00 | 0.00 |

TABLE 29
Field data for Construct 5 by Year by Tester. Numbers shown in bold are significantly different from the control at the p-value shown.
bu/a is bushels per acre. “All” indicates the average across all events or years. (T/C) % is the value for the transgenic hybrid combination (T)
expressed as a percent of the control (C). T − C is the transgenic hybrid combination minus the control. Oil, protein, and amino acid content are
shown as percent of seed dry weight.

| | | | | Yield | Oil | Prot | Arg | Cys | Lys | Met | Thr | Val |
|---|---|---|---|---|---|---|---|---|---|---|---|---|
| | | | | (bu/a) | (%) | (%) | (%) | (%) | (%) | (%) | (%) | (%) |
| Event | Year | Tester | Description | p <= 0.10 | p <= 0.15 | p <= 0.15 | p <= 0.15 | p <= 0.15 | p <= 0.15 | p <= 0.15 | p <= 0.15 | p <= 0.15 |
| All | All | 1 | Construct (T) | 136.8 | 5.0 | 10.4 | 0.431 | 0.214 | 0.382 | 0.222 | 0.416 | 0.531 |
| All | All | 1 | Control (C) | 149.0 | 4.7 | 9.1 | 0.376 | 0.191 | 0.350 | 0.203 | 0.388 | 0.482 |
| All | All | 1 | T − C | −12.2 | 0.3 | 1.3 | 0.055 | 0.023 | 0.032 | 0.019 | 0.028 | 0.049 |
| All | All | 1 | (T/C) % | 92 | 106 | 114 | 115 | 112 | 109 | 109 | 107 | 110 |
| All | All | 1 | p-value | 0.16 | 0.00 | 0.00 | 0.00 | 0.00 | 0.00 | 0.00 | 0.00 | 0.00 |
| All | 1 | 1 | Construct (T) | 128.3 | 5.7 | 9.7 | 0.333 | 0.169 | 0.357 | 0.189 | 0.313 | 0.472 |
| All | 1 | 1 | Control (C) | 137.1 | 5.4 | 8.8 | 0.305 | 0.164 | 0.325 | 0.169 | 0.299 | 0.439 |
| All | 1 | 1 | T − C | −8.8 | 0.3 | 0.9 | 0.028 | 0.005 | 0.032 | 0.020 | 0.014 | 0.033 |
| All | 1 | 1 | (T/C) % | 94 | 106 | 110 | 109 | 103 | 110 | 112 | 105 | 108 |
| All | 1 | 1 | p-value | 0.02 | 0.04 | 0.01 | 0.02 | 0.58 | 0.15 | 0.00 | 0.12 | 0.02 |
| All | 2 | 1 | Construct (T) | 157.6 | 4.2 | 11.2 | 0.536 | 0.257 | 0.398 | 0.263 | 0.526 | 0.604 |
| All | 2 | 1 | Control (C) | 171.5 | 4.0 | 9.9 | 0.475 | 0.233 | 0.362 | 0.243 | 0.493 | 0.552 |
| All | 2 | 1 | T − C | −13.9 | 0.2 | 1.3 | 0.061 | 0.024 | 0.036 | 0.020 | 0.033 | 0.052 |
| All | 2 | 1 | (T/C) % | 92 | 105 | 113 | 113 | 110 | 110 | 108 | 107 | 109 |
| All | 2 | 1 | p-value | 0.00 | 0.00 | 0.00 | 0.00 | 0.01 | 0.01 | 0.01 | 0.00 | 0.00 |
| All | All | 2 | Construct (T) | 152.3 | 4.0 | 10.3 | 0.419 | 0.211 | 0.365 | 0.217 | 0.394 | 0.527 |
| All | All | 2 | Control (C) | 163.5 | 3.9 | 9.1 | 0.353 | 0.190 | 0.318 | 0.196 | 0.359 | 0.467 |
| All | All | 2 | T − C | −11.2 | 0.1 | 1.2 | 0.066 | 0.021 | 0.047 | 0.021 | 0.035 | 0.060 |
| All | All | 2 | (T/C) % | 93 | 103 | 113 | 119 | 111 | 115 | 111 | 110 | 113 |
| All | All | 2 | p-value | 0.01 | 0.08 | 0.00 | 0.00 | 0.00 | 0.00 | 0.00 | 0.00 | 0.00 |
| All | 1 | 2 | Construct (T) | 141.9 | 4.7 | 10.0 | 0.336 | 0.172 | 0.345 | 0.182 | 0.312 | 0.484 |
| All | 1 | 2 | Control (C) | 152.6 | 4.4 | 8.8 | 0.290 | 0.152 | 0.303 | 0.163 | 0.289 | 0.436 |
| All | 1 | 2 | T − C | −10.7 | 0.3 | 1.2 | 0.046 | 0.020 | 0.042 | 0.019 | 0.023 | 0.048 |
| All | 1 | 2 | (T/C) % | 93 | 107 | 114 | 116 | 113 | 114 | 112 | 108 | 111 |
| All | 1 | 2 | p-value | 0.01 | 0.02 | 0.00 | 0.00 | 0.05 | 0.00 | 0.02 | 0.01 | 0.00 |
| All | 2 | 2 | Construct (T) | 161.7 | 3.4 | 10.8 | 0.506 | 0.253 | 0.388 | 0.254 | 0.481 | 0.576 |
| All | 2 | 2 | Control (C) | 172.6 | 3.4 | 9.5 | 0.422 | 0.229 | 0.335 | 0.228 | 0.434 | 0.504 |
| All | 2 | 2 | T − C | −10.9 | 0.0 | 1.3 | 0.084 | 0.024 | 0.053 | 0.026 | 0.047 | 0.072 |
| All | 2 | 2 | (T/C) % | 94 | 100 | 114 | 120 | 110 | 116 | 111 | 111 | 114 |
| All | 2 | 2 | p-value | 0.10 | 0.55 | 0.02 | 0.02 | 0.00 | 0.00 | 0.04 | 0.01 | 0.03 |
| 5X | All | 1 | Event (T) | 137.3 | 4.8 | 10.6 | 0.463 | 0.218 | 0.395 | 0.233 | 0.450 | 0.543 |
| 5X | All | 1 | Control (C) | 151.6 | 4.7 | 9.3 | 0.391 | 0.198 | 0.353 | 0.208 | 0.398 | 0.494 |
| 5X | All | 1 | T − C | −14.3 | 0.1 | 1.3 | 0.072 | 0.020 | 0.042 | 0.025 | 0.052 | 0.049 |
| 5X | All | 1 | (T/C) % | 91 | 102 | 114 | 118 | 110 | 112 | 112 | 113 | 110 |
| 5X | All | 1 | p-value | 0.11 | 0.00 | 0.00 | 0.09 | 0.51 | 0.05 | 0.16 | 0.40 | 0.18 |
| 5X | 1 | 1 | Event (T) | 127.1 | 6.3 | 9.0 | 0.315 | 0.156 | 0.382 | 0.176 | 0.281 | 0.435 |
| 5X | 1 | 1 | Control (C) | 137.1 | 5.4 | 8.6 | 0.300 | 0.146 | 0.362 | 0.162 | 0.278 | 0.423 |
| 5X | 1 | 1 | T − C | −10.0 | 0.9 | 0.4 | 0.015 | 0.010 | 0.020 | 0.014 | 0.003 | 0.012 |
| 5X | 1 | 1 | (T/C) % | 93 | 117 | 105 | 105 | 107 | 106 | 109 | 101 | 103 |
| 5X | 1 | 1 | p-value | 0.51 | 0.00 | 0.44 | 0.64 | 0.33 | 0.61 | 0.05 | 0.84 | 0.62 |
| 5X | 2 | 1 | Event (T) | 152.8 | 4.2 | 11.2 | 0.512 | 0.238 | 0.399 | 0.252 | 0.506 | 0.578 |
| 5X | 2 | 1 | Control (C) | 175.0 | 4.0 | 9.8 | 0.470 | 0.231 | 0.362 | 0.242 | 0.492 | 0.548 |
| 5X | 2 | 1 | T − C | −22.2 | 0.2 | 1.4 | 0.042 | 0.007 | 0.037 | 0.010 | 0.014 | 0.030 |
| 5X | 2 | 1 | (T/C) % | 87 | 105 | 114 | 109 | 103 | 110 | 104 | 103 | 105 |
| 5X | 2 | 1 | p-value | 0.00 | 0.04 | 0.05 | 0.22 | 0.61 | 0.04 | 0.32 | 0.50 | 0.27 |
| 5X | All | 2 | Event (T) | 151.2 | 4.0 | 10.4 | 0.427 | 0.212 | 0.373 | 0.219 | 0.396 | 0.529 |
| 5X | All | 2 | Control (C) | 165.8 | 3.9 | 9.1 | 0.355 | 0.191 | 0.319 | 0.196 | 0.359 | 0.468 |
| 5X | All | 2 | T − C | −14.6 | 0.1 | 1.3 | 0.072 | 0.021 | 0.054 | 0.023 | 0.037 | 0.061 |
| 5X | All | 2 | (T/C) % | 91 | 102 | 114 | 120 | 111 | 117 | 112 | 110 | 113 |
| 5X | All | 2 | p-value | 0.07 | 0.30 | 0.00 | 0.00 | 0.02 | 0.00 | 0.00 | 0.00 | 0.00 |
| 5X | 1 | 2 | Event (T) | 145.7 | 4.7 | 10.2 | 0.349 | 0.175 | 0.353 | 0.190 | 0.316 | 0.492 |
| 5X | 1 | 2 | Control (C) | 152.6 | 4.4 | 8.8 | 0.290 | 0.152 | 0.303 | 0.163 | 0.289 | 0.436 |
| 5X | 1 | 2 | T − C | −6.9 | 0.3 | 1.4 | 0.059 | 0.023 | 0.050 | 0.027 | 0.027 | 0.056 |
| 5X | 1 | 2 | (T/C) % | 95 | 107 | 116 | 120 | 115 | 117 | 117 | 109 | 113 |
| 5X | 1 | 2 | p-value | 0.35 | 0.06 | 0.00 | 0.00 | 0.05 | 0.02 | 0.03 | 0.01 | 0.00 |
| 5X | 2 | 2 | Event (T) | 159.0 | 3.4 | 11.0 | 0.523 | 0.256 | 0.399 | 0.258 | 0.485 | 0.580 |
| 5X | 2 | 2 | Control (C) | 172.6 | 3.4 | 9.5 | 0.422 | 0.229 | 0.335 | 0.228 | 0.434 | 0.504 |
| 5X | 2 | 2 | T − C | −13.6 | 0.0 | 1.5 | 0.101 | 0.027 | 0.064 | 0.030 | 0.051 | 0.076 |
| 5X | 2 | 2 | (T/C) % | 92 | 100 | 116 | 124 | 112 | 119 | 113 | 112 | 115 |
| 5X | 2 | 2 | p-value | 0.22 | 0.96 | 0.11 | 0.20 | 0.21 | 0.02 | 0.02 | 0.21 | 0.22 |
| 5Y | All | 1 | Event (T) | 135.2 | 5.0 | 10.6 | 0.440 | 0.214 | 0.394 | 0.224 | 0.430 | 0.557 |
| 5Y | All | 1 | Control (C) | 148.3 | 4.7 | 9.0 | 0.372 | 0.190 | 0.348 | 0.203 | 0.385 | 0.478 |
| 5Y | All | 1 | T − C | −13.1 | 0.3 | 1.6 | 0.068 | 0.024 | 0.046 | 0.021 | 0.045 | 0.079 |
| 5Y | All | 1 | (T/C) % | 91 | 106 | 118 | 118 | 113 | 113 | 110 | 112 | 117 |
| 5Y | All | 1 | p-value | 0.26 | 0.01 | 0.00 | 0.00 | 0.03 | 0.01 | 0.01 | 0.00 | 0.00 |
| 5Y | 1 | 1 | Event (T) | 137.6 | 5.7 | 9.7 | 0.342 | 0.162 | 0.404 | 0.184 | 0.306 | 0.472 |
| 5Y | 1 | 1 | Control (C) | 137.1 | 5.3 | 8.6 | 0.297 | 0.148 | 0.350 | 0.162 | 0.282 | 0.427 |
| 5Y | 1 | 1 | T − C | 0.5 | 0.4 | 1.1 | 0.045 | 0.014 | 0.054 | 0.022 | 0.024 | 0.045 |
| 5Y | 1 | 1 | (T/C) % | 100 | 108 | 113 | 115 | 109 | 115 | 114 | 109 | 111 |
| 5Y | 1 | 1 | p-value | 0.97 | 0.10 | 0.01 | 0.01 | 0.19 | 0.07 | 0.04 | 0.08 | 0.04 |
| 5Y | 2 | 1 | Event (T) | 153.6 | 4.3 | 11.2 | 0.548 | 0.258 | 0.410 | 0.265 | 0.538 | 0.619 |
| 5Y | 2 | 1 | Control (C) | 175.0 | 4.0 | 9.8 | 0.470 | 0.231 | 0.362 | 0.242 | 0.492 | 0.548 |
| 5Y | 2 | 1 | T − C | −21.4 | 0.3 | 1.4 | 0.078 | 0.027 | 0.048 | 0.023 | 0.046 | 0.071 |
| 5Y | 2 | 1 | (T/C) % | 88 | 108 | 114 | 117 | 112 | 113 | 110 | 109 | 113 |
| 5Y | 2 | 1 | p-value | 0.02 | 0.07 | 0.07 | 0.15 | 0.09 | 0.17 | 0.08 | 0.18 | 0.10 |
| 5Y | All | 2 | Event (T) | 149.5 | 4.0 | 10.2 | 0.411 | 0.205 | 0.375 | 0.206 | 0.387 | 0.524 |
| 5Y | All | 2 | Control (C) | 165.8 | 3.9 | 9.1 | 0.355 | 0.191 | 0.319 | 0.196 | 0.359 | 0.468 |
| 5Y | All | 2 | T − C | −16.3 | 0.1 | 1.1 | 0.056 | 0.014 | 0.056 | 0.010 | 0.028 | 0.056 |
| 5Y | All | 2 | (T/C) % | 90 | 103 | 112 | 116 | 107 | 118 | 105 | 108 | 112 |
| 5Y | All | 2 | p-value | 0.04 | 0.45 | 0.00 | 0.00 | 0.10 | 0.00 | 0.26 | 0.02 | 0.00 |
| 5Y | 1 | 2 | Event (T) | 145.4 | 4.6 | 10.1 | 0.351 | 0.172 | 0.366 | 0.180 | 0.314 | 0.492 |
| 5Y | 1 | 2 | Control (C) | 152.6 | 4.4 | 8.8 | 0.290 | 0.152 | 0.303 | 0.163 | 0.289 | 0.436 |
| 5Y | 1 | 2 | T − C | −7.2 | 0.2 | 1.3 | 0.061 | 0.020 | 0.063 | 0.017 | 0.025 | 0.056 |
| 5Y | 1 | 2 | (T/C) % | 95 | 105 | 115 | 121 | 113 | 121 | 110 | 109 | 113 |
| 5Y | 1 | 2 | p-value | 0.37 | 0.18 | 0.00 | 0.00 | 0.10 | 0.00 | 0.11 | 0.04 | 0.00 |
| 5Y | 2 | 2 | Event (T) | 162.3 | 3.4 | 10.3 | 0.471 | 0.236 | 0.387 | 0.237 | 0.458 | 0.555 |
| 5Y | 2 | 2 | Control (C) | 172.6 | 3.4 | 9.5 | 0.422 | 0.229 | 0.335 | 0.228 | 0.434 | 0.504 |
| 5Y | 2 | 2 | T − C | −10.3 | 0.0 | 0.8 | 0.049 | 0.007 | 0.052 | 0.009 | 0.024 | 0.051 |
| 5Y | 2 | 2 | (T/C) % | 94 | 100 | 108 | 112 | 103 | 116 | 104 | 106 | 110 |
| 5Y | 2 | 2 | p-value | 0.23 | 0.38 | 0.22 | 0.16 | 0.58 | 0.00 | 0.53 | 0.22 | 0.16 |
| 5Z | All | 1 | Event (T) | 135.2 | 5.1 | 9.6 | 0.400 | 0.198 | 0.361 | 0.218 | 0.392 | 0.492 |
| 5Z | All | 1 | Control (C) | 148.3 | 4.7 | 9.0 | 0.372 | 0.190 | 0.348 | 0.203 | 0.385 | 0.478 |
| 5Z | All | 1 | T − C | −13.1 | 0.4 | 0.6 | 0.028 | 0.008 | 0.013 | 0.015 | 0.007 | 0.014 |
| 5Z | All | 1 | (T/C) % | 91 | 109 | 106 | 108 | 104 | 104 | 107 | 102 | 103 |
| 5Z | All | 1 | p-value | 0.23 | 0.01 | 0.09 | 0.20 | 0.50 | 0.46 | 0.15 | 0.68 | 0.49 |
| 5Z | 1 | 1 | Event (T) | 137.6 | 5.7 | 9.7 | 0.342 | 0.162 | 0.404 | 0.184 | 0.306 | 0.472 |
| 5Z | 1 | 1 | Control (C) | 137.1 | 5.3 | 8.6 | 0.297 | 0.148 | 0.350 | 0.162 | 0.282 | 0.427 |
| 5Z | 1 | 1 | T − C | 0.5 | 0.4 | 1.1 | 0.045 | 0.014 | 0.054 | 0.022 | 0.024 | 0.045 |
| 5Z | 1 | 1 | (T/C) % | 100 | 108 | 113 | 115 | 109 | 115 | 114 | 109 | 111 |
| 5Z | 1 | 1 | p-value | 0.97 | 0.10 | 0.01 | 0.01 | 0.19 | 0.07 | 0.04 | 0.08 | 0.04 |
| 5Z | 2 | 1 | Event (T) | 157.1 | 4.4 | 10.4 | 0.518 | 0.245 | 0.408 | 0.259 | 0.510 | 0.580 |
| 5Z | 2 | 1 | Control (C) | 175.0 | 4.0 | 9.8 | 0.470 | 0.231 | 0.362 | 0.242 | 0.492 | 0.548 |
| 5Z | 2 | 1 | T − C | −17.9 | 0.4 | 0.6 | 0.048 | 0.014 | 0.046 | 0.017 | 0.018 | 0.032 |
| 5Z | 2 | 1 | (T/C) % | 90 | 110 | 106 | 110 | 106 | 113 | 107 | 104 | 106 |
| 5Z | 2 | 1 | p-value | 0.01 | 0.05 | 0.00 | 0.08 | 0.07 | 0.22 | 0.03 | 0.12 | 0.07 |
| 5Z | All | 2 | Event (T) | 151.5 | 4.1 | 10.3 | 0.402 | 0.200 | 0.367 | 0.206 | 0.383 | 0.514 |
| 5Z | All | 2 | Control (C) | 165.8 | 3.9 | 9.1 | 0.355 | 0.191 | 0.319 | 0.196 | 0.359 | 0.468 |
| 5Z | All | 2 | T − C | −14.3 | 0.2 | 1.2 | 0.047 | 0.009 | 0.048 | 0.010 | 0.024 | 0.046 |
| 5Z | All | 2 | (T/C) % | 91 | 105 | 113 | 113 | 105 | 115 | 105 | 107 | 110 |
| 5Z | All | 2 | p-value | 0.13 | 0.08 | 0.00 | 0.00 | 0.28 | 0.00 | 0.20 | 0.02 | 0.00 |
| 5Z | 1 | 2 | Event (T) | 145.4 | 4.6 | 10.1 | 0.351 | 0.172 | 0.366 | 0.180 | 0.314 | 0.492 |
| 5Z | 1 | 2 | Control (C) | 152.6 | 4.4 | 8.8 | 0.290 | 0.152 | 0.303 | 0.163 | 0.289 | 0.436 |
| 5Z | 1 | 2 | T − C | −7.2 | 0.2 | 1.3 | 0.061 | 0.020 | 0.063 | 0.017 | 0.025 | 0.056 |
| 5Z | 1 | 2 | (T/C) % | 95 | 105 | 115 | 121 | 113 | 121 | 110 | 109 | 113 |
| 5Z | 1 | 2 | p-value | 0.37 | 0.18 | 0.00 | 0.00 | 0.10 | 0.00 | 0.11 | 0.04 | 0.00 |
| 5Z | 2 | 2 | Event (T) | 163.2 | 3.5 | 10.8 | 0.494 | 0.241 | 0.389 | 0.233 | 0.473 | 0.566 |
| 5Z | 2 | 2 | Control (C) | 172.6 | 3.4 | 9.5 | 0.422 | 0.229 | 0.335 | 0.228 | 0.434 | 0.504 |
| 5Z | 2 | 2 | T − C | −9.4 | 0.1 | 1.3 | 0.072 | 0.012 | 0.054 | 0.005 | 0.039 | 0.062 |
| 5Z | 2 | 2 | (T/C) % | 95 | 103 | 114 | 117 | 105 | 116 | 102 | 109 | 112 |
| 5Z | 2 | 2 | p-value | 0.65 | 0.19 | 0.37 | 0.06 | 0.83 | 0.07 | 0.83 | 0.21 | 0.19 |
| 5A1 | All | 1 | Event (T) | 138.7 | 5.0 | 10.0 | 0.432 | 0.207 | 0.398 | 0.210 | 0.410 | 0.518 |
| 5A1 | All | 1 | Control (C) | 148.0 | 4.7 | 9.1 | 0.373 | 0.190 | 0.348 | 0.203 | 0.386 | 0.479 |
| 5A1 | All | 1 | T − C | −9.3 | 0.3 | 0.9 | 0.059 | 0.017 | 0.050 | 0.007 | 0.024 | 0.039 |
| 5A1 | All | 1 | (T/C) % | 94 | 106 | 110 | 116 | 109 | 114 | 103 | 106 | 108 |
| 5A1 | All | 1 | p-value | 0.31 | 0.00 | 0.00 | 0.00 | 0.10 | 0.00 | 0.38 | 0.04 | 0.02 |
| 5A1 | 1 | 1 | Event (T) | 138.1 | 5.8 | 9.0 | 0.313 | 0.138 | 0.412 | 0.154 | 0.282 | 0.425 |
| 5A1 | 1 | 1 | Control (C) | 137.1 | 5.4 | 8.6 | 0.300 | 0.146 | 0.362 | 0.162 | 0.278 | 0.423 |
| 5A1 | 1 | 1 | T − C | 1.0 | 0.4 | 0.4 | 0.013 | −0.008 | 0.050 | −0.008 | 0.004 | 0.002 |
| 5A1 | 1 | 1 | (T/C) % | 101 | 107 | 105 | 104 | 95 | 114 | 95 | 101 | 100 |
| 5A1 | 1 | 1 | p-value | 0.95 | 0.01 | 0.35 | 0.49 | 0.43 | 0.11 | 0.24 | 0.70 | 0.92 |
| 5A1 | 2 | 1 | Event (T) | 149.3 | 4.3 | 10.8 | 0.536 | 0.252 | 0.409 | 0.252 | 0.518 | 0.592 |
| 5A1 | 2 | 1 | Control (C) | 175.0 | 4.0 | 9.8 | 0.470 | 0.231 | 0.362 | 0.242 | 0.492 | 0.548 |
| 5A1 | 2 | 1 | T − C | −25.7 | 0.3 | 1.0 | 0.066 | 0.021 | 0.047 | 0.010 | 0.026 | 0.044 |
| 5A1 | 2 | 1 | (T/C) % | 85 | 108 | 110 | 114 | 109 | 113 | 104 | 105 | 108 |
| 5A1 | 2 | 1 | p-value | 0.04 | 0.00 | 0.21 | 0.11 | 0.11 | 0.01 | 0.22 | 0.17 | 0.16 |
| 5A1 | All | 2 | Event (T) | 142.5 | 4.0 | 10.5 | 0.424 | 0.210 | 0.367 | 0.220 | 0.400 | 0.531 |
| 5A1 | All | 2 | Control (C) | 165.8 | 3.9 | 9.1 | 0.355 | 0.191 | 0.319 | 0.196 | 0.359 | 0.468 |
| 5A1 | All | 2 | T − C | −23.3 | 0.1 | 1.3 | 0.069 | 0.019 | 0.048 | 0.024 | 0.041 | 0.063 |
| 5A1 | All | 2 | (T/C) % | 86 | 103 | 115 | 119 | 110 | 115 | 112 | 111 | 113 |
| 5A1 | All | 2 | p-value | 0.01 | 0.19 | 0.00 | 0.00 | 0.03 | 0.00 | 0.00 | 0.00 | 0.00 |
| 5A1 | 1 | 2 | Event (T) | 132.5 | 4.7 | 10.2 | 0.323 | 0.159 | 0.344 | 0.169 | 0.311 | 0.476 |
| 5A1 | 1 | 2 | Control (C) | 152.6 | 4.4 | 8.8 | 0.290 | 0.152 | 0.303 | 0.163 | 0.289 | 0.436 |
| 5A1 | 1 | 2 | T − C | −20.1 | 0.3 | 1.4 | 0.033 | 0.007 | 0.041 | 0.006 | 0.022 | 0.040 |
| 5A1 | 1 | 2 | (T/C) % | 87 | 107 | 116 | 111 | 105 | 114 | 104 | 108 | 109 |
| 5A1 | 1 | 2 | p-value | 0.02 | 0.08 | 0.00 | 0.05 | 0.64 | 0.02 | 0.63 | 0.06 | 0.03 |
| 5A1 | 2 | 2 | Event (T) | 152.7 | 3.4 | 11.1 | 0.535 | 0.268 | 0.396 | 0.278 | 0.496 | 0.597 |
| 5A1 | 2 | 2 | Control (C) | 172.6 | 3.4 | 9.5 | 0.422 | 0.229 | 0.335 | 0.228 | 0.434 | 0.504 |
| 5A1 | 2 | 2 | T − C | −19.9 | 0.0 | 1.6 | 0.113 | 0.039 | 0.061 | 0.050 | 0.062 | 0.093 |
| 5A1 | 2 | 2 | (T/C) % | 88 | 100 | 117 | 127 | 117 | 118 | 122 | 114 | 118 |
| 5A1 | 2 | 2 | p-value | 0.03 | 0.98 | 0.08 | 0.01 | 0.01 | 0.01 | 0.05 | 0.02 | 0.02 |
| 5B1 | All | 1 | Event (T) | 127.7 | 4.9 | 10.6 | 0.438 | 0.215 | 0.375 | 0.222 | 0.422 | 0.540 |
| 5B1 | All | 1 | Control (C) | 148.3 | 4.7 | 9.0 | 0.372 | 0.190 | 0.348 | 0.203 | 0.385 | 0.478 |
| 5B1 | All | 1 | T − C | −20.6 | 0.2 | 1.6 | 0.066 | 0.025 | 0.027 | 0.019 | 0.037 | 0.062 |
| 5B1 | All | 1 | (T/C) % | 86 | 104 | 117 | 118 | 113 | 108 | 109 | 110 | 113 |
| 5B1 | All | 1 | p-value | 0.05 | 0.06 | 0.00 | 0.00 | 0.01 | 0.06 | 0.02 | 0.00 | 0.00 |
| 5B1 | 1 | 1 | Event (T) | 109.7 | 5.7 | 9.9 | 0.347 | 0.182 | 0.369 | 0.190 | 0.313 | 0.472 |
| 5B1 | 1 | 1 | Control (C) | 137.1 | 5.4 | 8.8 | 0.305 | 0.164 | 0.343 | 0.171 | 0.296 | 0.434 |
| 5B1 | 1 | 1 | T − C | −27.4 | 0.3 | 1.1 | 0.042 | 0.018 | 0.026 | 0.019 | 0.017 | 0.038 |
| 5B1 | 1 | 1 | (T/C) % | 80 | 106 | 112 | 114 | 111 | 18 | 111 | 106 | 109 |
| 5B1 | 1 | 1 | p-value | 0.08 | 0.20 | 0.01 | 0.01 | 0.16 | 0.26 | 0.03 | 0.20 | 0.02 |
| 5B1 | 2 | 1 | Event (T) | 156.6 | 4.0 | 11.3 | 0.529 | 0.252 | 0.3078 | 0.259 | 0.535 | 0.612 |
| 5B1 | 2 | 1 | Control (C) | 175.0 | 4.0 | 9.8 | 0.470 | 0.231 | 0.362 | 0.242 | 0.492 | 0.548 |
| 5B1 | 2 | 1 | T − C | −18.4 | 0.0 | 1.5 | 0.059 | 0.021 | 0.016 | 0.017 | 0.043 | 0.064 |
| 5B1 | 2 | 1 | (T/C) % | 89 | 100 | 115 | 113 | 109 | 104 | 107 | 109 | 112 |
| 5B1 | 2 | 1 | p-value | 0.00 | 0.40 | 0.14 | 0.09 | 0.00 | 0.09 | 0.05 | 0.12 | 0.06 |
| 5B1 | All | 2 | Event (T) | 147.1 | 3.9 | 9.6 | 0.388 | 0.203 | 0.335 | 0.216 | 0.384 | 0.506 |
| 5B1 | All | 2 | Control (C) | 165.8 | 3.9 | 9.1 | 0.355 | 0.191 | 0.319 | 0.196 | 0.359 | 0.468 |
| 5B1 | All | 2 | T − C | −18.7 | 0.0 | 0.5 | 0.033 | 0.012 | 0.016 | 0.020 | 0.025 | 0.038 |
| 5B1 | All | 2 | (T/C) % | 89 | 100 | 105 | 109 | 106 | 105 | 110 | 107 | 108 |
| 5B1 | All | 2 | p-value | 0.04 | 0.69 | 0.14 | 0.06 | 0.23 | 0.30 | 0.02 | 0.03 | 0.02 |
| 5B1 | 1 | 2 | Event (T) | 130.2 | 4.8 | 9.6 | 0.340 | 0.176 | 0.351 | 0.178 | 0.310 | 0.482 |
| 5B1 | 1 | 2 | Control (C) | 152.6 | 4.4 | 8.8 | 0.290 | 0.152 | 0.303 | 0.163 | 0.289 | 0.436 |
| 5B1 | 1 | 2 | T − C | −22.4 | 0.4 | 0.8 | 0.050 | 0.024 | 0.048 | 0.015 | 0.021 | 0.046 |
| 5B1 | 1 | 2 | (T/C) % | 85 | 109 | 109 | 117 | 116 | 116 | 109 | 107 | 111 |
| 5B1 | 1 | 2 | p-value | 0.01 | 0.03 | 0.12 | 0.01 | 0.10 | 0.02 | 0.18 | 0.10 | 0.02 |
| 5B1 | 2 | 2 | Event (T) | 168.2 | 3.4 | 10.0 | 0.456 | 0.245 | 0.331 | 0.260 | 0.459 | 0.545 |
| 5B1 | 2 | 2 | Control (C) | 172.6 | 3.4 | 9.5 | 0.418 | 0.228 | 0.333 | 0.234 | 0.427 | 0.499 |
| 5B1 | 2 | 2 | T − C | −4.4 | 0.0 | 0.5 | 0.038 | 0.017 | −0.002 | 0.026 | 0.032 | 0.046 |
| 5B1 | 2 | 2 | (T/C) % | 97 | 100 | 105 | 109 | 107 | 99 | 111 | 107 | 109 |
| 5B1 | 2 | 2 | p-value | 0.38 | 0.31 | 0.42 | 0.82 | 0.08 | 0.71 | 0.05 | 0.54 | 0.53 |
| 5C1 | All | 1 | Event (T) | 149.0 | 4.8 | 11.2 | 0.458 | 0.241 | 0.379 | 0.236 | 0.439 | 0.571 |
| 5C1 | All | 1 | Control (C) | 148.3 | 4.7 | 9.0 | 0.372 | 0.190 | 0.348 | 0.203 | 0.385 | 0.478 |
| 5C1 | All | 1 | T − C | 0.7 | 0.1 | 2.1 | 0.086 | 0.051 | 0.031 | 0.033 | 0.054 | 0.093 |
| 5C1 | All | 1 | (T/C) % | 100 | 102 | 123 | 123 | 127 | 109 | 116 | 114 | 119 |
| 5C1 | All | 1 | p-value | 0.95 | 0.51 | 0.00 | 0.00 | 0.00 | 0.06 | 0.00 | 0.00 | 0.00 |
| 5C1 | 1 | 1 | Event (T) | 131.5 | 5.5 | 10.4 | 0.343 | 0.182 | 0.373 | 0.189 | 0.314 | 0.485 |
| 5C1 | 1 | 1 | Control (C) | 137.1 | 5.3 | 8.6 | 0.297 | 0.148 | 0.350 | 0.162 | 0.282 | 0.427 |
| 5C1 | 1 | 1 | T − C | −5.6 | 0.2 | 1.8 | 0.046 | 0.034 | 0.023 | 0.027 | 0.032 | 0.058 |
| 5C1 | 1 | 1 | (T/C) % | 96 | 104 | 121 | 115 | 123 | 107 | 117 | 111 | 114 |
| 5C1 | 1 | 1 | p-value | 0.73 | 0.51 | 0.00 | 0.05 | 0.10 | 0.38 | 0.00 | 0.09 | 0.04 |
| 5C1 | 2 | 1 | Event (T) | 176.4 | 4.2 | 11.3 | 0.527 | 0.265 | 0.378 | 0.270 | 0.528 | 0.609 |
| 5C1 | 2 | 1 | Control (C) | 175.0 | 4.0 | 9.8 | 0.470 | 0.231 | 0.362 | 0.242 | 0.492 | 0.548 |
| 5C1 | 2 | 1 | T − C | 1.4 | 0.2 | 1.5 | 0.057 | 0.034 | 0.016 | 0.028 | 0.036 | 0.061 |
| 5C1 | 2 | 1 | (T/C) % | 101 | 105 | 115 | 112 | 115 | 104 | 112 | 107 | 111 |
| 5C1 | 2 | 1 | p-value | 0.63 | 0.04 | 0.05 | 0.02 | 0.06 | 0.17 | 0.01 | 0.13 | 0.08 |
| 5C1 | All | 2 | Event (T) | 161.5 | 4.1 | 10.3 | 0.422 | 0.220 | 0.353 | 0.220 | 0.401 | 0.534 |
| 5C1 | All | 2 | Control (C) | 165.8 | 3.9 | 9.1 | 0.355 | 0.191 | 0.319 | 0.196 | 0.359 | 0.468 |
| 5C1 | All | 2 | T − C | −4.3 | 0.2 | 1.1 | 0.067 | 0.029 | 0.034 | 0.024 | 0.042 | 0.066 |
| 5C1 | All | 2 | (T/C) % | 97 | 105 | 112 | 119 | 115 | 111 | 112 | 112 | 114 |
| 5C1 | All | 2 | p-value | 0.63 | 0.13 | 0.00 | 0.00 | 0.00 | 0.01 | 0.00 | 0.00 | 0.00 |
| 5C1 | 1 | 2 | Event (T) | 160.2 | 4.7 | 10.0 | 0.339 | 0.190 | 0.316 | 0.193 | 0.319 | 0.489 |
| 5C1 | 1 | 2 | Control (C) | 152.6 | 4.4 | 8.8 | 0.290 | 0.152 | 0.303 | 0.163 | 0.289 | 0.436 |
| 5C1 | 1 | 2 | T − C | 7.6 | 0.3 | 1.2 | 0.049 | 0.038 | 0.013 | 0.030 | 0.030 | 0.053 |
| 5C1 | 1 | 2 | (T/C) % | 105 | 107 | 114 | 117 | 125 | 104 | 118 | 110 | 112 |
| 5C1 | 1 | 2 | p-value | 0.36 | 0.08 | 0.00 | 0.01 | 0.01 | 0.44 | 0.01 | 0.02 | 0.00 |
| 5C1 | 2 | 2 | Event (T) | 163.8 | 3.5 | 11.0 | 0.531 | 0.268 | 0.400 | 0.261 | 0.502 | 0.596 |
| 5C1 | 2 | 2 | Control (C) | 172.6 | 3.4 | 9.5 | 0.422 | 0.229 | 0.335 | 0.228 | 0.434 | 0.504 |
| 5C1 | 2 | 2 | T − C | −8.8 | 0.1 | 1.5 | 0.109 | 0.039 | 0.065 | 0.033 | 0.068 | 0.092 |
| 5C1 | 2 | 2 | (T/C) % | 95 | 103 | 116 | 126 | 117 | 119 | 114 | 116 | 118 |
| 5C1 | 2 | 2 | p-value | 0.03 | 0.35 | 0.00 | 0.00 | 0.03 | 0.01 | 0.20 | 0.00 | 0.00 |

TABLE 30
Summary of field data for Construct 6. Numbers shown in bold are significantly different from the control
at the p-value shown. bu/a is bushels per acre. “All” indicates the average across all events or
testers. (T/C) % is the value for the transgenic hybrid combination (T) expressed as a percent of the control (C).

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | (T/C) % | 103 | 102 | 101 | 100 | 102 | 98 | 101 | 100 | 101 |
| 6D1 | (T/C) % | 103 | 102 | 94 | 96 | 99 | 101 | 95 | 96 | 96 |
| 6E1 | (T/C) % | 102 | 102 | 100 | 96 | 103 | 97 | 101 | 97 | 98 |
| 6F1 | (T/C) % | 94 | 96 | 109 | 104 | 113 | 92 | 107 | 104 | 106 |
| 6G1 | (T/C) % | 105 | 104 | 104 | 105 | 105 | 97 | 103 | 102 | 105 |
| 6H1 | (T/C) % | 105 | 104 | 102 | 105 | 108 | 106 | 100 | 101 | 102 |
| 6I1 | (T/C) % | 96 | 102 | 99 | 97 | 101 | 93 | 101 | 98 | 99 |
| 6J1 | (T/C) % | 113 | 102 | 101 | 100 | 103 | 94 | 104 | 99 | 102 |
| 6K1 | (T/C) % | 108 | 100 | 101 | 100 | 96 | 99 | 96 | 100 | 102 |
| 6L1 | (T/C) % | 107 | 100 | 104 | 104 | 99 | 106 | 99 | 103 | 104 |
| 6M1 | (T/C) % | 90 | 102 | 82 | 98 | 96 | 112 | 97 | 93 | 93 |

TABLE 31
Field data for Construct 6. Numbers shown in bold are significantly different from the control at the
p-value shown. bu/a is bushels per acre. “All” indicates the average across all events or testers.
(T/C) % is the value for the transgenic hybrid combination (T) expressed as a percent of the control (C).
T − C is the transgenic hybrid combination minus the control. Oil, protein, and amino acid content
are shown as percent of seed dry weight.

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | Construct (T) | 146.4 | 4.9 | 9.0 | 0.296 | 0.151 | 0.325 | 0.170 | 0.300 | 0.437 |
| All | Control (C) | 142.4 | 4.8 | 8.9 | 0.295 | 0.148 | 0.332 | 0.169 | 0.301 | 0.432 |
| All | T − C | 4.0 | 0.1 | 0.1 | 0.001 | 0.003 | −0.007 | 0.001 | −0.001 | 0.005 |
| All | (T/C) % | 103 | 102 | 101 | 100 | 102 | 98 | 101 | 100 | 101 |
| All | p-value | 0.42 | 0.53 | 0.55 | 0.81 | 0.52 | 0.25 | 0.86 | 0.94 | 0.57 |
| 6D1 | Event (T) | 147.3 | 5.0 | 8.3 | 0.280 | 0.147 | 0.332 | 0.160 | 0.285 | 0.407 |
| 6D1 | Control (C) | 142.4 | 4.9 | 8.8 | 0.291 | 0.149 | 0.329 | 0.169 | 0.298 | 0.425 |
| 6D1 | T − C | 4.9 | 0.1 | −0.5 | −0.011 | −0.002 | 0.003 | −0.009 | −0.013 | −0.018 |
| 6D1 | (T/C) % | 103 | 102 | 94 | 96 | 99 | 101 | 95 | 96 | 96 |
| 6D1 | p-value | 0.60 | 0.68 | 0.04 | 0.48 | 0.86 | 0.81 | 0.24 | 0.20 | 0.33 |
| 6E1 | Event (T) | 145.4 | 4.9 | 8.9 | 0.283 | 0.152 | 0.319 | 0.172 | 0.292 | 0.422 |
| 6E1 | Control (C) | 142.4 | 4.8 | 8.9 | 0.294 | 0.148 | 0.330 | 0.170 | 0.300 | 0.431 |
| 6E1 | T − C | 3.0 | 0.1 | 0.0 | −0.011 | 0.004 | −0.011 | 0.002 | −0.008 | −0.009 |
| 6E1 | (T/C) % | 102 | 102 | 100 | 96 | 103 | 97 | 101 | 97 | 98 |
| 6E1 | p-value | 0.74 | 0.82 | 0.81 | 0.39 | 0.66 | 0.34 | 0.72 | 0.37 | 0.55 |
| 6F1 | Event (T) | 133.2 | 4.7 | 9.8 | 0.304 | 0.162 | 0.299 | 0.180 | 0.313 | 0.457 |
| 6F1 | Control (C) | 142.4 | 4.9 | 9.0 | 0.292 | 0.143 | 0.324 | 0.168 | 0.300 | 0.433 |
| 6F1 | T − C | −9.2 | −0.2 | 0.8 | 0.012 | 0.019 | −0.025 | 0.012 | 0.013 | 0.024 |
| 6F1 | (T/C) % | 94 | 96 | 109 | 104 | 113 | 92 | 107 | 104 | 106 |
| 6F1 | p-value | 0.31 | 0.55 | 0.01 | 0.47 | 0.10 | 0.06 | 0.19 | 0.31 | 0.27 |
| 6G1 | Event (T) | 149.8 | 5.0 | 9.3 | 0.310 | 0.156 | 0.322 | 0.174 | 0.308 | 0.452 |
| 6G1 | Control (C) | 142.4 | 4.8 | 8.9 | 0.295 | 0.148 | 0.332 | 0.169 | 0.301 | 0.432 |
| 6G1 | T − C | 7.4 | 0.2 | 0.4 | 0.015 | 0.008 | −0.010 | 0.005 | 0.007 | 0.020 |
| 6G1 | (T/C) % | 105 | 104 | 104 | 105 | 105 | 97 | 103 | 102 | 105 |
| 6G1 | p-value | 0.42 | 0.18 | 0.17 | 0.26 | 0.36 | 0.45 | 0.51 | 0.42 | 0.23 |
| 6H1 | Event (T) | 149.9 | 5.0 | 9.2 | 0.306 | 0.154 | 0.340 | 0.168 | 0.304 | 0.440 |
| 6H1 | Control (C) | 142.4 | 4.8 | 9.0 | 0.291 | 0.143 | 0.321 | 0.168 | 0.300 | 0.432 |
| 6H1 | T − C | 7.5 | 0.2 | 0.2 | 0.015 | 0.011 | 0.019 | 0.000 | 0.004 | 0.008 |
| 6H1 | (T/C) % | 105 | 104 | 102 | 105 | 108 | 106 | 100 | 101 | 102 |
| 6H1 | p-value | 0.42 | 0.58 | 0.54 | 0.30 | 0.25 | 0.09 | 1.00 | 0.69 | 0.67 |
| 6I1 | Event (T) | 137.0 | 4.9 | 8.8 | 0.287 | 0.149 | 0.310 | 0.170 | 0.296 | 0.426 |
| 6I1 | Control (C) | 142.4 | 4.8 | 8.9 | 0.295 | 0.148 | 0.332 | 0.169 | 0.301 | 0.432 |
| 6I1 | T − C | −5.4 | 0.1 | −0.1 | −0.008 | 0.001 | −0.022 | 0.001 | −0.005 | −0.006 |
| 6I1 | (T/C) % | 96 | 102 | 99 | 97 | 101 | 93 | 101 | 98 | 99 |
| 6I1 | p-value | 0.56 | 0.80 | 0.68 | 0.46 | 0.87 | 0.04 | 0.93 | 0.56 | 0.72 |
| 6J1 | Event (T) | 161.4 | 4.9 | 9.0 | 0.296 | 0.153 | 0.311 | 0.176 | 0.298 | 0.439 |
| 6J1 | Control (C) | 142.4 | 4.8 | 8.9 | 0.295 | 0.148 | 0.332 | 0.169 | 0.301 | 0.432 |
| 6J1 | T − C | 19.0 | 0.1 | 0.1 | 0.001 | 0.005 | −0.021 | 0.007 | −0.003 | 0.007 |
| 6J1 | (T/C) % | 113 | 102 | 101 | 100 | 103 | 94 | 104 | 99 | 102 |
| 6J1 | p-value | 0.04 | 0.60 | 0.77 | 0.93 | 0.54 | 0.08 | 0.32 | 0.80 | 0.65 |
| 6K1 | Event (T) | 153.9 | 4.8 | 9.0 | 0.295 | 0.142 | 0.327 | 0.163 | 0.299 | 0.439 |
| 6K1 | Control (C) | 142.4 | 4.8 | 8.9 | 0.294 | 0.148 | 0.330 | 0.170 | 0.300 | 0.431 |
| 6K1 | T − C | 11.5 | 0.0 | 0.1 | 0.001 | −0.006 | −0.003 | −0.007 | −0.001 | 0.008 |
| 6K1 | (T/C) % | 108 | 100 | 101 | 100 | 96 | 99 | 96 | 100 | 102 |
| 6K1 | p-value | 0.21 | 0.74 | 0.85 | 0.91 | 0.54 | 0.80 | 0.39 | 0.92 | 0.63 |
| 6L1 | Event (T) | 152.4 | 4.8 | 9.3 | 0.307 | 0.146 | 0.353 | 0.167 | 0.310 | 0.451 |
| 6L1 | Control (C) | 142.4 | 4.8 | 8.9 | 0.295 | 0.148 | 0.332 | 0.169 | 0.301 | 0.432 |
| 6L1 | T − C | 10.0 | 0.0 | 0.4 | 0.012 | −0.002 | 0.021 | −0.002 | 0.009 | 0.019 |
| 6L1 | (T/C) % | 107 | 100 | 104 | 104 | 99 | 106 | 99 | 103 | 104 |
| 6L1 | p-value | 0.28 | 0.96 | 0.15 | 0.32 | 0.88 | 0.07 | 0.78 | 0.28 | 0.24 |
| 6M1 | Event (T) | 130.5 | 4.7 | 7.8 | 0.299 | 0.137 | 0.375 | 0.165 | 0.287 | 0.420 |
| 6M1 | Control (C) | 145.5 | 4.6 | 9.5 | 0.305 | 0.143 | 0.335 | 0.170 | 0.307 | 0.453 |
| 6M1 | T − C | −15.0 | 0.1 | −1.7 | −0.006 | −0.006 | 0.040 | −0.005 | −0.020 | −0.033 |
| 6M1 | (T/C) % | 90 | 102 | 82 | 98 | 96 | 112 | 97 | 93 | 93 |
| 6M1 | p-value | 0.24 | 0.87 | 0.03 | 0.82 | 0.75 | 0.27 | 0.78 | 0.41 | 0.36 |

TABLE 32
Summary of field data for Construct 7. Numbers shown in bold are significantly different from the control
at the p-value shown. bu/a is bushels per acre. “All” indicates the average across all events or
testers. (T/C) % is the value for the transgenic hybrid combination (T) expressed as a percent of the control (C).

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | (T/C) % | 100 | 102 | 100 | 99 | 97 | 100 | 97 | 100 | 100 |
| 7N1 | (T/C) % | 101 | 100 | 100 | 105 | 101 | 102 | 96 | 101 | 104 |
| 7O1 | (T/C) % | 101 | 104 | 99 | 100 | 98 | 99 | 95 | 100 | 101 |
| 7P1 | (T/C) % | 104 | 100 | 106 | 99 | 99 | 97 | 101 | 102 | 102 |
| 7Q1 | (T/C) % | 104 | 104 | 100 | 97 | 94 | 99 | 96 | 99 | 100 |
| 7R1 | (T/C) % | 86 | 102 | 101 | 103 | 96 | 105 | 98 | 99 | 98 |
| 7S1 | (T/C) % | 101 | 100 | 104 | 104 | 103 | 101 | 103 | 103 | 103 |
| 7T1 | (T/C) % | 106 | 100 | 102 | 98 | 97 | 99 | 98 | 98 | 99 |
| 7U1 | (T/C) % | 99 | 102 | 96 | 95 | 93 | 99 | 92 | 96 | 97 |
| 7V1 | (T/C) % | 100 | 100 | 102 | 99 | 100 | 95 | 101 | 100 | 100 |
| 7W1 | (T/C) % | 102 | 104 | 100 | 102 | 94 | 104 | 96 | 101 | 100 |

TABLE 33
Field data for Construct 7. Numbers shown in bold are significantly different from the control at the
p-value shown. bu/a is bushels per acre. “All” indicates the average across all events or testers.
(T/C) % is the value for the transgenic hybrid combination (T) expressed as a percent of the control (C).
T − C is the transgenic hybrid combination minus the control. Oil, protein, and amino acid content
are shown as percent of seed dry weight.

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | Construct (T) | 150.0 | 4.7 | 8.5 | 0.292 | 0.154 | 0.333 | 0.165 | 0.292 | 0.431 |
| All | Control (C) | 149.3 | 4.6 | 8.5 | 0.295 | 0.159 | 0.334 | 0.170 | 0.293 | 0.432 |
| All | T − C | 0.7 | 0.1 | 0.0 | −0.003 | −0.005 | −0.001 | −0.005 | −0.001 | −0.001 |
| All | (T/C) % | 100 | 102 | 100 | 99 | 97 | 100 | 97 | 100 | 100 |
| All | p-value | 0.89 | 0.32 | 0.78 | 0.66 | 0.24 | 0.89 | 0.19 | 0.73 | 0.85 |
| 7N1 | Event (T) | 150.2 | 4.6 | 8.4 | 0.304 | 0.159 | 0.338 | 0.162 | 0.294 | 0.444 |
| 7N1 | Control (C) | 149.3 | 4.6 | 8.4 | 0.290 | 0.157 | 0.332 | 0.168 | 0.290 | 0.428 |
| 7N1 | T − C | 0.9 | 0.0 | 0.0 | 0.014 | 0.002 | 0.006 | −0.006 | 0.004 | 0.016 |
| 7N1 | (T/C) % | 101 | 100 | 100 | 105 | 101 | 102 | 96 | 101 | 104 |
| 7N1 | p-value | 0.92 | 0.93 | 0.91 | 0.31 | 0.71 | 0.64 | 0.23 | 0.71 | 0.31 |
| 7O1 | Event (T) | 151.1 | 4.8 | 8.3 | 0.290 | 0.154 | 0.329 | 0.160 | 0.289 | 0.431 |
| 7O1 | Control (C) | 149.3 | 4.6 | 8.4 | 0.290 | 0.157 | 0.332 | 0.168 | 0.290 | 0.428 |
| 7O1 | T − C | 1.8 | 0.2 | −0.1 | 0.000 | −0.003 | −0.003 | −0.008 | −0.001 | 0.003 |
| 7O1 | (T/C) % | 101 | 104 | 99 | 100 | 98 | 99 | 95 | 100 | 101 |
| 7O1 | p-value | 0.83 | 0.13 | 0.89 | 0.98 | 0.66 | 0.81 | 0.12 | 0.93 | 0.82 |
| 7P1 | Event (T) | 155.5 | 4.6 | 8.9 | 0.287 | 0.156 | 0.322 | 0.170 | 0.295 | 0.435 |
| 7P1 | Control (C) | 149.3 | 4.6 | 8.4 | 0.290 | 0.157 | 0.332 | 0.168 | 0.290 | 0.428 |
| 7P1 | T − C | 6.2 | 0.0 | 0.5 | −0.003 | −0.001 | −0.010 | 0.002 | 0.005 | 0.007 |
| 7P1 | (T/C) % | 104 | 100 | 106 | 99 | 99 | 97 | 101 | 102 | 102 |
| 7P1 | p-value | 0.48 | 0.76 | 0.21 | 0.81 | 0.90 | 0.41 | 0.72 | 0.67 | 0.66 |
| 7Q1 | Event (T) | 154.8 | 4.8 | 8.5 | 0.285 | 0.150 | 0.331 | 0.164 | 0.291 | 0.430 |
| 7Q1 | Control (C) | 149.3 | 4.6 | 8.5 | 0.295 | 0.159 | 0.334 | 0.170 | 0.293 | 0.432 |
| 7Q1 | T − C | 5.5 | 0.2 | 0.0 | −0.010 | −0.009 | −0.003 | −0.006 | −0.002 | −0.002 |
| 7Q1 | (T/C) % | 104 | 104 | 100 | 97 | 94 | 99 | 96 | 99 | 100 |
| 7Q1 | p-value | 0.53 | 0.31 | 0.99 | 0.45 | 0.15 | 0.83 | 0.25 | 0.81 | 0.87 |
| 7R1 | Event (T) | 128.9 | 4.7 | 8.5 | 0.299 | 0.150 | 0.349 | 0.165 | 0.288 | 0.419 |
| 7R1 | Control (C) | 149.3 | 4.6 | 8.4 | 0.290 | 0.157 | 0.332 | 0.168 | 0.290 | 0.428 |
| 7R1 | T − C | −20.4 | 0.1 | 0.1 | 0.009 | −0.007 | 0.017 | −0.003 | −0.002 | −0.009 |
| 7R1 | (T/C) % | 86 | 102 | 101 | 103 | 96 | 105 | 98 | 99 | 98 |
| 7R1 | p-value | 0.02 | 0.47 | 0.84 | 0.55 | 0.27 | 0.24 | 0.61 | 0.84 | 0.59 |
| 7S1 | Event (T) | 151.1 | 4.6 | 8.8 | 0.306 | 0.164 | 0.336 | 0.175 | 0.302 | 0.447 |
| 7S1 | Control (C) | 149.3 | 4.6 | 8.5 | 0.295 | 0.159 | 0.334 | 0.170 | 0.293 | 0.432 |
| 7S1 | T − C | 1.8 | 0.0 | 0.3 | 0.011 | 0.005 | 0.002 | 0.005 | 0.009 | 0.015 |
| 7S1 | (T/C) % | 101 | 100 | 104 | 104 | 103 | 101 | 103 | 103 | 103 |
| 7S1 | p-value | 0.84 | 0.86 | 0.37 | 0.47 | 0.48 | 0.83 | 0.33 | 0.43 | 0.35 |
| 7T1 | Event (T) | 158.3 | 4.6 | 8.6 | 0.284 | 0.152 | 0.329 | 0.165 | 0.285 | 0.422 |
| 7T1 | Control (C) | 149.3 | 4.6 | 8.4 | 0.290 | 0.157 | 0.332 | 0.168 | 0.290 | 0.428 |
| 7T1 | T − C | 9.0 | 0.0 | 0.2 | −0.006 | −0.005 | −0.003 | −0.003 | −0.005 | −0.006 |
| 7T1 | (T/C) % | 106 | 100 | 102 | 98 | 97 | 99 | 98 | 98 | 99 |
| 7T1 | p-value | 0.31 | 0.61 | 0.64 | 0.63 | 0.42 | 0.79 | 0.55 | 0.64 | 0.70 |
| 7U1 | Event (T) | 147.1 | 4.7 | 8.2 | 0.279 | 0.148 | 0.331 | 0.156 | 0.282 | 0.419 |
| 7U1 | Control (C) | 149.3 | 4.6 | 8.5 | 0.295 | 0.159 | 0.334 | 0.170 | 0.293 | 0.432 |
| 7U1 | T − C | −2.2 | 0.1 | −0.3 | −0.016 | −0.011 | −0.003 | −0.014 | −0.011 | −0.013 |
| 7U1 | (T/C) % | 99 | 102 | 96 | 95 | 93 | 99 | 92 | 96 | 97 |
| 7U1 | p-value | 0.80 | 0.42 | 0.39 | 0.30 | 0.15 | 0.83 | 0.02 | 0.30 | 0.42 |
| 7V1 | Event (T) | 149.5 | 4.6 | 8.7 | 0.291 | 0.159 | 0.317 | 0.172 | 0.294 | 0.431 |
| 7V1 | Control (C) | 149.3 | 4.6 | 8.5 | 0.295 | 0.159 | 0.334 | 0.170 | 0.293 | 0.432 |
| 7V1 | T − C | 0.2 | 0.0 | 0.2 | −0.004 | 0.000 | −0.017 | 0.002 | 0.001 | −0.001 |
| 7V1 | (T/C) % | 100 | 100 | 102 | 99 | 100 | 95 | 101 | 100 | 100 |
| 7V1 | p-value | 0.98 | 0.96 | 0.50 | 0.75 | 0.98 | 0.18 | 0.64 | 0.96 | 0.92 |
| 7W1 | Event (T) | 152.2 | 4.8 | 8.4 | 0.296 | 0.148 | 0.346 | 0.161 | 0.293 | 0.428 |
| 7W1 | Control (C) | 149.3 | 4.6 | 8.4 | 0.290 | 0.157 | 0.332 | 0.168 | 0.290 | 0.428 |
| 7W1 | T − C | 2.9 | 0.2 | 0.0 | 0.006 | −0.009 | 0.014 | −0.007 | 0.003 | 0.000 |
| 7W1 | (T/C) % | 102 | 104 | 100 | 102 | 94 | 104 | 96 | 101 | 100 |
| 7W1 | p-value | 0.75 | 0.07 | 0.99 | 0.64 | 0.12 | 0.24 | 0.13 | 0.73 | 0.99 |

TABLE 34
Summary of field data for Construct 8. Numbers shown in bold are significantly different from the control
at the p-value shown. bu/a is bushels per acre. “All” indicates the average across all events or testers.
(T/C) % is the value for the transgenic hybrid combination (T) expressed as a percent of the control (C).

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | (T/C) % | 98 | 100 | 102 | 101 | 97 | 100 | 92 | 101 | 102 |
| 8X1 | (T/C) % | 93 | 100 | 103 | 101 | 92 | 101 | 90 | 101 | 102 |
| 8Y1 | (T/C) % | 97 | 98 | 103 | 107 | 102 | 104 | 94 | 102 | 103 |
| 8Z1 | (T/C) % | 105 | 98 | 102 | 97 | 92 | 95 | 95 | 100 | 100 |
| 8A2 | (T/C) % | 106 | 98 | 103 | 102 | 96 | 102 | 92 | 102 | 103 |
| 8B2 | (T/C) % | 97 | 98 | 106 | 107 | 112 | 104 | 100 | 106 | 108 |
| 8C2 | (T/C) % | 92 | 100 | 101 | 97 | 97 | 96 | 95 | 100 | 100 |

TABLE 35
Field data for Construct 8. Numbers shown in bold are significantly different from the control
at the p-value shown. bu/a is bushels per acre. “All” indicates the average across all
events or testers. (T/C) % is the value for the transgenic hybrid combination (T) expressed as a
percent of the control (C). T − C is the transgenic hybrid combination minus the control.
Oil, protein, and amino acid content are shown as percent of seed dry weight.

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | Construct (T) | 141.2 | 4.7 | 9.0 | 0.291 | 0.138 | 0.299 | 0.166 | 0.290 | 0.430 |
| All | Control (C) | 143.8 | 4.7 | 8.8 | 0.289 | 0.143 | 0.299 | 0.180 | 0.286 | 0.421 |
| All | T − C | −2.6 | 0.0 | 0.2 | 0.002 | −0.005 | 0.000 | −0.014 | 0.004 | 0.009 |
| All | (T/C) % | 98 | 100 | 102 | 101 | 97 | 100 | 92 | 101 | 102 |
| All | p-value | 0.73 | 0.67 | 0.27 | 0.84 | 0.47 | 0.97 | 0.11 | 0.52 | 0.33 |
| 8X1 | Event (T) | 133.2 | 4.7 | 9.1 | 0.292 | 0.131 | 0.301 | 0.162 | 0.290 | 0.431 |
| 8X1 | Control (C) | 143.8 | 4.7 | 8.8 | 0.289 | 0.143 | 0.299 | 0.180 | 0.286 | 0.421 |
| 8X1 | T − C | −10.6 | 0.0 | 0.3 | 0.003 | −0.012 | 0.002 | −0.018 | 0.004 | 0.010 |
| 8X1 | (T/C) % | 93 | 100 | 103 | 101 | 92 | 101 | 90 | 101 | 102 |
| 8X1 | p-value | 0.34 | 0.88 | 0.26 | 0.85 | 0.18 | 0.88 | 0.22 | 0.64 | 0.44 |
| 8Y1 | Event (T) | 139.6 | 4.7 | 8.9 | 0.302 | 0.140 | 0.309 | 0.160 | 0.290 | 0.428 |
| 8Y1 | Control (C) | 143.8 | 4.8 | 8.6 | 0.282 | 0.137 | 0.296 | 0.170 | 0.283 | 0.414 |
| 8Y1 | T − C | −4.2 | −0.1 | 0.3 | 0.020 | 0.003 | 0.013 | −0.010 | 0.007 | 0.014 |
| 8Y1 | (T/C) % | 97 | 98 | 103 | 107 | 102 | 104 | 94 | 102 | 103 |
| 8Y1 | p-value | 0.69 | 0.34 | 0.11 | 0.17 | 0.80 | 0.44 | 0.48 | 0.51 | 0.36 |
| 8Z1 | Event (T) | 150.5 | 4.6 | 9.0 | 0.281 | 0.132 | 0.285 | 0.171 | 0.286 | 0.421 |
| 8Z1 | Control (C) | 143.8 | 4.7 | 8.8 | 0.289 | 0.143 | 0.299 | 0.180 | 0.286 | 0.421 |
| 8Z1 | T − C | 6.7 | −0.1 | 0.2 | −0.008 | −0.011 | −0.014 | −0.009 | 0.000 | 0.000 |
| 8Z1 | (T/C) % | 105 | 98 | 102 | 97 | 92 | 95 | 95 | 100 | 100 |
| 8Z1 | p-value | 0.54 | 0.46 | 0.53 | 0.58 | 0.25 | 0.32 | 0.55 | 0.99 | 0.97 |
| 8A2 | Event (T) | 152.6 | 4.6 | 9.1 | 0.294 | 0.137 | 0.304 | 0.165 | 0.291 | 0.433 |
| 8A2 | Control (C) | 143.8 | 4.7 | 8.8 | 0.289 | 0.143 | 0.299 | 0.180 | 0.286 | 0.421 |
| 8A2 | T − C | 8.8 | −0.1 | 0.3 | 0.005 | −0.006 | 0.005 | −0.015 | 0.005 | 0.012 |
| 8A2 | (T/C) % | 106 | 98 | 103 | 102 | 96 | 102 | 92 | 102 | 103 |
| 8A2 | p-value | 0.40 | 0.40 | 0.27 | 0.73 | 0.55 | 0.76 | 0.30 | 0.58 | 0.38 |
| 8B2 | Event (T) | 139.0 | 4.7 | 9.1 | 0.303 | 0.154 | 0.309 | 0.170 | 0.301 | 0.447 |
| 8B2 | Control (C) | 143.8 | 4.8 | 8.6 | 0.282 | 0.137 | 0.296 | 0.170 | 0.283 | 0.414 |
| 8B2 | T − C | −4.8 | −0.1 | 0.5 | 0.021 | 0.017 | 0.013 | 0.000 | 0.018 | 0.033 |
| 8B2 | (T/C) % | 97 | 98 | 106 | 107 | 112 | 104 | 100 | 106 | 108 |
| 8B2 | p-value | 0.65 | 0.50 | 0.06 | 0.24 | 0.22 | 0.51 | 0.99 | 0.11 | 0.07 |
| 8C2 | Event (T) | 132.6 | 4.7 | 8.9 | 0.280 | 0.138 | 0.288 | 0.171 | 0.285 | 0.423 |
| 8C2 | Control (C) | 143.8 | 4.7 | 8.8 | 0.289 | 0.143 | 0.299 | 0.180 | 0.286 | 0.421 |
| 8C2 | T − C | −11.2 | 0.0 | 0.1 | −0.009 | −0.005 | −0.011 | −0.009 | −0.001 | 0.002 |
| 8C2 | (T/C) % | 92 | 100 | 101 | 97 | 97 | 96 | 95 | 100 | 100 |
| 8C2 | p-value | 0.26 | 0.98 | 0.77 | 0.52 | 0.62 | 0.48 | 0.57 | 0.85 | 0.85 |

TABLE 36
Summary of field data for Construct 9. Numbers shown in bold are significantly different from the control
at the p-value shown. bu/a is bushels per acre. “All” indicates the average across all events or testers.
(T/C) % is the value for the transgenic hybrid combination (T) expressed as a percent of the control (C).

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | (T/C) % | 101 | 98 | 100 | 102 | 102 | 103 | 104 | 101 | 101 |
| 9D2 | (T/C) % | 98 | 96 | 105 | 105 | 104 | 103 | 103 | 104 | 106 |
| 9E2 | (T/C) % | 101 | 98 | 99 | 102 | 99 | 108 | 104 | 102 | 102 |
| 9F2 | (T/C) % | 99 | 100 | 103 | 108 | 109 | 107 | 105 | 103 | 105 |
| 9G2 | (T/C) % | 97 | 98 | 97 | 100 | 103 | 101 | 110 | 96 | 97 |
| 9H2 | (T/C) % | 102 | 98 | 100 | 100 | 102 | 103 | 101 | 100 | 100 |
| 9I2 | (T/C) % | 104 | 98 | 97 | 94 | 95 | 97 | 101 | 98 | 97 |

TABLE 37
Field data for Construct 9. Numbers shown in bold are significantly different from the control
at the p-value shown. bu/a is bushels per acre. “All” indicates the average across all
events or testers. (T/C) % is the value for the transgenic hybrid combination (T) expressed as a
percent of the control (C). T − C is the transgenic hybrid combination minus the control.
Oil, protein, and amino acid content are shown as percent of seed dry weight.

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | Construct (T) | 153.4 | 5.0 | 8.7 | 0.301 | 0.154 | 0.361 | 0.155 | 0.298 | 0.435 |
| All | Control (C) | 152.4 | 5.1 | 8.7 | 0.296 | 0.151 | 0.349 | 0.149 | 0.296 | 0.429 |
| All | T − C | 1.0 | −0.1 | 0.0 | 0.005 | 0.003 | 0.012 | 0.006 | 0.002 | 0.006 |
| All | (T/C) % | 101 | 98 | 100 | 102 | 102 | 103 | 104 | 101 | 101 |
| All | p-value | 0.88 | 0.53 | 0.88 | 0.61 | 0.56 | 0.21 | 0.28 | 0.75 | 0.63 |
| 9D2 | Event (T) | 149.3 | 4.8 | 9.2 | 0.314 | 0.159 | 0.362 | 0.154 | 0.312 | 0.460 |
| 9D2 | Control (C) | 152.4 | 5.0 | 8.8 | 0.299 | 0.153 | 0.350 | 0.149 | 0.299 | 0.435 |
| 9D2 | T − C | −3.1 | −0.2 | 0.4 | 0.015 | 0.006 | 0.012 | 0.005 | 0.013 | 0.025 |
| 9D2 | (T/C) % | 98 | 96 | 105 | 105 | 104 | 103 | 103 | 104 | 106 |
| 9D2 | p-value | 0.75 | 0.28 | 0.32 | 0.45 | 0.56 | 0.46 | 0.69 | 0.37 | 0.26 |
| 9E2 | Event (T) | 154.3 | 5.0 | 8.6 | 0.301 | 0.150 | 0.376 | 0.155 | 0.301 | 0.439 |
| 9E2 | Control (C) | 153.5 | 5.1 | 8.7 | 0.296 | 0.151 | 0.349 | 0.149 | 0.296 | 0.429 |
| 9E2 | T − C | 0.8 | −0.1 | −0.1 | 0.005 | −0.001 | 0.027 | 0.006 | 0.005 | 0.010 |
| 9E2 | (T/C) % | 101 | 98 | 99 | 102 | 99 | 108 | 104 | 102 | 102 |
| 9E2 | p-value | 0.93 | 0.79 | 0.89 | 0.74 | 0.84 | 0.07 | 0.46 | 0.65 | 0.61 |
| 9F2 | Event (T) | 152.2 | 5.1 | 9.0 | 0.320 | 0.164 | 0.372 | 0.156 | 0.305 | 0.450 |
| 9F2 | Control (C) | 153.5 | 5.1 | 8.7 | 0.296 | 0.151 | 0.349 | 0.149 | 0.296 | 0.429 |
| 9F2 | T − C | −1.3 | 0.0 | 0.3 | 0.024 | 0.013 | 0.023 | 0.007 | 0.009 | 0.021 |
| 9F2 | (T/C) % | 99 | 100 | 103 | 108 | 109 | 107 | 105 | 103 | 105 |
| 9F2 | p-value | 0.89 | 0.91 | 0.44 | 0.19 | 0.18 | 0.12 | 0.42 | 0.44 | 0.32 |
| 9G2 | Event (T) | 148.3 | 4.9 | 8.5 | 0.300 | 0.157 | 0.355 | 0.164 | 0.288 | 0.423 |
| 9G2 | Control (C) | 152.4 | 5.0 | 8.8 | 0.299 | 0.153 | 0.350 | 0.149 | 0.299 | 0.435 |
| 9G2 | T − C | −4.1 | −0.1 | −0.3 | 0.001 | 0.004 | 0.005 | 0.015 | −0.011 | −0.012 |
| 9G2 | (T/C) % | 97 | 98 | 97 | 100 | 103 | 101 | 110 | 96 | 97 |
| 9G2 | p-value | 0.68 | 0.70 | 0.49 | 0.95 | 0.69 | 0.75 | 0.20 | 0.39 | 0.55 |
| 9H2 | Event (T) | 157.1 | 5.1 | 8.6 | 0.290 | 0.152 | 0.357 | 0.150 | 0.293 | 0.424 |
| 9H2 | Control (C) | 153.5 | 5.2 | 8.6 | 0.289 | 0.149 | 0.348 | 0.149 | 0.293 | 0.422 |
| 9H2 | T − C | 3.6 | −0.1 | 0.0 | 0.001 | 0.003 | 0.009 | 0.001 | 0.000 | 0.002 |
| 9H2 | (T/C) % | 102 | 98 | 100 | 100 | 102 | 103 | 101 | 100 | 100 |
| 9H2 | p-value | 0.72 | 0.82 | 0.97 | 0.95 | 0.65 | 0.65 | 0.86 | 0.99 | 0.92 |
| 9I2 | Event (T) | 159.1 | 4.9 | 8.4 | 0.279 | 0.146 | 0.338 | 0.152 | 0.288 | 0.414 |
| 9I2 | Control (C) | 152.4 | 5.0 | 8.7 | 0.297 | 0.154 | 0.350 | 0.151 | 0.295 | 0.429 |
| 9I2 | T − C | 6.7 | −0.1 | −0.3 | −0.018 | −0.008 | −0.012 | 0.001 | −0.007 | −0.015 |
| 9I2 | (T/C) % | 104 | 98 | 97 | 94 | 95 | 97 | 101 | 98 | 97 |
| 9I2 | p-value | 0.46 | 0.93 | 0.36 | 0.28 | 0.38 | 0.47 | 0.93 | 0.59 | 0.38 |

TABLE 38
Summary of field data for Construct 10. Numbers shown in bold are significantly different from the control
at the p-value shown. bu/a is bushels per acre. “All” indicates the average across all events or testers.
(T/C) % is the value for the transgenic hybrid combination (T) expressed as a percent of the control (C).

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | (T/C) % | 96 | 98 | 101 | 101 | 102 | 99 | 95 | 99 | 99 |
| 10J2 | (T/C) % | 91 | 96 | 98 | 99 | 99 | 100 | 95 | 98 | 98 |
| 10K2 | (T/C) % | 104 | 94 | 101 | 103 | 104 | 97 | 96 | 102 | 102 |
| 10L2 | (T/C) % | 98 | 98 | 101 | 102 | 103 | 99 | 96 | 101 | 100 |
| 10M2 | (T/C) % | 94 | 102 | 100 | 97 | 98 | 95 | 91 | 96 | 95 |
| 10N2 | (T/C) % | 92 | 100 | 102 | 103 | 104 | 103 | 97 | 100 | 100 |

TABLE 39
Field data for Construct 10. Numbers shown in bold are significantly different from the control
at the p-value shown. bu/a is bushels per acre. “All” indicates the average across all
events or testers. (T/C) % is the value for the transgenic hybrid combination (T) expressed as a
percent of the control (C). T − C is the transgenic hybrid combination minus the control.
Oil, protein, and amino acid content are shown as percent of seed dry weight.

| | | Yield | Oil | Prot | Arg | Cys | Lys | Met | Thr | Val |
|---|---|---|---|---|---|---|---|---|---|---|
| | | (bu/a) | (%) | (%) | (%) | (%) | (%) | (%) | (%) | (%) |
| Event | Description | p <= 0.10 | p <= 0.15 | p <= 0.15 | p <= 0.15 | p <= 0.15 | p <= 0.15 | p <= 0.15 | p <= 0.15 | p <= 0.15 |
| All | Construct (T) | 143.0 | 4.8 | 8.6 | 0.284 | 0.149 | 0.340 | 0.139 | 0.279 | 0.412 |
| All | Control (C) | 148.2 | 4.9 | 8.5 | 0.282 | 0.146 | 0.344 | 0.146 | 0.281 | 0.416 |
| All | T − C | −5.2 | −0.1 | 0.1 | 0.002 | 0.003 | −0.004 | −0.007 | −0.002 | −0.004 |
| All | (T/C) % | 96 | 98 | 101 | 101 | 102 | 99 | 95 | 99 | 99 |
| All | p-value | 0.43 | 0.69 | 0.86 | 0.80 | 0.54 | 0.65 | 0.17 | 0.75 | 0.64 |
| 10J2 | Event (T) | 135.0 | 4.7 | 8.3 | 0.280 | 0.145 | 0.343 | 0.139 | 0.275 | 0.409 |
| 10J2 | Control (C) | 148.2 | 4.9 | 8.5 | 0.282 | 0.146 | 0.344 | 0.146 | 0.281 | 0.416 |
| 10J2 | T − C | −13.2 | −0.2 | −0.2 | −0.002 | −0.001 | −0.001 | −0.007 | −0.006 | −0.007 |
| 10J2 | (T/C) % | 91 | 96 | 98 | 99 | 99 | 100 | 95 | 98 | 98 |
| 10J2 | p-value | 0.18 | 0.60 | 0.61 | 0.81 | 0.99 | 0.92 | 0.37 | 0.31 | 0.49 |
| 10K2 | Event (T) | 154.2 | 4.6 | 8.6 | 0.291 | 0.152 | 0.332 | 0.140 | 0.286 | 0.426 |
| 10K2 | Control (C) | 148.2 | 4.9 | 8.5 | 0.282 | 0.146 | 0.344 | 0.146 | 0.281 | 0.416 |
| 10K2 | T − C | 6.0 | −0.3 | 0.1 | 0.009 | 0.006 | −0.012 | −0.006 | 0.005 | 0.010 |
| 10K2 | (T/C) % | 104 | 94 | 101 | 103 | 104 | 97 | 96 | 102 | 102 |
| 10K2 | p-value | 0.55 | 0.34 | 0.71 | 0.45 | 0.42 | 0.46 | 0.50 | 0.48 | 0.44 |
| 10L2 | Event (T) | 145.7 | 4.7 | 8.6 | 0.287 | 0.151 | 0.339 | 0.142 | 0.285 | 0.417 |
| 10L2 | Control (C) | 148.2 | 4.8 | 8.5 | 0.282 | 0.146 | 0.344 | 0.148 | 0.281 | 0.416 |
| 10L2 | T − C | −2.5 | −0.1 | 0.1 | 0.005 | 0.005 | −0.005 | −0.006 | 0.004 | 0.001 |
| 10L2 | (T/C) % | 98 | 98 | 101 | 102 | 103 | 99 | 96 | 101 | 100 |
| 10L2 | p-value | 0.80 | 0.78 | 0.83 | 0.70 | 0.58 | 0.78 | 0.54 | 0.72 | 0.96 |
| 10M2 | Event (T) | 138.8 | 5.0 | 8.5 | 0.273 | 0.143 | 0.328 | 0.133 | 0.269 | 0.394 |
| 10M2 | Control (C) | 148.2 | 4.9 | 8.5 | 0.282 | 0.146 | 0.344 | 0.146 | 0.281 | 0.416 |
| 10M2 | T − C | −9.4 | 0.1 | 0.0 | −0.009 | −0.003 | −0.016 | −0.013 | −0.012 | −0.022 |
| 10M2 | (T/C) % | 94 | 102 | 100 | 97 | 98 | 95 | 91 | 96 | 95 |
| 10M2 | p-value | 0.34 | 0.72 | 0.99 | 0.27 | 0.68 | 0.26 | 0.11 | 0.03 | 0.02 |
| 10N2 | Event (T) | 140.7 | 4.9 | 8.7 | 0.290 | 0.152 | 0.356 | 0.142 | 0.282 | 0.415 |
| 10N2 | Control (C) | 153.3 | 4.9 | 8.5 | 0.282 | 0.146 | 0.344 | 0.146 | 0.281 | 0.416 |
| 10N2 | T − C | −12.6 | 0.0 | 0.2 | 0.008 | 0.006 | 0.012 | −0.004 | 0.001 | −0.001 |
| 10N2 | (T/C) % | 92 | 100 | 102 | 103 | 104 | 103 | 97 | 100 | 100 |
| 10N2 | p-value | 0.17 | 0.88 | 0.53 | 0.39 | 0.33 | 0.41 | 0.63 | 0.82 | 0.93 |

TABLE 40
Summary of field data for Construct 13. Numbers shown in bold are significantly different from the
control at the p-value shown. bu/a is bushels per acre. All events are an average of two testers,
except event 13S2 which had one tester. “All” indicates the average across all events or testers.
(T/C) % is the value for the transgenic hybrid combination (T) expressed as a percent of the control (C).

| | | Yield | Oil | Prot | Arg | Cys | Ile | Lys | Met | Thr | Val |
|---|---|---|---|---|---|---|---|---|---|---|---|
| | Descrip- | (bu/a) | (%) | (%) | (%) | (%) | (%) | (%) | (%) | (%) | (%) |
| Event | tion | p <= 0.10 | p <= 0.15 | p <= 0.15 | p <= 0.15 | p <= 0.15 | P <= 0.05 | p <= 0.15 | p <= 0.15 | p <= 0.15 | p <= 0.15 |
| All | (T/C) % | 100 | 103 | 106 | 107 | 102 | 101 | 110 | 102 | 103 | 104 |
| 13O2 | (T/C) % | 97 | 103 | 107 | 106 | 98 | 98 | 111 | 100 | 101 | 103 |
| 13P2 | (T/C) % | 99 | 103 | 103 | 104 | 100 | 101 | 108 | 101 | 102 | 103 |
| 13Q2 | (T/C) % | 105 | 103 | 102 | 102 | 98 | 102 | 102 | 101 | 101 | 103 |
| 13R2 | (T/C) % | 96 | 103 | 110 | 115 | 109 | 105 | 117 | 105 | 106 | 110 |
| 13S2 | (T/C) % | 111 | 97 | 106 | 108 | 105 | 99 | 113 | 102 | 102 | 103 |

TABLE 41
Field data for Construct 13. Numbers shown in bold are significantly different from the control at the
p-value shown. bu/a is bushels per acre. “All” indicates the average across all events or testers.
(T/C) % is the value for the transgenic hybrid combination (T) expressed as a percent of the control (C).
T − C is the transgenic hybrid combination minus the control. Oil, protein, and amino acid content
are shown as percent of seed dry weight.

| | | Yield | Oil | Prot | Arg | Cys | Ile | Lys | Met | Thr | Val |
|---|---|---|---|---|---|---|---|---|---|---|---|
| | | (bu/a) | (%) | (%) | (%) | (%) | (%) | (%) | (%) | (%) | (%) |
| Event | Description | p <= 0.10 | p <= 0.15 | p <= 0.15 | p <= 0.15 | p <= 0.15 | P <= 0.05 | p <= 0.15 | p <= 0.15 | p <= 0.15 | p <= 0.15 |
| All | Construct (T) | 173.2 | 3.6 | 10.8 | 0.481 | 0.233 | 0.387 | 0.363 | 0.246 | 0.474 | 0.569 |
| All | Control (C) | 172.5 | 3.5 | 10.2 | 0.449 | 0.229 | 0.383 | 0.329 | 0.242 | 0.462 | 0.545 |
| All | T − C | 0.7 | 0.1 | 0.6 | 0.032 | 0.004 | 0.004 | 0.034 | 0.004 | 0.012 | 0.024 |
| All | (T/C) % | 100 | 103 | 106 | 107 | 102 | 101 | 110 | 102 | 103 | 104 |
| All | p-value | 0.91 | 0.15 | 0.01 | 0.01 | 0.60 | 0.66 | 0.00 | 0.48 | 0.12 | 0.03 |
| 13O2 | Event (T) | 167.4 | 3.6 | 10.9 | 0.473 | 0.223 | 0.373 | 0.366 | 0.237 | 0.466 | 0.557 |
| 13O2 | Control (C) | 172.3 | 3.5 | 10.2 | 0.447 | 0.228 | 0.382 | 0.329 | 0.238 | 0.461 | 0.543 |
| 13O2 | T − C | −4.9 | 0.1 | 0.7 | 0.026 | −0.005 | −0.009 | 0.037 | −0.001 | 0.005 | 0.014 |
| 13O2 | (T/C) % | 97 | 103 | 107 | 106 | 98 | 98 | 111 | 100 | 101 | 103 |
| 13O2 | p-value | 0.59 | 0.09 | 0.01 | 0.04 | 0.60 | 0.40 | 0.00 | 0.89 | 0.61 | 0.29 |
| 13P2 | Event (T) | 171.4 | 3.6 | 10.5 | 0.467 | 0.229 | 0.384 | 0.356 | 0.245 | 0.472 | 0.557 |
| 13P2 | Control (C) | 172.5 | 3.5 | 10.2 | 0.450 | 0.228 | 0.382 | 0.329 | 0.242 | 0.463 | 0.543 |
| 13P2 | T − C | −1.1 | 0.1 | 0.3 | 0.017 | 0.001 | 0.002 | 0.027 | 0.003 | 0.009 | 0.014 |
| 13P2 | (T/C) % | 99 | 103 | 103 | 104 | 100 | 101 | 108 | 101 | 102 | 103 |
| 13P2 | p-value | 0.90 | 0.06 | 0.19 | 0.17 | 0.90 | 0.81 | 0.03 | 0.67 | 0.32 | 0.25 |
| 13Q2 | Event (T) | 180.4 | 3.6 | 10.3 | 0.454 | 0.223 | 0.388 | 0.338 | 0.242 | 0.464 | 0.557 |
| 13Q2 | Control (C) | 172.3 | 3.5 | 10.1 | 0.445 | 0.227 | 0.379 | 0.331 | 0.240 | 0.460 | 0.541 |
| 13Q2 | T − C | 8.1 | 0.1 | 0.2 | 0.009 | −0.004 | 0.009 | 0.007 | 0.002 | 0.004 | 0.016 |
| 13Q2 | (T/C) % | 105 | 103 | 102 | 102 | 98 | 102 | 102 | 101 | 101 | 103 |
| 13Q2 | p-value | 0.40 | 0.03 | 0.56 | 0.54 | 0.65 | 0.47 | 0.60 | 0.86 | 0.70 | 0.27 |
| 13R2 | Event (T) | 165.7 | 3.6 | 11.2 | 0.516 | 0.251 | 0.400 | 0.385 | 0.255 | 0.490 | 0.600 |
| 13R2 | Control (C) | 172.4 | 3.5 | 10.2 | 0.449 | 0.230 | 0.381 | 0.328 | 0.242 | 0.462 | 0.543 |
| 13R2 | T − C | −6.7 | 0.1 | 1.0 | 0.067 | 0.021 | 0.019 | 0.057 | 0.013 | 0.028 | 0.057 |
| 13R2 | (T/C) % | 96 | 103 | 110 | 115 | 109 | 105 | 117 | 105 | 106 | 110 |
| 13R2 | p-value | 0.46 | 0.43 | 0.00 | 0.00 | 0.02 | 0.09 | 0.00 | 0.15 | 0.00 | 0.00 |
| 13S2 | Event (T) | 184.2 | 3.7 | 10.9 | 0.511 | 0.247 | 0.386 | 0.386 | 0.251 | 0.494 | 0.570 |
| 13S2 | Control (C) | 165.5 | 3.8 | 10.3 | 0.473 | 0.235 | 0.388 | 0.343 | 0.245 | 0.485 | 0.552 |
| 13S2 | T − C | 18.7 | −0.1 | 0.6 | 0.038 | 0.012 | −0.002 | 0.043 | 0.006 | 0.009 | 0.018 |
| 13S2 | (T/C) % | 111 | 97 | 106 | 108 | 105 | 99 | 113 | 102 | 102 | 103 |
| 13S2 | p-value | 0.08 | 0.03 | 0.22 | 0.10 | 0.44 | 0.89 | 0.03 | 0.65 | 0.52 | 0.42 |

1. An expression cassette conferring increased content in one or more of protein, oil, or one or more amino acids in a plant, plant cell, or plant part relative to a corresponding wild-type plant, plant cell, or plant part, comprising:
(a) a promoter that is functional in a plant;
(b) a nucleic acid molecule encoding a polypeptide having pyruvate kinase activity which is heterologous and operably linked to said promoter; and
(c) a rice intron,
wherein the nucleic acid molecule comprises:
(i) the nucleotide sequence of SEQ ID NO: 1, 3, 5, 7, 9, 11 or 13;
(ii) a nucleotide sequence encoding the amino acid sequence of SEQ ID NO: 2, 4, 6, 8, 10, 12 or 14;
(iii) a nucleotide sequence having at least 60% identity to the nucleotide sequence of SEQ ID NO: 1, 3, 5, 7, 9, 11 or 13 and encoding a polypeptide having a Pfam:PF00224 pyruvate kinase barrel domain and a Pfam:PF02887 pyruvate kinase alpha/beta domain;
(iv) a nucleotide sequence encoding an amino acid sequence having at least 60% identity to the amino acid sequence of SEQ ID NO: 2, 4, 6, 8, 10, 12 or 14 and having a Pfam:PF00224 pyruvate kinase barrel domain and a Pfam:PF02887 pyruvate kinase alpha/beta domain;
(v) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 109 to 449 of SEQ ID NO: 2 or the amino acid residues 98 to 439 of SEQ ID NO: 10, and wherein the Pfam:PF02887 pyruvate kinase alpha/beta domain has at least 80% identity to the amino acid residues 462 to 578 of SEQ ID NO: 2 or the amino acid residues 452 to 566 of SEQ ID NO: 10; or
(vi) a nucleotide sequence encoding an amino acid sequence having at least 60% identity to the amino acid sequence of SEQ ID NO: 2, 4, 6, 8, 10, 12 or 14, wherein said amino acid sequence further comprises the amino acid sequence of SEQ ID NO: 102 and 103.

2. An expression cassette conferring increased content in one or more of protein, oil, or one or more amino acids in a plant, plant cell, or plant part relative to a corresponding wild-type plant, plant cell, or plant part, comprising:
(a) a promoter that is functional in a plant;
(b) a nucleic acid molecule encoding a polypeptide having pyruvate kinase activity which is heterologous and operably linked to said promoter; and
(c) an intron,
wherein the promoter is an endosperm-specific or endosperm-preferential promoter or an embryo-specific or embryo-preferential promoter,
and wherein the nucleic acid molecule comprises:
(i) the nucleotide sequence of SEQ ID NO: 1, 3, 5, 7, 9, 11 or 13;
(ii) a nucleotide sequence encoding the amino acid sequence of SEQ ID NO: 2, 4, 6, 8, 10, 12 or 14;
(iii) a nucleotide sequence having at least 60% identity to the nucleotide sequence of SEQ ID NO: 1, 3, 5, 7, 9, 11 or 13 and encoding a polypeptide having a Pfam:PF00224 pyruvate kinase barrel domain and a Pfam:PF02887 pyruvate kinase alpha/beta domain;
(iv) a nucleotide sequence encoding an amino acid sequence having at least 60% identity to the amino acid sequence of SEQ ID NO: 2, 4, 6, 8, 10, 12 or 14 and having a Pfam:PF00224 pyruvate kinase barrel domain and a Pfam:PF02887 pyruvate kinase alpha/beta domain;
(v) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 109 to 449 of SEQ ID NO: 2 or the amino acid residues 98 to 439 of SEQ ID NO: 10, and wherein the Pfam:PF02887 pyruvate kinase alpha/beta domain has at least 80% identity to the amino acid residues 462 to 578 of SEQ ID NO: 2 or the amino acid residues 452 to 566 of SEQ ID NO: 10; or
(vi) a nucleotide sequence encoding an amino acid sequence having at least 60% identity to the amino acid sequence of SEQ ID NO: 2, 4, 6, 8, 10, 12 or 14, wherein said amino acid sequence further comprises the amino acid sequence of SEQ ID NO: 102 and 103.

3. The expression cassette of claim 1, wherein the promoter is a seed-specific or seed-preferential promoter, an endosperm-specific or endosperm-preferential promoter, or an embryo-specific or embryo-preferential promoter.

4. The expression cassette of claim 1, wherein the promoter is a seed-specific or seed-preferential promoter comprising:
(a) the nucleotide sequence of SEQ ID NO: 104 or 105;
(b) a nucleotide sequence having at least 95% identity to the nucleotide sequence of SEQ ID NO: 104 or 105, wherein said nucleotide sequence has seed-specific or seed-preferential expression activity; or
(c) a fragment of the nucleotide sequence of SEQ ID NO: 104 or 105, wherein the fragment has seed-specific or seed-preferential expression activity.

5. The expression cassette of claim 1, wherein the promoter is an endosperm-specific or endosperm-preferential promoter comprising:
(a) the nucleotide sequence of SEQ ID NO: 106 or 107;
(b) a nucleotide sequence having at least 95% identity to the nucleotide sequence of SEQ ID NO: 106 or 107, wherein said nucleotide sequence has endosperm-specific or endosperm-preferential expression activity; or
(c) a fragment of the nucleotide sequence of SEQ ID NO: 106 or 107, wherein the fragment has endosperm-specific or endosperm-preferential expression activity.

6. The expression cassette of claim 1, wherein the promoter is an embryo-specific or embryo-preferential promoter comprising:
(a) the nucleotide sequence of SEQ ID NO: 108;
(b) a nucleotide sequence having at least 95% identity to the nucleotide sequence of SEQ ID NO: 108, wherein said nucleotide sequence has embryo-specific or embryo-preferential expression activity; or
(c) a fragment of the nucleotide sequence of SEQ ID NO: 108, wherein the fragment has embryo-specific or embryo-preferential expression activity.

7. The expression cassette of claim 1, wherein the nucleic acid molecule comprises:
(a) a nucleotide sequence having at least 95% identity to the nucleotide sequence of SEQ ID NO: 1, 3, 5, 7, 9, 11 or 13; or
(b) a nucleotide sequence encoding an amino acid sequence having at least 95% identity to the amino acid sequence of SEQ ID NO: 2, 4, 6, 8, 10, 12 or 14.

8. The expression cassette of claim 1, wherein the nucleic acid molecule comprises:
(a) the nucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, or 83; or
(b) a nucleotide sequence encoding the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, or 84.

9. The expression cassette of claim 2, wherein the intron is a monocot intron.

10. The expression cassette of claim 9, wherein the monocot intron is a rice intron.

11. The expression cassette of claim 1, wherein the rice intron is an intron of the rice Metallothionin1 gene or an intron of the rice MADS3 gene.

12. The expression cassette of claim 11, wherein the intron of the rice Metallothionin1 gene comprises the nucleotide sequence of SEQ ID NO: 111 or a nucleotide sequence having at least 90% identity to the nucleotide sequence of SEQ ID NO: 111, or the intron of the rice MADS3 gene comprises the nucleotide sequence of SEQ ID NO: 112 or a nucleotide sequence having at least 90% identity to the nucleotide sequence of SEQ ID NO: 112.

13. The expression cassette of claim 1, wherein the promoter comprises the nucleotide sequence of SEQ ID NO: 106, the nucleic acid molecule encoding a polypeptide having pyruvate kinase activity comprises a nucleotide sequence encoding the amino acid sequence of SEQ ID NO: 2, and the rice intron is an intron of the rice Metallothionin1 gene comprising the nucleotide sequence of SEQ ID NO: 111, and wherein said expression cassette further comprises a nucleotide sequence encoding a plastid-targeting peptide comprising the amino acid sequence of SEQ ID NO: 114, and a terminator comprising the nucleotide sequence of SEQ ID NO: 115.

14. The expression cassette of claim 1, wherein the promoter comprises the nucleotide sequence of SEQ ID NO: 106, the nucleic acid molecule encoding a polypeptide having pyruvate kinase activity comprises a nucleotide sequence encoding the amino acid sequence of SEQ ID NO: 2, and the rice intron is an intron of the rice MADS3 gene comprising the nucleotide sequence of SEQ ID NO: 112, and wherein said expression cassette further comprises a nucleotide sequence encoding a plastid-targeting peptide comprising the amino acid sequence of SEQ ID NO: 114, and a terminator comprising the nucleotide sequence of SEQ ID NO: 115.

15. An expression cassette conferring increased content in one or more of protein, oil, or one or more amino acids in a plant, plant cell, or plant part relative to a corresponding wild-type plant, plant cell, or plant part, comprising:
(a) a promoter that is functional in a plant;
(b) a nucleic acid molecule encoding a polypeptide having pyruvate kinase activity which is heterologous and operably linked to said promoter; and
(c) the first intron of the rice Metallothionin1 gene,
wherein the nucleic acid molecule comprises:
(i) the nucleotide sequence of SEQ ID NO: 87 or 89;
(ii) a nucleotide sequence encoding the amino acid sequence of SEQ ID NO: 88 or 90;
(iii) a nucleotide sequence having at least 75% identity to the nucleotide sequence of SEQ ID NO: 87 or 89 and encoding a polypeptide having a Pfam:PF00224 pyruvate kinase barrel domain and a Pfam:PF02887 pyruvate kinase alpha/beta domain;
(iv) a nucleotide sequence encoding an amino acid sequence having at least 75% identity to the amino acid sequence of SEQ ID NO: 88 or 90 and having a Pfam:PF00224 pyruvate kinase barrel domain and a Pfam:PF02887 pyruvate kinase alpha/beta domain;
(v) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 5 to 350 of SEQ ID NO: 88, and wherein the Pfam:PF02887 pyruvate kinase alpha/beta domain has at least 80% identity to the amino acid residues 362 to 478 of SEQ ID NO: 88; or
(vi) a nucleotide sequence encoding an amino acid sequence having at least 75% identity to the amino acid sequence of SEQ ID NO: 88 or 90, wherein said amino acid sequence further comprises the amino acid sequence of SEQ ID NO: 102 and 103.

16. The expression cassette of claim 15, wherein the promoter is a constitutive promoter, a seed-specific or seed-preferential promoter, an endosperm-specific or endosperm-preferential promoter, or an embryo-specific or embryo-preferential promoter.

17. The expression cassette of claim 15, wherein the first intron of the rice Metallothionin1 gene comprises the nucleotide sequence of SEQ ID NO: 111 or a nucleotide sequence having at least 90% identity to the nucleotide sequence of SEQ ID NO: 111.

18. An expression cassette conferring increased content in one or more of protein, oil, or one or more amino acids in a plant, plant cell, or plant part relative to a corresponding wild-type plant, plant cell, or plant part, comprising:
(a) a constitutive promoter that is functional in a plant;
(b) a nucleic acid molecule encoding a polypeptide having pyruvate kinase activity which is heterologous and operably linked to said promoter; and
(c) an intron,
wherein the nucleic acid molecule comprises:
(i) the nucleotide sequence of SEQ ID NO: 87 or 89;
(ii) a nucleotide sequence encoding the amino acid sequence of SEQ ID NO: 88 or 90;
(iii) a nucleotide sequence having at least 75% identity to the nucleotide sequence of SEQ ID NO: 87 or 89 and encoding a polypeptide having a Pfam:PF00224 pyruvate kinase barrel domain and a Pfam:PF02887 pyruvate kinase alpha/beta domain;
(iv) a nucleotide sequence encoding an amino acid sequence having at least 75% identity to the amino acid sequence of SEQ ID NO: 88 or 90 and having a Pfam:PF00224 pyruvate kinase barrel domain and a Pfam:PF02887 pyruvate kinase alpha/beta domain;
(v) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 5 to 350 of SEQ ID NO: 88, and wherein the Pfam:PF02887 pyruvate kinase alpha/beta domain has at least 80% identity to the amino acid residues 362 to 478 of SEQ ID NO: 88; or
(vi) a nucleotide sequence encoding an amino acid sequence having at least 75% identity to the amino acid sequence of SEQ ID NO: 88 or 90, wherein said amino acid sequence further comprises the amino acid sequence of SEQ ID NO: 102 and 103,
and wherein the constitutive promoter comprises:
(a) the nucleotide sequence of SEQ ID NO: 109 or 110;
(b) a nucleotide sequence having at least 95% identity to the nucleotide sequence of SEQ ID NO: 109 or 110, wherein said nucleotide sequence has constitutive expression activity; or
(c) a fragment of the nucleotide sequence of SEQ ID NO: 109 or 110, wherein the fragment has constitutive expression activity.

19. The expression cassette of claim 18, wherein the intron is a monocot intron.

20. The expression cassette of claim 18, wherein the intron is an intron of the rice Metallothionin1 gene or an intron of the rice MADS3 gene.

21. The expression cassette of claim 20, wherein the intron of the rice Metallothionin1 gene comprises the nucleotide sequence of SEQ ID NO: 111 or a nucleotide sequence having at least 90% identity to the nucleotide sequence of SEQ ID NO: 111, or the intron of the rice MADS3 gene comprises the nucleotide sequence of SEQ ID NO: 112 or a nucleotide sequence having at least 90% identity to the nucleotide sequence of SEQ ID NO: 112.

22. The expression cassette of claim 1, further comprising a nucleotide sequence encoding a transit peptide targeting the polypeptide having pyruvate kinase activity to a plastid, wherein said nucleotide sequence is heterologous in relation to the nucleic acid molecule encoding the polypeptide having pyruvate kinase activity.

23. The expression cassette of claim 22, wherein the transit peptide is a plastid-targeting peptide from a ferredoxin gene.

24. The expression cassette of claim 22, wherein the nucleotide sequence encoding a transit peptide comprises:
(a) the nucleotide sequence of SEQ ID NO: 113 or 120;
(b) a nucleotide sequence having at least 95% identity to the sequence of SEQ ID NO: 113 or 120;
(b) a nucleotide sequence encoding the amino acid sequence of SEQ ID NO: 114; or
(c) a nucleotide sequence encoding a peptide having at least 95% identity to the amino acid sequence of SEQ ID NO: 114.

25. The expression cassette of claim 1, wherein expression of the nucleic acid molecule encoding a polypeptide having pyruvate kinase activity in a plant, plant cell, or plant part confers increased content in one or more of protein, oil, or one or more amino acids in said plant, plant cell, or plant part relative to a corresponding wild-type plant, plant cell, or plant part.

26. The expression cassette of claim 1, wherein the expression of the nucleic acid molecule encoding a polypeptide having pyruvate kinase activity in a plant, plant cell, or plant part confers increased content of protein, oil, and one or more amino acids in said plant, plant cell, or plant part relative to a corresponding wild-type plant, plant cell, or plant part.

27. An expression cassette conferring increased content of protein, oil, and one or more amino acids in a plant, plant cell, or plant part relative to a corresponding wild-type plant, plant cell, or plant part, comprising:
(a) a seed-specific or seed-preferential promoter; and
(b) a nucleic acid molecule encoding a polypeptide having pyruvate kinase activity which is heterologous and operably linked to said seed-specific or seed-preferential promoter,
wherein the nucleic acid molecule comprises:
(i) the nucleotide sequence of SEQ ID NO: 1, 3, 5, 7, 9, 11 or 13;
(ii) a nucleotide sequence encoding the amino acid sequence of SEQ ID NO: 2, 4, 6, 8, 10, 12 or 14;
(iii) a nucleotide sequence having at least 60% identity to the nucleotide sequence of SEQ ID NO: 1, 3, 5, 7, 9, 11 or 13 and encoding a polypeptide having a Pfam:PF00224 pyruvate kinase barrel domain and a Pfam:PF02887 pyruvate kinase alpha/beta domain;
(iv) a nucleotide sequence encoding an amino acid sequence having at least 60% identity to the amino acid sequence of SEQ ID NO: 2, 4, 6, 8, 10, 12 or 14 and having a Pfam:PF00224 pyruvate kinase barrel domain and a Pfam:PF02887 pyruvate kinase alpha/beta domain;
(v) a nucleotide sequence encoding an amino acid sequence comprising a Pfam:PF00224 pyruvate kinase barrel domain and a Pfam:PF02887 pyruvate kinase alpha/beta domain, wherein the Pfam:PF00224 pyruvate kinase barrel domain has at least 80% identity to the amino acid residues 109 to 449 of SEQ ID NO: 2 or the amino acid residues 98 to 439 of SEQ ID NO: 10, and wherein the Pfam:PF02887 pyruvate kinase alpha/beta domain has at least 80% identity to the amino acid residues 462 to 578 of SEQ ID NO: 2 or the amino acid residues 452 to 566 of SEQ ID NO: 10; or
(vi) a nucleotide sequence encoding an amino acid sequence having at least 60% identity to the amino acid sequence of SEQ ID NO: 2, 4, 6, 8, 10, 12 or 14, wherein said amino acid sequence further comprises the amino acid sequence of SEQ ID NO: 102 and 103,
and wherein expression of the nucleic acid molecule in a plant, plant cell, or plant part confers increased content of protein, oil, and one or more amino acids in said plant, plant cell, or plant part relative to a corresponding wild-type plant, plant cell, or plant part.

28. The expression cassette of claim 27, further comprising an intron.

29. The expression cassette of claim 27, wherein the seed-specific or seed-preferential promoter is an endosperm-specific or endosperm-preferential promoter or an embryo-specific or embryo-preferential promoter.

30. The expression cassette of claim 27, further comprising a nucleotide sequence encoding a transit peptide targeting the polypeptide having pyruvate kinase activity to a plastid, wherein said nucleotide sequence is heterologous in relation to the nucleic acid molecule encoding the polypeptide having pyruvate kinase activity.

31. The expression cassette of claim 30, wherein the transit peptide is a plastid-targeting peptide from a ferredoxin gene.

32. The expression cassette of claim 1, further comprising a terminator.

33. The expression cassette of claim 32, wherein said terminator comprises the nucleotide sequence of SEQ ID NO: 115 or 116, or a nucleotide sequence having at least 90% identity to the nucleotide sequence of SEQ ID NO: 115 or 116.

34. A recombinant construct comprising at least one expression cassette of claim 1.

35. A vector comprising one or more expression cassette of claim 1 or a recombinant construct comprising the expression cassette.

36. A microorganism comprising the expression cassette of claim 1, a recombinant construct comprising the expression cassette, or a vector comprising the expression cassette or the recombinant construct.

37. A plant, plant cell, or plant part, comprising the expression cassette of claim 1 or a recombinant construct comprising the expression cassette, wherein the plant, plant cell, or plant part has increased content in one or more of protein, oil, or one or more amino acids relative to a corresponding wild-type plant, plant cell, or plant part.

38. The plant, plant cell, or plant part of claim 37, wherein the plant, plant cell, or plant part has increased content of protein, oil, and one or more amino acids relative to a corresponding wild-type plant, plant cell, or plant part.

39. The plant, plant cell, or plant part of claim 37, wherein the plant is a monocotyledonous plant or the plant cell or plant part is from a monocotyledonous plant.

40. The plant, plant cell, or plant part of claim 39, wherein the plant is a maize plant or the plant cell or plant part is from a maize plant.

41. The plant part of claim 1, wherein the plant part is a seed.

42. A food or feed composition comprising the plant, plant cell, or plant part of claim 37.

43. The food or feed composition of claim 42, wherein the food or feed composition is not supplemented with additional protein, oil, or amino acids, or wherein the food or feed composition has reduced supplementation with protein, oil, or amino acids relative to a food or feed composition comprising a corresponding wild-type plant, plant cell, or plant part.

44. The feed composition of claim 42, wherein the feed composition is formulated to meet the dietary requirements of swine, poultry, cattle, or companion animals.

45. A method for producing a transgenic plant, plant cell, or plant part having increased content in one or more of protein, oil, or one or more amino acids relative to a corresponding wild-type plant, plant cell, or plant part, comprising:
(a) transforming a plant, plant cell, or plant part with the expression cassette of claim 1, a recombinant construct comprising the expression cassette, or a vector comprising the expression cassette or recombinant construct; and
(b) optionally regenerating from the plant cell or plant part a transgenic plant,
wherein the transgenic plant, plant cell, or plant part has increased content in one or more of protein, oil, or one or more amino acids relative to a corresponding wild-type plant, plant cell, or plant part.

46. A method for increasing the content of one or more of protein, oil, or one or more amino acids in a plant, plant cell, or plant part relative to a corresponding wild-type plant, plant cell, or plant part, comprising:
(a) obtaining the plant, plant cell, or plant part of claim 37; and
(b) selecting a plant, plant cell, or plant part with increased content in one or more of protein, oil, or one or more amino acids.

47. The method of claim 45, wherein the plant is a monocotyledonous plant or the plant cell or plant part is from a monocotyledonous plant.

48. The method of claim 47, wherein the plant is a maize plant or the plant cell or plant part is from a maize plant.

49. The method of claim 45, wherein the content of one or more amino acids in said plant, plant cell, or plant part is increased relative to a corresponding wild-type plant, plant cell, or plant part.

50. The method of claim 45, wherein the content of protein in said plant, plant cell, or plant part is increased relative to a corresponding wild-type plant, plant cell, or plant part.

51. The method of claim 45, wherein the content of oil in said plant, plant cell, or plant part is increased relative to a corresponding wild-type plant, plant cell, or plant part.

52. The method of claim 45, wherein the content of protein, oil and one or more amino acids in said plant, plant cell, or plant part is increased relative to a corresponding wild-type plant, plant cell, or plant part.

53. The method of claim 45, wherein said plant, plant cell, or plant part has an increased content of one or more amino acids selected from the group consisting of arginine, cysteine, isoleucine, lysine, methionine, threonine, and valine.

54. The method of claim 53, wherein the content of at least two amino acids is increased.

55. A method of producing a food or feed composition comprising:
(a) obtaining the plant, plant cell, or plant part of claim 37 having increased content in one or more of protein, oil, or one or more amino acids relative to a corresponding wild-type plant, plant cell, or plant part;
(b) producing a food or feed composition comprising said plant, plant cell, or plant part.

56. A method for producing a hybrid maize plant or seed comprising:
(a) crossing a first inbred parent maize plant with a second inbred parent maize plant;
(b) harvesting a resultant hybrid maize seed; and
(c) optionally growing a hybrid maize plant from the resultant hybrid maize seed,
wherein said first inbred parent maize plant, and optionally said second inbred parent maize plant, comprises the expression cassette of claim 1 or a recombinant construct comprising the expression cassette.

57. A hybrid maize plant or seed produced by the method of claim 56.

58. A plant produced by growing the hybrid maize seed of claim 57.

59. A plant breeding program comprising utilizing the plant, plant cell, or plant part of claim 37 as a source of plant breeding material, wherein said plant, plant cell, or plant part has increased content in one or more of protein, oil, or one or more amino acids relative to a corresponding wild-type plant, plant cell, or plant part.

60. A plant, plant cell, or plant part obtained from the plant breeding program of claim 59.

61. A method for developing a maize plant in a maize plant breeding program using plant breeding techniques comprising employing a maize plant, or its parts, as a source of plant breeding material, wherein said maize plant, or its parts, comprises the expression cassette of claim 1 or a recombinant construct comprising the expression cassette.

62. A maize plant obtained from the method of claim 61.

63. A method of plant breeding, comprising:
(a) obtaining the hybrid maize plant of claim 57;
(b) crossing said hybrid maize plant with a different maize plant; and
(c) selecting a resultant progeny with increased content in one or more of protein, oil, or one or more amino acids.

64. A method for producing grain with increased content in one or more of protein, oil, or one or more amino acids, comprising:
(a) interplanting a first plant and at least one second plant, wherein the first plant comprises the expression cassette of claim 1 or a recombinant construct comprising the expression cassette;
(b) growing said first plant and said at least one second plant to obtain preferential inheritance of increased content in one or more of protein, oil, or one or more amino acids in a resultant progeny of said first plant and said at least one second plant; and
(c) harvesting grain from said resultant progeny.

65. Grain produced by the method of claim 64, wherein the grain has increased content in one or more of protein, oil, or one or more amino acids relative to a corresponding wild-type grain.

66. The grain of claim 65, wherein the one or more amino acids is selected from the group consisting of arginine, cysteine, isoleucine, lysine, methionine, threonine, and valine.

67. The grain of claim 65, wherein the grain is corn.

68. A method for producing a maize plant with increased content in one or more of protein, oil, or one or more amino acids, comprising:
(a) growing a progeny plant obtained from crossing a maize plant comprising the expression cassette of claim 1 or a recombinant construct comprising the expression cassette with a second maize plant;
(b) crossing said progeny plant with itself or a different maize plant to produce a resultant seed;
(c) growing said resultant seed to obtain a progeny plant of a subsequent generation; and
(d) crossing said progeny plant of a subsequent generation with itself or a different maize plant; and
(e) repeating steps (b) to (d) for additional 0-5 generations to produce a maize plant with increased content in one or more of protein, oil, or one or more amino acids.

69. The method of claim 68, wherein the maize plant produced is an inbred maize plant.

70. The method of claim 68, further comprising crossing the inbred maize plant with a second, distinct inbred maize plant to produce an F1 hybrid maize plant.
